(19) United States
(12) Patent Application Publication
(10) Pub. No.: US 2023/0074558 A1
(43) Pub. Date: Mar. 9, 2023

(54) DIMERIC COMPOUNDS AS STING AGONISTS

(71) Applicant: Mersana Therapeutics, Inc., Cambridge, MA (US)

(72) Inventors: Jeremy R. DUVALL, Topsfield, MA (US); Keith W. BENTLEY, Everett, MA (US); Ha Thi DO, Dallas, TX (US); Brian D. JONES, Brookline, MA (US); Nabeelah I. KAUSER, Watertown, MA (US); Eugene W. KELLEHER, Wellesley, MA (US); Soumya S. RAY, Quincy, MA (US); Joshua D. THOMAS, Natick, MA (US); Dorin TOADER, Cambridge, MA (US); Liping YANG, Arlington, MA (US)

(21) Appl. No.: 17/782,796

(22) PCT Filed: Dec. 4, 2020
(86) PCT No.: PCT/US2020/063371

§ 371 Date: Jun. 6, 2022

Related U.S. Application Data
(60) Provisional application No. 63/104,015, filed on Oct. 22, 2020.
(60) Provisional application No. 62/944,595, filed on Dec. 6, 2019.

(51) Int. Cl. A61K 47/54

(52) U.S. Cl. CPC A61K 47/545

(57) ABSTRACT

The present disclosure relates to dimeric STING agonists of Formulae (I), (II), (III), (IV), (V), and (VI), and pharmaceutically acceptable salts thereof. The present disclosure also relates to methods of preparing the compounds and methods of using the compounds.

### RELATED APPLICATIONS

This application claims priority to, and the benefit of, U.S. Provisional Application Nos. 62/994,595, filed Dec. 6, 2019, and 63/104,015, filed Oct. 22, 2020. The contents of each of these applications are hereby incorporated by reference in their entireties.

### BACKGROUND

Stimulator of Interferon Genes (STING) is a receptor in the endoplasmic reticulum that propagates innate immune sensing of cytosolic pathogen derived- and self-DNA. STING is a 378 amino acid protein, which mainly contains three structural domains: (i) N-terminal transmembrane domain (aa 1-154); (ii) central globular domain (aa 155-341); and (iii) C-terminal tail (aa 342-379). STING may form symmetrical dimers combined with its ligands in V-shaped conformation, while not completely covering the bound ligands. A STING agonist can bind into the pocket region of STING. However, the STING activation process is easily inhibited in some severe disease conditions, resulting in the inactivation of the STING pathway. Therefore, screening and designing potent STING agonists is of great importance for cancer immune therapy and other infectious diseases treatments, including, but not limited to, obesity, liver injury, sugar-lipid metabolism, and virus infection. Specific targeting of immune pathways presents opportunities for cancer therapy, potentially offering greater specificity than cell population-based therapeutic approaches.

### SUMMARY

In some aspects, the present disclosure provides a compound of Formula (I), (II), (III), (IV), (V) or (VI):

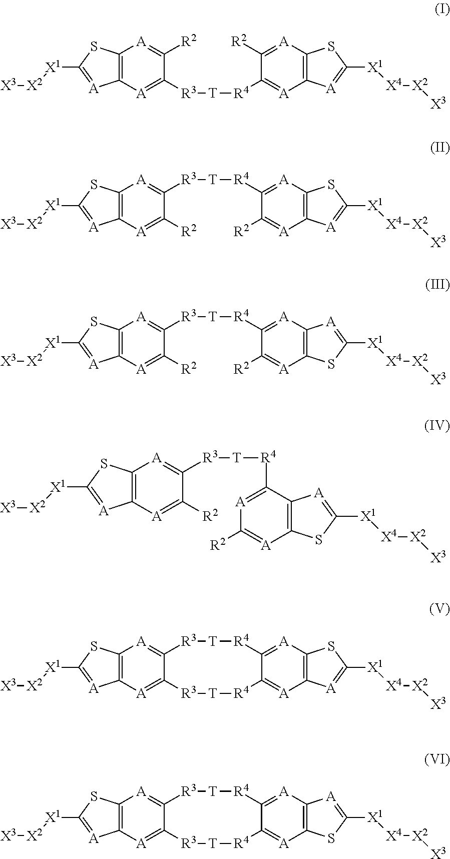

or a pharmaceutically acceptable salt thereof, wherein:

each A independently is —C(R¹)— or —N—;

each R¹ independently is hydrogen, halogen, OR⁶, N(R⁶)₂, CN, or C₁-C₆ alkyl, wherein the C₁-C₆ alkyl is optionally substituted with one or more halogen, OR⁶, N(R⁶)₂, C(═O)OR⁶, or C(═O)N(R⁶)₂;

each R² independently is hydrogen, halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C₁-C₆ alkyl, C₂-C₆ alkenyl, or C₂-C₆ alkynyl is optionally substituted with one more halogen or OR⁶;

R³ and R⁴ each independently are absent, C₁-C₆ alkyl, —N(R_{S})—, or —O—;

each T independently is absent, -Tₐ-C₂-C₆ alkyl-T_{b}-, —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S};

Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; wherein the C₁-C₆ alkyl is optionally substituted with one or more halogen; and

each R_{S} independently is H or C₁-C₆ alkyl optionally substituted with one or more halogen;

each R⁶ independently is H or C₁-C₆ alkyl optionally substituted with one or more halogen;

each X¹ independently is —C(═O)—, —CH₂—, —CHF—, or —CF₂—;

each X² independently is —(C(R⁸)₂)₁₋₃—, wherein each R⁸ independently: (a) is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂; or (2) together with another R⁸ and the one or more atoms to which they are attached, form C₃-C₆ cycloalkyl or 3- to 6-membered heterocycloalkyl;

each X³ independently is C(═O)OR⁶, C(═O)SR⁶, C(═S)OR⁶,

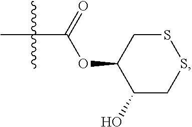 [SMILES: *C(=O)O[C@H]1CSSC[C@@H]1O]

SO₂R⁶, C(═O)N(R⁹)₂, or CN;

each R⁹ independently is hydrogen, C(═O)OR⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₁-C₆ alkyl)-OR⁶, O(C₆-C₁₀ aryl), S(C₁-C₆ alkyl), S(C₆-C₁₀ aryl), S(═O)₂R⁶, S(═O)₂OR⁶, P(═O)(R⁶)₂, C₁-C₆ alkyl, C₂-C₆ alkenyl, C₂-C₆ alkynyl, C₃-C₈ cycloalkyl, C₆-C₁₀ aryl, 3-8 membered heterocycloalkyl, or 3-10 membered heteroaryl; and

X⁴ is absent or —NR¹¹, wherein each R¹¹ independently is H or C₁-C₆ alkyl optionally substituted with one or more halogen;

provided that:

(1) when one of R₃ and R₄ is C₁ alkyl, and T is —N(R_{S})— or —O—, then the other one of R₃ and R₄ is absent;

(2) when one of R₃ and R₄ is C₁-C₂ alkyl, and T is absent, then the other one of R₃ and R₄ is absent;

(3) when one of R₃ and R₄ is C₂-C₆ alkyl, then T is not —N(R_{S})— or —O—;

(4) when at least one of R₃ and R₄ is C₃-C₆ alkyl, then T is not absent;

(5) when X⁴ is —NR¹¹, at least one A is —N—; and/or

(6) when T is -Tₐ-C₂-C₆ alkyl-T_{b}, and X⁴ is absent, then R₁ is CN.

In some aspects, the present disclosure provides an isotopic derivative of a compound described herein.

In some aspects, the present disclosure provides a method of preparing a compound described herein.

In some aspects, the present disclosure provides an intermediate suitable for use in a method for preparing a compound described herein.

In some aspects, the present disclosure provides a pharmaceutical composition comprising an effective amount of a compound described herein and a pharmaceutically acceptable carrier.

In some aspects, the present disclosure provides a method of treating or preventing a STING mediated disease or disorder in a subject, comprising administering to the subject a therapeutically effective amount of a compound described herein.

In some aspects, the present disclosure provides a method of inducing an immune response in a subject, comprising administering to the subject a therapeutically effective amount of a compound described herein.

In some aspects, the present disclosure provides a method of inducing STING-dependent type I interferon production in a subject, comprising administering to the subject a therapeutically effective amount of a compound described herein.

In some aspects, the present disclosure provides a method of inducing STING-dependent cytokine production in a subject, comprising administering to the subject a therapeutically effective amount of a compound described herein.

In some aspects, the present disclosure provides a method of treating or preventing a cell proliferation disorder in a subject, comprising administering to the subject a therapeutically effective amount of a compound described herein.

In some aspects, the present disclosure provides a compound described herein for use in treating or preventing a STING mediated disease or disorder in a subject.

In some aspects, the present disclosure provides a compound described herein for inducing an immune response in a subject.

In some aspects, the present disclosure provides a compound described herein for inducing STING-dependent type I interferon production in a subject.

In some aspects, the present disclosure provides a compound described herein for inducing STING-dependent cytokine production in a subject.

In some aspects, the present disclosure provides a compound described herein for treating or preventing a cell proliferation disorder in a subject.

In some aspects, the present disclosure provides a compound described herein for use in the manufacture of a medicament for treating or preventing a STING mediated disease or disorder in a subject.

In some aspects, the present disclosure provides a compound described herein for use in the manufacture of a medicament for inducing an immune response in a subject.

In some aspects, the present disclosure provides a compound described herein for use in the manufacture of a medicament for inducing STING-dependent type I interferon production in a subject.

In some aspects, the present disclosure provides a compound described herein for use in the manufacture of a medicament for inducing STING-dependent cytokine production in a subject.

In some aspects, the present disclosure provides a compound described herein for use in the manufacture of a medicament for treating or preventing a cell proliferation disorder in a subject.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

Without wishing to be bound by theory, the compounds of the present disclosure may modulate the activity of STING, and accordingly, may provide a beneficial therapeutic impact in treatment of diseases, disorders and/or conditions in which modulation of STING (Stimulator of Interferon Genes) is beneficial, including, but not limited to, inflammation, allergic and autoimmune diseases, infectious diseases, cancer, pre-cancerous syndromes, and as vaccine adjuvants.

### Compounds of the Present Disclosure

In some aspects, the present disclosure provides a compound of Formula (I), (II), (III), (IV), (V) or (VI):

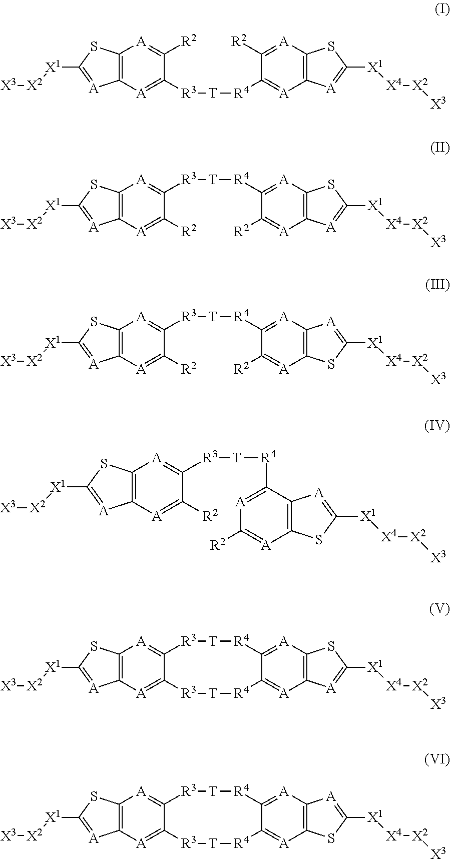

or a pharmaceutically acceptable salt thereof, wherein:

each A independently is —C(R¹)— or —N—;

each R¹ independently is hydrogen, halogen, OR⁶, N(R⁶)₂, CN, or C₁-C₆ alkyl, wherein the C₁-C₆ alkyl is optionally substituted with one or more halogen, OR⁶, N(R⁶)₂, C(═O)OR⁶, or C(═O)N(R⁶)₂;

each R² independently is hydrogen, halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C₁-C₆ alkyl, C₂-C₆ alkenyl, or C₂-C₆ alkynyl is optionally substituted with one more halogen or OR⁶;

R³ and R⁴ each independently are absent, C₁-C₆ alkyl, —N(R_{S})—, or —O—;

each T independently is absent, -Tₐ-C₂-C₆ alkyl-T_{b}-, —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S};

Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; wherein the C₁-C₆ alkyl is optionally substituted with one or more halogen; and

each R_{S} independently is H or C₁-C₆ alkyl optionally substituted with one or more halogen;

each R⁶ independently is H or C₁-C₆ alkyl optionally substituted with one or more halogen;

each X¹ independently is —C(═O)—, —CH₂—, —CHF—, or —CF₂—;

each X² independently is —(C(R⁸)₂)₁₋₃—, wherein each R⁸ independently: (a) is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂; or (2) together with another R⁸ and the one or more atoms to which they are attached, form C₃-C₆ cycloalkyl or 3- to 6-membered heterocycloalkyl;

each X³ independently is C(═O)OR⁶, C(═O)SR⁶, C(═S)OR⁶,

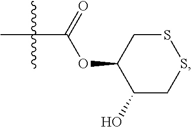 [SMILES: *C(=O)O[C@H]1CSSC[C@@H]1O]

SO₂R⁶, C(═O)N(R⁹)₂, or CN;

X⁴ is absent or —NR¹¹, wherein each R¹¹ independently is H or C₁-C₆ alkyl optionally substituted with one or more halogen; and

each R⁹ independently is hydrogen, C(═O)OR⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆ alkyl), S(C₆-C₁₀ aryl), S(═O)₂R⁶, S(═O)₂OR⁶, P(═O)(R⁶)₂, C₁-C₆ alkyl, C₂-C₆ alkenyl, C₂-C₆ alkynyl, C₃-C₈ cycloalkyl, C₆-C₁₀ aryl, 3-8 membered heterocycloalkyl, or 3-10 membered heteroaryl;

provided that: (1) when one of R₃ and R₄ is C₁ alkyl, and T is —N(R_{S})— or —O—, then the other one of R₃ and R₄ is absent;

(2) when one of R₃ and R₄ is C₁-C₂ alkyl, and T is absent, then the other one of R₃ and R₄ is absent;

(3) when one of R₃ and R₄ is C₂-C₆ alkyl, then T is not —N(R_{S})— or —O—;

(4) when at least one of R₃ and R₄ is C₃-C₆ alkyl, then T is not absent;

(5) when X⁴ is —NR¹¹, at least one A is —N—; and/or

(6) when T is -Tₐ-C₂-C₆ alkyl-T_{b}, and X⁴ is absent, then R₁ is CN.

In some embodiments, the compound is not

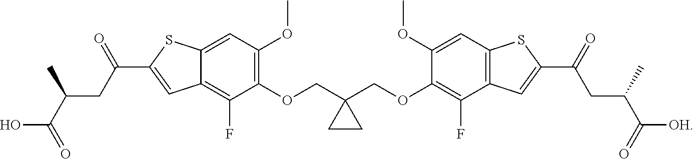 [SMILES: COc1cc2sc(C(=O)C[C@H](C)C(=O)O)cc2c(F)c1OCC1(COc2c(OC)cc3sc(C(=O)C[C@H](C)C(=O)O)cc3c2F)CC1]

In some embodiments, when T is -Tₐ-(C₃-C₁₂ cycloalkyl)-T_{b}- or -Tₐ-(3- to 12-membered heterocycloalkyl)-T_{b}-, then the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl.

In some embodiments, the compound is of Formula (I), (II), (III), (IV), (V), or (VI), or a pharmaceutically acceptable salt thereof, wherein:

each A independently is —C(R¹)— or —N—;

each R¹ independently is hydrogen, halogen, OR⁶, N(R⁶)₂, CN, or C₁-C₆ alkyl, wherein the C₁-C₆ alkyl is optionally substituted with one or more halogen, OR⁶, N(R⁶)₂, C(═O)OR⁶, or C(═O)N(R⁶)₂;

each R² independently is hydrogen, halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C₁-C₆ alkyl, C₂-C₆ alkenyl, or C₂-C₆ alkynyl is optionally substituted with one more halogen or OR⁶;

R³ and R⁴ each independently are absent, C₁-C₆ alkyl, —N(R_{S})—, or —O—;

each T independently is -Tₐ-C₂-C₆ alkyl-T_{b}-, —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S};

Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; wherein the C₁-C₆ alkyl it optionally substituted with one or more halogen; and

each R_{S} independently is H or C₁-C₆ alkyl optionally substituted with one or more halogen;

each R⁶ independently is H or C₁-C₆ alkyl optionally substituted with one or more halogen;

each X¹ independently is —C(═O)—, —CH₂—, —CHF—, or —CF₂—;

each X² independently is —(C(R⁸)₂)₁₋₃—, wherein each R⁸ independently: (a) is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂; or (2) together with another R⁸ and the one or more atoms to which they are attached, form C₃-C₆ cycloalkyl or 3- to 6-membered heterocycloalkyl;

each X³ independently is C(═O)OR⁶, C(═O)SR⁶, C(═S)OR⁶,

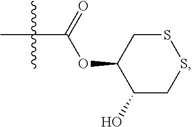 [SMILES: *C(=O)O[C@H]1CSSC[C@@H]1O]

SO₂R⁶, C(═O)N(R⁹)₂, or CN;

each R⁹ independently is hydrogen, C(═O)OR⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆ alkyl), S(C₆-C₁₀ aryl), S(═O)₂R⁶, S(═O)₂OR⁶, P(═O)(R⁶)₂, C₁-C₆ alkyl, C₂-C₆ alkenyl, C₂-C₆ alkynyl, C₃-C₈ cycloalkyl, C₆-C₁₀ aryl, 3-8 membered heterocycloalkyl, or 3-10 membered heteroaryl; and

X⁴ is absent or —NR¹¹, wherein each R¹¹ independently is H or C₁-C₆ alkyl optionally substituted with one or more halogen;

provided that:

(1) when one of R₃ and R₄ is C₁ alkyl, and T is —N(R_{S})— or —O—, then the other one of R₃ and R₄ is absent;

(2) when one of R₃ and R₄ is C₂-C₆ alkyl, then T is not —N(R_{S})— or —O—;

(3) when X⁴ is —NR¹¹, at least one A is —N—; and/or

(4) when T is -Tₐ-C₂-C₆ alkyl-T_{b}, and X⁴ is absent, then R₁ is CN.

In some embodiments, the compound is of Formula (I), (II), (III), (IV), (V), or (VI), or a pharmaceutically acceptable salt thereof, wherein:

each A independently is —C(R¹)— or —N—;

each R¹ independently is hydrogen, halogen, OR⁶, N(R⁶)₂, CN, or C₁-C₆ alkyl, wherein the C₁-C₆ alkyl is optionally substituted with one or more halogen, OR⁶, N(R⁶)₂, C(═O)OR⁶, or C(═O)N(R⁶)₂;

each R² independently is hydrogen, halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C₁-C₆ alkyl, C₂-C₆ alkenyl, or C₂-C₆ alkynyl is optionally substituted with one more halogen or OR⁶;

R³ and R⁴ each independently are absent;

each T independently is -Tₐ-C₂-C₆ alkyl-T_{b}-, —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S};

Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; wherein the C₁-C₆ alkyl it optionally substituted with one or more halogen; and

each R_{S} independently is H or C₁-C₆ alkyl optionally substituted with one or more halogen;

each R⁶ independently is H or C₁-C₆ alkyl optionally substituted with one or more halogen;

each X¹ independently is —C(═O)—, —CH₂—, —CHF—, or —CF₂—;

each X² independently is —(C(R⁸)₂)₁₋₃—, wherein each R⁸ independently: (a) is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂; or (2) together with another R⁸ and the one or more atoms to which they are attached, form C₃-C₆ cycloalkyl or 3- to 6-membered heterocycloalkyl;

each X³ independently is C(═O)OR⁶, C(═O)SR⁶, C(═S)OR⁶,

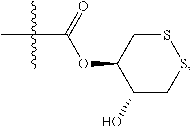 [SMILES: *C(=O)O[C@H]1CSSC[C@@H]1O]

SO₂R⁶, C(═O)N(R⁹)₂, or CN;

each R⁹ independently is hydrogen, C(═O)OR⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆ alkyl), S(C₆-C₁₀ aryl), S(═O)₂R⁶, S(═O)₂OR⁶, P(═O)(R⁶)₂, C₁-C₆ alkyl, C₂-C₆ alkenyl, C₂-C₆ alkynyl, C₃-C₈ cycloalkyl, C₆-C₁₀ aryl, 3-8 membered heterocycloalkyl, or 3-10 membered heteroaryl; and

X⁴ is absent or —NR¹¹, wherein each R¹¹ independently is H or C₁-C₆ alkyl optionally substituted with one or more halogen;

provided that:

(1) when X⁴ is —NR¹¹, at least one A is —N—; and/or

(2) when T is -Tₐ-C₂-C₆ alkyl-T_{b}, and X⁴ is absent, then R₁ is CN.

In some embodiments, the compound is of Formula (I), (II), (III), (IV), (V), or (VI), or a pharmaceutically acceptable salt thereof, wherein:

each A independently is —C(R¹)— or —N—;

each R¹ independently is hydrogen, halogen, OR⁶, N(R⁶)₂, CN, or C₁-C₆ alkyl, wherein the C₁-C₆ alkyl is optionally substituted with one or more halogen, OR⁶, N(R⁶)₂, C(═O)OR⁶, or C(═O)N(R⁶)₂;

each R² independently is hydrogen, halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C1-C6 alkyl, C2-C6 alkenyl, or C₂-C₆ alkynyl is optionally substituted with one more halogen or OR⁶;

R³ and R⁴ each independently are absent;

each T independently is -Tₐ-C₂-C₆ alkyl-T_{b}-, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)—T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S};

Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; wherein the C₁-C₆ alkyl it optionally substituted with one or more halogen; and

each R_{S} independently is H or C₁-C₆ alkyl optionally substituted with one or more halogen;

each R⁶ independently is H or C₁-C₆ alkyl optionally substituted with one or more halogen;

each X¹ independently is —C(═O)—, —CH₂—, —CHF—, or —CF₂—;

each X² independently is —(C(R⁸)₂)₁₋₃—, wherein each R⁸ independently: (a) is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂; or (2) together with another R⁸ and the one or more atoms to which they are attached, form C₃-C₆ cycloalkyl or 3- to 6-membered heterocycloalkyl;

each X³ independently is C(═O)OR⁶, C(═O)SR⁶, C(═S)OR⁶,

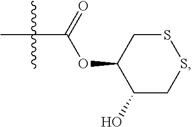 [SMILES: *C(=O)O[C@H]1CSSC[C@@H]1O]

SO₂R⁶, C(═O)N(R⁹)₂, or CN;

each R⁹ independently is hydrogen, C(═O)OR⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆ alkyl), S(C₆-C₁₀ aryl), S(═O)₂R⁶, S(═O)₂OR⁶, P(═O)(R⁶)₂, C₁-C₆ alkyl, C₂-C₆ alkenyl, C₂-C₆ alkynyl, C₃-C₈ cycloalkyl, C₆-C₁₀ aryl, 3-8 membered heterocycloalkyl, or 3-10 membered heteroaryl; and

X⁴ is absent or —NR¹¹, wherein each R¹¹ independently is H or C₁-C₆ alkyl optionally substituted with one or more halogen;

provided that:

(1) when X⁴ is —NR¹¹, at least one A is —N—; and/or

(2) when T is -Tₐ-C₂-C₆ alkyl-T_{b}, and X⁴ is absent, then R₁ is CN.

In some embodiments, the compound is of Formula (I), (II), (III), (IV), (V), or (VI), or a pharmaceutically acceptable salt thereof, wherein:

each A independently is —C(R¹)— or —N—;

each R¹ independently is hydrogen, halogen, OR⁶, N(R⁶)₂, CN, or C₁-C₆ alkyl, wherein the C₁-C₆ alkyl is optionally substituted with one or more halogen, OR⁶, N(R⁶)₂, C(═O)OR⁶, or C(═O)N(R⁶)₂;

each R² independently is hydrogen, halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C₁-C₆ alkyl, C₂-C₆ alkenyl, or C₂-C₆ alkynyl is optionally substituted with one more halogen or OR⁶;

R³ and R⁴ each independently are absent;

each T independently is -Tₐ-C₂-C₆ alkenyl-T_{b}-, -Tₐ-C(═O)-T_{b}, -Tₐ-C(═CH₂)-T_{b}-, or -Tₐ-(C₆-C₁₂ aryl)-T_{b}-, wherein the C₂-C₆ alkenyl or C₆-C₁₂ aryl is optionally substituted with one or more halo, —OR_{S}, —N(R_{S})₂, or —C(═O)OR_{S};

Tₐ and T_{b} each independently are —N(R_{S})—, —O—, —(C₁-C₆ alkyl)-O—, or —O—(C₁-C₆ alkyl)-O—; wherein in the C₁-C₆ alkyl it optionally substituted with one or more halogen;

each R_{S} independently is H or C₁-C₆ alkyl optionally substituted with one or more halogen;

each R⁶ independently is H or C₁-C₆ alkyl optionally substituted with one or more halogen;

each X¹ independently is —C(═O)—, —CH₂—, —CHF—, or —CF₂—;

each X² independently is —(C(R⁸)₂)₁₋₃—, wherein each R⁸ independently: (a) is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂; or (2) together with another R⁸ and the one or more atoms to which they are attached, form C₃-C₆ cycloalkyl or 3- to 6-membered heterocycloalkyl;

each X³ independently is C(═O)OR⁶, C(═O)SR⁶, C(═S)OR⁶,

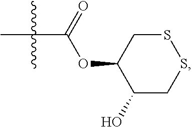 [SMILES: *C(=O)O[C@H]1CSSC[C@@H]1O]

SO₂R⁶, C(═O)N(R⁹)₂, or CN;

each R⁹ independently is hydrogen, C(═O)OR⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆ alkyl), S(C₆-C₁₀ aryl), S(═O)₂R⁶, S(═O)₂OR⁶, P(═O)(R⁶)₂, C₁-C₆ alkyl, C₂-C₆ alkenyl, C₂-C₆ alkynyl, C₃-C₅ cycloalkyl, C₆-C₁₀ aryl, 3-8 membered heterocycloalkyl, or 3-10 membered heteroaryl; and

X⁴ is absent or —NR¹¹, wherein each R¹¹ independently is H or C₁-C₆ alkyl optionally substituted with one or more halogen;

provided that:

(1) when X⁴ is —NR¹¹, at least one A is —N—; and/or

(2) when T is -Tₐ-C₂-C₆ alkyl-T_{b}, and X⁴ is absent, then R₁ is CN.

In some embodiments, the compound is of Formula (I), (II), (III), (IV), (V), or (VI), or a pharmaceutically acceptable salt thereof, wherein:

each A independently is —N—;

each R¹ independently is hydrogen, halogen, OR⁶, N(R⁶)₂, CN, or C₁-C₆ alkyl, wherein the C₁-C₆ alkyl is optionally substituted with one or more halogen, OR⁶, N(R⁶)₂, C(═O)OR⁶, or C(═O)N(R⁶)₂;

each R² independently is hydrogen, halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C₁-C₆ alkyl, C₂-C₆ alkenyl, or C₂-C₆ alkynyl is optionally substituted with one more halogen or OR⁶;

R³ and R⁴ each independently are absent, C₁-C₆ alkyl, —N(R_{S})—, or —O—;

each T independently is -Tₐ-C₂-C₆ alkyl-T_{b}-, —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S};

Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; wherein the C₁-C₆ alkyl it optionally substituted with one or more halogen; and

each R_{S} independently is H or C₁-C₆ alkyl optionally substituted with one or more halogen;

each R⁶ independently is H or C₁-C₆ alkyl optionally substituted with one or more halogen;

each X¹ independently is —C(═O)—, —CH₂—, —CHF—, or —CF₂—;

each X² independently is —(C(R⁸)₂)₁₋₃—, wherein each R⁸ independently: (a) is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂; or (2) together with another R⁸ and the one or more atoms to which they are attached, form C₃-C₆ cycloalkyl or 3- to 6-membered heterocycloalkyl;

each X³ independently is C(═O)OR⁶, C(═O)SR⁶, C(═S)OR⁶,

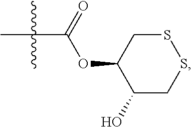 [SMILES: *C(=O)O[C@H]1CSSC[C@@H]1O]

SO₂R⁶, C(═O)N(R⁹)₂, or CN;

each R⁹ independently is hydrogen, C(═O)OR⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆ alkyl), S(C₆-C₁₀ aryl), S(═O)₂R⁶, S(═O)₂OR⁶, P(═O)(R⁶)₂, C₁-C₆ alkyl, C₂-C₆ alkenyl, C₂-C₆ alkynyl, C₃-C₈ cycloalkyl, C₆-C₁₀ aryl, 3-8 membered heterocycloalkyl, or 3-10 membered heteroaryl; and

X⁴ is —NR¹¹, wherein each R¹¹ independently is H or C₁-C₆ alkyl optionally substituted with one or more halogen;

provided that:

(1) when X⁴ is —NR¹¹, at least one A is —N—; and/or

(2) when T is -Tₐ-C₂-C₆ alkyl-T_{b}, and X⁴ is absent, then R₁ is CN.

In aspects, the present disclosure provides a compound of Formula (I), (II), (III), (IV), (V) or (VI):

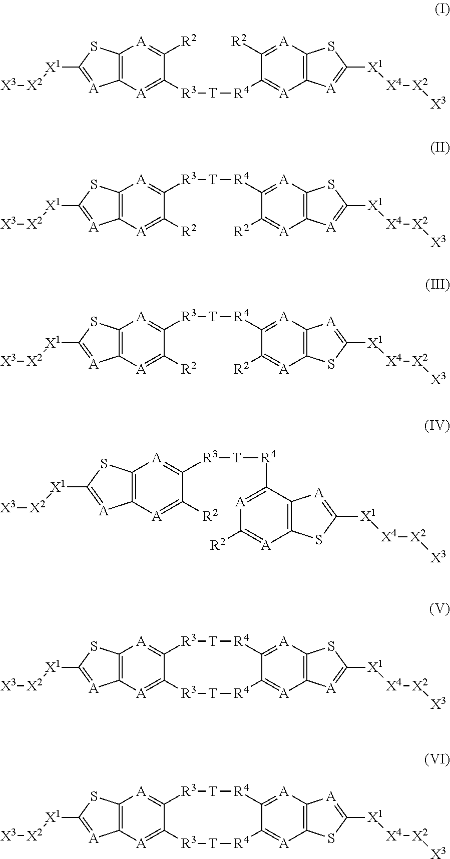

or a pharmaceutically acceptable salt thereof, wherein:

each A independently is —C(R¹)— or —N—;

each R¹ independently is hydrogen, halogen, OR⁶, N(R⁶)₂, or C₁-C₆ alkyl, wherein the C₁-C₆ alkyl is optionally substituted with one or more halogen, OR⁶, N(R⁶)₂, C(═O)OR⁶, or C(═O)N(R⁶)₂;

each R² independently is hydrogen, halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C₁-C₆ alkyl, C₂-C₆ alkenyl, or C₂-C₆ alkynyl is optionally substituted with one more halogen or OR⁶;

R³ and R⁴ each independently are absent, C₁-C₆ alkyl, —N(R_{S})—, or —O—;

each T independently is absent, —N(R_{S})—, —O—, -Tₐ-N(R_{S})—N(R_{S})-T_{b}-, -Tₐ-C₂-C₆ alkenyl-T_{b}-Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}, and wherein the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;

Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; and

each R_{S} independently is H or C₁-C₆ alkyl optionally substituted with one or more halogen;

each R⁶ independently is H or C₁-C₆ alkyl optionally substituted with one or more halogen;

each X¹ independently is —C(═O)—, —CH₂—, —CHF—, or —CF₂—;

each X² independently is —(C(R⁸)₂)₁₋₃—, wherein each R⁸ independently: (a) is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂; or (2) together with another R⁸ and the one or more atoms to which they are attached, form C₃-C₆ cycloalkyl or 3- to 6-membered heterocycloalkyl;

each X³ independently is C(═O)OR⁶, C(═O)SR⁶, C(═S)OR₆,

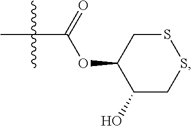 [SMILES: *C(=O)O[C@H]1CSSC[C@@H]1O]

SO₂R⁶, C(═O)N(R⁹)₂, or CN; and

each R⁹ independently is hydrogen, C(═O)OR⁶, or S(═O)₂R⁶;

provided that:

(1) when one of R₃ and R₄ is C₁ alkyl, and T is —N(R_{S})— or —O—, then the other one of R₃ and R₄ is absent;

(2) when one of R₃ and R₄ is C₁-C₂ alkyl, and T is absent, then the other one of R₃ and R₄ is absent;

(3) when one of R₃ and R₄ is C₂-C₆ alkyl, then T is not —N(R_{S})— or —O—; and/or

(4) when at least one of R₃ and R₄ is C₃-C₆ alkyl, then T is not absent.

It is understood that, for a compound of Formula (I), (II), (III), (IV), (V), or (VI), variables R¹, R², R³, R⁴, R⁶, R⁸, R⁹, R¹¹, A, X¹, X², X³, X⁴, T, Tₐ, T_{b}, and R_{S} can each be, where applicable, selected from the groups described herein, and any group described herein for any of variables R¹, R², R³, R⁴, R⁶, R⁸, R⁹, R¹¹, A, X¹, X², X³, X⁴, T, Tₐ, T_{b}, and R_{S} can be combined, where applicable, with any group described herein for one or more of the remainder of variables R¹, R², R³, R⁴, R⁶, R⁸, R⁹, R¹¹, A, X¹, X², X³, X⁴, T, Tₐ, T_{b}, and R_{S}.

In some embodiments, in the compound of Formula (I), (II), (III), (IV), (V), or (VI), variables R¹, R², R⁶, R⁸, R⁹, R¹¹, A, X¹, X², X³, and X⁴, each independently are selected from each other, with the proviso that at least one of R¹, R², R⁶, R⁸, and R⁹ is not H.

In some embodiments, (1) when one of R₃ and R₄ is C₁ alkyl, and T is —N(R_{S})— or —O—, then the other one of R₃ and R₄ is absent; (2) when one of R₃ and R₄ is C₁-C₂ alkyl, and T is absent, then the other one of R₃ and R₄ is absent; (3) when one of R₃ and R₄ is C₂-C₆ alkyl, then T is not —N(R_{S})— or —O—; (4) when at least one of R₃ and R₄ is C₃-C₆ alkyl, then T is not absent; 5) when X⁴ is —NR¹¹, at least one A is —N—; and (6) when T is -Tₐ-C₂-C₆ alkyl-T_{b}, and X⁴ is absent, then R₁ is CN.

In some embodiments, the compound is of Formula (I) or the pharmaceutically acceptable salt thereof.

In some embodiments, each

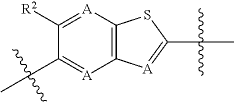

independently is

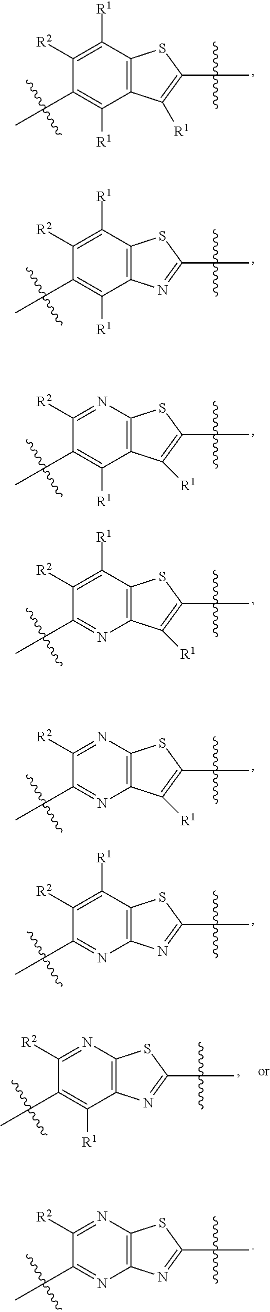

In some embodiments, each

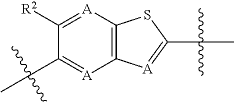

independently is

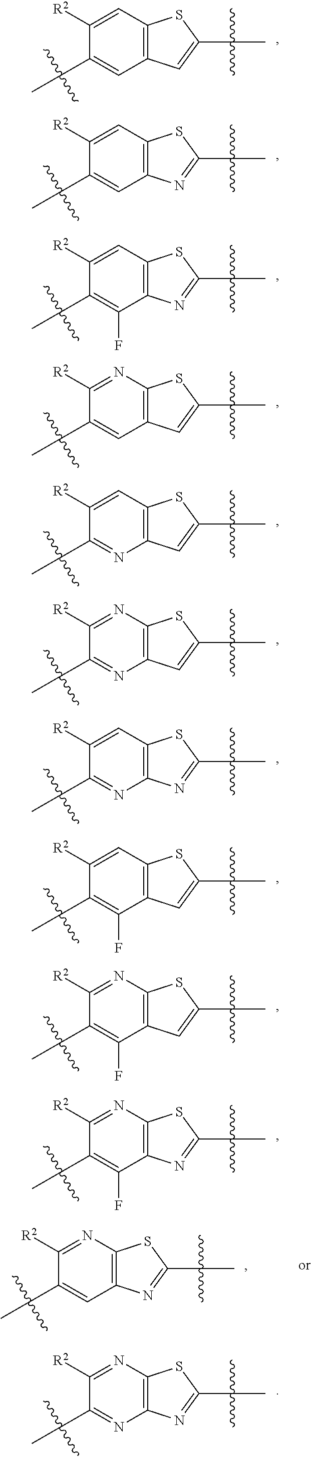

In some embodiments, the compound is of Formula (II) or the pharmaceutically acceptable salt thereof.

In some embodiments, each

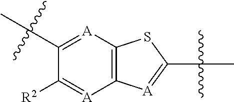

independently is

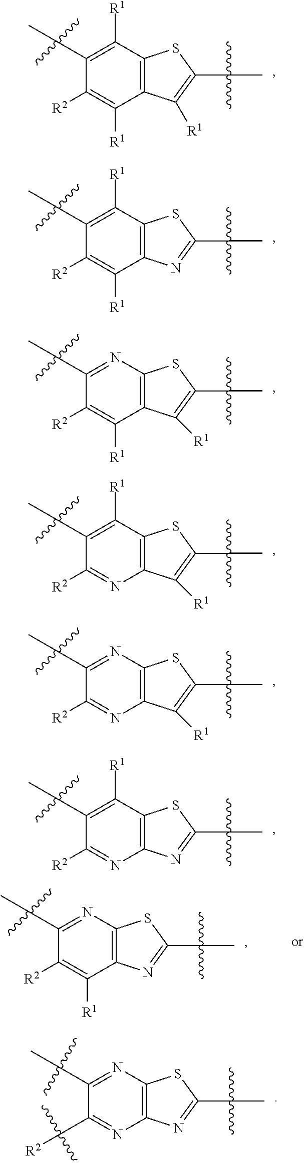

In some embodiments each

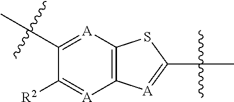

independently is

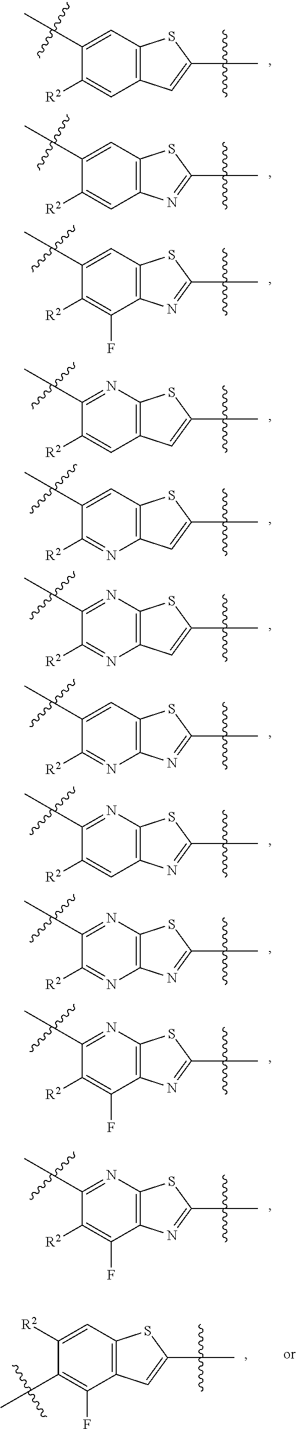

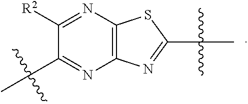

In some embodiments, the compound is of Formula (III) or the pharmaceutically acceptable salt thereof.

In some embodiments, each

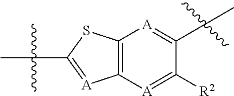

independently is

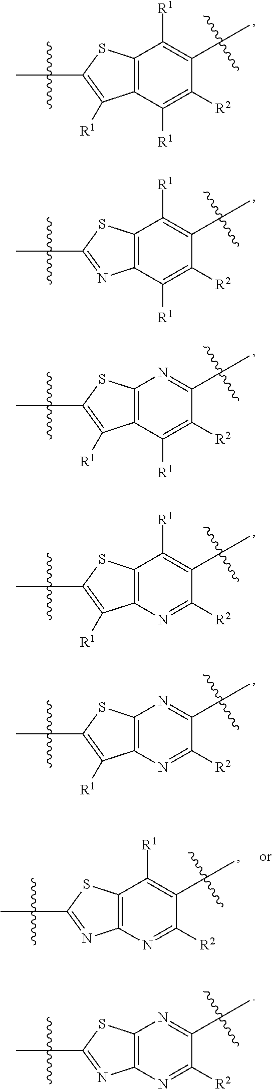

In some embodiments, each

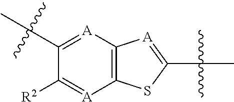

independently is

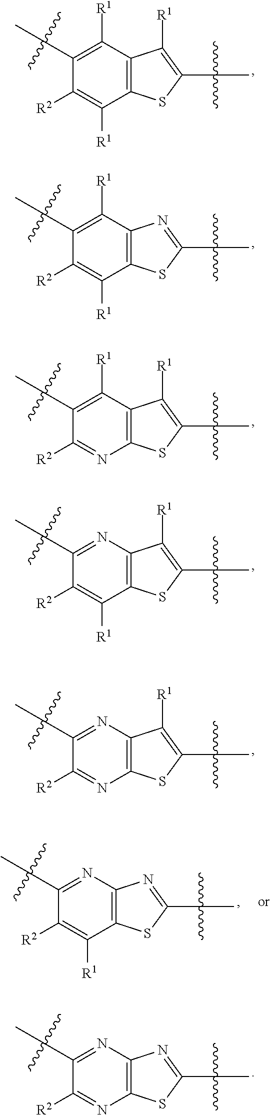

In some embodiments, each

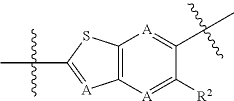

independently is

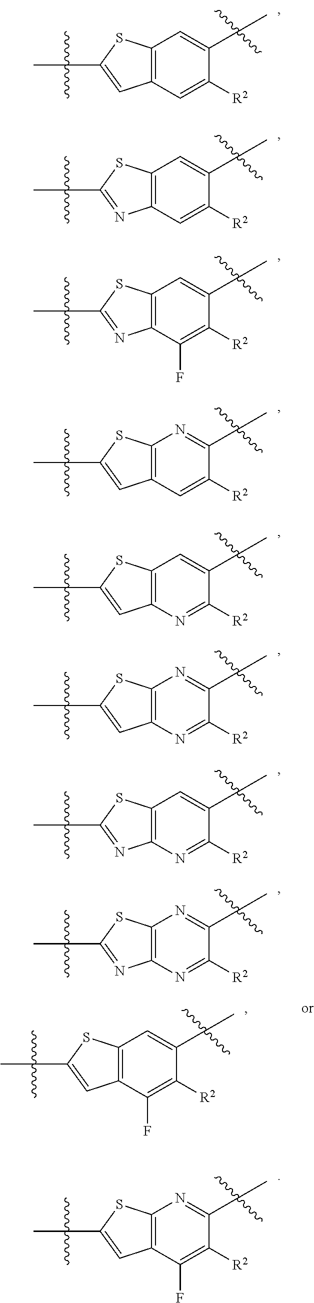

In some embodiments, each

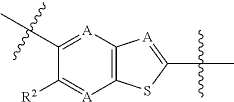

independently is

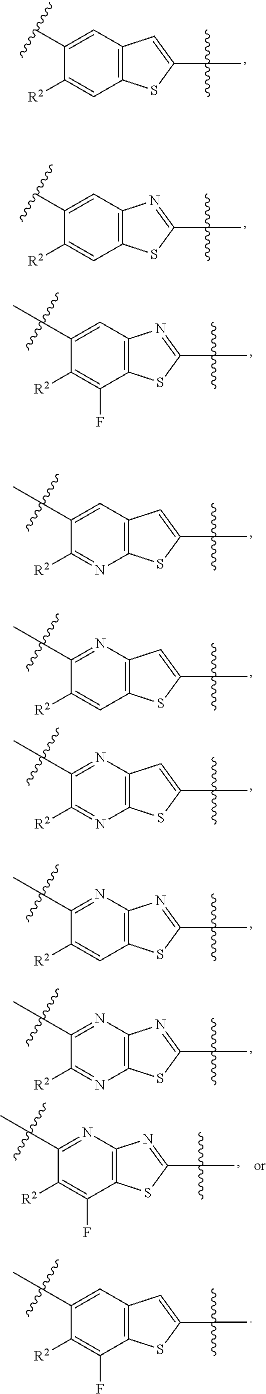

In some embodiments, the compound is of Formula (IV) or the pharmaceutically acceptable salt thereof.

In some embodiments, each

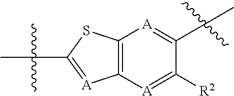

independently is

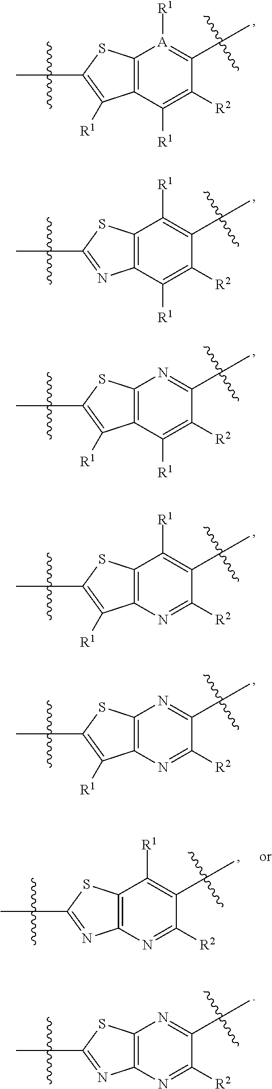

In some embodiments, each

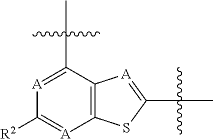

independently is,

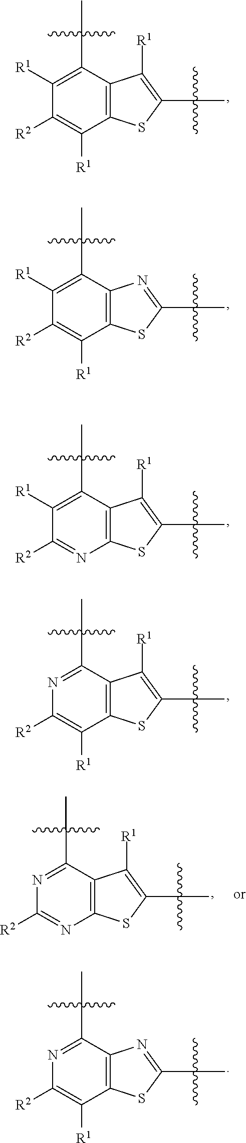

In some embodiments, each

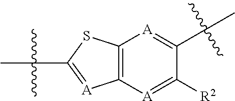

independently is

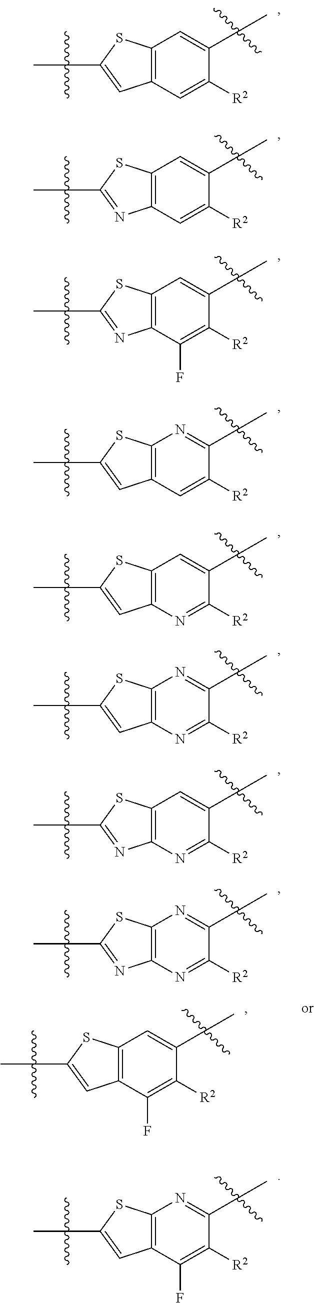

In some embodiments, each

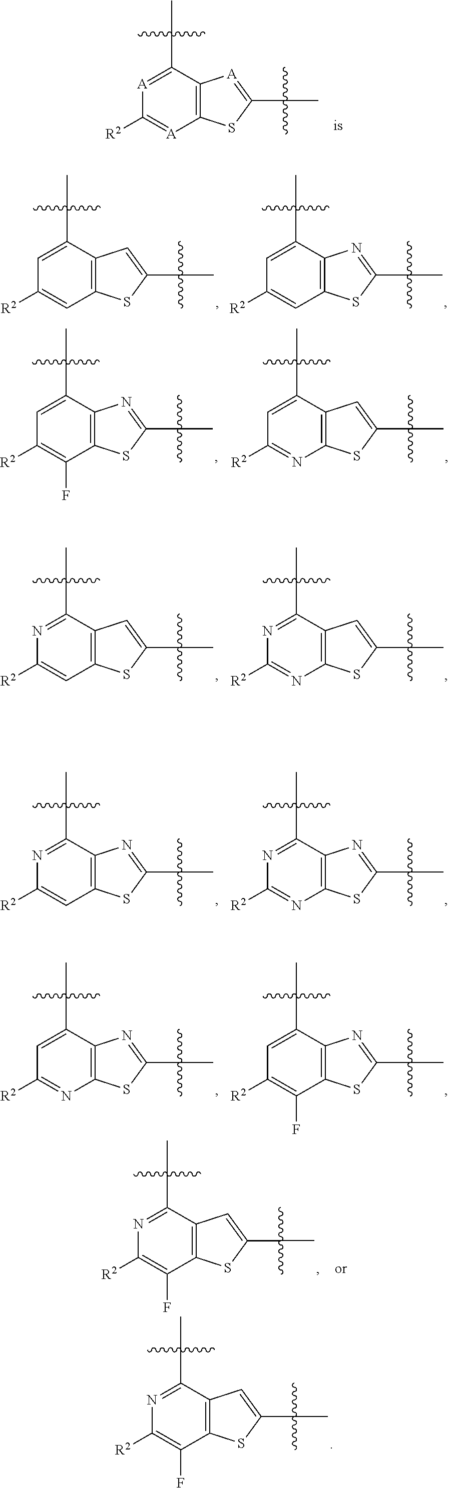

In some embodiments, the compound is of Formula (V) or the pharmaceutically acceptable salt thereof.

In some embodiments, each

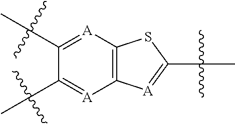

independently is

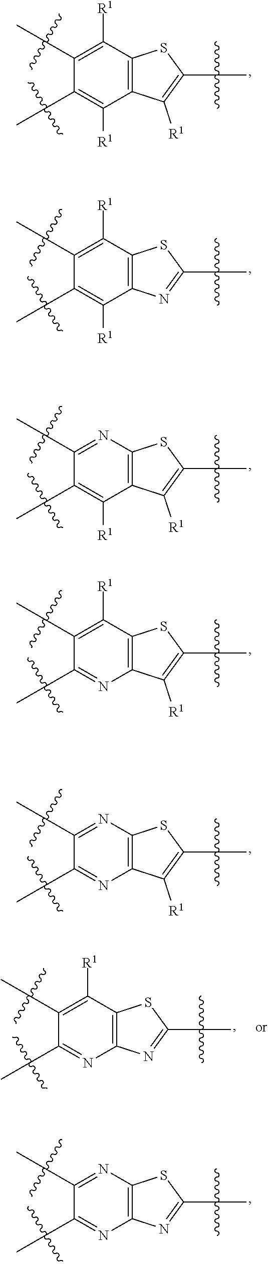

In some embodiments, each

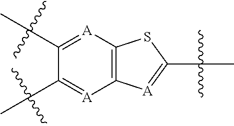

independently is

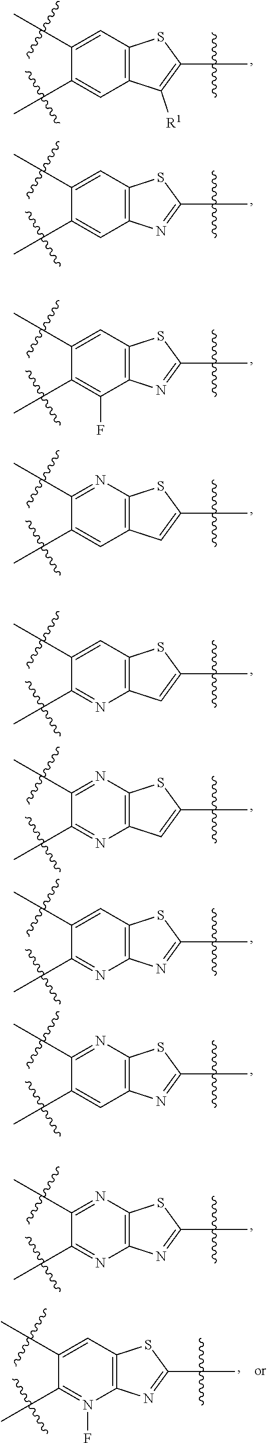

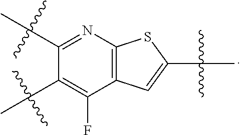 [SMILES: *c1cc2c(F)c(*)c(*)nc2s1]

In some embodiments, the compound is of Formula (VI) or the pharmaceutically acceptable salt thereof.

In some embodiments, each

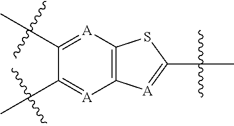

independently is

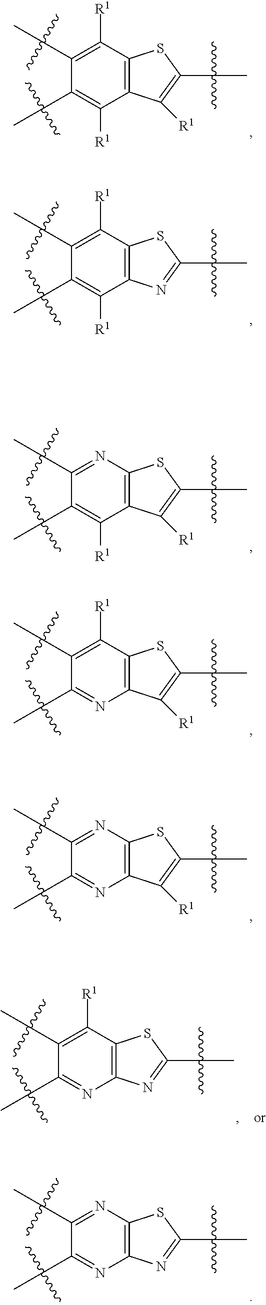

In some embodiments, each

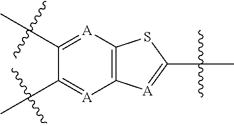

independently is

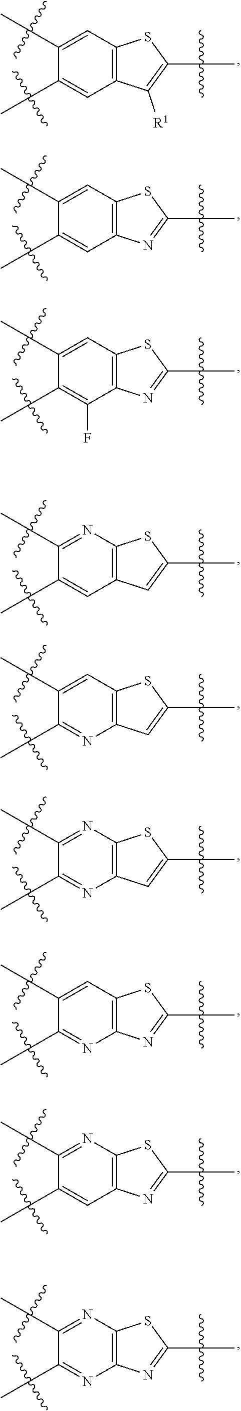

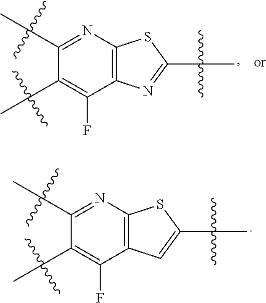 [SMILES: *c1nc2c(F)c(*)c(*)nc2s1 ; *c1cc2c(F)c(*)c(*)nc2s1]

In some embodiments, at least one A is —C(R¹)—.

In some embodiments, at least one A is —CH— or —CF—.

In some embodiments, each A is —C(R¹)—.

In some embodiments, each A independently is —CH— or —CF—.

In some embodiments, at least one A is —N—.

In some embodiments, at least two A is —N—.

In some embodiments, two A are —N—, and the other A each independently are —C(R¹)—.

In some embodiments, two A are —N—, and the other A each independently are —CH— or —CF—.

In some embodiments, at least one R¹ is hydrogen.

In some embodiments, at least one R¹ is halogen, OR⁶, N(R⁶)₂, CN, or C₁-C₆ alkyl, wherein the C₁-C₆ alkyl is optionally substituted with one or more halogen, OR⁶, N(R⁶)₂, C(═O)OR⁶, or C(═O)N(R⁶)₂.

In some embodiments, at least one R¹ is halogen.

In some embodiments, at least one R¹ is F.

In some embodiments, at least one R¹ is CN.

In some embodiments, each R¹ independently is hydrogen, halogen, CN or C₁-C₃ alkyl, wherein the C₁-C₃ alkyl is optionally substituted with one or more halogen.

In some embodiments, each R¹ independently is hydrogen or halogen.

In some embodiments, each R¹ independently is hydrogen or F.

In some embodiments, each R¹ independently is hydrogen or CN.

In some embodiments, at least one R² is halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C₁-C₆ alkyl, C₂-C₆ alkenyl, or C₂-C₆ alkynyl is optionally substituted with one more halogen or OR⁶.

In some embodiments, each R² independently is hydrogen, halogen, OR⁶, N(R⁶)₂, C₁-C₃ alkyl, or C₂-C₃ alkenyl, wherein the C₁-C₃ alkyl or C₂-C₃ alkenyl is optionally substituted with one more halogen.

In some embodiments, at least one R² is OR⁶.

In some embodiments, each R² is OR⁶.

In some embodiments, each R² independently is hydrogen, Br, Cl, OCH₃, OCH₂F, OCHF₂, or OCF₃, CH₃, CH₂CH₃, or CH═CH₂.

In some embodiments, each R² independently is hydrogen, OCH₃, OCHF₂, or CH₃.

In some embodiments, each R² independently is hydrogen or OCH₃.

In some embodiments, at least one R² is hydrogen.

In some embodiments, each R² is hydrogen.

In some embodiments, at least one R² is OCH₃.

In some embodiments, each R² is OCH₃.

In some embodiments, at least one of R³ and R⁴ is absent.

In some embodiments, one of R³ and R⁴ is absent, and the other one of R³ and R⁴ is C₁-C₆ alkyl, —N(R_{S})—, or —O—.

In some embodiments, one of R³ and R⁴ is absent, and the other one of R³ and R⁴ is methyl.

In some embodiments, one of R³ and R⁴ is absent, the other one of R³ and R⁴ is methyl, and T is —N(R_{S})— or —O—.

In some embodiments, one of R³ and R⁴ is absent, and the other one of R³ and R⁴ is methyl or ethyl.

In some embodiments, one of R³ and R⁴ is absent, the other one of R³ and R⁴ is methyl or ethyl, and T is absent.

In some embodiments, R³ and R⁴ each are absent.

In some embodiments, R³ and R⁴ each independently are C₁-C₆ alkyl, —N(R_{S})—, or —O—.

In some embodiments, R³ and R⁴ each are absent, and each T independently is absent —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}; wherein in the -Tₐ-(C₃-C₁₂ cycloalkyl)-T_{b}- or -Tₐ-(3- to 12-membered heterocycloalkyl)-T_{b}-, the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl.

In some embodiments, R³ and R⁴ each are absent, and each T independently is absent, —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}, and wherein the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl.

In some embodiments, R₃ and R₄ each independently are absent, C₁-C₆ alkyl, —N(R_{S})—, or —O—; and each T independently is NH—, —N(C₁₋₆ alkyl)-, —O—, C₂-C₆ alkenyl, C₂-C₆ alkynyl, —C₀₋₆alkyl-C(═O)—C₀₋₆ alkyl-, —C₀₋₆ alkyl-C(═CH₂)—C₀₋₆ alkyl-, —C₀₋₆ alkyl-C(═O)—C(═O)—C₀₋₆ alkyl-, —C₀₋₆ alkyl-C(═S)—C₀₋₆alkyl-, —C₀₋₆ alkyl-S(═O)₂—C₀₋₆ alkyl-, —C₀₋₆ alkyl-S(═O)—C₀₋₆ alkyl-, —C₀₋₆ alkyl-P(═O)(—OH)—C₀₋₆ alkyl-, —C₀₋₆ alkyl-C₃-C₁₂ cycloalkyl-C₀₋₆ alkyl-, —C₀₋₆ alkyl-C₆-C₁₂ aryl-C₀₋₆ alkyl-, —C₀₋₆ alkyl-(3- to 12-membered heterocyclyl)-C₀₋₆ alkyl-, —C₀₋₆ alkyl-(5- to 12-membered heteroaryl)-C₀₋₆ alkyl-, —C₀₋₆ alkyl-O-(5- to 12-membered heteroaryl)-O—C₀₋₆ alkyl-, —C₀₋₆ alkyl-O—C(═O)—NH—C₀₋₆ alkyl-, —C₀₋₆ alkyl-O—C(═O)—C₀₋₆ alkyl-, —C₀₋₆ alkyl-NH—C(═O)—C₀₋₆ alkyl-, —O—C(═O)—O—, —NH—C(═O)—NH—, or —NH—C(═S)—NH—; wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}; provided that: (1) when one of R₃ and R₄ is C₁ alkyl, and T is —NH—, —N(C₁₋₆ alkyl)-, or —O—, then the other one of R₃ and R₄ is absent; and/or (2) when one of one of R₃ and R₄ is C₂-C₆ alkyl, then T is not —NH—, —N(C₁₋₆ alkyl)-, or —O—.

In some embodiments, R³ and R⁴ each independently are absent, C₁-C₆ alkyl, —N(R_{S})—, or —O—;

each T independently is —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}-, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C1-C6 alkyl)-(3- to 12-membered heterocycloalkyl)-(C1-C6 alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}; wherein in -Tₐ-(C₃-C₁₂ cycloalkyl)-T_{b}- or -Tₐ-(3- to 12-membered heterocycloalkyl)-T_{b}-, the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;

Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; wherein the C₁-C₆ alkyl it optionally substituted with one or more halogen; and

each R_{S} independently is H or C₁-C₆ alkyl optionally substituted with one or more halogen; provided that:

(1) when one of R₃ and R₄ is C₁ alkyl, and T is —N(R_{S})— or —O—, then the other one of R₃ and R₄ is absent; and/or

(2) when one of R₃ and R₄ is C₂-C₆ alkyl, then T is not —N(R_{S})— or —O—.

In some embodiments, R³ and R⁴ each independently are absent;

each T independently is —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}; wherein in -Tₐ-(C₃-C₁₂ cycloalkyl)-T_{b}- or -Tₐ-(3- to 12-membered heterocycloalkyl)-T_{b}-, the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;

Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; wherein the C₁-C₆ alkyl it optionally substituted with one or more halogen; and

each R_{S} independently is H or C1-C6 alkyl optionally substituted with one or more halogen.

In some embodiments, R³ and R⁴ each independently are absent;

each T independently is -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}-, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}; wherein in -Tₐ-(C₃-C₁₂ cycloalkyl)-T_{b}- or -Tₐ-(3- to 12-membered heterocycloalkyl)-T_{b}-, the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;

Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; wherein the C₁-C₆ alkyl it optionally substituted with one or more halogen; and each R_{S} independently is H or C₁-C₆ alkyl optionally substituted with one or more halogen.

In some embodiments, R³ and R⁴ each independently are absent;

each T independently is -Tₐ-C₂-C₆ alkenyl-T_{b}-, -Tₐ-C(═O)-T_{b}, -Tₐ-C(═CH₂)-T_{b}-, or -Tₐ-(C₆-C₁₂ aryl)-T_{b}-, wherein the C₂-C₆ alkenyl or C₆-C₁₂ aryl is optionally substituted with one or more halo, —OR_{S}, —N(R_{S})₂, or —C(═O)OR_{S};

Tₐ and T_{b} each independently are —N(R_{S})—, —O—, —(C₁-C₆ alkyl)-O—, or —O—(C₁-C₆ alkyl)-O—; wherein in the C1-C6 alkyl it optionally substituted with one or more halogen; and

each R_{S} independently is H or C₁-C₆ alkyl optionally substituted with one or more halogen.

In some embodiments, R³ and R⁴ each are absent, and each T independently is absent, -Tₐ-C₂-C₄ alkyl-T_{b}-, —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}, and wherein the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl.

In some embodiments, each

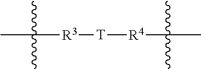

independently is:

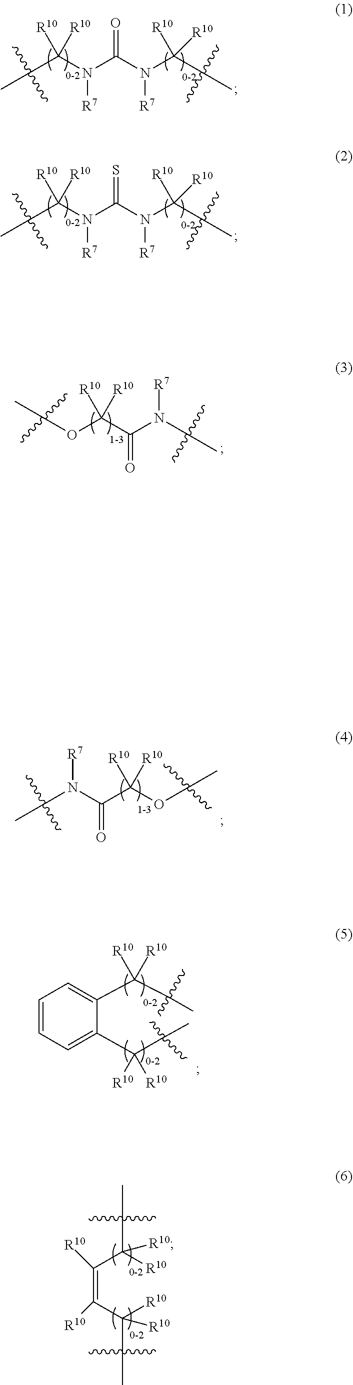

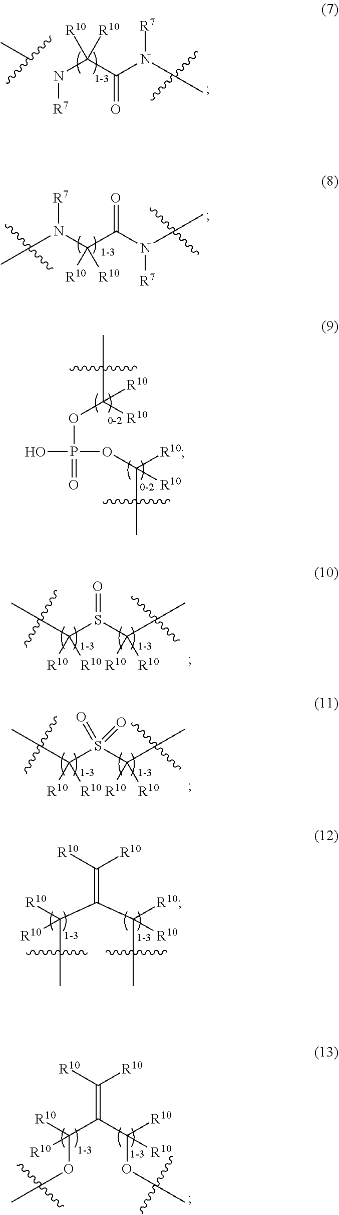

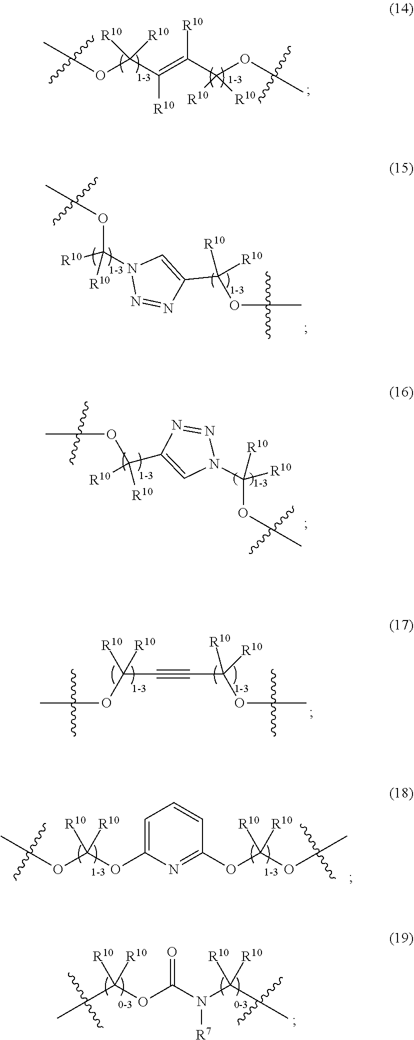

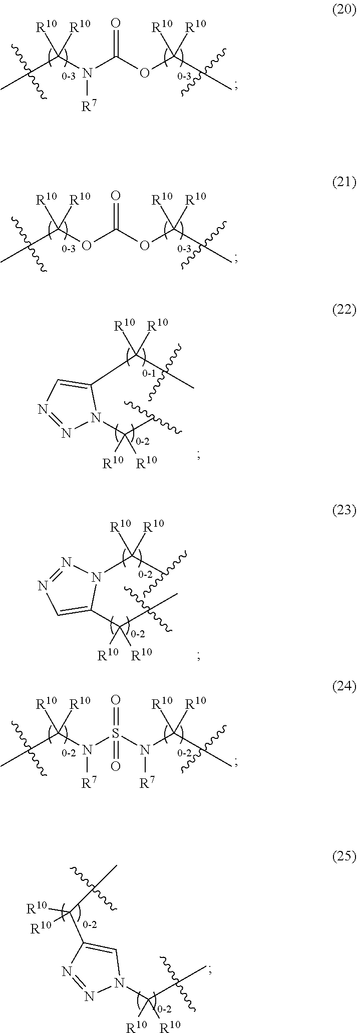

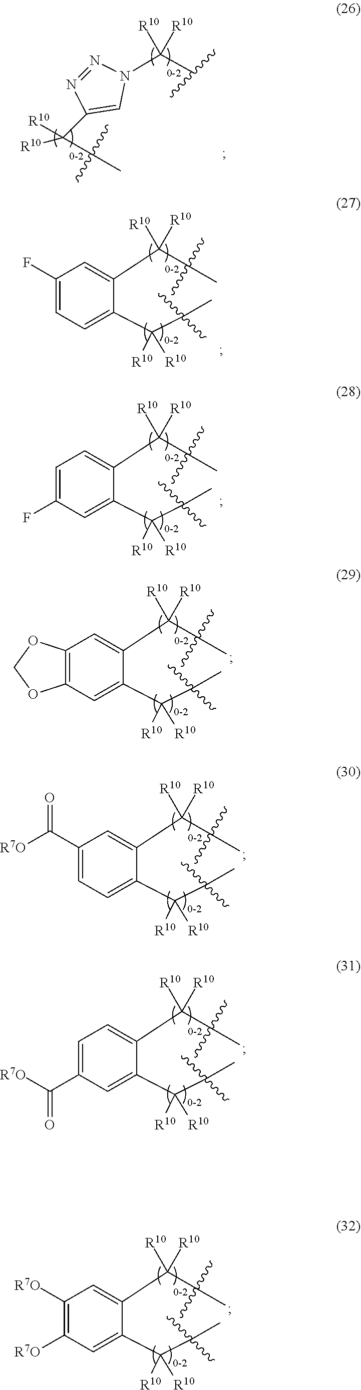

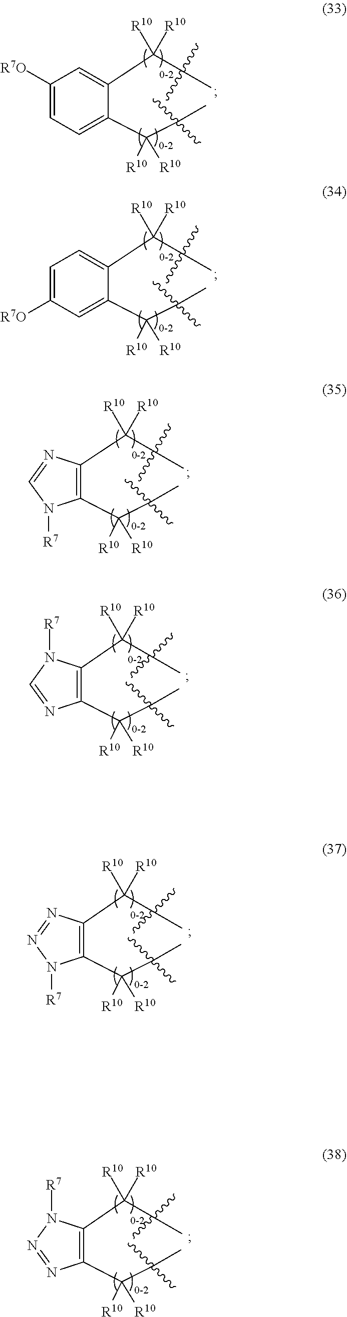

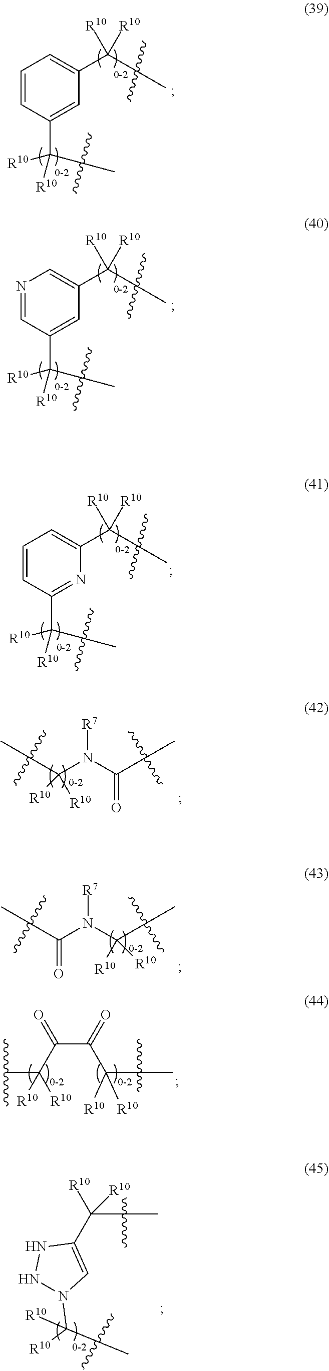

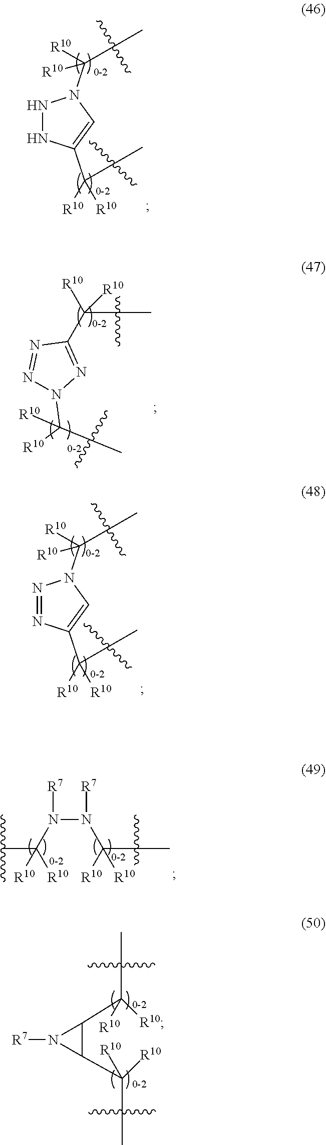

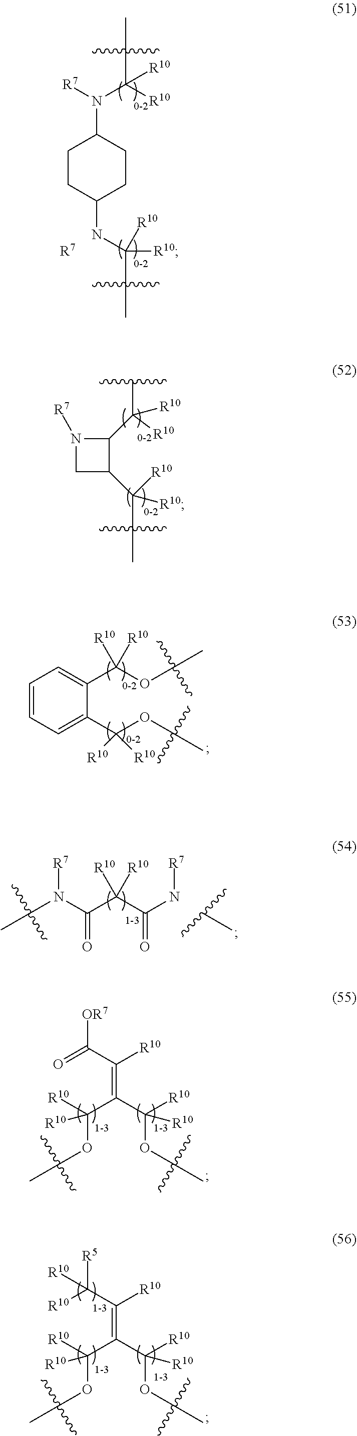

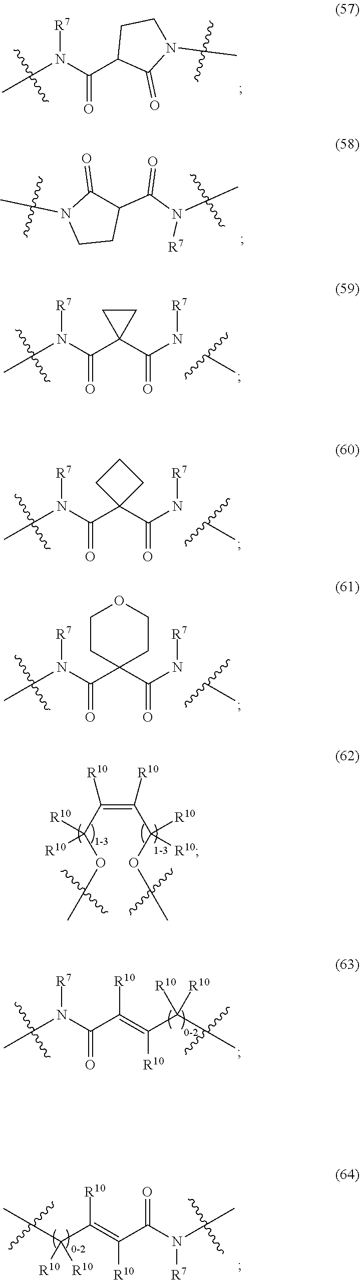

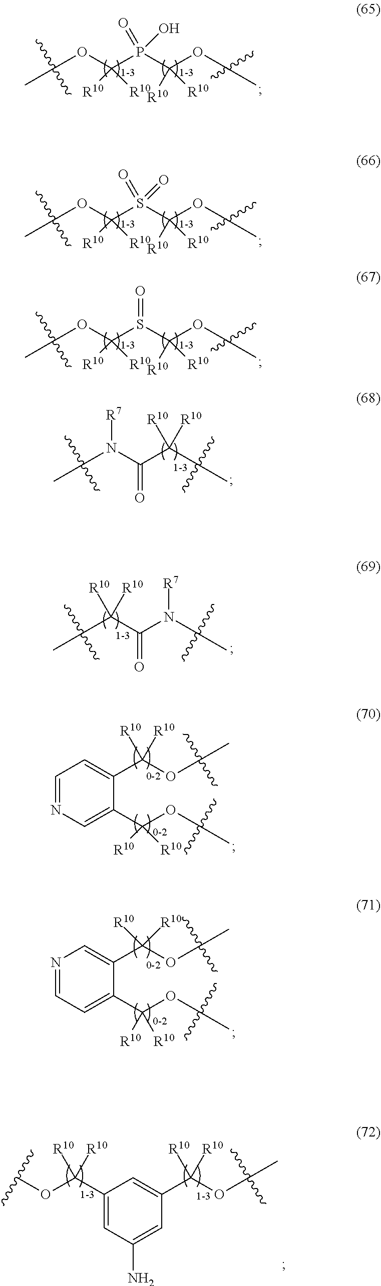

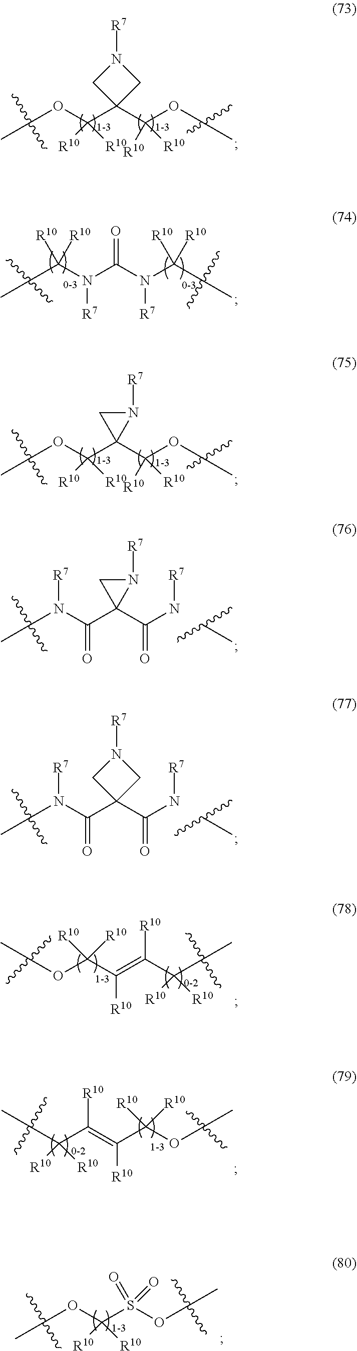

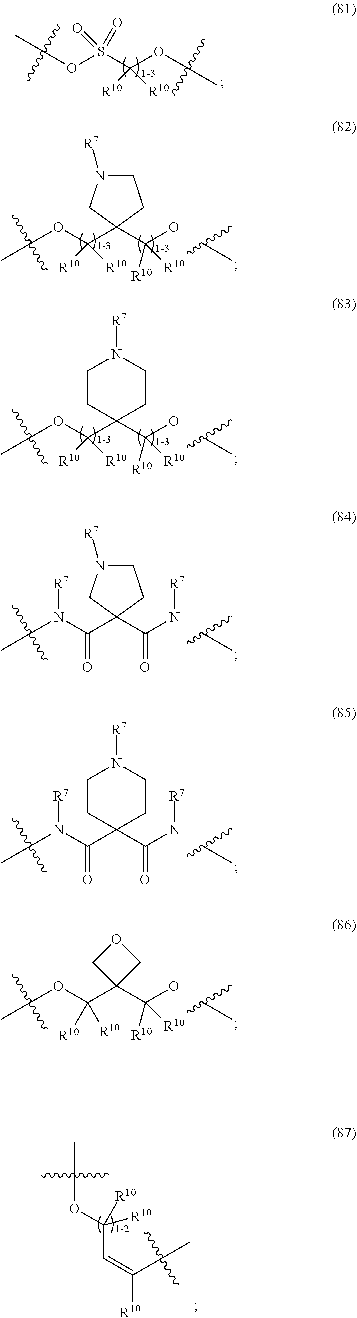

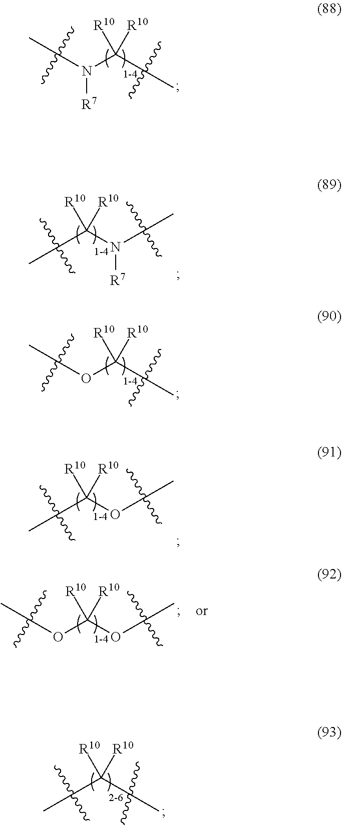

wherein:

each R⁵ is independently —OR⁷, NR⁷ or —C(O)OR⁷

each R⁷ is independently hydrogen or C₁₋₂ alkyl; and

each R¹⁰ is independently hydrogen, C₁₋₂ alkyl or halogen.

In some embodiments, each

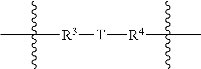

independently is:

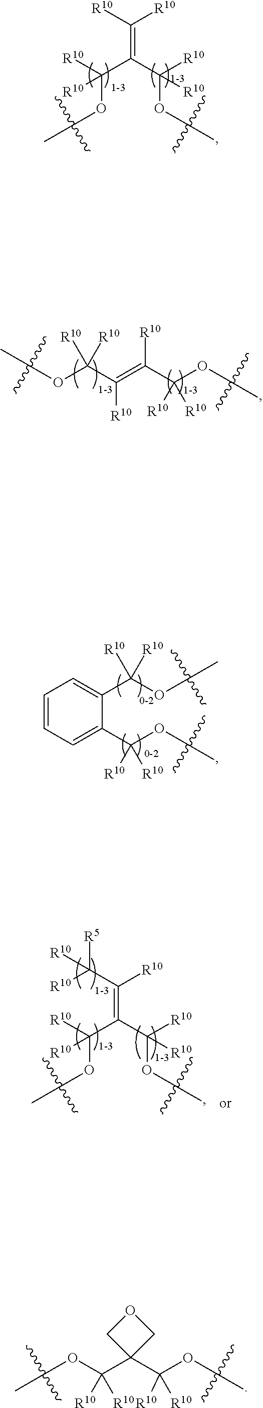

In some embodiments, when (i) X⁴ is absent, and R¹ is CN; or (ii) when X⁴ is —NR¹¹ and at least one A is —N—, each

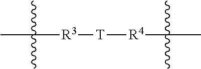

independently is:

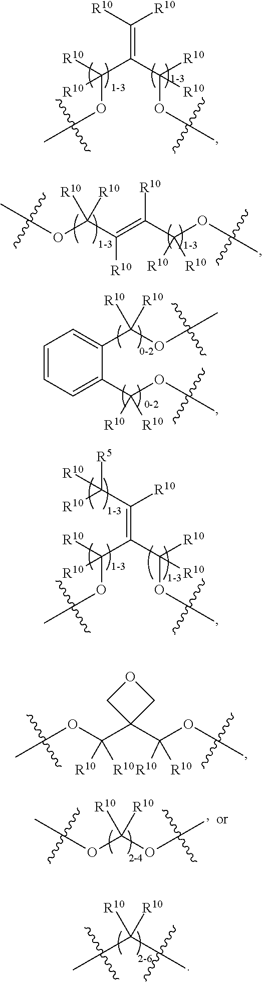

In some embodiments, each R⁷ is independently hydrogen or methyl.

In some embodiments, each R¹⁰ is independently hydrogen, methyl or fluorine.

In some embodiments, each R¹⁰ is hydrogen.

In some embodiments, each R¹⁰ is methyl.

In some embodiments, each R¹⁰ is fluorine.

In some embodiments, one R¹⁰ is hydrogen, and the other R¹⁰ is methyl or fluorine.

In some embodiments, one R¹⁰ is hydrogen, and the other R¹⁰ is fluorine.

In some embodiments, one R¹⁰ is hydrogen, and the other R¹⁰ is methyl.

In some embodiments, each

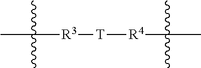

independently is:

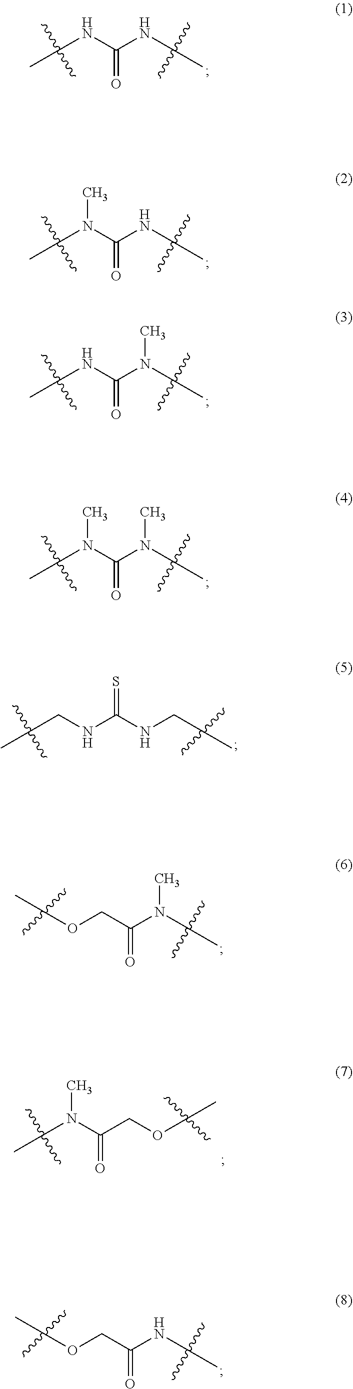

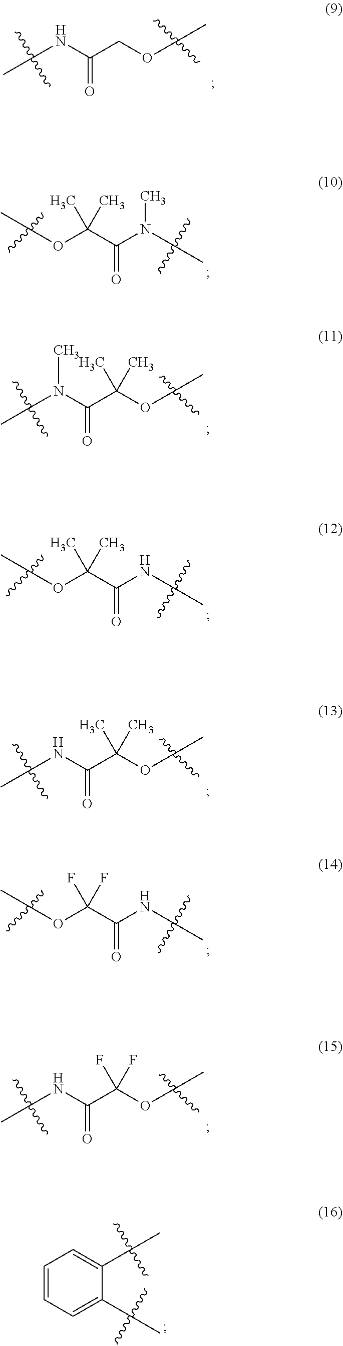

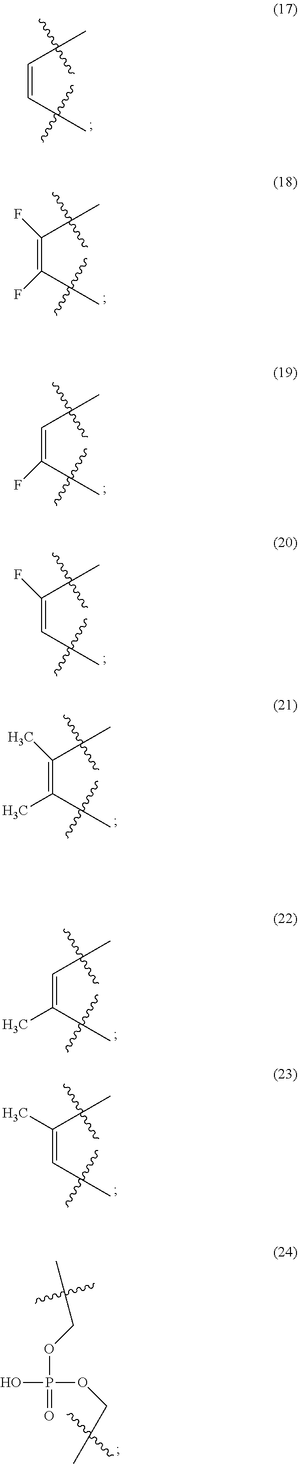

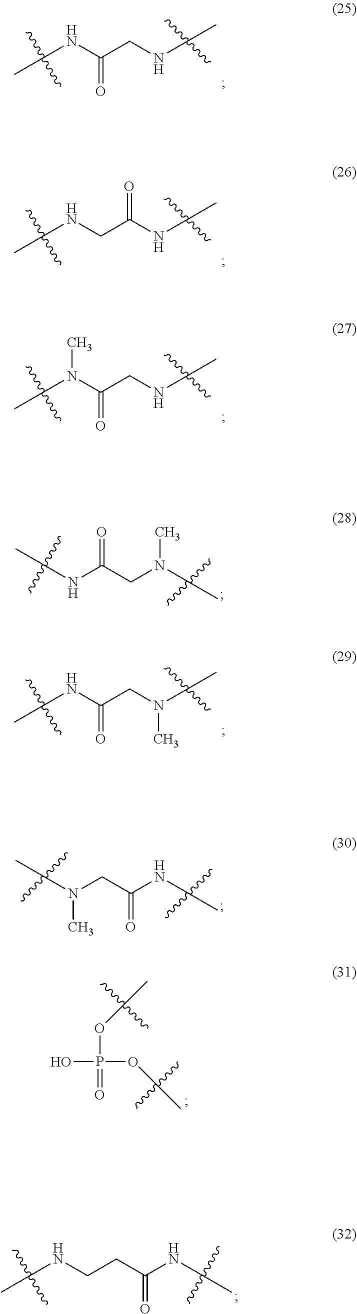

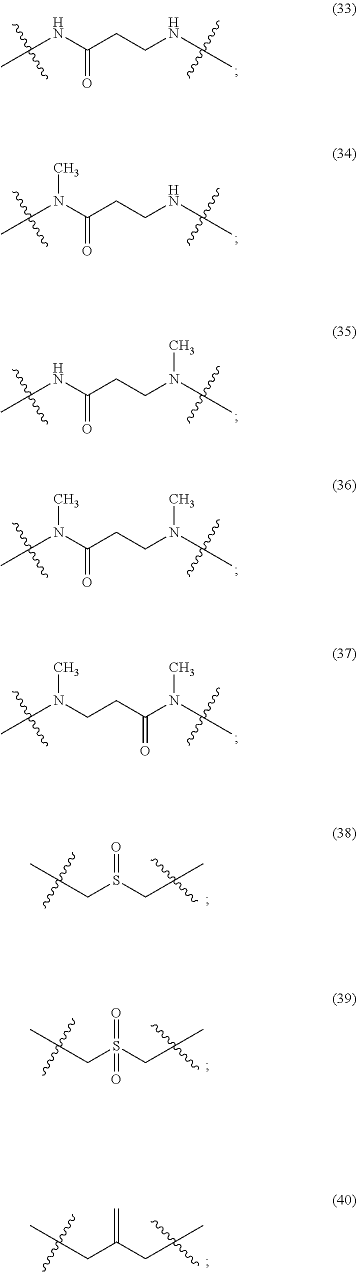

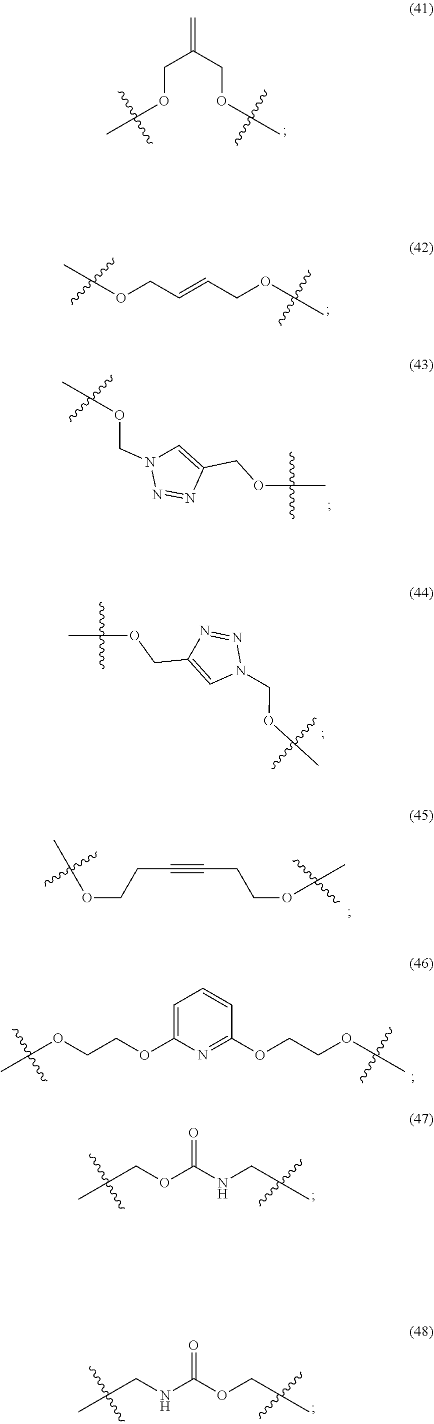

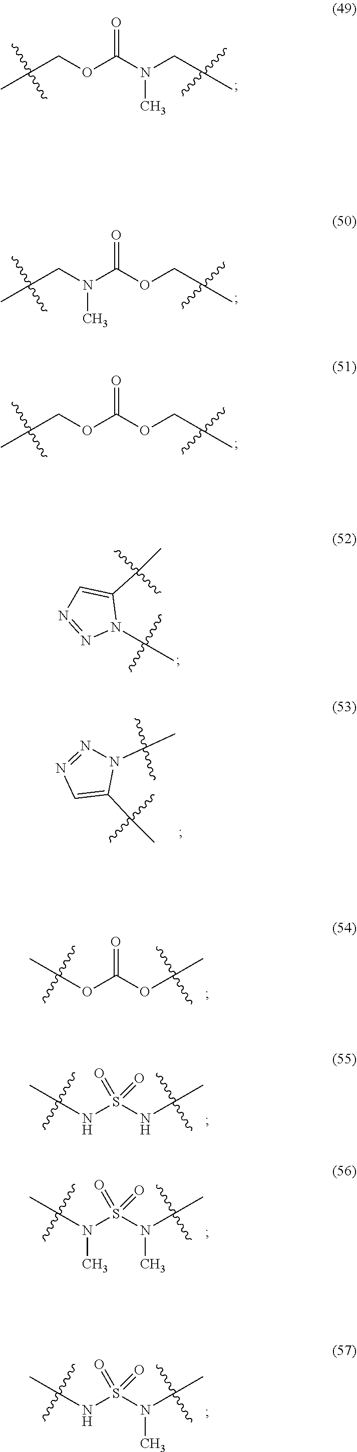

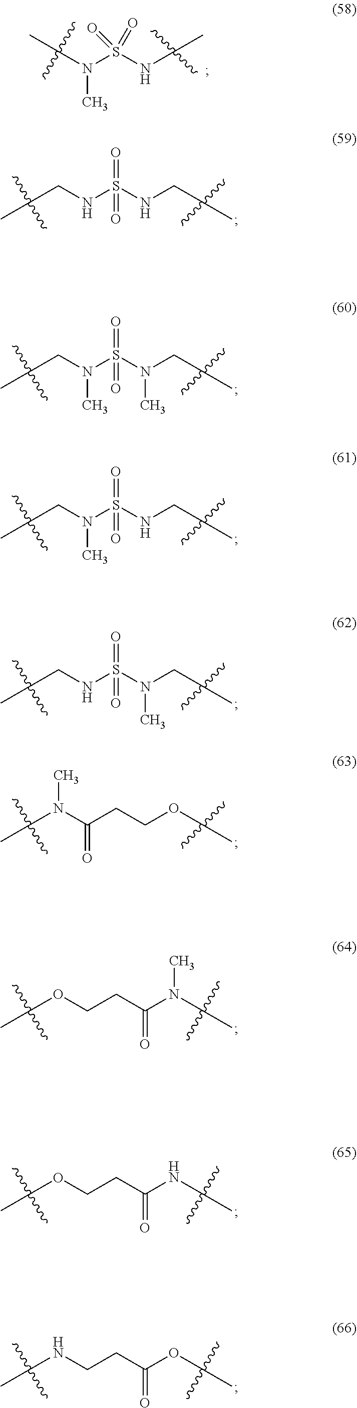

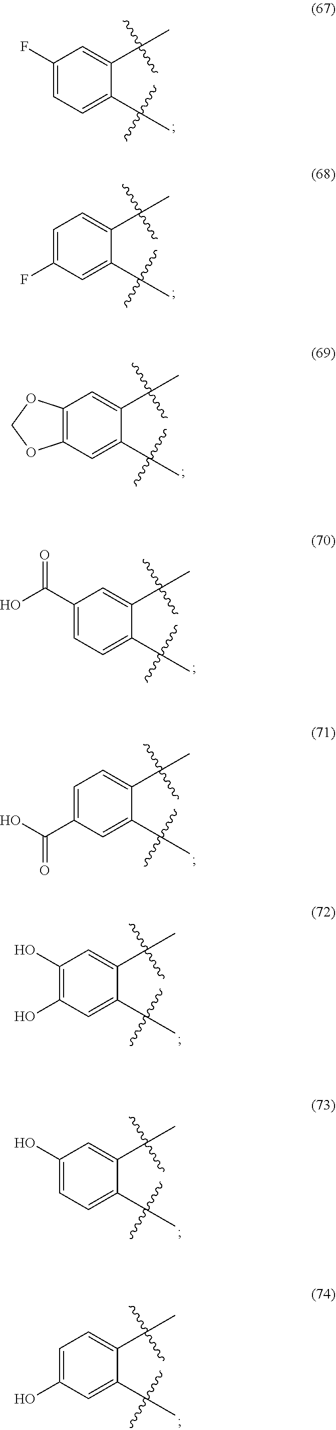

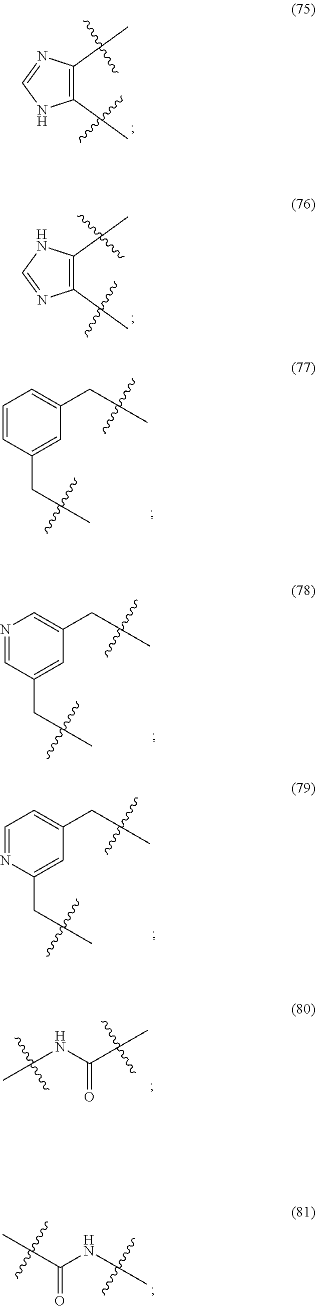

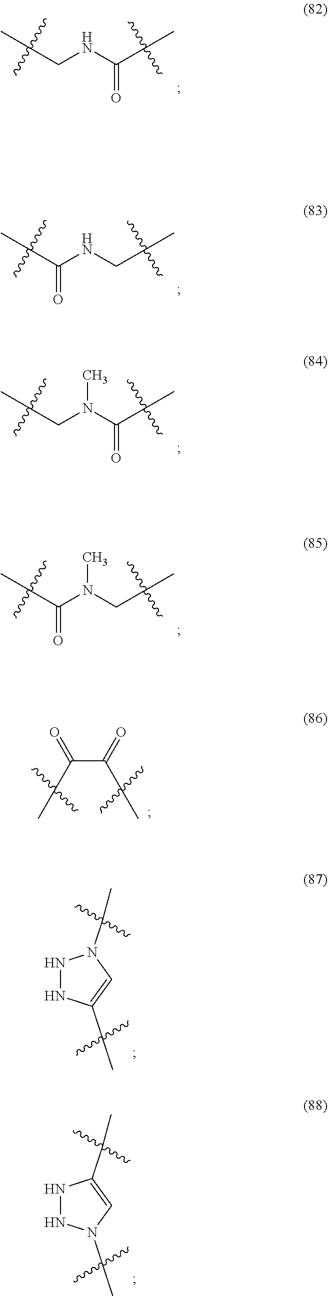

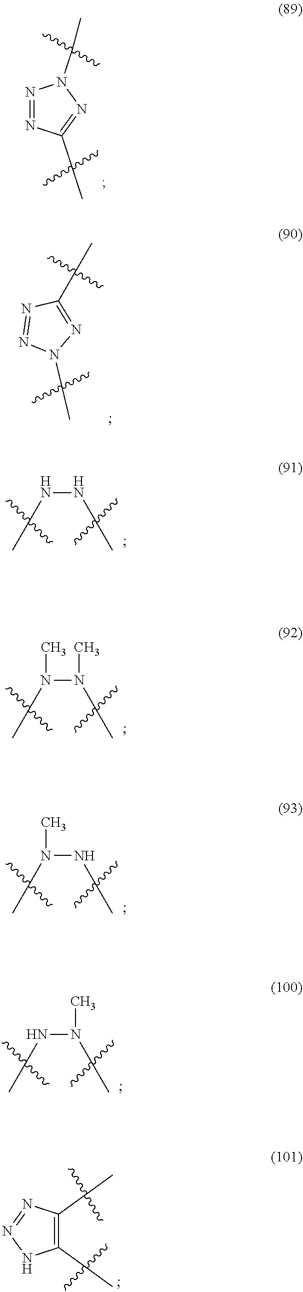

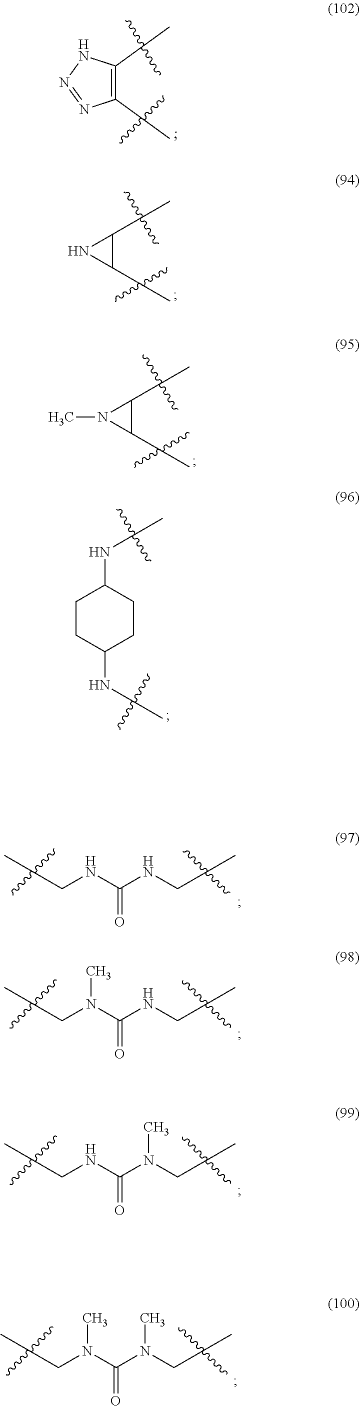

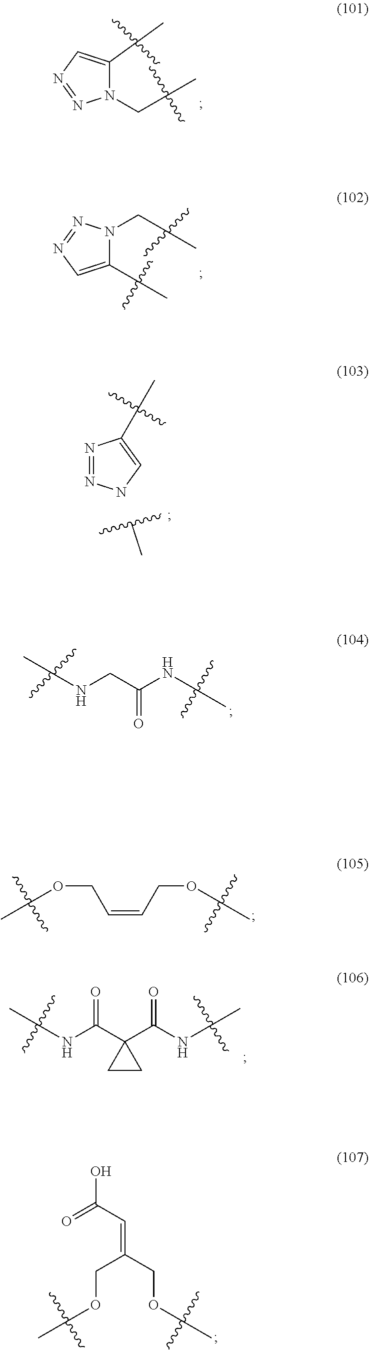

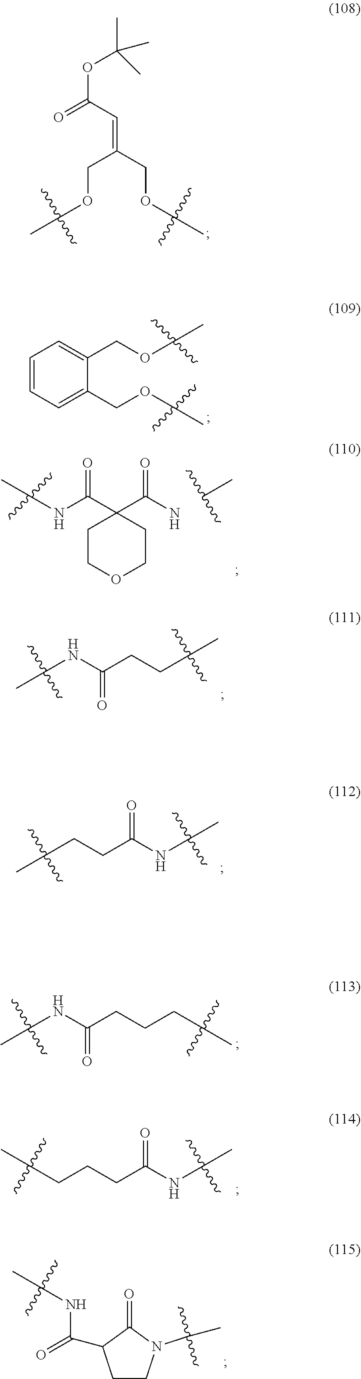

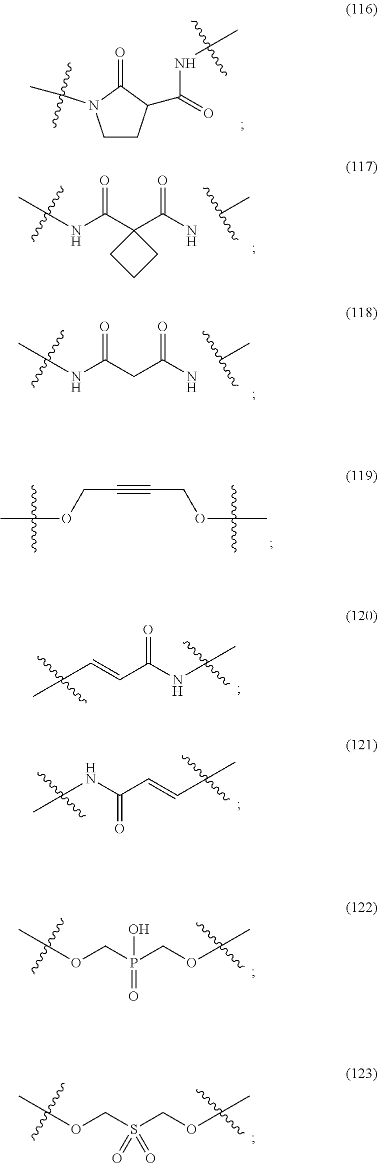

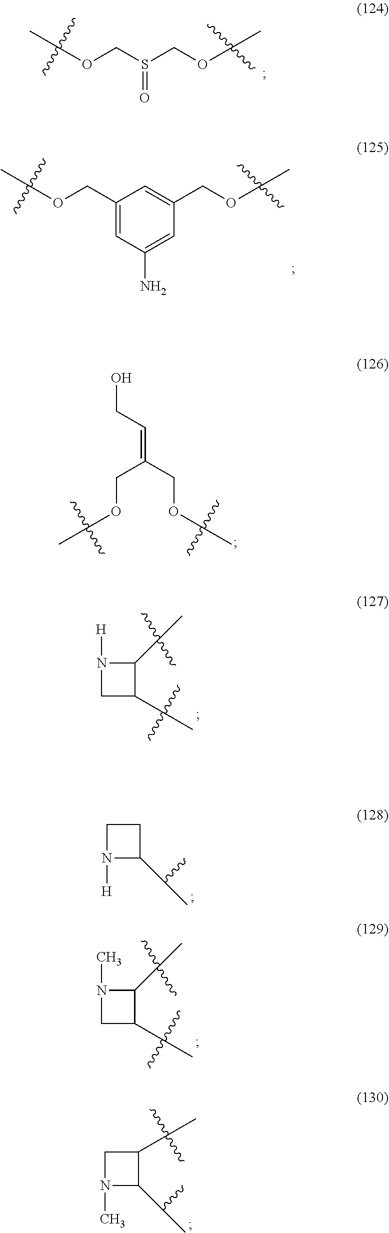

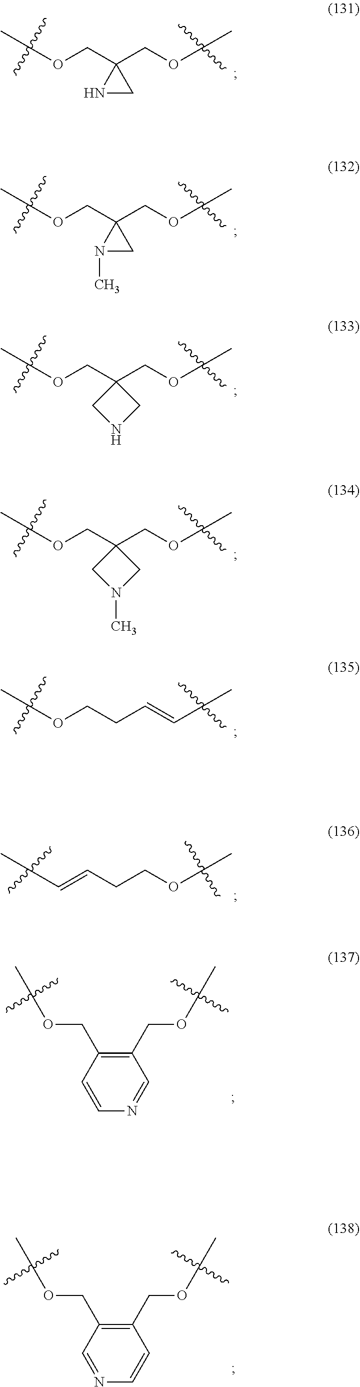

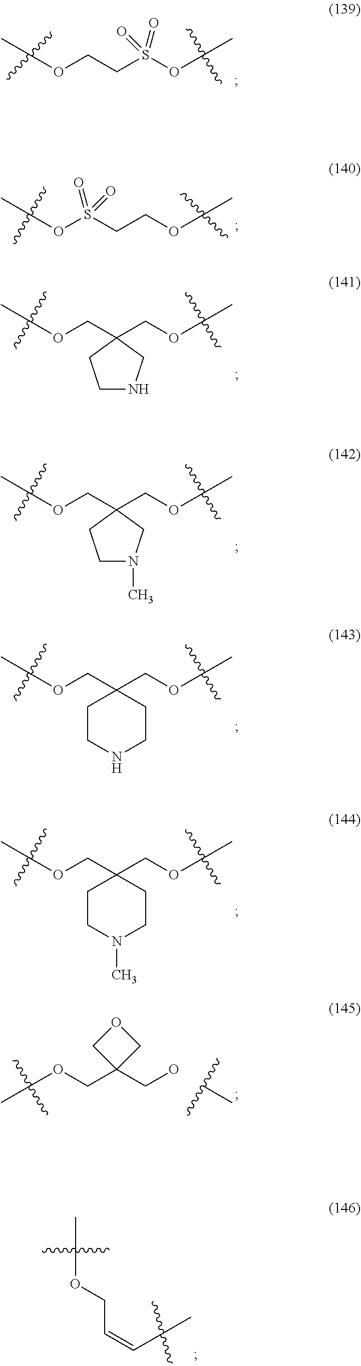

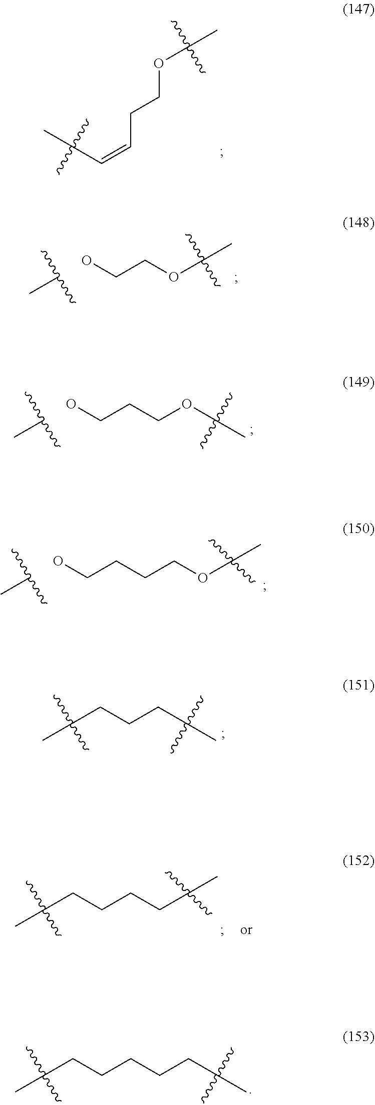

In some embodiments, each

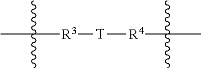

independently is:

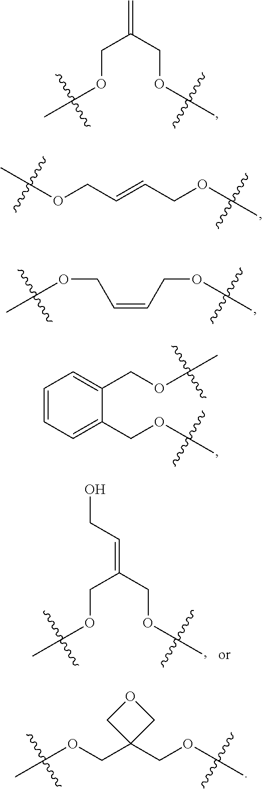

In some embodiments, when (i) X⁴ is absent, and R¹ is CN; or (ii) when X⁴ is —NR¹¹ and at least one A is —N—, each

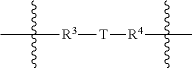

independently is:

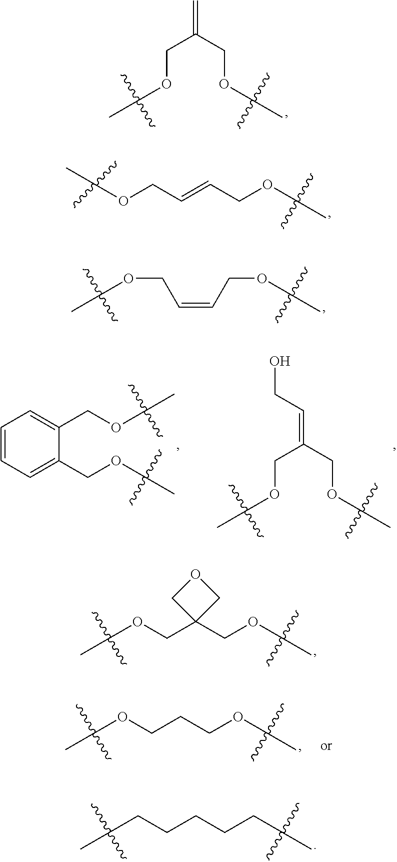

In some embodiments, at least one R⁶ is hydrogen.

In some embodiments, at least one R⁶ is C₁-C₆ alkyl optionally substituted with one or more halogen.

In some embodiments, at least one R⁶ is C₁-C₆ alkyl.

In some embodiments, each R⁶ independently is hydrogen or C₁-C₃ alkyl optionally substituted with one or more halogen.

In some embodiments, each R⁶ independently is hydrogen, CH₃, or CHF₂.

In some embodiments, each R⁶ independently is CH₃.

In some embodiments, at least one X¹ is —C(═O)—.

In some embodiments, at least one X¹ is —CH₂—, —CHF—, or —CF₂—.

In some embodiments, each X¹ independently is —C(═O)— or —CH₂—.

In some embodiments, each X¹ is —C(═O)—.

In some embodiments, each X² independently is —(C(R⁸)₂)₁₋₃—.

In some embodiments, each X² independently is —(CH₂)₁₋₃—.

In some embodiments, each X² independently is —C(R⁸)₂—.

In some embodiments, each X² independently is —CH₂—.

In some embodiments, each X² independently is —C(R⁸)₂C(R⁸)₂—.

In some embodiments, each X² independently is —CH₂CH₂—.

In some embodiments, each X² independently is —C(R⁸)₂C(R⁸)₂C(R⁸)₂—.

In some embodiments, each X² independently is —CH₂CH₂CH₂—.

In some embodiments, each X² independently is —(C(R⁸)₂)₁₋₃—, wherein at least one R⁸ is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂.

In some embodiments, each X² independently is —(C(R⁸)₂)₁₋₃—, wherein each R⁸ independently is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂.

In some embodiments, each X² independently is —(C(R⁸)₂)₁₋₃—, wherein at least two R⁸, together with the one or more atoms to which they are attached, form C₃-C₆ cycloalkyl or 3- to 6-membered heterocycloalkyl.

In some embodiments, each X² is —CH₂CHR⁸—, wherein R⁸ is hydrogen, C₁-C₃ alkyl, or C₃-C₆ cycloalkyl, wherein the C₁-C₃ alkyl is optionally substituted with one or more OH or O—(C₁-C₃ alkyl).

In some embodiments, each X² is —CH₂CHR⁸—, wherein R⁸ is hydrogen, CH₃, CH₂OH, CH₂CH₃, CH₂CH₂CH₃, CH(CH₃)₂, CH₂OCH₃, or cyclopropyl.

In some embodiments, each X² is —CHR⁸CHR⁸—, wherein each R⁸ independently: (1) is hydrogen, C₁-C₃ alkyl, or C₃-C₆ cycloalkyl, wherein the C₁-C₃ alkyl is optionally substituted with one or more OH or O—(C₁-C₃ alkyl); or (2) together with another R⁸ and the one or more atoms to which they are attached, form C₃-C₆ cycloalkyl or 3- to 6-membered heterocycloalkyl.

In some embodiments, each X² is —CHR⁸CHR⁸—, wherein each R⁸ independently: (1) is hydrogen or C₁-C₃ alkyl; or (2) together with another R⁸ and the one or more atoms to which they are attached, form C₃-C₆ cycloalkyl or 3- to 6-membered heterocycloalkyl.

In some embodiments, each X² is —CH₂C(R⁸)₂—, wherein each R⁸ independently (1) is hydrogen, C₁-C₃ alkyl, or C₃-C₆ cycloalkyl, wherein the C₁-C₃ alkyl is optionally substituted with one or more OH or O—(C₁-C₃ alkyl); or (2) together with another R⁸ and the atom to which they are attached, form C₃-C₆ cycloalkyl or 3- to 6-membered heterocycloalkyl.

In some embodiments, each X² is —CH₂C(R⁸)₂—, wherein each R⁸ independently (1) is hydrogen or C₁-C₃ alkyl; or (2) together with another R⁸ and the atom to which they are attached, form C₃-C₆ cycloalkyl or 3- to 6-membered heterocycloalkyl.

In some embodiments, each X³ independently is C(═O)OR⁶

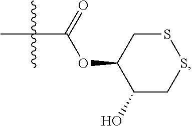 [SMILES: *C(=O)O[C@H]1CSSC[C@@H]1O]

SO₂R⁶, C(═O)N(R⁹)₂, or CN.

In some embodiments, each X³ independently is C(═O)OR⁶, C(═O)N(R⁹)₂, or CN.

In some embodiments, each X³ independently is C(═O)OH, C(═O)OCH₃, C(═O)NH₂, CN, C(═O)NH(OH), C(═O)NH—NH₂, C(═O)N(CH₃)(OH), or C(═O)NH—NH—CH₃.

In some embodiments, at least one X³ is C(═O)OR⁶.

In some embodiments, at least one X³ is C(═O)OH.

In some embodiments, each X³ is C(═O)OR⁶.

In some embodiments, each X³ is C(═O)OH.

In some embodiments, at least one X³ is C(═O)N(R⁹)₂.

In some embodiments, each X³ is C(═O)N(R⁹)₂.

In some embodiments, each X³ is C(═O)NH—OH.

In some embodiments, each X³ is C(═O)NH—OCH₃.

In some embodiments, each X³ is C(═O)NH—NH₂.

In some embodiments, each X³ is C(═O)N(CH₃)—OH.

In some embodiments, each X³ is C(═O)NH—NH—CH₃.

In some embodiments, X⁴ is absent.

In some embodiments, X⁴ is —N(R¹¹).

In some embodiments, X⁴ is —NH.

In some embodiments, X⁴ is —N(CH₃).

In some embodiments, X⁴ is —N(CH₂CH₃).

In some embodiments, each R⁹ independently is hydrogen, C(═O)OR⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆ alkyl), S(C₆-C₁₀ aryl), S(═O)₂R⁶, S(═O)₂OR⁶, P(═O)(R⁶)₂, C₁-C₆ alkyl, C₂-C₆ alkenyl, C₂-C₆ alkynyl, C₃-C₈ cycloalkyl, C₆-C₁₀ aryl, 3-8 membered heterocycloalkyl, or 3-10 membered heteroaryl.

In some embodiments, at least one R⁹ is hydrogen.

In some embodiments, each R⁹ is hydrogen.

In some embodiments, at least one R⁹ is C(═O)OR⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆ alkyl), S(C₆-C₁₀ aryl), S(═O)₂R⁶, S(═O)₂OR⁶, P(═O)(R⁶)₂, C₁-C₆ alkyl, C₂-C₆ alkenyl, C₂-C₆ alkynyl, C₃-C₅ cycloalkyl, C₆-C₁₀ aryl, 3-8 membered heterocycloalkyl, or 3-10 membered heteroaryl.

In some embodiments, at least one R⁹ is C(═O)OR⁶ or S(═O)₂R⁶.

In some embodiments, each R⁹ independently is C(═O)OR⁶ or S(═O)₂R⁶.

In some embodiments, at least one R⁹ is C(═O)OR⁶.

In some embodiments, at least one R⁹ is NH₂, NH(C₁-C₆ alkyl), or N(C₁-C₆ alkyl)₂.

In some embodiments, at least one R⁹ is (CH₂)₁₋₃—C(═O)OR⁶, OR⁶, O(C₁-C₆ alkyl), O(C₆-C₁₀ aryl) or O(C₁-C₆ alkyl)-OR⁶.

In some embodiments, at least one R⁹ is, (CH₂)₁₋₃—C(═O)OR⁶, OR⁶ or O(C₁-C₆ alkyl)-OR⁶,

In some embodiments, at least one R⁹ is OH or —OCH₃.

In some embodiments, at least one R⁹ is SR⁶, S(C₁-C₆ alkyl) or S(C₆-C₁₀ aryl),

In some embodiments, at least one R⁹ is S(═O)₂R⁶.

In some embodiments, at least one R⁹ is S(═O)₂OR⁶.

In some embodiments, at least one R⁹ is P(═O)(R⁶)₂,

In some embodiments, at least one R⁹ is C₁-C₆ alkyl, C₂-C₆ alkenyl, or C₂-C₆ alkynyl.

In some embodiments, at least one R⁹ is C₃-C₈ cycloalkyl, C₆-C₁₀ aryl, 3-8 membered heterocycloalkyl, or 3-10 membered heteroaryl.

In some embodiments, at least one R¹¹ is H.

In some embodiments, at least one R¹¹ is C₁-C₆ alkyl optionally substituted with one or more halogen.

In some embodiments, at least one R¹¹ is C₁-C₆ alkyl.

In some embodiments, at least one R¹¹ is C₁-C₆ alkyl substituted with one or more halogen.

In some embodiments, each X¹ is —C(═O)—, each X² independently is —(C(R⁸)₂)₁₋₃—, and each X³ is C(═O)OR⁶.

In some embodiments, each X¹ is —C(═O)—, each X² independently is —(CH₂)₁₋₃—, and each X³ is C(═O)OH.

In some embodiments, each X is —C(═O)—, each X² is —CH₂CH₂—, and each X³ is C(═O)OH.

In some embodiments, each X¹ is —C(═O)—, each X² independently is —(CH₂)₁₋₃—, each X³ is C(═O)OCH₃.

In some embodiments, each X¹ is —C(═O)—, each X² is —CH₂CH₂—, each X³ is C(═O)OCH₃.

In some embodiments, each X¹ is —C(═O)—, each X² independently is —(CH₂)₁₋₃—, and each X³ is C(═O)NH—OH.

In some embodiments, each X is —C(═O)—, each X² is —CH₂CH₂—, and each X³ is C(═O)NH—OH.

In some embodiments, each X¹ is —C(═O)—, each X² independently is —(CH₂)₁₋₃—, and each X³ is C(═O)NH—NH₂.

In some embodiments, each X is —C(═O)—, each X² is —CH₂CH₂—, and each X³ is C(═O)NH—NH₂. In some embodiments, each X¹ is —C(═O)—, each X² independently is —(C(R⁸)₂)₁₋₃—, each X³ is C(═O)OR⁶, and X⁴ is absent.

In some embodiments, each X¹ is —C(═O)—, each X² independently is —(CH₂)₁₋₃—, each X³ is C(═O)OH, and X⁴ is absent.

In some embodiments, each X¹ is —C(═O)—, each X² is —CH₂CH₂—, each X³ is C(═O)OH, and X⁴ is absent.

In some embodiments, each X¹ is —C(═O)—, each X² independently is —(CH₂)₁₋₃—, each X³ is C(═O)OCH₃, and X⁴ is absent.

In some embodiments, each X¹ is —C(═O)—, each X² is —CH₂CH₂—, each X³ is C(═O)OCH₃, and X⁴ is absent.

In some embodiments, each X¹ is —C(═O)—, each X² independently is —(CH₂)₁₋₃—, each X³ is C(═O)NH—OH, and X⁴ is absent.

In some embodiments, each X is —C(═O)—, each X² is —CH₂CH₂—, each X³ is C(═O)NH—OH, and X⁴ is absent.

In some embodiments, each X¹ is —C(═O)—, each X² independently is —(CH₂)₁₋₃—, each X³ is C(═O)NH—NH₂, and X⁴ is absent.

In some embodiments, each X¹ is —C(═O)—, each X² is —CH₂CH₂—, each X³ is C(═O)NH—NH₂, and X⁴ is absent.

In some embodiments, each —X¹—X²—X³ independently is —C(═O)—(C(R⁸)₂)₁₋₃—C(═O)OR⁶.

In some embodiments, each —X¹—X²—X³ independently is —C(═O)—CH₂CH₂—C(═O)OH.

In some embodiments, each —X¹—X²—X³ independently is —C(═O)—(C(R⁸)₂)₁₋₃—C(═O)N(R⁹)₂.

In some embodiments, each —X¹—X²—X³ independently is —C(═O)—CH₂CH₂—C(═O)NH—OH.

In some embodiments, each —X¹—X²—X³ independently is —C(═O)—(C(R⁸)₂)₁₋₃—C(═O)NH—NH₂.

In some embodiments, each —X¹—X⁴—X²—X³ independently is —C(═O)—N(R⁶)—(C(R⁸)₂)₁₋₃—C(═O)OR⁶.

In some embodiments, each —X¹—X⁴—X²—X³ independently is —C(═O)—NH—CH₂CH₂—C(═O)OH.

In some embodiments, each —X¹—X⁴—X²—X³ independently is —C(═O)—N(CH₃)—CH₂CH₂—C(═O)OH.

In some embodiments, each —X¹—X⁴—X²—X³ independently is —C(═O)—N(R⁶)—(C(R⁸)₂)₁₋₃—C(═O)N(R⁹)₂.

In some embodiments, each —X—X⁴—X²—X³ independently is —C(═O)—N(R⁶)—CH₂CH₂—C(═O)NH—OH.

In some embodiments, each —X—X⁴—X²—X³ independently is —C(═O)—NH—CH₂CH₂—C(═O)NH—OH.

In some embodiments, each —X—X⁴—X²—X³ independently is —C(═O)—N(CH₃)—CH₂CH₂—C(═O)NH—OH.

In some embodiments, each —X¹—X⁴—X²—X³ independently is —C(═O)—N(R⁶)—(C(R⁸)₂)₁₋₃—C(═O)NH—NH₂.

In some embodiments, the compound is of Formula (I-a), (II-a), (III-a), (IV-a), (V-a) or (VI-a):

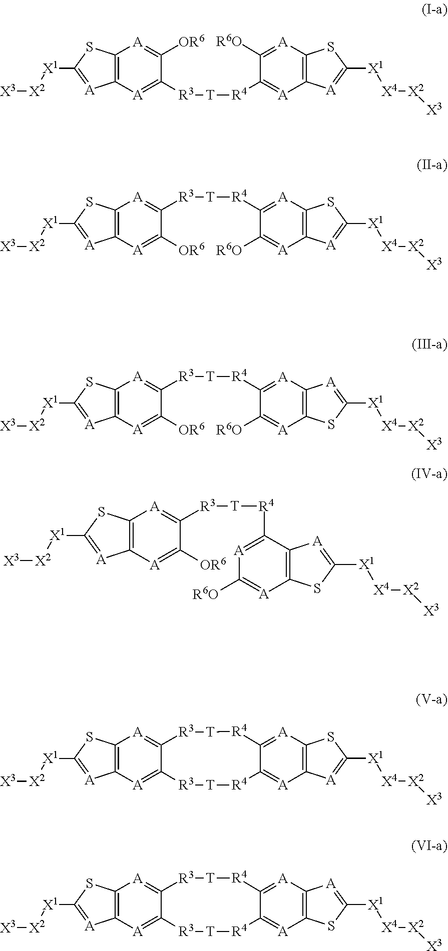

or a pharmaceutically acceptable salt thereof.

In some embodiments, the compound is of Formula (I-a) or a pharmaceutically acceptable salt thereof.

In some embodiments, the compound is of Formula (II-a) or a pharmaceutically acceptable salt thereof.

In some embodiments, the compound is of Formula (III-a) or a pharmaceutically acceptable salt thereof.

In some embodiments, the compound is of Formula (IV-a) or a pharmaceutically acceptable salt thereof.

In some embodiments, the compound is of Formula (V-a) or a pharmaceutically acceptable salt thereof.

In some embodiments, the compound is of Formula (VI-a) or a pharmaceutically acceptable salt thereof.

In some embodiments, the compound is of Formula (I-b), (II-b), (III-b), (IV-b), (V-b) or (VI-b):

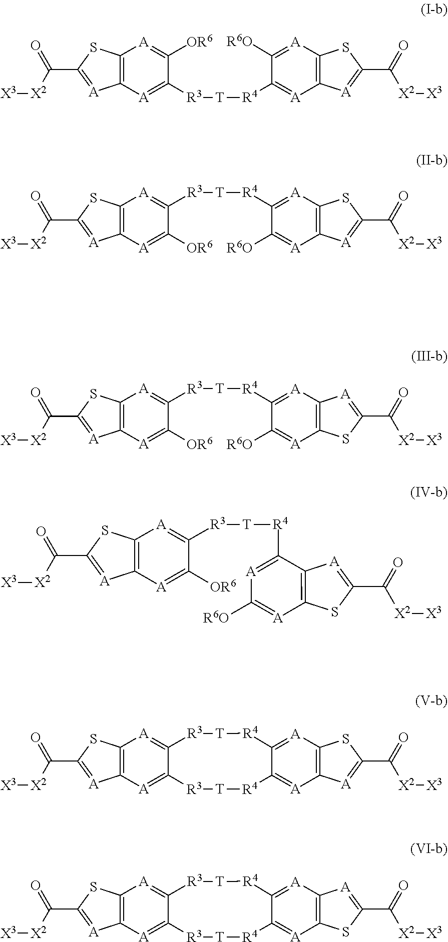

or a pharmaceutically acceptable salt thereof.

In some embodiments, the compound is of Formula (I-b) or a pharmaceutically acceptable salt thereof.

In some embodiments, the compound is of Formula (II-b) or a pharmaceutically acceptable salt thereof.

In some embodiments, the compound is of Formula (III-b) or a pharmaceutically acceptable salt thereof.

In some embodiments, the compound is of Formula (IV-b) or a pharmaceutically acceptable salt thereof.

In some embodiments, the compound is of Formula (V-b) or a pharmaceutically acceptable salt thereof.

In some embodiments, the compound is of Formula (VI-b) or a pharmaceutically acceptable salt thereof.

In some embodiments, the compound is of Formula (I-c), (II-c), (III-c), (IV-c), (V-c) or (VI-c):

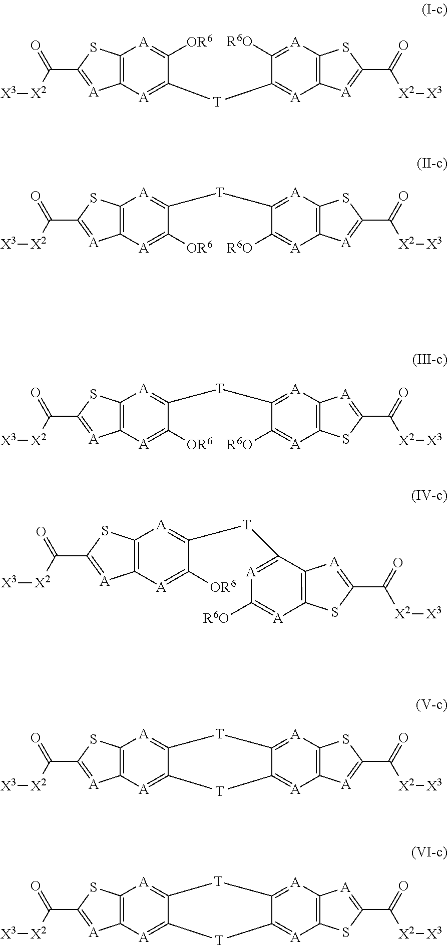

or a pharmaceutically acceptable salt thereof.

In some embodiments, the compound is of Formula (I-c) or a pharmaceutically acceptable salt thereof.

In some embodiments, the compound is of Formula (II-c) or a pharmaceutically acceptable salt thereof.

In some embodiments, the compound is of Formula (III-c) or a pharmaceutically acceptable salt thereof.

In some embodiments, the compound is of Formula (IV-c) or a pharmaceutically acceptable salt thereof.

In some embodiments, the compound is of Formula (V-c) or a pharmaceutically acceptable salt thereof.

In some embodiments, the compound is of Formula (VI-c) or a pharmaceutically acceptable salt thereof.

In some embodiments, the compound is of Formula (I-d), (II-d), (III-d), (IV-d), (V-d) or (VI-d):

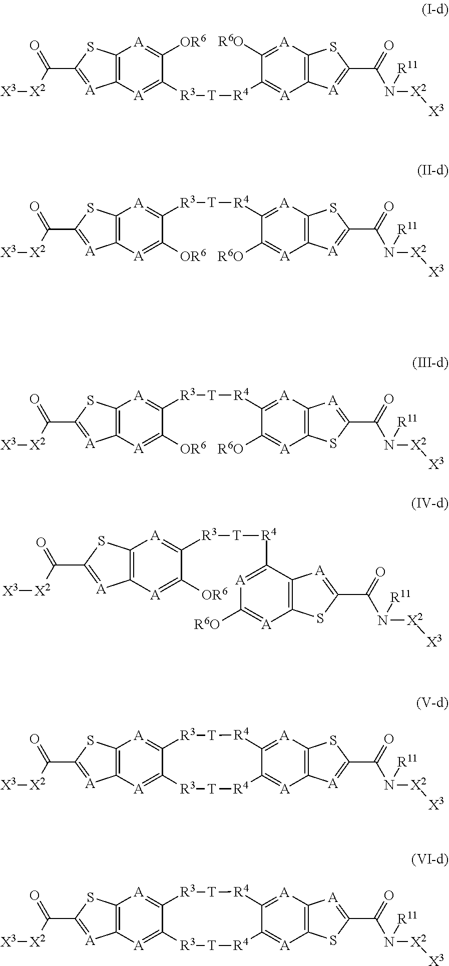

or a pharmaceutically acceptable salt thereof.

In some embodiments, the compound is of Formula (I-d) or a pharmaceutically acceptable salt thereof.

In some embodiments, the compound is of Formula (II-d) or a pharmaceutically acceptable salt thereof.

In some embodiments, the compound is of Formula (III-d) or a pharmaceutically acceptable salt thereof.

In some embodiments, the compound is of Formula (IV-d) or a pharmaceutically acceptable salt thereof.

In some embodiments, the compound is of Formula (V-d) or a pharmaceutically acceptable salt thereof.

In some embodiments, the compound is of Formula (VI-d) or a pharmaceutically acceptable salt thereof.

In some embodiments, the compound is of Formula (I-e, (II-e), (III-e), (IV-e), (V-e) or (VI-e):

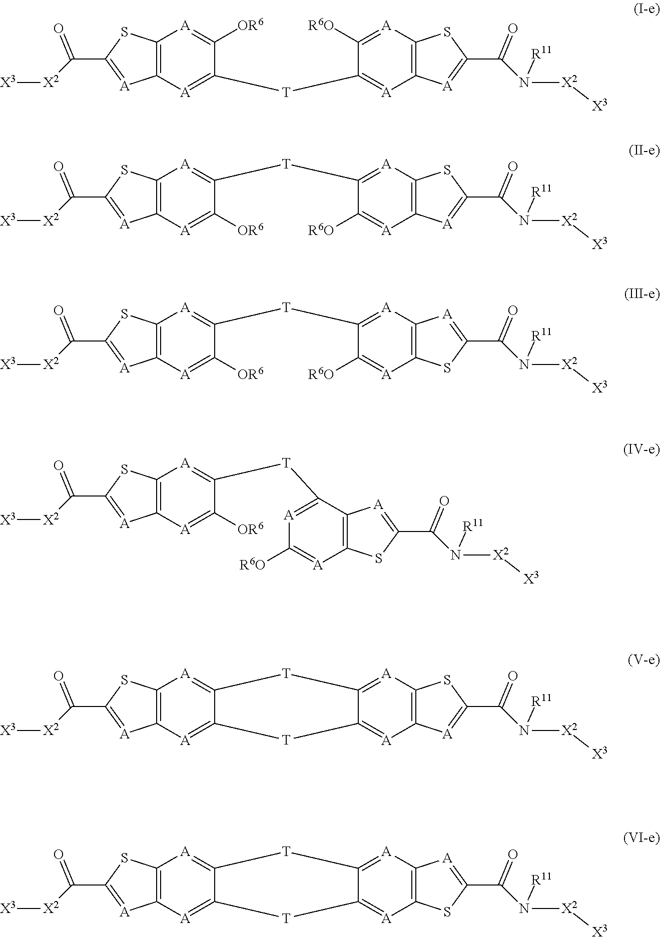

or a pharmaceutically acceptable salt thereof.

In some embodiments, the compound is of Formula (I-e) or a pharmaceutically acceptable salt thereof.

In some embodiments, the compound is of Formula (II-e) or a pharmaceutically acceptable salt thereof.

In some embodiments, the compound is of Formula (III-e) or a pharmaceutically acceptable salt thereof.

In some embodiments, the compound is of Formula (IV-e) or a pharmaceutically acceptable salt thereof.

In some embodiments, the compound is of Formula (V-e) or a pharmaceutically acceptable salt thereof.

In some embodiments, the compound is of Formula (VI-e) or a pharmaceutically acceptable salt thereof.

In some embodiments, the compound is selected from the compounds described in Table 1 and pharmaceutically acceptable salts thereof.

In some embodiments, the compound is selected from the compounds described in Table 1.

TABLE 1

| Example | Compd. | | LCMS |
|---|---|---|---|
| No. | No. | Structure | (M + H)⁺ |
| 1 | 6 | 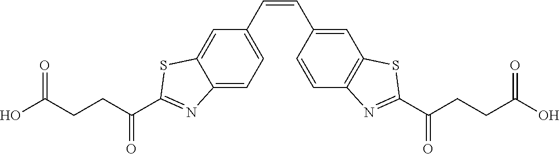 [SMILES: O=C(O)CCC(=O)c1nc2ccc(/C=C\c3ccc4nc(C(=O)CCC(=O)O)sc4c3)cc2s1] | 495.06 |
| 2 | 9 | 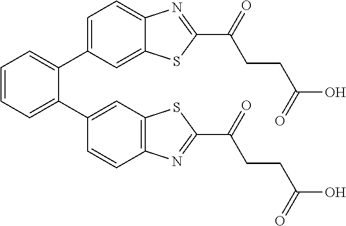 [SMILES: O=C(O)CCC(=O)c1nc2ccc(-c3ccccc3-c3ccc4nc(C(=O)CCC(=O)O)sc4c3)cc2s1] | 545.07 |
| 3 | 18 | 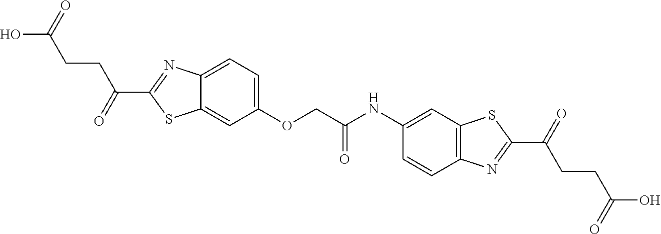 [SMILES: O=C(O)CCC(=O)c1nc2ccc(NC(=O)COc3ccc4nc(C(=O)CCC(=O)O)sc4c3)cc2s1] | 542.11 |
| | 18A | 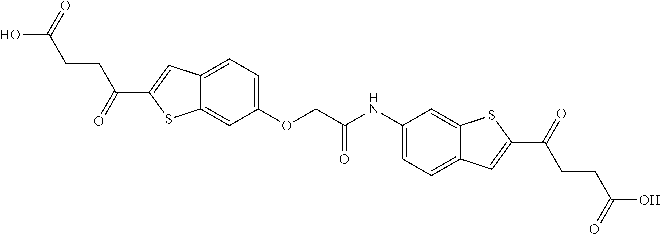 [SMILES: O=C(O)CCC(=O)c1cc2ccc(NC(=O)COc3ccc4cc(C(=O)CCC(=O)O)sc4c3)cc2s1] | |
| | 19 | 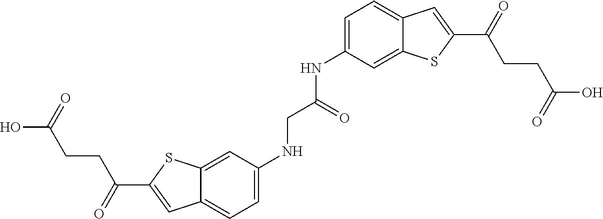 [SMILES: O=C(O)CCC(=O)c1cc2ccc(NCC(=O)Nc3ccc4cc(C(=O)CCC(=O)O)sc4c3)cc2s1] | |
| | 20 | 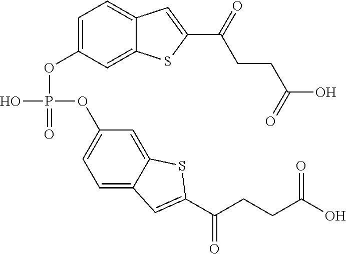 [SMILES: O=C(O)CCC(=O)c1cc2ccc(OP(=O)(O)Oc3ccc4cc(C(=O)CCC(=O)O)sc4c3)cc2s1] | |
| | 21 | 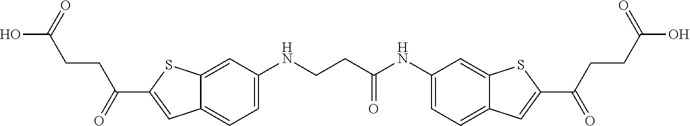 [SMILES: O=C(O)CCC(=O)c1cc2ccc(NCCC(=O)Nc3ccc4cc(C(=O)CCC(=O)O)sc4c3)cc2s1] | |
| | 22 | 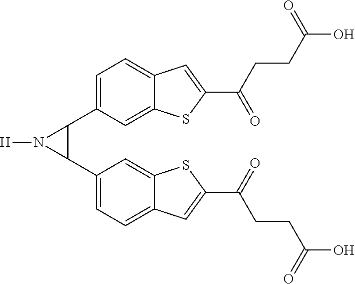 [SMILES: O=C(O)CCC(=O)c1cc2ccc(C3NC3c3ccc4cc(C(=O)CCC(=O)O)sc4c3)cc2s1] | |
| | 23 | 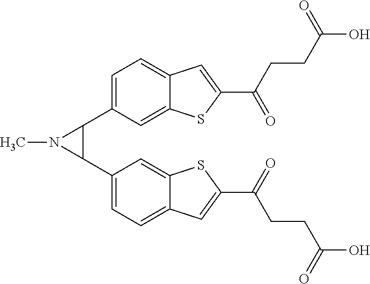 [SMILES: CN1C(c2ccc3cc(C(=O)CCC(=O)O)sc3c2)C1c1ccc2cc(C(=O)CCC(=O)O)sc2c1] | |
| 4 | 25 | 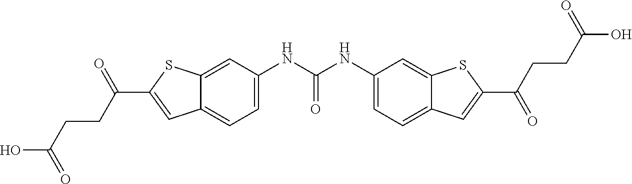 [SMILES: O=C(O)CCC(=O)c1cc2ccc(NC(=O)Nc3ccc4cc(C(=O)CCC(=O)O)sc4c3)cc2s1] | |
| | 28 | 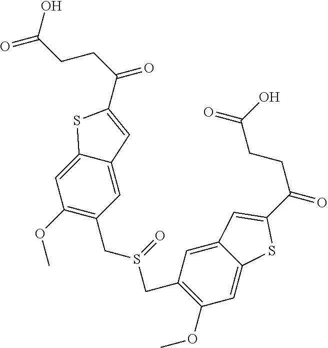 [SMILES: COc1cc2sc(C(=O)CCC(=O)O)cc2cc1CS(=O)Cc1cc2cc(C(=O)CCC(=O)O)sc2cc1OC] | |
| | 29 | 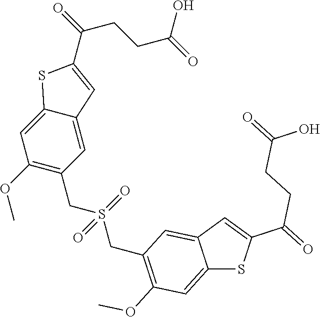 [SMILES: COc1cc2sc(C(=O)CCC(=O)O)cc2cc1CS(=O)(=O)Cc1cc2cc(C(=O)CCC(=O)O)sc2cc1OC] | |
| 30 | 30 | 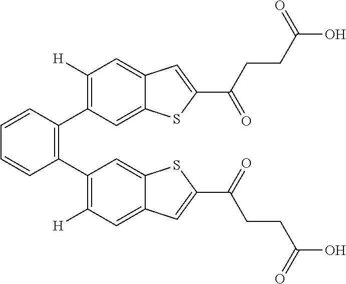 [SMILES: O=C(O)CCC(=O)c1cc2ccc(-c3ccccc3-c3ccc4cc(C(=O)CCC(=O)O)sc4c3)cc2s1] | |
| | 34 | 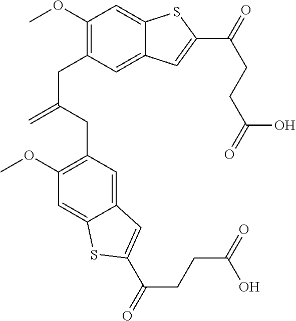 [SMILES: C=C(Cc1cc2cc(C(=O)CCC(=O)O)sc2cc1OC)Cc1cc2cc(C(=O)CCC(=O)O)sc2cc1OC] | |
| 6 | 36 | 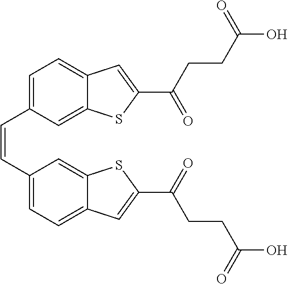 [SMILES: O=C(O)CCC(=O)c1cc2ccc(/C=C\c3ccc4cc(C(=O)CCC(=O)O)sc4c3)cc2s1] | |
| 14 | 40 | 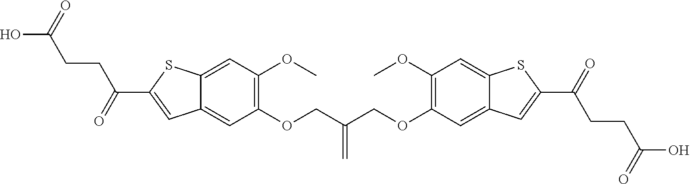 [SMILES: C=C(COc1cc2cc(C(=O)CCC(=O)O)sc2cc1OC)COc1cc2cc(C(=O)CCC(=O)O)sc2cc1OC] | 613.99 |
| | 44 | 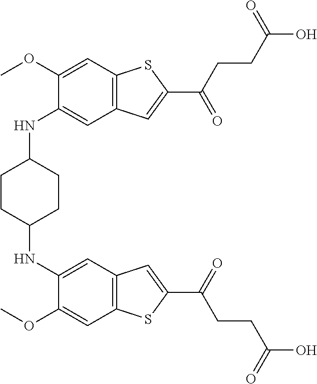 [SMILES: COc1cc2sc(C(=O)CCC(=O)O)cc2cc1NC1CCC(Nc2cc3cc(C(=O)CCC(=O)O)sc3cc2OC)CC1] | |
| | 45 | 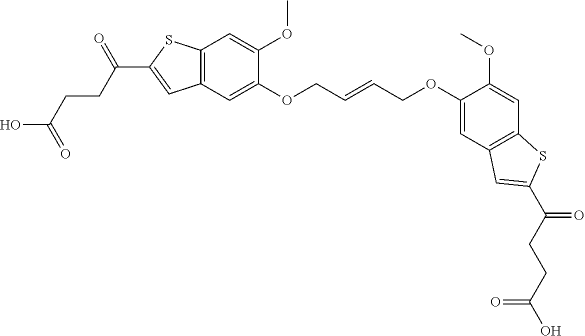 [SMILES: COc1cc2sc(C(=O)CCC(=O)O)cc2cc1OC/C=C/COc1cc2cc(C(=O)CCC(=O)O)sc2cc1OC] | |
| | 47 | 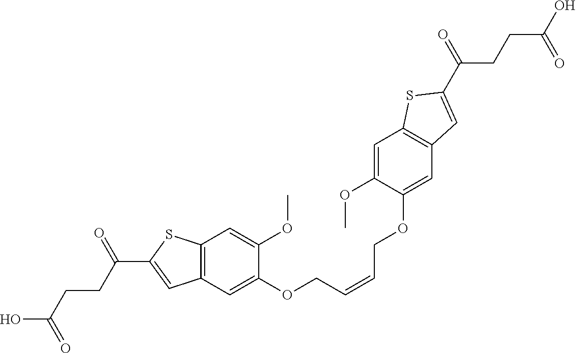 [SMILES: COc1cc2sc(C(=O)CCC(=O)O)cc2cc1OC/C=C\COc1cc2cc(C(=O)CCC(=O)O)sc2cc1OC] | |
| 7 | 48 | 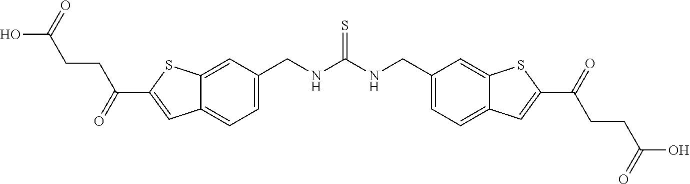 [SMILES: O=C(O)CCC(=O)c1cc2ccc(CNC(=S)NCc3ccc4cc(C(=O)CCC(=O)O)sc4c3)cc2s1] | 569.09 |
| | 48A | 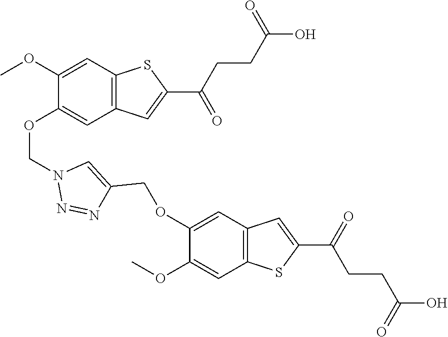 [SMILES: COc1cc2sc(C(=O)CCC(=O)O)cc2cc1OCc1cn(COc2cc3cc(C(=O)CCC(=O)O)sc3cc2OC)nn1] | |
| | 49 | 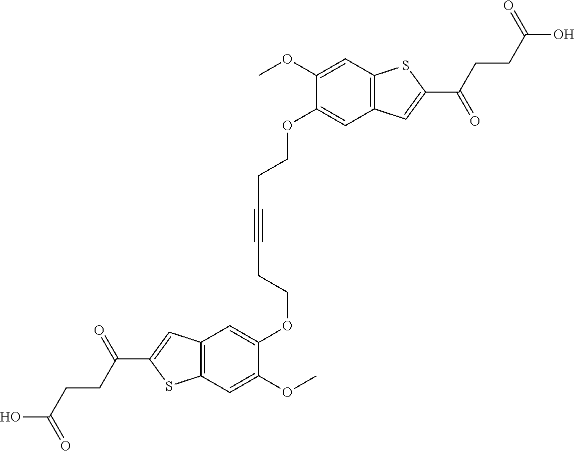 [SMILES: COc1cc2sc(C(=O)CCC(=O)O)cc2cc1OCCC#CCCOc1cc2cc(C(=O)CCC(=O)O)sc2cc1OC] | |
| | 50 | 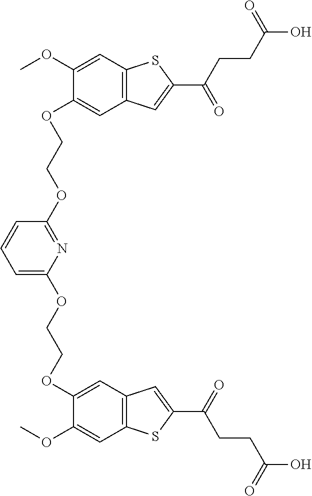 [SMILES: COc1cc2sc(C(=O)CCC(=O)O)cc2cc1OCCOc1cccc(OCCOc2cc3cc(C(=O)CCC(=O)O)sc3cc2OC)n1] | |
| | 51 | 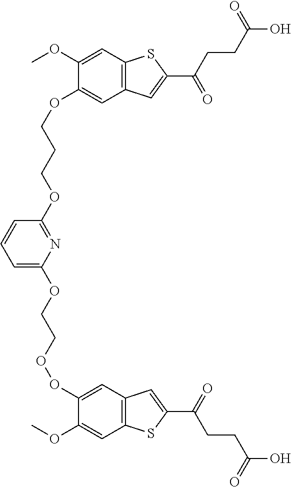 [SMILES: COc1cc2sc(C(=O)CCC(=O)O)cc2cc1OCCCOc1cccc(OCCOOc2cc3cc(C(=O)CCC(=O)O)sc3cc2OC)n1] | |
| | 52 | 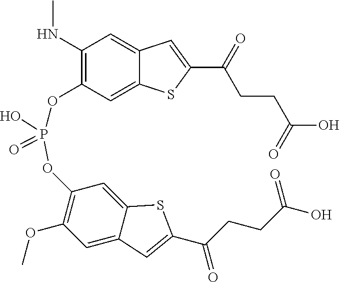 [SMILES: CNc1cc2cc(C(=O)CCC(=O)O)sc2cc1OP(=O)(O)Oc1cc2sc(C(=O)CCC(=O)O)cc2cc1OC] | |
| | 55 | 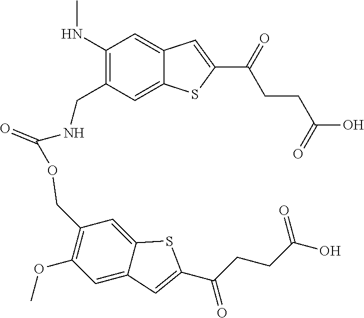 [SMILES: CNc1cc2cc(C(=O)CCC(=O)O)sc2cc1CNC(=O)OCc1cc2sc(C(=O)CCC(=O)O)cc2cc1OC] | |
| | 56 | 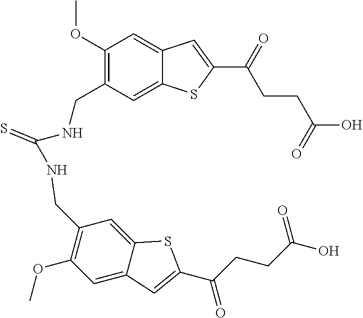 [SMILES: COc1cc2cc(C(=O)CCC(=O)O)sc2cc1CNC(=S)NCc1cc2sc(C(=O)CCC(=O)O)cc2cc1OC] | |
| | 57 | 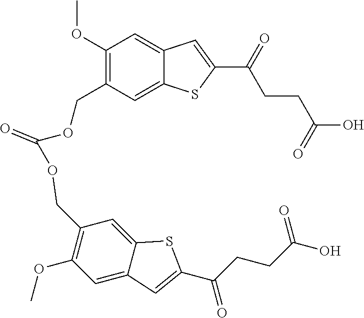 [SMILES: COc1cc2cc(C(=O)CCC(=O)O)sc2cc1COC(=O)OCc1cc2sc(C(=O)CCC(=O)O)cc2cc1OC] | |
| 8 | 58 | 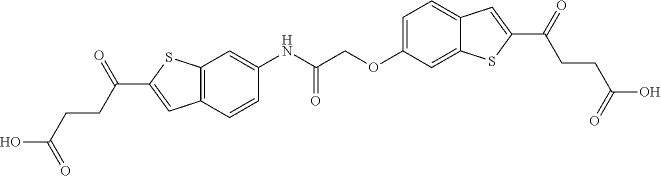 [SMILES: O=C(O)CCC(=O)c1cc2ccc(NC(=O)COc3ccc4cc(C(=O)CCC(=O)O)sc4c3)cc2s1] | |
| | 58A | 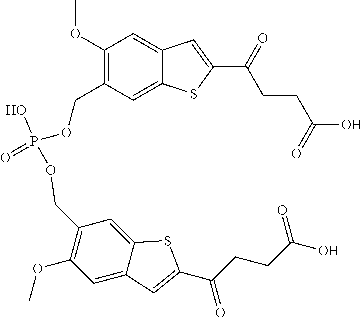 [SMILES: COc1cc2cc(C(=O)CCC(=O)O)sc2cc1COP(=O)(O)OCc1cc2sc(C(=O)CCC(=O)O)cc2cc1OC] | |
| | 59 | 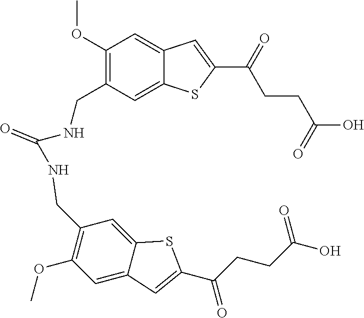 [SMILES: COc1cc2cc(C(=O)CCC(=O)O)sc2cc1CNC(=O)NCc1cc2sc(C(=O)CCC(=O)O)cc2cc1OC] | |
| | 81 | 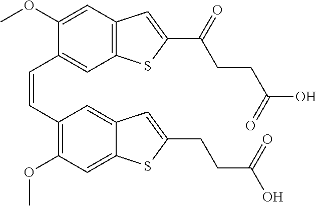 [SMILES: COc1cc2cc(C(=O)CCC(=O)O)sc2cc1/C=C\c1cc2cc(CCC(=O)O)sc2cc1OC] | |
| | 82 | 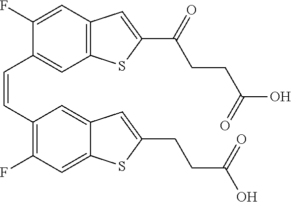 [SMILES: O=C(O)CCC(=O)c1cc2cc(F)c(/C=C\c3cc4cc(CCC(=O)O)sc4cc3F)cc2s1] | |
| | 100 | 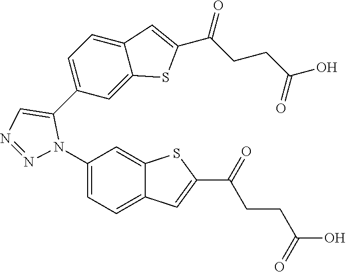 [SMILES: O=C(O)CCC(=O)c1cc2ccc(-c3cnnn3-c3ccc4cc(C(=O)CCC(=O)O)sc4c3)cc2s1] | |
| | 103 | 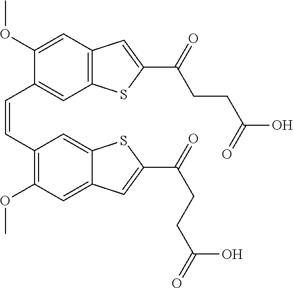 [SMILES: COc1cc2cc(C(=O)CCC(=O)O)sc2cc1/C=C\c1cc2sc(C(=O)CCC(=O)O)cc2cc1OC] | |
| | 105 | 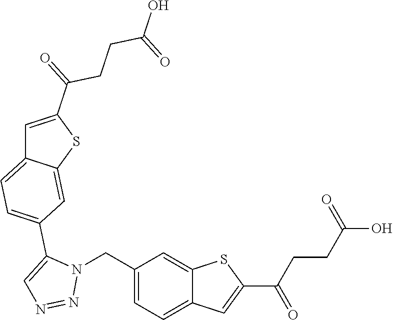 [SMILES: O=C(O)CCC(=O)c1cc2ccc(Cn3nncc3-c3ccc4cc(C(=O)CCC(=O)O)sc4c3)cc2s1] | |
| | 106 | 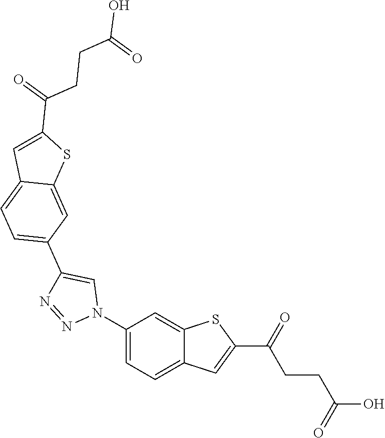 [SMILES: O=C(O)CCC(=O)c1cc2ccc(-c3cn(-c4ccc5cc(C(=O)CCC(=O)O)sc5c4)nn3)cc2s1] | |
| 8 | 107 | 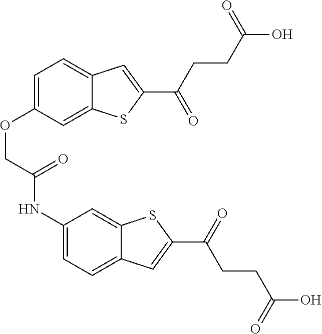 [SMILES: O=C(O)CCC(=O)c1cc2ccc(NC(=O)COc3ccc4cc(C(=O)CCC(=O)O)sc4c3)cc2s1] | 540.04 |
| | 108 | 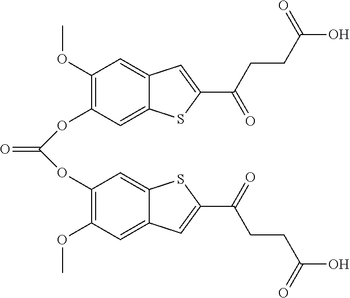 [SMILES: COc1cc2cc(C(=O)CCC(=O)O)sc2cc1OC(=O)Oc1cc2sc(C(=O)CCC(=O)O)cc2cc1OC] | |
| | 109 | 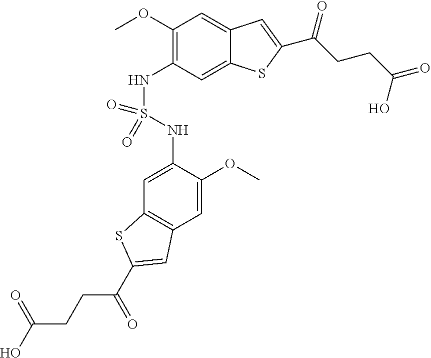 [SMILES: COc1cc2cc(C(=O)CCC(=O)O)sc2cc1NS(=O)(=O)Nc1cc2sc(C(=O)CCC(=O)O)cc2cc1OC] | |
| | 110 | 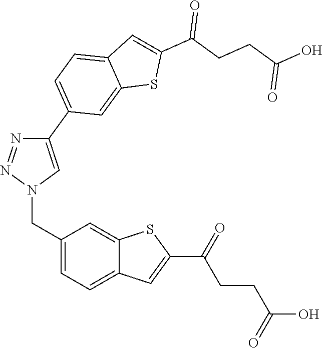 [SMILES: O=C(O)CCC(=O)c1cc2ccc(Cn3cc(-c4ccc5cc(C(=O)CCC(=O)O)sc5c4)nn3)cc2s1] | |
| | 112 | 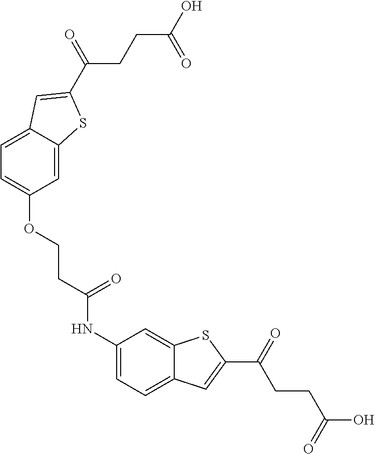 [SMILES: O=C(O)CCC(=O)c1cc2ccc(NC(=O)CCOc3ccc4cc(C(=O)CCC(=O)O)sc4c3)cc2s1] | |
| | 113 | 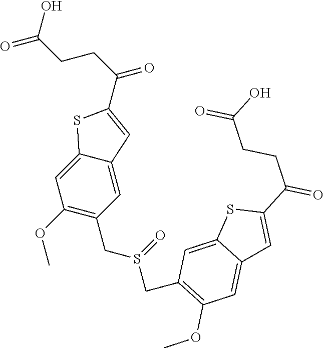 [SMILES: COc1cc2cc(C(=O)CCC(=O)O)sc2cc1CS(=O)Cc1cc2cc(C(=O)CCC(=O)O)sc2cc1OC] | |
| | 115 | 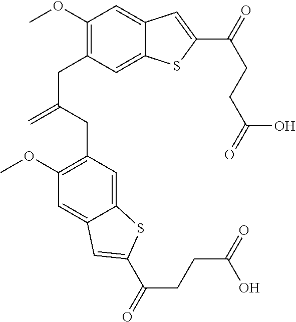 [SMILES: C=C(Cc1cc2sc(C(=O)CCC(=O)O)cc2cc1OC)Cc1cc2sc(C(=O)CCC(=O)O)cc2cc1OC] | |
| | 116 | 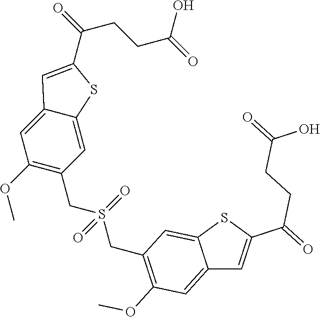 [SMILES: COc1cc2cc(C(=O)CCC(=O)O)sc2cc1CS(=O)(=O)Cc1cc2sc(C(=O)CCC(=O)O)cc2cc1OC] | |
| | 123 | 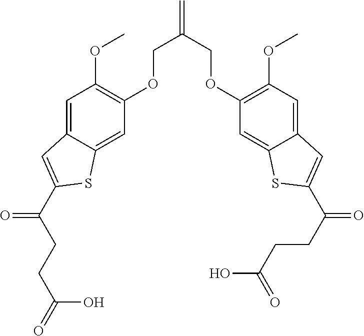 [SMILES: C=C(COc1cc2sc(C(=O)CCC(=O)O)cc2cc1OC)COc1cc2sc(C(=O)CCC(=O)O)cc2cc1OC] | |
| | 126 | 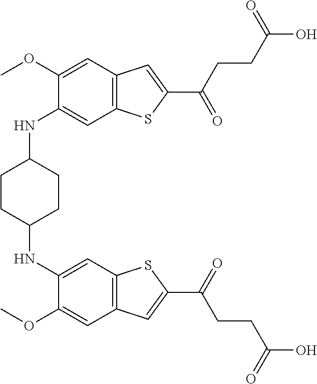 [SMILES: COc1cc2cc(C(=O)CCC(=O)O)sc2cc1NC1CCC(Nc2cc3sc(C(=O)CCC(=O)O)cc3cc2OC)CC1] | |
| | 127 | 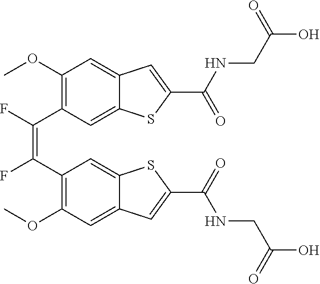 [SMILES: COc1cc2cc(C(=O)NCC(=O)O)sc2cc1/C(F)=C(/F)c1cc2sc(C(=O)NCC(=O)O)cc2cc1OC] | |
| | 128 | 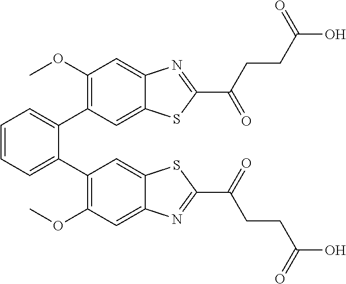 [SMILES: COc1cc2nc(C(=O)CCC(=O)O)sc2cc1-c1ccccc1-c1cc2sc(C(=O)CCC(=O)O)nc2cc1OC] | |
| | 131 | 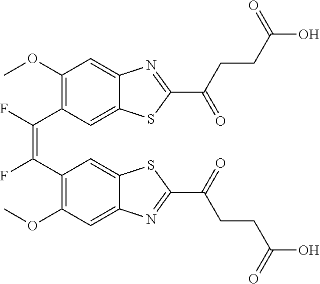 [SMILES: COc1cc2nc(C(=O)CCC(=O)O)sc2cc1/C(F)=C(/F)c1cc2sc(C(=O)CCC(=O)O)nc2cc1OC] | |
| | 132 | 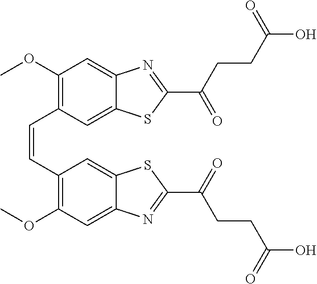 [SMILES: COc1cc2nc(C(=O)CCC(=O)O)sc2cc1/C=C\c1cc2sc(C(=O)CCC(=O)O)nc2cc1OC] | |
| | 133 | 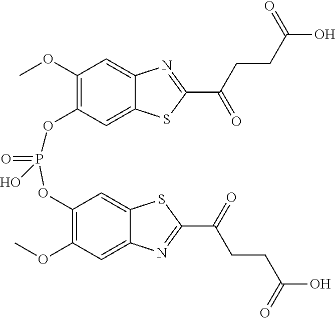 [SMILES: COc1cc2nc(C(=O)CCC(=O)O)sc2cc1OP(=O)(O)Oc1cc2sc(C(=O)CCC(=O)O)nc2cc1OC] | |
| | 134 | 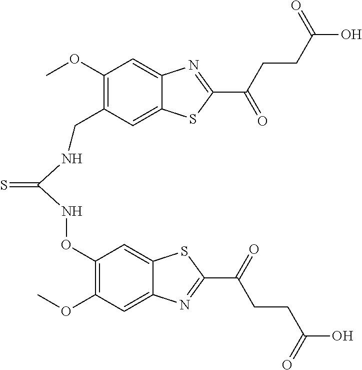 [SMILES: COc1cc2nc(C(=O)CCC(=O)O)sc2cc1CNC(=S)NOc1cc2sc(C(=O)CCC(=O)O)nc2cc1OC] | |
| | 135 | 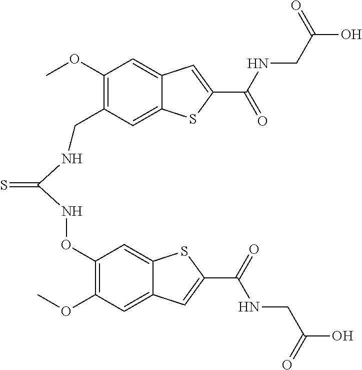 [SMILES: COc1cc2cc(C(=O)NCC(=O)O)sc2cc1CNC(=S)NOc1cc2sc(C(=O)NCC(=O)O)cc2cc1OC] | |
| | 144 | 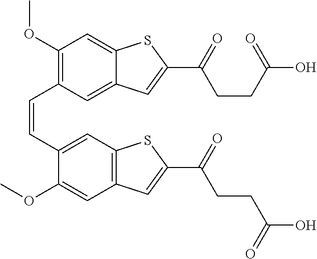 [SMILES: COc1cc2cc(C(=O)CCC(=O)O)sc2cc1/C=C\c1cc2cc(C(=O)CCC(=O)O)sc2cc1OC] | |
| | 146 | 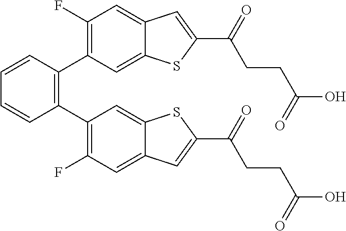 [SMILES: O=C(O)CCC(=O)c1cc2cc(F)c(-c3ccccc3-c3cc4sc(C(=O)CCC(=O)O)cc4cc3F)cc2s1] | |
| | 147 | 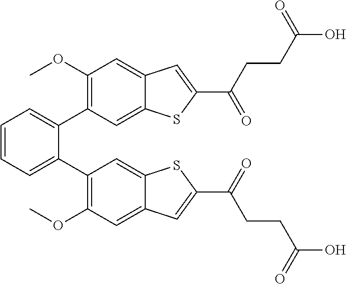 [SMILES: COc1cc2cc(C(=O)CCC(=O)O)sc2cc1-c1ccccc1-c1cc2sc(C(=O)CCC(=O)O)cc2cc1OC] | |
| | 148 | 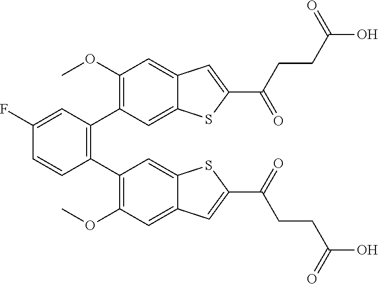 [SMILES: COc1cc2cc(C(=O)CCC(=O)O)sc2cc1-c1ccc(F)cc1-c1cc2sc(C(=O)CCC(=O)O)cc2cc1OC] | |
| | 149 | 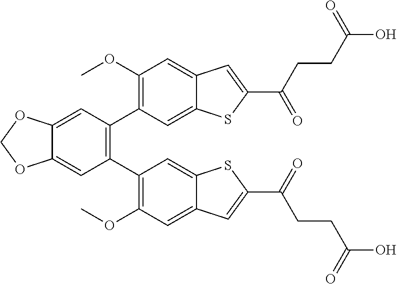 [SMILES: COc1cc2cc(C(=O)CCC(=O)O)sc2cc1-c1cc2c(cc1-c1cc3sc(C(=O)CCC(=O)O)cc3cc1OC)OCO2] | |
| | 150 | 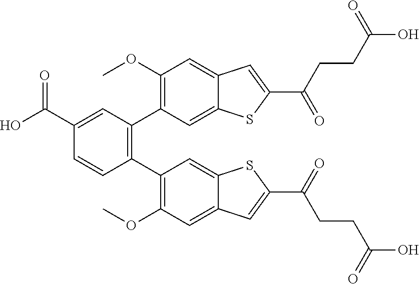 [SMILES: COc1cc2cc(C(=O)CCC(=O)O)sc2cc1-c1ccc(C(=O)O)cc1-c1cc2sc(C(=O)CCC(=O)O)cc2cc1OC] | |
| | 151 | 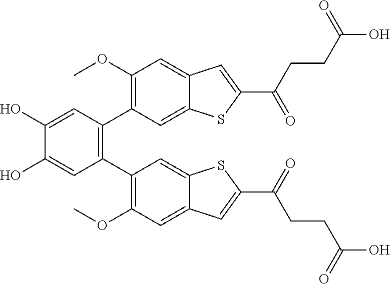 [SMILES: COc1cc2cc(C(=O)CCC(=O)O)sc2cc1-c1cc(O)c(O)cc1-c1cc2sc(C(=O)CCC(=O)O)cc2cc1OC] | |
| | 152 | 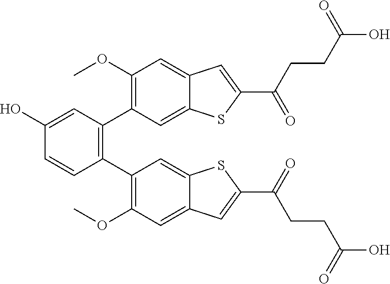 [SMILES: COc1cc2cc(C(=O)CCC(=O)O)sc2cc1-c1ccc(O)cc1-c1cc2sc(C(=O)CCC(=O)O)cc2cc1OC] | |
| | 153 | 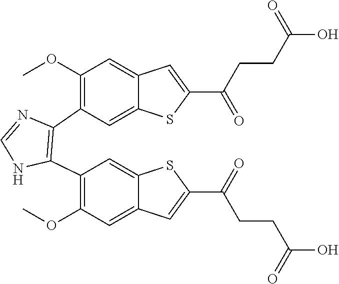 [SMILES: COc1cc2cc(C(=O)CCC(=O)O)sc2cc1-c1nc[nH]c1-c1cc2sc(C(=O)CCC(=O)O)cc2cc1OC] | |
| | 154 | 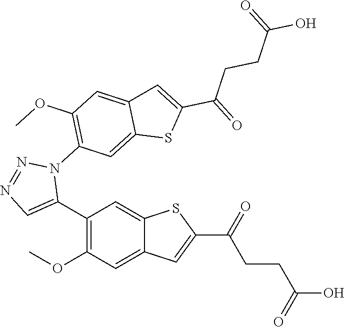 [SMILES: COc1cc2cc(C(=O)CCC(=O)O)sc2cc1-c1cnnn1-c1cc2sc(C(=O)CCC(=O)O)cc2cc1OC] | |
| | 155 | 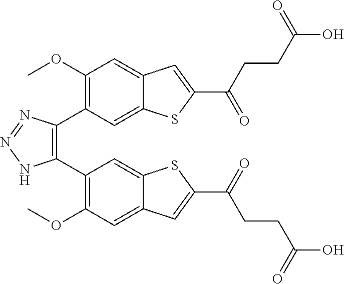 [SMILES: COc1cc2cc(C(=O)CCC(=O)O)sc2cc1-c1nn[nH]c1-c1cc2sc(C(=O)CCC(=O)O)cc2cc1OC] | |
| | 156 | 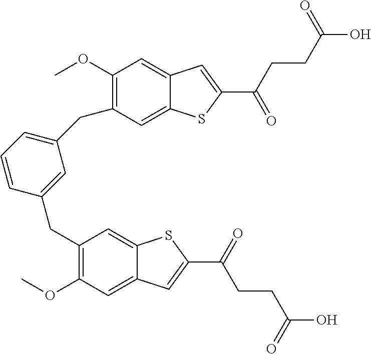 [SMILES: COc1cc2cc(C(=O)CCC(=O)O)sc2cc1Cc1cccc(Cc2cc3sc(C(=O)CCC(=O)O)cc3cc2OC)c1] | |
| | 157 | 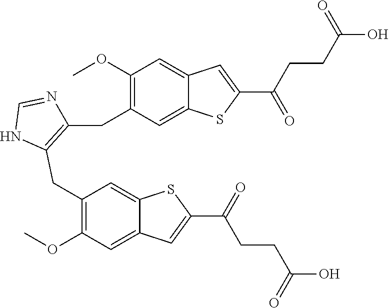 [SMILES: COc1cc2cc(C(=O)CCC(=O)O)sc2cc1Cc1nc[nH]c1Cc1cc2sc(C(=O)CCC(=O)O)cc2cc1OC] | |
| | 158 | 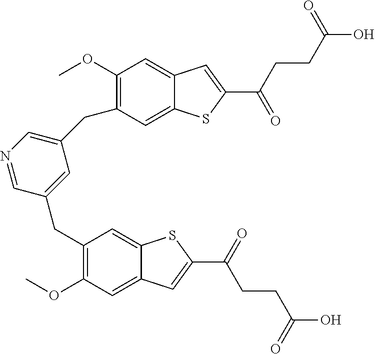 [SMILES: COc1cc2cc(C(=O)CCC(=O)O)sc2cc1Cc1cncc(Cc2cc3sc(C(=O)CCC(=O)O)cc3cc2OC)c1] | |
| | 159 | 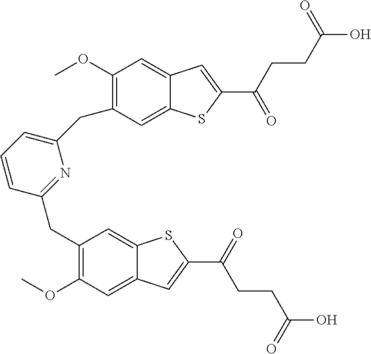 [SMILES: COc1cc2cc(C(=O)CCC(=O)O)sc2cc1Cc1cccc(Cc2cc3sc(C(=O)CCC(=O)O)cc3cc2OC)n1] | |
| | 160 | 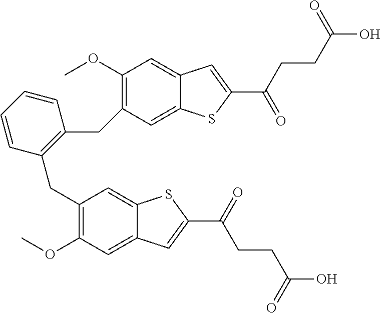 [SMILES: COc1cc2cc(C(=O)CCC(=O)O)sc2cc1Cc1ccccc1Cc1cc2sc(C(=O)CCC(=O)O)cc2cc1OC] | |
| | 165 | 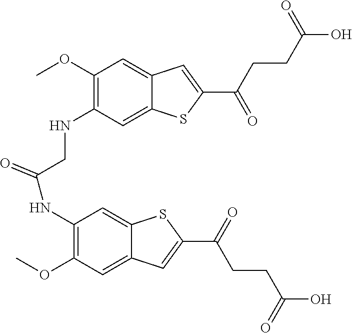 [SMILES: COc1cc2cc(C(=O)CCC(=O)O)sc2cc1NCC(=O)Nc1cc2sc(C(=O)CCC(=O)O)cc2cc1OC] | |
| | 166 | 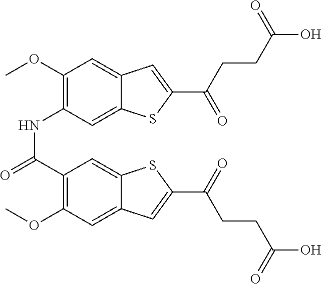 [SMILES: COc1cc2cc(C(=O)CCC(=O)O)sc2cc1NC(=O)c1cc2sc(C(=O)CCC(=O)O)cc2cc1OC] | |
| | 167 | 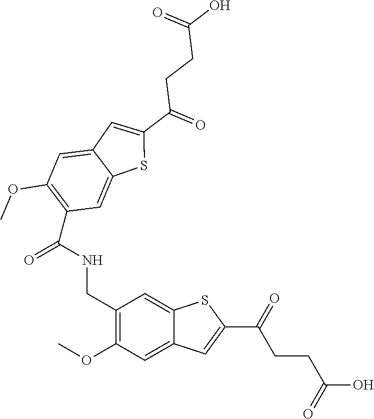 [SMILES: COc1cc2cc(C(=O)CCC(=O)O)sc2cc1CNC(=O)c1cc2sc(C(=O)CCC(=O)O)cc2cc1OC] | |
| | 168 | 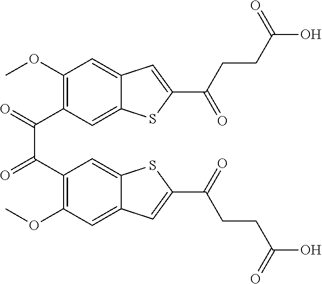 [SMILES: COc1cc2cc(C(=O)CCC(=O)O)sc2cc1C(=O)C(=O)c1cc2sc(C(=O)CCC(=O)O)cc2cc1OC] | |
| | 170 | 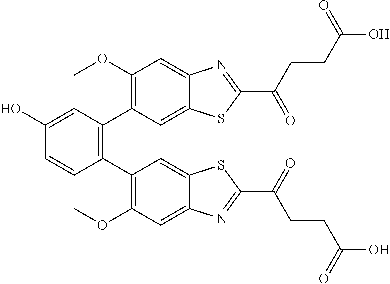 [SMILES: COc1cc2nc(C(=O)CCC(=O)O)sc2cc1-c1ccc(O)cc1-c1cc2sc(C(=O)CCC(=O)O)nc2cc1OC] | |
| | 171 | 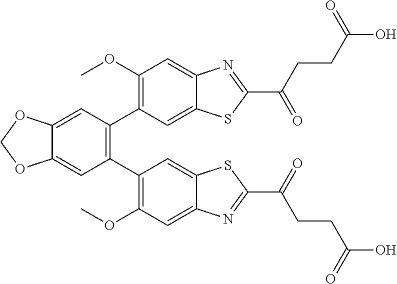 [SMILES: COc1cc2nc(C(=O)CCC(=O)O)sc2cc1-c1cc2c(cc1-c1cc3sc(C(=O)CCC(=O)O)nc3cc1OC)OCO2] | |
| | 172 | 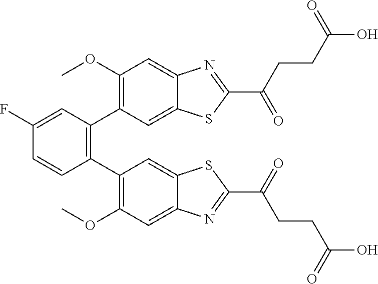 [SMILES: COc1cc2nc(C(=O)CCC(=O)O)sc2cc1-c1ccc(F)cc1-c1cc2sc(C(=O)CCC(=O)O)nc2cc1OC] | |
| | 173 | 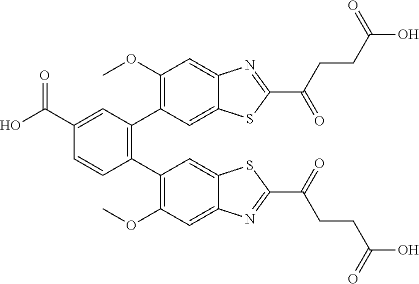 [SMILES: COc1cc2nc(C(=O)CCC(=O)O)sc2cc1-c1ccc(C(=O)O)cc1-c1cc2sc(C(=O)CCC(=O)O)nc2cc1OC] | |
| | 174 | 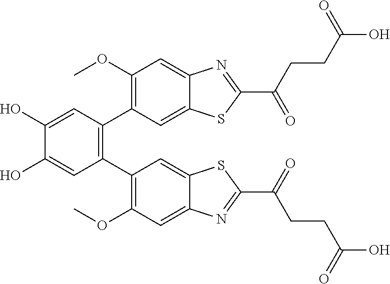 [SMILES: COc1cc2nc(C(=O)CCC(=O)O)sc2cc1-c1cc(O)c(O)cc1-c1cc2sc(C(=O)CCC(=O)O)nc2cc1OC] | |
| | 175 | 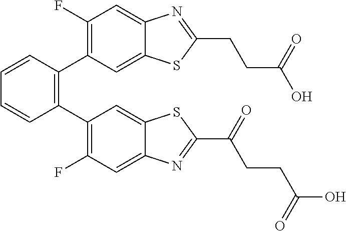 [SMILES: O=C(O)CCC(=O)c1nc2cc(F)c(-c3ccccc3-c3cc4sc(CCC(=O)O)nc4cc3F)cc2s1] | |
| | 176 | 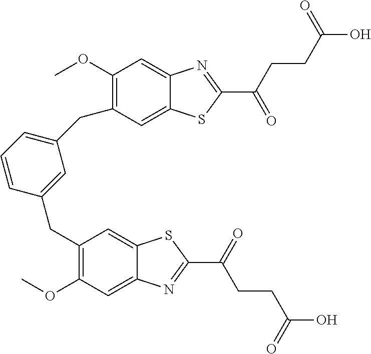 [SMILES: COc1cc2nc(C(=O)CCC(=O)O)sc2cc1Cc1cccc(Cc2cc3sc(C(=O)CCC(=O)O)nc3cc2OC)c1] | |
| | 177 | 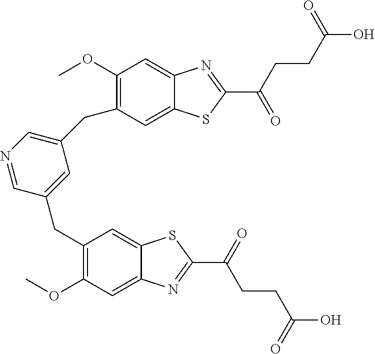 [SMILES: COc1cc2nc(C(=O)CCC(=O)O)sc2cc1Cc1cncc(Cc2cc3sc(C(=O)CCC(=O)O)nc3cc2OC)c1] | |
| | 178 | 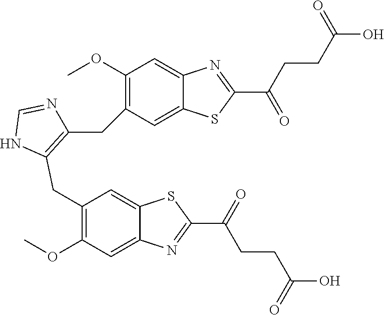 [SMILES: COc1cc2nc(C(=O)CCC(=O)O)sc2cc1Cc1nc[nH]c1Cc1cc2sc(C(=O)CCC(=O)O)nc2cc1OC] | |
| | 179 | 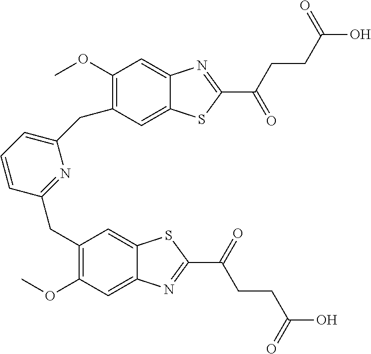 [SMILES: COc1cc2nc(C(=O)CCC(=O)O)sc2cc1Cc1cccc(Cc2cc3sc(C(=O)CCC(=O)O)nc3cc2OC)n1] | |
| | 180 | 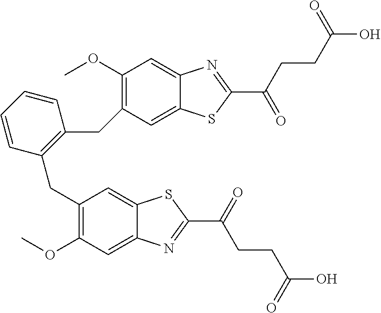 [SMILES: COc1cc2nc(C(=O)CCC(=O)O)sc2cc1Cc1ccccc1Cc1cc2sc(C(=O)CCC(=O)O)nc2cc1OC] | |
| | 181 | 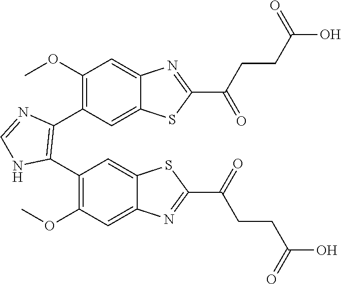 [SMILES: COc1cc2nc(C(=O)CCC(=O)O)sc2cc1-c1nc[nH]c1-c1cc2sc(C(=O)CCC(=O)O)nc2cc1OC] | |
| | 182 | 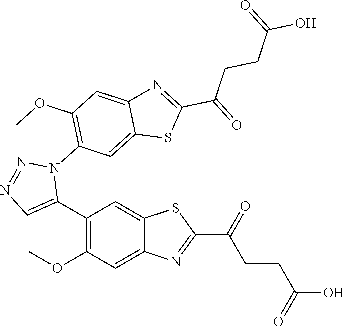 [SMILES: COc1cc2nc(C(=O)CCC(=O)O)sc2cc1-c1cnnn1-c1cc2sc(C(=O)CCC(=O)O)nc2cc1OC] | |
| | 183 | 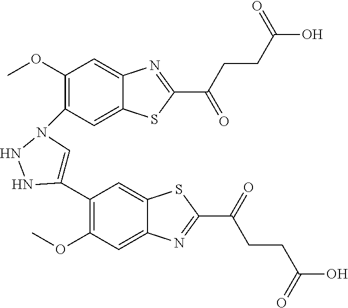 [SMILES: COc1cc2nc(C(=O)CCC(=O)O)sc2cc1C1=CN(c2cc3sc(C(=O)CCC(=O)O)nc3cc2OC)NN1] | |
| | 184 | 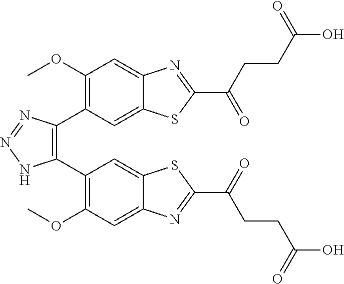 [SMILES: COc1cc2nc(C(=O)CCC(=O)O)sc2cc1-c1nn[nH]c1-c1cc2sc(C(=O)CCC(=O)O)nc2cc1OC] | |
| | 185 | 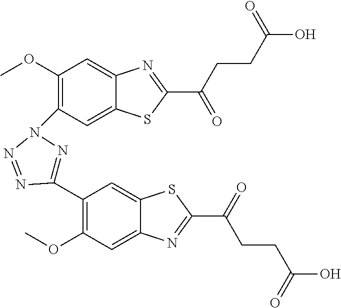 [SMILES: COc1cc2nc(C(=O)CCC(=O)O)sc2cc1-c1nnn(-c2cc3sc(C(=O)CCC(=O)O)nc3cc2OC)n1] | |
| | 188 | 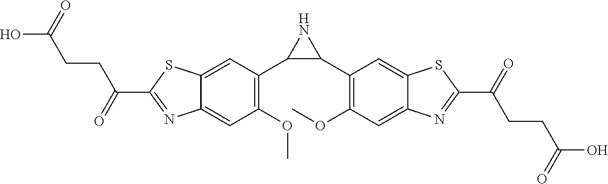 [SMILES: COc1cc2nc(C(=O)CCC(=O)O)sc2cc1C1NC1c1cc2sc(C(=O)CCC(=O)O)nc2cc1OC] | |
| | 189 | 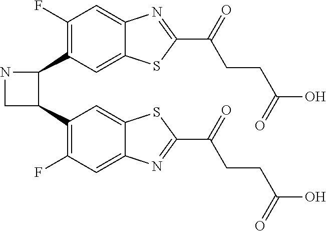 [SMILES: O=C(O)CCC(=O)c1nc2cc(F)c([C@H]3CN[C@H]3c3cc4sc(C(=O)CCC(=O)O)nc4cc3F)cc2s1] | |
| | 190 | 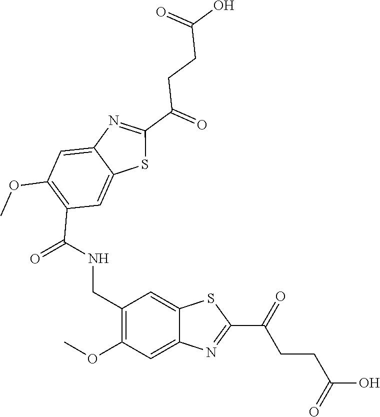 [SMILES: COc1cc2nc(C(=O)CCC(=O)O)sc2cc1CNC(=O)c1cc2sc(C(=O)CCC(=O)O)nc2cc1OC] | |
| | 191 | 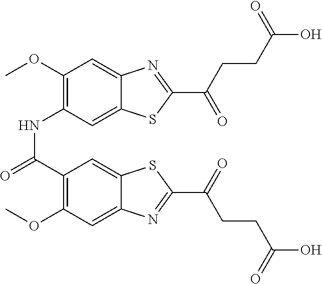 [SMILES: COc1cc2nc(C(=O)CCC(=O)O)sc2cc1NC(=O)c1cc2sc(C(=O)CCC(=O)O)nc2cc1OC] | |
| | 192 | 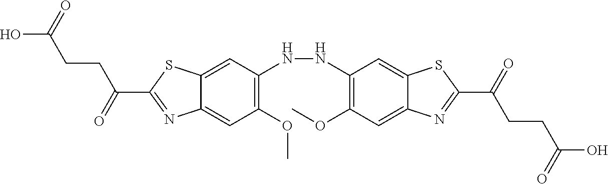 [SMILES: COc1cc2nc(C(=O)CCC(=O)O)sc2cc1NNc1cc2sc(C(=O)CCC(=O)O)nc2cc1OC] | |
| | 194 | 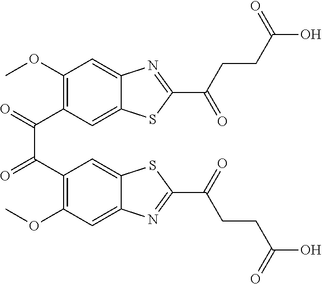 [SMILES: COc1cc2nc(C(=O)CCC(=O)O)sc2cc1C(=O)C(=O)c1cc2sc(C(=O)CCC(=O)O)nc2cc1OC] | |
| | 196 | 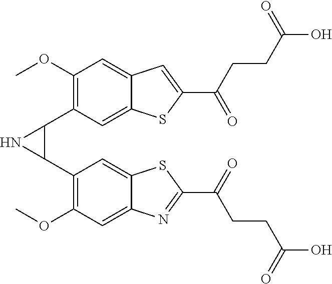 [SMILES: COc1cc2cc(C(=O)CCC(=O)O)sc2cc1C1NC1c1cc2sc(C(=O)CCC(=O)O)nc2cc1OC] | |
| | 199 | 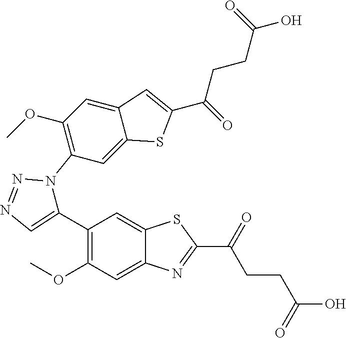 [SMILES: COc1cc2nc(C(=O)CCC(=O)O)sc2cc1-c1cnnn1-c1cc2sc(C(=O)CCC(=O)O)cc2cc1OC] | |
| | 200 | 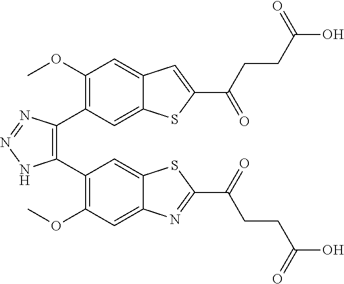 [SMILES: COc1cc2cc(C(=O)CCC(=O)O)sc2cc1-c1nn[nH]c1-c1cc2sc(C(=O)CCC(=O)O)nc2cc1OC] | |
| | 201 | 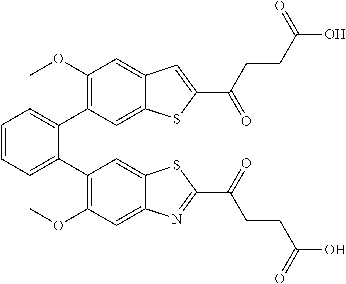 [SMILES: COc1cc2cc(C(=O)CCC(=O)O)sc2cc1-c1ccccc1-c1cc2sc(C(=O)CCC(=O)O)nc2cc1OC] | |
| 9 | 202 | 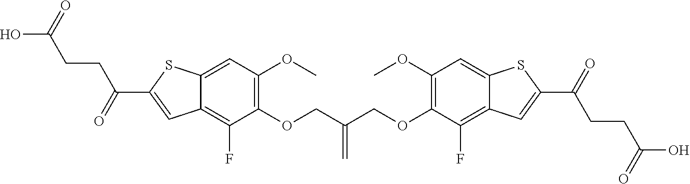 [SMILES: C=C(COc1c(OC)cc2sc(C(=O)CCC(=O)O)cc2c1F)COc1c(OC)cc2sc(C(=O)CCC(=O)O)cc2c1F] | 648.97 |
| 10 | 203 | 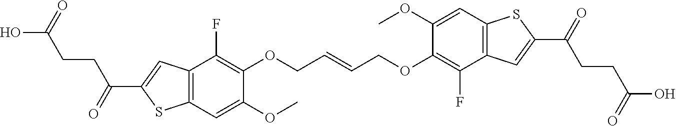 [SMILES: COc1cc2sc(C(=O)CCC(=O)O)cc2c(F)c1OC/C=C/COc1c(OC)cc2sc(C(=O)CCC(=O)O)cc2c1F] | 648.97 |
| 11 | 204 | 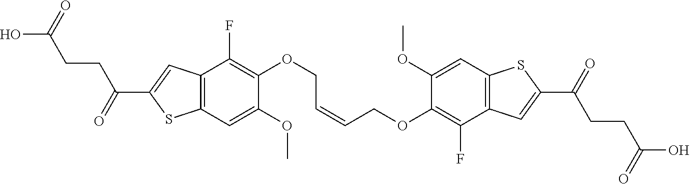 [SMILES: COc1cc2sc(C(=O)CCC(=O)O)cc2c(F)c1OC/C=C\COc1c(OC)cc2sc(C(=O)CCC(=O)O)cc2c1F] | 648.97 |
| | 205 | 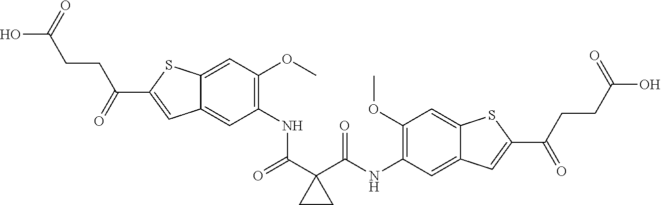 [SMILES: COc1cc2sc(C(=O)CCC(=O)O)cc2cc1NC(=O)C1(C(=O)Nc2cc3cc(C(=O)CCC(=O)O)sc3cc2OC)CC1] | |
| | 206 | 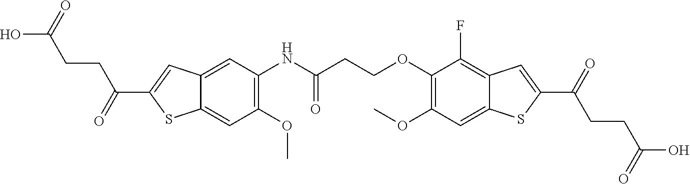 [SMILES: COc1cc2sc(C(=O)CCC(=O)O)cc2cc1NC(=O)CCOc1c(OC)cc2sc(C(=O)CCC(=O)O)cc2c1F] | |
| 15 | 207 | 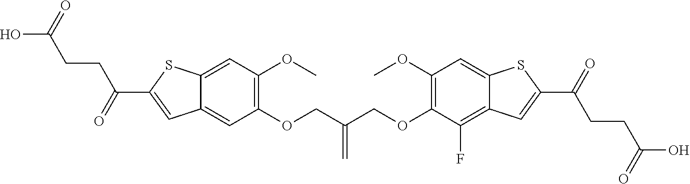 [SMILES: C=C(COc1cc2cc(C(=O)CCC(=O)O)sc2cc1OC)COc1c(OC)cc2sc(C(=O)CCC(=O)O)cc2c1F] | 630.98 |
| | 208 | 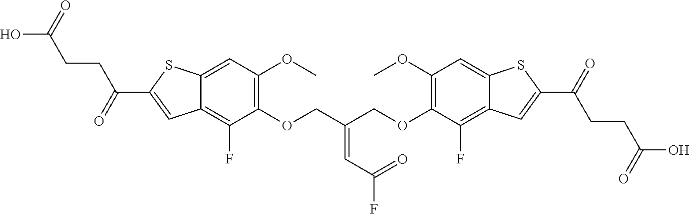 [SMILES: COc1cc2sc(C(=O)CCC(=O)O)cc2c(F)c1OCC(=CC(=O)F)COc1c(OC)cc2sc(C(=O)CCC(=O)O)cc2c1F] | 689.96 |
| | 209 | 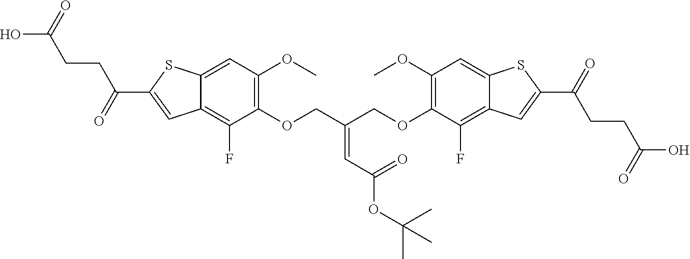 [SMILES: COc1cc2sc(C(=O)CCC(=O)O)cc2c(F)c1OCC(=CC(=O)OC(C)(C)C)COc1c(OC)cc2sc(C(=O)CCC(=O)O)cc2c1F] | 746.98 |
| 12 | 210 | 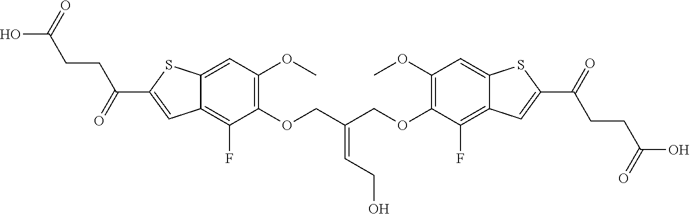 [SMILES: COc1cc2sc(C(=O)CCC(=O)O)cc2c(F)c1OCC(=CCO)COc1c(OC)cc2sc(C(=O)CCC(=O)O)cc2c1F] | 676.95 |
| 13 | 211 | 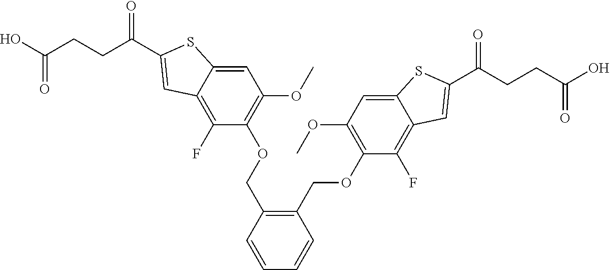 [SMILES: COc1cc2sc(C(=O)CCC(=O)O)cc2c(F)c1OCc1ccccc1COc1c(OC)cc2sc(C(=O)CCC(=O)O)cc2c1F] | 698.97 |
| | 212 | 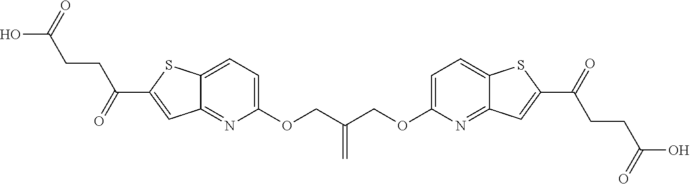 [SMILES: C=C(COc1ccc2sc(C(=O)CCC(=O)O)cc2n1)COc1ccc2sc(C(=O)CCC(=O)O)cc2n1] | |
| 16 | 213 | 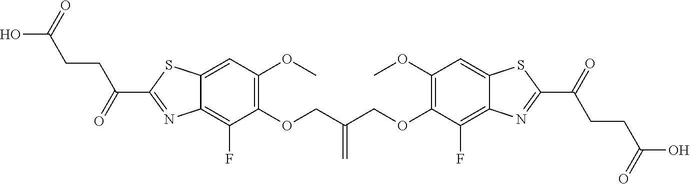 [SMILES: C=C(COc1c(OC)cc2sc(C(=O)CCC(=O)O)nc2c1F)COc1c(OC)cc2sc(C(=O)CCC(=O)O)nc2c1F] | 651.07 |
| | 214 | 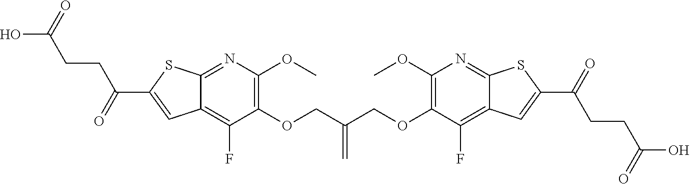 [SMILES: C=C(COc1c(OC)nc2sc(C(=O)CCC(=O)O)cc2c1F)COc1c(OC)nc2sc(C(=O)CCC(=O)O)cc2c1F] | |
| | 215 | 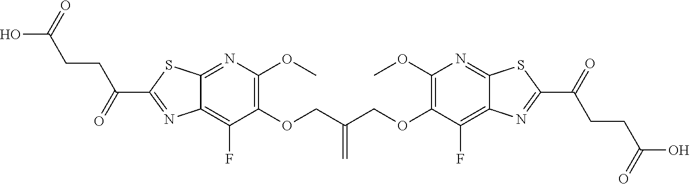 [SMILES: C=C(COc1c(OC)nc2sc(C(=O)CCC(=O)O)nc2c1F)COc1c(OC)nc2sc(C(=O)CCC(=O)O)nc2c1F] | |
| | 216 | 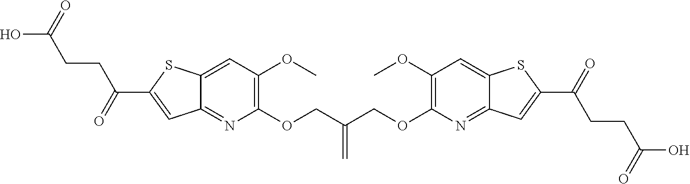 [SMILES: C=C(COc1nc2cc(C(=O)CCC(=O)O)sc2cc1OC)COc1nc2cc(C(=O)CCC(=O)O)sc2cc1OC] | |
| | 217 | 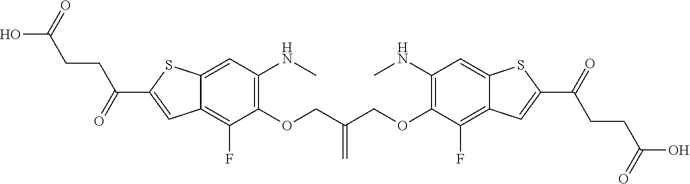 [SMILES: C=C(COc1c(NC)cc2sc(C(=O)CCC(=O)O)cc2c1F)COc1c(NC)cc2sc(C(=O)CCC(=O)O)cc2c1F] | |
| | 218 | 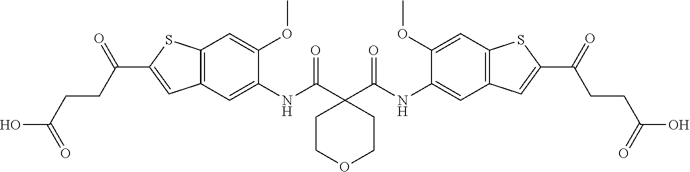 [SMILES: COc1cc2sc(C(=O)CCC(=O)O)cc2cc1NC(=O)C1(C(=O)Nc2cc3cc(C(=O)CCC(=O)O)sc3cc2OC)CCOCC1] | |
| | 219 | 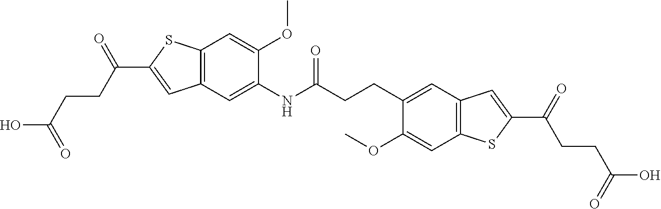 [SMILES: COc1cc2sc(C(=O)CCC(=O)O)cc2cc1CCC(=O)Nc1cc2cc(C(=O)CCC(=O)O)sc2cc1OC] | |
| | 220 | 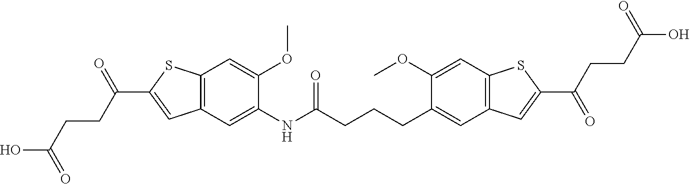 [SMILES: COc1cc2sc(C(=O)CCC(=O)O)cc2cc1CCCC(=O)Nc1cc2cc(C(=O)CCC(=O)O)sc2cc1OC] | |
| | 221 | 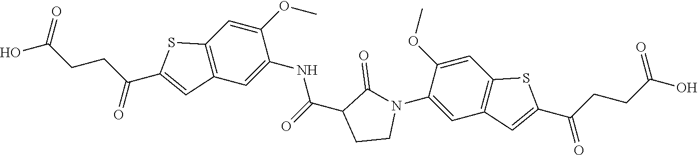 [SMILES: COc1cc2sc(C(=O)CCC(=O)O)cc2cc1NC(=O)C1CCN(c2cc3cc(C(=O)CCC(=O)O)sc3cc2OC)C1=O] | |
| | 222 | 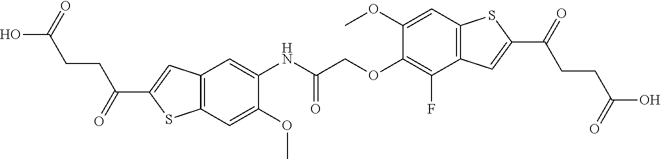 [SMILES: COc1cc2sc(C(=O)CCC(=O)O)cc2cc1NC(=O)COc1c(OC)cc2sc(C(=O)CCC(=O)O)cc2c1F] | |
| | 223 | 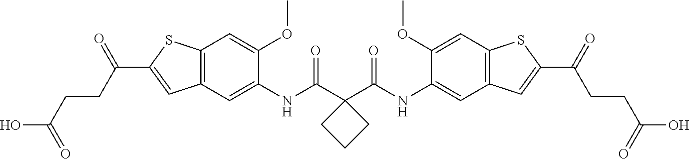 [SMILES: COc1cc2sc(C(=O)CCC(=O)O)cc2cc1NC(=O)C1(C(=O)Nc2cc3cc(C(=O)CCC(=O)O)sc3cc2OC)CCC1] | |
| | 224 | 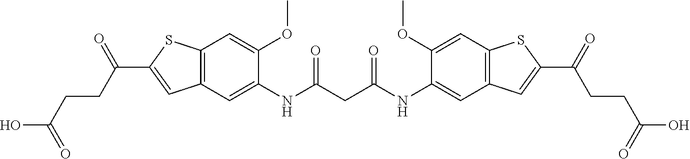 [SMILES: COc1cc2sc(C(=O)CCC(=O)O)cc2cc1NC(=O)CC(=O)Nc1cc2cc(C(=O)CCC(=O)O)sc2cc1OC] | |
| | 225 | 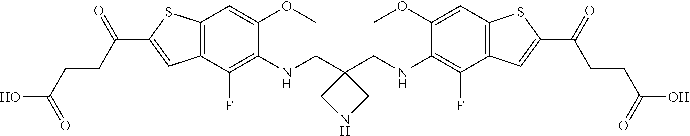 [SMILES: COc1cc2sc(C(=O)CCC(=O)O)cc2c(F)c1NCC1(CNc2c(OC)cc3sc(C(=O)CCC(=O)O)cc3c2F)CNC1] | |
| | 226 | 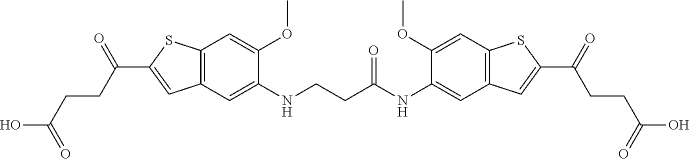 [SMILES: COc1cc2sc(C(=O)CCC(=O)O)cc2cc1NCCC(=O)Nc1cc2cc(C(=O)CCC(=O)O)sc2cc1OC] | |
| | 227 | 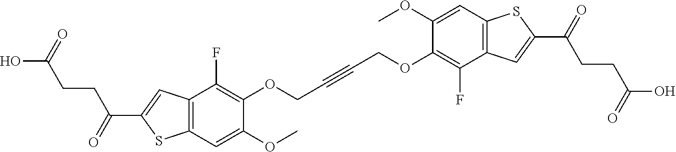 [SMILES: COc1cc2sc(C(=O)CCC(=O)O)cc2c(F)c1OCC#CCOc1c(OC)cc2sc(C(=O)CCC(=O)O)cc2c1F] | |
| | 228 | 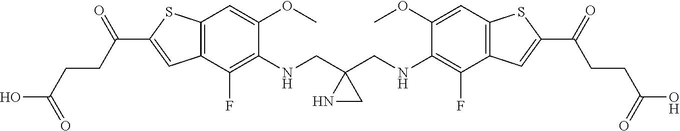 [SMILES: COc1cc2sc(C(=O)CCC(=O)O)cc2c(F)c1NCC1(CNc2c(OC)cc3sc(C(=O)CCC(=O)O)cc3c2F)CN1] | |
| | 229 | 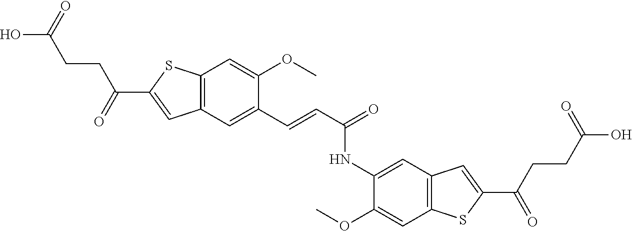 [SMILES: COc1cc2sc(C(=O)CCC(=O)O)cc2cc1/C=C/C(=O)Nc1cc2cc(C(=O)CCC(=O)O)sc2cc1OC] | |
| | 231 | 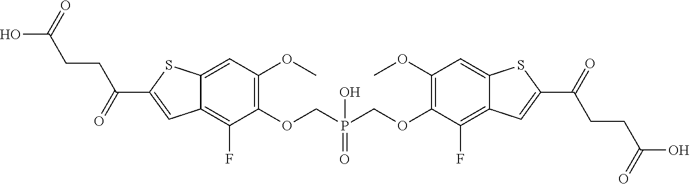 [SMILES: COc1cc2sc(C(=O)CCC(=O)O)cc2c(F)c1OCP(=O)(O)COc1c(OC)cc2sc(C(=O)CCC(=O)O)cc2c1F] | |
| | 232 | 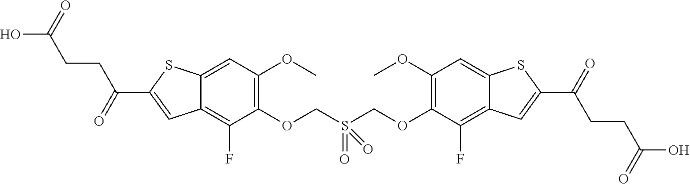 [SMILES: COc1cc2sc(C(=O)CCC(=O)O)cc2c(F)c1OCS(=O)(=O)COc1c(OC)cc2sc(C(=O)CCC(=O)O)cc2c1F] | |
| | 233 | 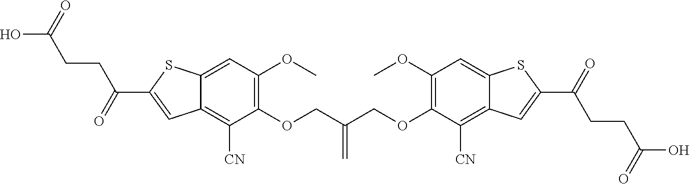 [SMILES: C=C(COc1c(OC)cc2sc(C(=O)CCC(=O)O)cc2c1C#N)COc1c(OC)cc2sc(C(=O)CCC(=O)O)cc2c1C#N] | |
| | 234 | 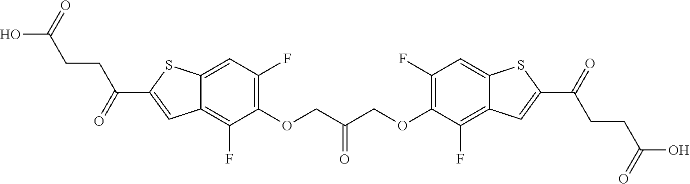 [SMILES: O=C(O)CCC(=O)c1cc2c(F)c(OCC(=O)COc3c(F)cc4sc(C(=O)CCC(=O)O)cc4c3F)c(F)cc2s1] | |
| | 235 | 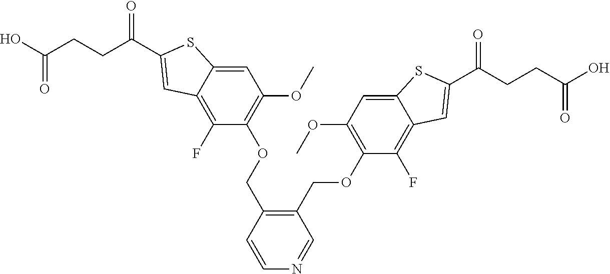 [SMILES: COc1cc2sc(C(=O)CCC(=O)O)cc2c(F)c1OCc1ccncc1COc1c(OC)cc2sc(C(=O)CCC(=O)O)cc2c1F] | |
| | 236 | 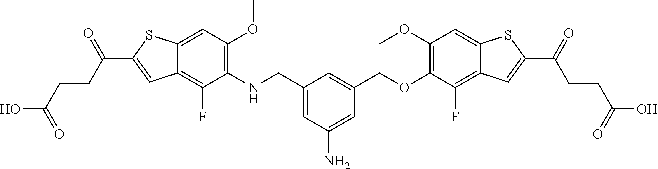 [SMILES: COc1cc2sc(C(=O)CCC(=O)O)cc2c(F)c1NCc1cc(N)cc(COc2c(OC)cc3sc(C(=O)CCC(=O)O)cc3c2F)c1] | |
| | 254 | 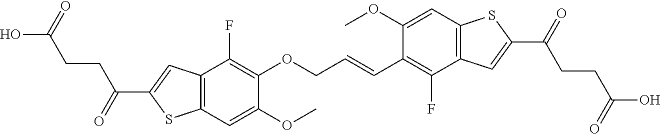 [SMILES: COc1cc2sc(C(=O)CCC(=O)O)cc2c(F)c1/C=C/COc1c(OC)cc2sc(C(=O)CCC(=O)O)cc2c1F] | |
| | 255 | 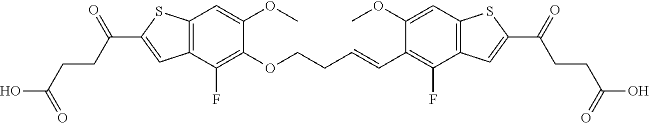 [SMILES: COc1cc2sc(C(=O)CCC(=O)O)cc2c(F)c1/C=C/CCOc1c(OC)cc2sc(C(=O)CCC(=O)O)cc2c1F] | |
| | 256 | 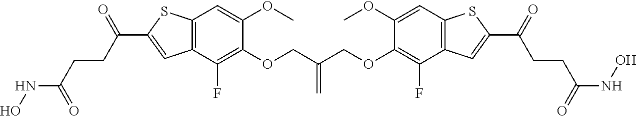 [SMILES: C=C(COc1c(OC)cc2sc(C(=O)CCC(=O)NO)cc2c1F)COc1c(OC)cc2sc(C(=O)CCC(=O)NO)cc2c1F] | |
| | 257 | 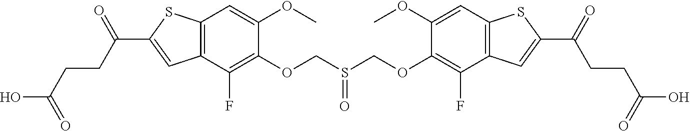 [SMILES: COc1cc2sc(C(=O)CCC(=O)O)cc2c(F)c1OCS(=O)COc1c(OC)cc2sc(C(=O)CCC(=O)O)cc2c1F] | |
| | 258 | 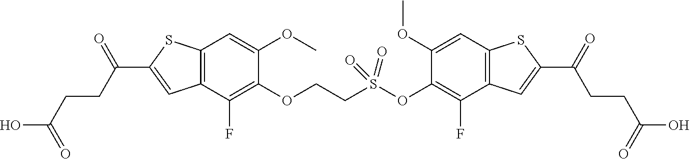 [SMILES: COc1cc2sc(C(=O)CCC(=O)O)cc2c(F)c1OCCS(=O)(=O)Oc1c(OC)cc2sc(C(=O)CCC(=O)O)cc2c1F] | |
| | 259 | 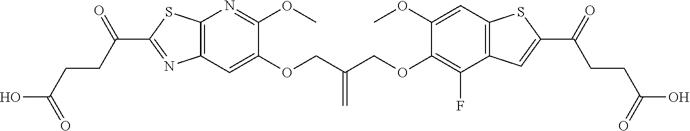 [SMILES: C=C(COc1cc2nc(C(=O)CCC(=O)O)sc2nc1OC)COc1c(OC)cc2sc(C(=O)CCC(=O)O)cc2c1F] | |
| | 260 | 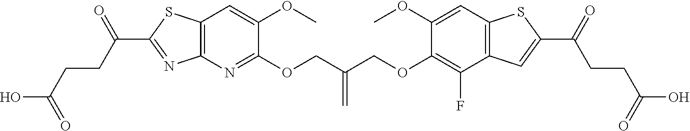 [SMILES: C=C(COc1nc2nc(C(=O)CCC(=O)O)sc2cc1OC)COc1c(OC)cc2sc(C(=O)CCC(=O)O)cc2c1F] | |
| | 261 | 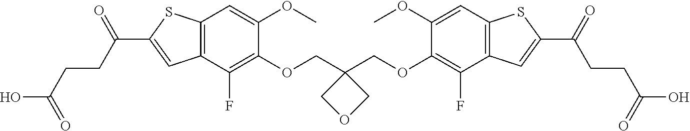 [SMILES: COc1cc2sc(C(=O)CCC(=O)O)cc2c(F)c1OCC1(COc2c(OC)cc3sc(C(=O)CCC(=O)O)cc3c2F)COC1] | |
| 17 | 262 | 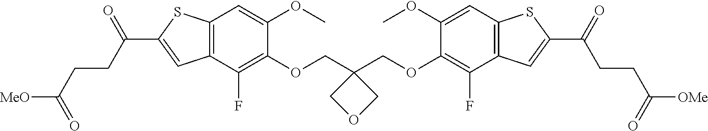 [SMILES: COC(=O)CCC(=O)c1cc2c(F)c(OCC3(COc4c(OC)cc5sc(C(=O)CCC(=O)OC)cc5c4F)COC3)c(OC)cc2s1] | 707.05 |
| 16 | 263 | 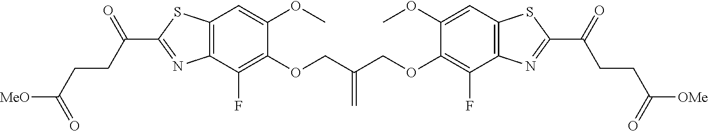 [SMILES: C=C(COc1c(OC)cc2sc(C(=O)CCC(=O)OC)nc2c1F)COc1c(OC)cc2sc(C(=O)CCC(=O)OC)nc2c1F] | 679.07 |
| | | 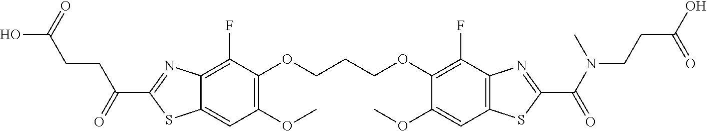 [SMILES: COc1cc2sc(C(=O)CCC(=O)O)nc2c(F)c1OCCCOc1c(OC)cc2sc(C(=O)N(C)CCC(=O)O)nc2c1F] | |
| | | 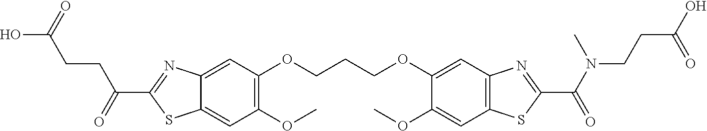 [SMILES: COc1cc2sc(C(=O)CCC(=O)O)nc2cc1OCCCOc1cc2nc(C(=O)N(C)CCC(=O)O)sc2cc1OC] | |
| | | 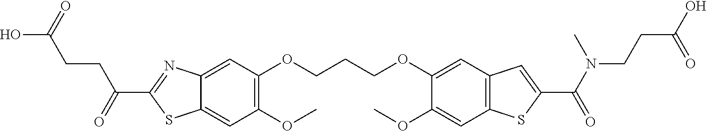 [SMILES: COc1cc2sc(C(=O)N(C)CCC(=O)O)cc2cc1OCCCOc1cc2nc(C(=O)CCC(=O)O)sc2cc1OC] | |
| | | 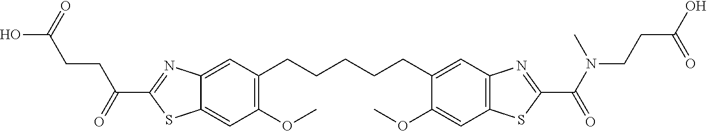 [SMILES: COc1cc2sc(C(=O)CCC(=O)O)nc2cc1CCCCCc1cc2nc(C(=O)N(C)CCC(=O)O)sc2cc1OC] | |
| | | 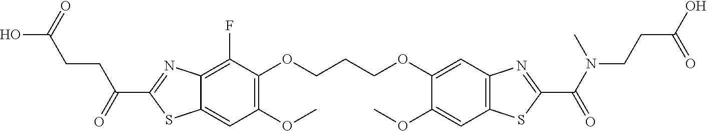 [SMILES: COc1cc2sc(C(=O)N(C)CCC(=O)O)nc2cc1OCCCOc1c(OC)cc2sc(C(=O)CCC(=O)O)nc2c1F] | |
| | | 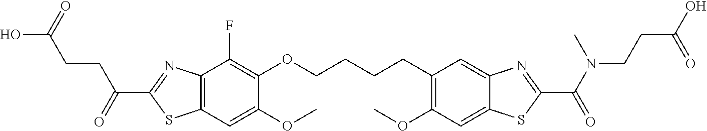 [SMILES: COc1cc2sc(C(=O)N(C)CCC(=O)O)nc2cc1CCCCOc1c(OC)cc2sc(C(=O)CCC(=O)O)nc2c1F] | |
| | | 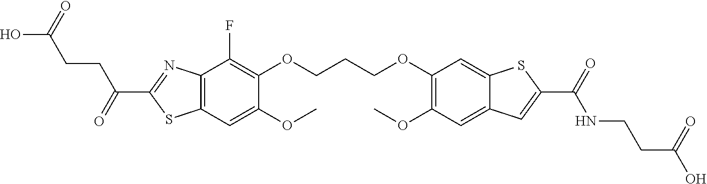 [SMILES: COc1cc2cc(C(=O)NCCC(=O)O)sc2cc1OCCCOc1c(OC)cc2sc(C(=O)CCC(=O)O)nc2c1F] | |
| | | 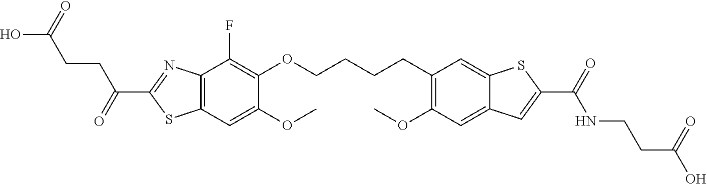 [SMILES: COc1cc2cc(C(=O)NCCC(=O)O)sc2cc1CCCCOc1c(OC)cc2sc(C(=O)CCC(=O)O)nc2c1F] | |
| | | 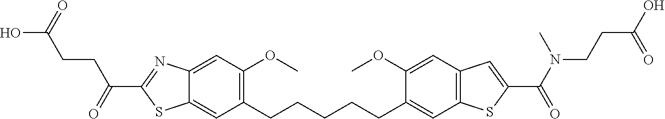 [SMILES: COc1cc2cc(C(=O)N(C)CCC(=O)O)sc2cc1CCCCCc1cc2sc(C(=O)CCC(=O)O)nc2cc1OC] | |
| | | 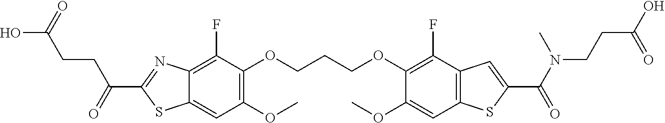 [SMILES: COc1cc2sc(C(=O)N(C)CCC(=O)O)cc2c(F)c1OCCCOc1c(OC)cc2sc(C(=O)CCC(=O)O)nc2c1F] | |
| | | 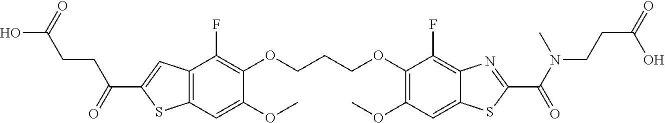 [SMILES: COc1cc2sc(C(=O)CCC(=O)O)cc2c(F)c1OCCCOc1c(OC)cc2sc(C(=O)N(C)CCC(=O)O)nc2c1F] | |

In some embodiments, the compound is:

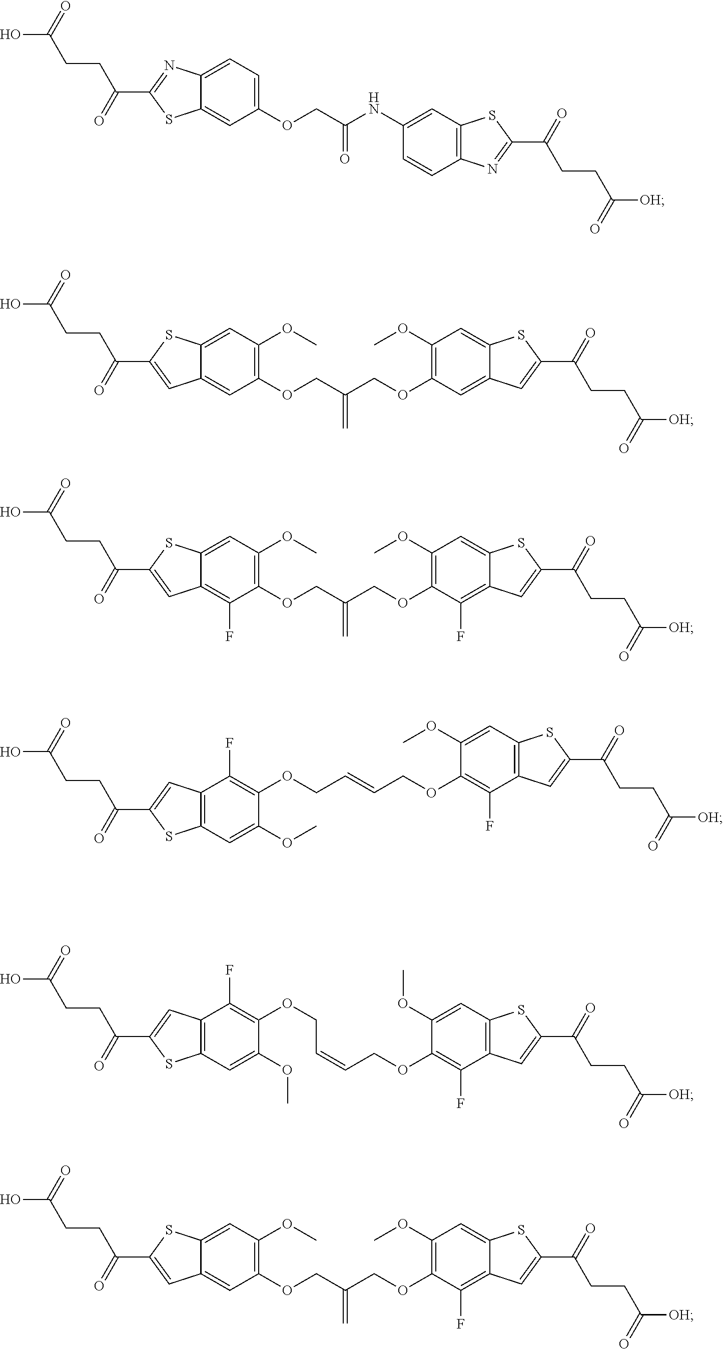 [SMILES: O=C(O)CCC(=O)c1nc2ccc(NC(=O)COc3ccc4nc(C(=O)CCC(=O)O)sc4c3)cc2s1 ; C=C(COc1cc2cc(C(=O)CCC(=O)O)sc2cc1OC)COc1cc2cc(C(=O)CCC(=O)O)sc2cc1OC ; C=C(COc1c(OC)cc2sc(C(=O)CCC(=O)O)cc2c1F)COc1c(OC)cc2sc(C(=O)CCC(=O)O)cc2c1F ; COc1cc2sc(C(=O)CCC(=O)O)cc2c(F)c1OC/C=C/COc1c(OC)cc2sc(C(=O)CCC(=O)O)cc2c1F ; COc1cc2sc(C(=O)CCC(=O)O)cc2c(F)c1OC/C=C\COc1c(OC)cc2sc(C(=O)CCC(=O)O)cc2c1F ; C=C(COc1cc2cc(C(=O)CCC(=O)O)sc2cc1OC)COc1c(OC)cc2sc(C(=O)CCC(=O)O)cc2c1F]

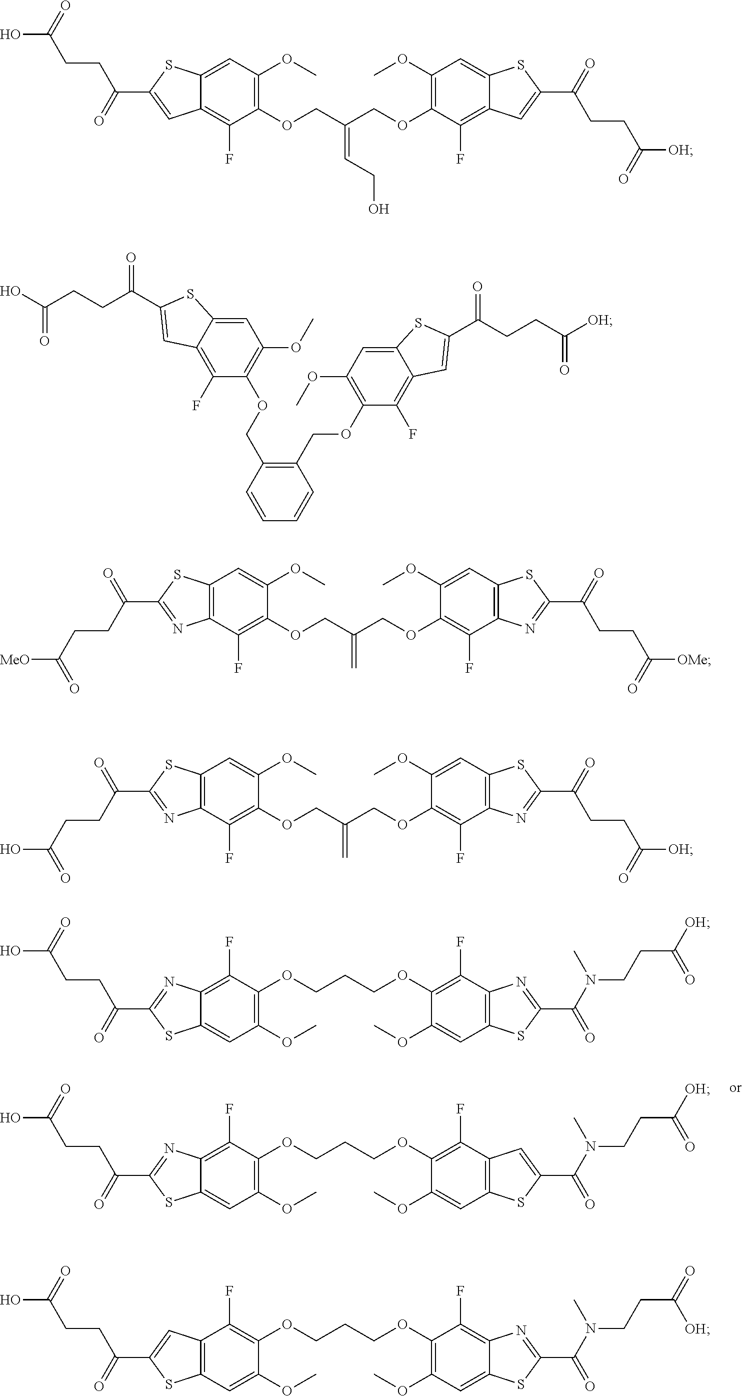

or pharmaceutically acceptable salts thereof.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I).

In some embodiments, the compound is a deuterium labeled compound of any one of the compounds described in Table 1 and prodrugs and pharmaceutically acceptable salts thereof.

In some embodiments, the compound is a deuterium labeled compound of any one of the compounds described in Table 1 and pharmaceutically acceptable salts thereof.

In some embodiments, the compound is a deuterium labeled compound of any one of the prodrugs of the compounds described in Table 1 and pharmaceutically acceptable salts thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

### Methods of Synthesizing Compounds

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of the present disclosure has been synthesized by any one of the processes defined herein, the processes may then further comprise the additional steps of: (i) removing any protecting groups present; (ii) converting the compound of the present disclosure into another compound of the present disclosure; (iii) forming a pharmaceutically acceptable salt, hydrate thereof, and/or (iv) forming a prodrug thereof.

The resultant compound of the present disclosure can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

Several methods for preparing the compounds of Formula (I), compounds of Formula (II), compounds of Formula (III), compounds of Formula (IV), compounds of Formula (V), compounds of Formula (VI), and pharmaceutically acceptable salts thereof, are described in, for example, the following Schemes and Examples. Starting materials and intermediates are purchased from commercial sources, made from known procedures, or are otherwise illustrated.

In some cases the order of carrying out the steps of the reaction schemes may be varied to facilitate the reaction or to avoid unwanted reaction products.

### Pharmaceutical Compositions and Kits

In some aspects, the present disclosure provides a pharmaceutical composition comprising an effective amount of a compound of the present disclosure and a pharmaceutically acceptable carrier.

In some embodiments, the pharmaceutical composition comprises at least one compound selected from Table 1.

In some embodiments, the pharmaceutical composition further comprises an active agent, e.g., being selected from the group consisting of STING agonist compounds, anti-viral compounds, antigens, adjuvants, CTLA-4 and PD-1 pathway antagonists and other immunomodulatory agents, lipids, liposomes, peptides, anti-cancer and chemotherapeutic agents.

In some aspects, the present disclosure provides a pharmaceutical kit comprising an effective amount of a compound of the present disclosure.

In some embodiments, the pharmaceutical kit comprises at least one compound selected from Table 1.

In some embodiments, the pharmaceutical kit further comprises an active agent, e.g., being selected from the group consisting of STING agonist compounds, anti-viral compounds, antigens, adjuvants, CTLA-4 and PD-1 pathway antagonists and other immunomodulatory agents, lipids, liposomes, peptides, anti-cancer and chemotherapeutic agents.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy is an amount sufficient to treat or prevent an inflammasome related condition referred to herein, slow its progression and/or reduce the symptoms associated with the condition.

An effective amount of a compound of the present disclosure for use in therapy is an amount sufficient to treat an inflammasome related condition referred to herein, slow its progression and/or reduce the symptoms associated with the condition.

### Conjugates

Without wishing to be bound by theory, the compound of the present disclosure may be used as a candidate for a conjugate (e.g., an antibody-STING agonist conjugate).

In some aspects, the present disclosure provides a conjugate comprising a compound disclosed herein.

In some embodiments, the conjugate is an antibody-drug conjugate.

In some embodiments, the conjugate further comprises an antibody, wherein the compound is attached to the antibody.

In some embodiments, the conjugate further comprises a linker, wherein the compound is attached to the antibody via the linker.

In some aspects, the present disclosure provides a conjugate comprising a compound disclosed herein, an antibody, and a linker, wherein the compound is attached to the antibody via the linker. In some embodiments, a compound disclosed herein has, or is modified to include, a group reactive with a conjugation point on an antibody.

In some embodiments, the linker is a polymeric linker.

In some embodiments, the linker is a non-polymeric linker.

In some aspects, the present disclosure provides use of a compound disclosed herein in the manufacture of a conjugate (e.g., an antibody-STING agonist conjugate). In some aspects, the present disclosure provides the use of an antibody-STING agonist conjugate indicated above for the manufacture of a medicament.

### Methods of Use

In some aspects, the present disclosure provides a method of treating or preventing a STING mediated disease or disorder in a subject in need thereof, comprising administering to the subject a therapeutically effective amount of a compound of the present disclosure or a pharmaceutically acceptable salt thereof.

In some aspects, the present disclosure provides a method of treating a STING mediated disease or disorder in a subject in need thereof, comprising administering to the subject a therapeutically effective amount of a compound of the present disclosure or a pharmaceutically acceptable salt thereof.

In some aspects, the present disclosure provides a method of inducing an immune response in a subject, comprising administering to the subject a therapeutically effective amount of a compound of the present disclosure or a pharmaceutically acceptable salt thereof.

In some aspects, the present disclosure provides a method of inducing STING-dependent type I interferon production in a subject, comprising administering to the subject a therapeutically effective amount of a compound of the present disclosure or a pharmaceutically acceptable salt thereof.

In some aspects, the present disclosure provides a method for treatment or prevention of diseases and disorders in which modulation of STING is beneficial. Such STING mediated diseases and disorders include inflammation, allergic and autoimmune diseases, infectious diseases, cancer and precancerous syndromes. The compounds of the disclosure are also useful as an immunogenic composition or vaccine adjuvant.

In some aspects, the present disclosure provides a method of inducing STING-dependent cytokine production in a subject, comprising administering to the subject a therapeutically effective amount of a compound of the present disclosure or a pharmaceutically acceptable salt thereof.

In some aspects, the present disclosure provides a method of treating or preventing a cell proliferation disorder in a subject, comprising administering to the subject a therapeutically effective amount of a compound of the present disclosure or a pharmaceutically acceptable salt thereof.

In some aspects, the present disclosure provides a method of treating a cell proliferation disorder in a subject, comprising administering to the subject a therapeutically effective amount of a compound of the present disclosure or a pharmaceutically acceptable salt thereof.

In some aspects, the present disclosure provides a compound of the present disclosure or a pharmaceutically acceptable salt thereof for treating or preventing a STING mediated disease or disorder in a subject.

In some aspects, the present disclosure provides a compound of the present disclosure or a pharmaceutically acceptable salt thereof for treating a STING mediated disease or disorder in a subject.

In some aspects, the present disclosure provides a compound of the present disclosure or a pharmaceutically acceptable salt thereof for inducing an immune response in a subject.

In some aspects, the present disclosure provides a compound of the present disclosure or a pharmaceutically acceptable salt thereof for inducing STING-dependent type I interferon production in a subject.

In some aspects, the present disclosure provides a compound of the present disclosure or a pharmaceutically acceptable salt thereof for inducing STING-dependent cytokine production in a subject.

In some aspects, the present disclosure provides a compound of the present disclosure or a pharmaceutically acceptable salt thereof for treating or preventing a cell proliferation disorder in a subject.

In some aspects, the present disclosure provides a compound of the present disclosure or a pharmaceutically acceptable salt thereof for treating a cell proliferation disorder in a subject.

In some aspects, the present disclosure provides a compound of the present disclosure or a pharmaceutically acceptable salt thereof for use in the manufacture of a medicament for treating or preventing a STING mediated disease or disorder in a subject.

In some aspects, the present disclosure provides a compound of the present disclosure or a pharmaceutically acceptable salt thereof for use in the manufacture of a medicament for treating a STING mediated disease or disorder in a subject.

In some aspects, the present disclosure provides a compound of the present disclosure or a pharmaceutically acceptable salt thereof for use in the manufacture of a medicament for inducing an immune response in a subject.

In some aspects, the present disclosure provides a compound of the present disclosure or a pharmaceutically acceptable salt thereof for use in the manufacture of a medicament for inducing STING-dependent type I interferon production in a subject.

In some aspects, the present disclosure provides a compound of the present disclosure or a pharmaceutically acceptable salt thereof for use in the manufacture of a medicament for inducing STING-dependent cytokine production in a subject.

In some aspects, the present disclosure provides a compound of the present disclosure or a pharmaceutically acceptable salt thereof for use in the manufacture of a medicament for treating or preventing a cell proliferation disorder in a subject.

In some aspects, the present disclosure provides a compound of the present disclosure or a pharmaceutically acceptable salt thereof for use in the manufacture of a medicament for treating a cell proliferation disorder in a subject.

In some aspects, the present disclosure provides a method of treating or preventing a STING mediated disease or disorder in a subject, comprising administering to the subject a therapeutically effective amount of a conjugate disclosed herein.

In some aspects, the present disclosure provides a method of treating a STING mediated disease or disorder in a subject, comprising administering to the subject a therapeutically effective amount of a conjugate disclosed herein.

In some aspects, the present disclosure provides a method of inducing an immune response in a subject, comprising administering to the subject a therapeutically effective amount of a conjugate disclosed herein.

In some aspects, the present disclosure provides a method of inducing STING-dependent type I interferon production in a subject, comprising administering to the subject a therapeutically effective amount of a conjugate disclosed herein.

In some aspects, the present disclosure provides a method of inducing STING-dependent cytokine production in a subject, comprising administering to the subject a therapeutically effective amount of a conjugate disclosed herein.

In some aspects, the present disclosure provides a method of treating or preventing a cell proliferation disorder in a subject, comprising administering to the subject a therapeutically effective amount of a conjugate disclosed herein.

In some aspects, the present disclosure provides a method of treating a cell proliferation disorder in a subject, comprising administering to the subject a therapeutically effective amount of a conjugate disclosed herein.

In some aspects, the present disclosure provides a conjugate disclosed herein for treating or preventing a STING mediated disease or disorder in a subject.

In some aspects, the present disclosure provides a conjugate disclosed herein for treating a STING mediated disease or disorder in a subject.

In some aspects, the present disclosure provides a conjugate disclosed herein for inducing an immune response in a subject.

In some aspects, the present disclosure provides a conjugate disclosed herein for inducing STING-dependent type I interferon production in a subject.

In some aspects, the present disclosure provides a conjugate disclosed herein for inducing STING-dependent cytokine production in a subject.

In some aspects, the present disclosure provides a conjugate disclosed herein for treating or preventing a cell proliferation disorder in a subject.

In some aspects, the present disclosure provides a conjugate disclosed herein for treating a cell proliferation disorder in a subject.

In some aspects, the present disclosure provides a conjugate disclosed herein for use in the manufacture of a medicament for treating or preventing a STING mediated disease or disorder in a subject.

In some aspects, the present disclosure provides a conjugate disclosed herein for use in the manufacture of a medicament for treating a STING mediated disease or disorder in a subject.

In some aspects, the present disclosure provides a conjugate disclosed herein for use in the manufacture of a medicament for inducing an immune response in a subject.

In some aspects, the present disclosure provides a conjugate disclosed herein for use in the manufacture of a medicament for inducing STING-dependent type I interferon production in a subject.

In some aspects, the present disclosure provides a conjugate disclosed herein for use in the manufacture of a medicament for inducing STING-dependent cytokine production in a subject.

In some aspects, the present disclosure provides a conjugate disclosed herein for use in the manufacture of a medicament for treating or preventing a cell proliferation disorder in a subject.

In some aspects, the present disclosure provides a conjugate disclosed herein for use in the manufacture of a medicament for treating a cell proliferation disorder in a subject.

In some embodiments, the STING mediated disease or disorder is cancer.

In some embodiments, the cell proliferation disorder is cancer.

In some embodiments, a second active agent is administered to the subject.

In some embodiments, the compound of the present disclosure and the second active agent are administered simultaneously.

In some embodiments, the compound of the present disclosure and the second active agent are administered sequentially.

In some embodiments, the compound of the present disclosure and the second active agent are administered in alternation.

In some embodiments, the compound of the present disclosure is administered before the administration of the second active agent.

In some embodiments, the compound of the present disclosure is administered after the administration of the second active agent.

In some embodiments, the compound of the present disclosure and the second active agent are administered in separate pharmaceutical compositions.

In some embodiments, the compound of the present disclosure and the second active agent are administered in a single pharmaceutical composition.

In some embodiments, the second active agent is selected from the group consisting of STING agonist compounds, anti-viral compounds, antigens, adjuvants, CTLA-4 and PD-1 pathway antagonists and other immunomodulatory agents, lipids, liposomes, peptides, anti-cancer and chemotherapeutic agents.

Compounds described herein having therapeutic applications, such as the compounds of Formula (I), compounds of Formula (II), compounds of Formula (III), compounds of Formula (IV), compounds of Formula (V), compounds of Formula (VI), and pharmaceutically acceptable salts thereof, may be administered to a patient for the purpose of inducing an immune response, inducing STING-dependent cytokine production and/or inducing anti-tumor activity. The term “administration” and variants thereof (e.g., “administering” a compound) means providing the compound to the individual in need of treatment. When a compound is provided in combination with one or more additional active agents (e.g., antiviral agents useful for treating HCV infection or anti-tumor agents for treating cancers), “administration” and its variants are each understood to include concurrent and sequential provision of the compound or salt and other agents.

The compounds disclosed herein may be STING agonists. These compounds are potentially useful in treating diseases or disorders including, but not limited to, cell proliferation disorders. Cell-proliferation disorders include, but are not limited to, cancers, benign papillomatosis, gestational trophoblastic diseases, and benign neoplastic diseases, such as skin papilloma (warts) and genital papilloma.

In some embodiments, the disease or disorder to be treated is a cell proliferation disorder. In some embodiments, the cell proliferation disorder is cancer. In particular embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary (i.e., cancers in which a metastasized cancer is found but the original cancer site is not known). In particular embodiments, the cancer is present in an adult patient; in additional embodiments, the cancer is present in a pediatric patient. In particular embodiments, the cancer is AIDS-related.

In some embodiments, the cancer is brain or spinal cancers. In some embodiments, the cancer is anaplastic astrocytomas, glioblastomas, astrocytomas, or estheosioneuroblastomas (also known as olfactory blastomas). In some embodiments, the brain cancer is astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astrocytoma/malignant glioma, visual pathway and hypothalmic glioma, or primary central nervous system lymphoma. In specific embodiments, the brain cancer is glioma, glioblastoma multiforme, paraganglioma, or supratentorial primordial neuroectodermal tumors (sPNET).

In some embodiments, the cancer is head or neck cancer, including, but not limited to, nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), or cancers of the eye or ocular cancers. In some embodiments, the ocular cancer is intraocular melanoma or retinoblastoma.

In some embodiments, the cancer is leukemia or cancers of the blood. In some embodiments, the cancer is myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del (5q)-associated high risk MDS or AML, blast-phase chronic myelogenous leukemia, angioimmunoblastic lymphoma, acute lymphoblastic leukemia, Langerans cell histiocytosis, hairy cell leukemia, or plasma cell neoplasms, including, but not limited to plasmacytomas or multiple myelomas. Leukemias referenced herein may be acute or chronic.

In some embodiments, the cancer is skin cancers. In some embodiments, the skin cancer is melanoma, squamous cell cancers, or basal cell cancers.

In some embodiments, the cancer is cancers of the reproductive system. In some embodiments, the cancer is breast cancers, cervical cancers, vaginal cancers, ovarian cancers, prostate cancers, penile cancers, or testicular cancers. In some embodiments, the breast cancer is ductal carcinomas or phyllodes tumors. In some embodiments, the breast cancer may be male breast cancer or female breast cancer. In some embodiments, the cervical cancer is squamous cell carcinomas or adenocarcinomas. In some embodiments, the ovarian cancer is epithelial cancers.

In some embodiments, the cancer is cancers of the gastro-intestinal system. In some embodiments, the cancer is esophageal cancers, gastric cancers (also known as stomach cancers), gastrointestinal carcinoid tumors, pancreatic cancers, gallbladder cancers, colorectal cancers, or anal cancer. In some embodiments, the cancer is esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, or anal squamous cell carcinomas.

In some embodiments, the cancer is liver or bile duct cancer. In some embodiments, the cancer is liver cancer (also known as hepatocellular carcinoma). In some embodiments, the cancer is bile duct cancer (also known as cholangiocarcinoma); in some embodiments, the bile duct cancer is intrahepatic cholangiocarcinoma or extrahepatic cholangiocarcinoma.

In some embodiments, the cancer is kidney or bladder cancers. In some embodiments, the kidney cancer is renal cell cancer, Wilms tumors, or transitional cell cancers. In some embodiments, the bladder cancer is urothelial carcinoma (a transitional cell carcinoma), squamous cell carcinomas, or adenocarcinomas.

In some embodiments, the cancer is bone cancer. In some embodiments, the bone cancer is osteosarcoma, malignant fibrous histiocytoma of bone, Ewing sarcoma, or chordoma (cancer of the bone along the spine).

In some embodiments, the cancer is lung cancer. In some embodiments, the lung cancer is non-small cell lung cancer (NSCLC), small cell lung cancers, bronchial tumors, or pleuropulmonary blastomas.

In some embodiments, the cancer is malignant mesothelioma. In some embodiments, the cancer is epithelial mesothelioma or sarcomatoids.

In some embodiments, the cancer is sarcoma. In some embodiments, the sarcoma is central chondrosarcoma, central and periosteal chondroma, fibrosarcoma, clear cell sarcoma of tendon sheaths, or Kaposi's sarcoma.

In some embodiments, the cancer is lymphoma. In some embodiments, the cancer is Hodgkin lymphoma (e.g., Reed-Sternberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, or primary central nervous system lymphomas.

In some embodiments, the cancer is glandular cancer. In some embodiments, the cancer is adrenocortical cancer (also known as adrenocortical carcinoma or adrenal cortical carcinoma), pheochromocytomas, paragangliomas, pituitary tumors, thymoma, or thymic carcinomas.

In some embodiments, the cancer is thyroid cancer. In some embodiments, the thyroid cancer is medullary thyroid carcinomas, papillary thyroid carcinomas, or follicular thyroid carcinomas.

In some embodiments, the cancer is germ cell tumors. In some embodiments, the cancer is malignant extracranial germ cell tumors or malignant extragonadal germ cell tumors. In some embodiments, the malignant extragonadal germ cell tumors are non-seminomas or seminomas.

In some embodiments, the cancer is heart tumors. In some embodiments, the heart tumor is malignant teratoma, lymphoma, rhabdomyosarcoma, angiosarcoma, chondrosarcoma, infantile fibrosarcoma, or synovial sarcoma.

In some embodiments, the cell-proliferation disorder is benign papillomatosis, benign neoplastic diseases or gestational trophoblastic diseases. In some embodiments, the benign neoplastic disease is skin papilloma (warts) or genital papilloma. In some embodiments, the gestational trophoblastic disease is hydatidiform moles, gestational trophoblastic neoplasia (e.g., invasive moles, choriocarcinomas, placental-site trophoblastic tumors, or epithelioid trophoblastic tumors).

The amount of a compound administered to a patient is an amount sufficient to induce an immune response and/or to induce STING-dependent type I interferon production in the patient. In some embodiments, the amount of a compound can be an “effective amount” or “therapeutically effective amount,” such that the subject compound is administered in an amount that will elicit, respectively, a biological or medical (i.e., intended to treat) response of a tissue, system, animal, or human that is being sought by a researcher, veterinarian, medical doctor, or other clinician. An effective amount does not necessarily include considerations of toxicity and safety related to the administration of a compound.

An effective amount of a compound will vary with the particular compound chosen (e.g., considering the potency, efficacy, and/or half-life of the compound); the route of administration chosen; the condition being treated; the severity of the condition being treated; the age, size, weight, and physical condition of the patient being treated; the medical history of the patient being treated; the duration of the treatment; the nature of a concurrent therapy; the desired therapeutic effect; and like factors and can be routinely determined by the skilled artisan.

The compounds disclosed herein may be administered by any suitable route including oral and parenteral administration. Parenteral administration is typically by injection or infusion and includes intravenous, intramuscular, and subcutaneous injection or infusion.

The compounds disclosed herein may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound disclosed herein depend on the pharmacokinetic properties of that compound, such as absorption, distribution and half-life, which can be determined by a skilled artisan. In addition, suitable dosing regimens, including the duration such regimens are administered, for a compound disclosed herein depend on the disease or condition being treated, the severity of the disease or condition, the age and physical condition of the patient being treated, the medical history of the patient being treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as the individual patient needs change. Typical daily dosages may vary depending upon the particular route of administration chosen.

Some embodiments of the present disclosure provide for a method of treating a cell proliferation disorder comprising administration of a therapeutically effective amount of a compound of Formula (I), compound of Formula (II), compound of Formula (III), compound of Formula (IV), compound of Formula (V), compound of Formula (VI), and pharmaceutically acceptable salts thereof, to a patient in need of treatment thereof. In some embodiments, the disease or disorder to be treated is a cell proliferation disorder. In some embodiments, the cell proliferation disorder is cancer. In other embodiments, the cancer is brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, or cancers of unknown primary.

In some embodiments, a compound of Formula (I), compound of Formula (II), compound of Formula (III), compound of Formula (IV), compound of Formula (V), compound of Formula (VI), or a pharmaceutically acceptable salt thereof, is used in a therapy. The compound may be useful in a method of inducing an immune response and/or inducing STING-dependent type I interferon production in a patient, such as a mammal in need of such inhibition, comprising administering an effective amount of the compound to the patient.

In some embodiments, a pharmaceutical composition comprising at least one compound of Formula (I), at least one compound of Formula (II), at least one compound of Formula (III), at least one compound of Formula (IV), at least one compound of Formula (V), at least one compound of Formula (VI), or at least one pharmaceutically acceptable salt thereof, is used in potential treatment to induce an immune response and/or to induce STING-dependent type I interferon production.

In some embodiments disclosed herein a compound of Formula (I), a compound of Formula (II), a compound of Formula (III), a compound of Formula (IV), a compound of Formula (V), a compound of Formula (VI), or a pharmaceutically acceptable salt thereof, is used in the manufacture of a medicament to induce an immune response and/or to induce STING-dependent type I interferon production. In some embodiments, the disease or disorder to be treated is a cell proliferation disorder. In some embodiments, the cell proliferation disorder is cancer. In other embodiments, the cancer brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, or cancers of unknown primary.

### Combination Therapies

The compounds of Formula (I), compounds of Formula (II), compounds of Formula (III), compounds of Formula (IV), compounds of Formula (V), compounds of Formula (VI), and/or pharmaceutically acceptable salts thereof, may be administered in combination with one or more additional active agents. In embodiments, one or more compounds of Formula (I), compounds of Formula (II), compounds of Formula (III), compounds of Formula (IV), compounds of Formula (V), compounds of Formula (VI), or one or more pharmaceutically acceptable salts thereof, and the one or more additional active agents may be co-administered. The additional active agent(s) may be administered in a single dosage form with the compound of Formula (I), compound of Formula (II), compound of Formula (III), compound of Formula (IV), compound of Formula (V), compound of Formula (VI), or pharmaceutically acceptable salt thereof, or the additional active agent(s) may be administered in separate dosage form(s) from the dosage form containing the compound of Formula (I), compound of Formula (II), compound of Formula (III), compound of Formula (IV), compound of Formula (V), compound of Formula (VI), or pharmaceutically acceptable salt thereof.

The additional active agent(s) may be provided as a pharmaceutically acceptable salt, wherein appropriate.

The additional active agent(s) may be one or more agents of STING agonist compounds, anti-viral compounds, antigens, adjuvants, anti-cancer agents, CTLA-4, LAG-3 and PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, or immunomodulatory agents including but not limited to anti-cancer vaccines. It will be understood that such additional active agent(s) may be provided as a pharmaceutically acceptable salt. It will be understood the descriptions of the above additional active agents may be overlapping. It will also be understood that the treatment combinations are subject to optimization, and it is understood that the best combination to use of the compounds of Formula (I), compounds of Formula (II), compounds of Formula (III), compounds of Formula (IV), compounds of Formula (V), compounds of Formula (VI), or pharmaceutically acceptable salts thereof, and one or more additional active agents will be determined based on the individual patient needs.

A compound disclosed herein may be used in combination with one or more other active agents, including but not limited to, other anti-cancer agents that are used in the prevention, treatment, control, amelioration, or reduction of risk of a particular disease or condition (e.g., cell proliferation disorders). In some embodiments, a compound disclosed herein is combined with one or more other anti-cancer agents for use in the prevention, treatment, control amelioration, or reduction of risk of a particular disease or condition for which the compounds disclosed herein are useful. Such other active agents may be administered, by a route and in an amount commonly used therefor, contemporaneously or sequentially with a compound of the present disclosure.

When a compound disclosed herein is used contemporaneously with one or more other active agents, a composition containing such other active agents in addition to the compound disclosed herein is contemplated. Accordingly, the compositions of the present disclosure include those that also contain one or more other active ingredients, in addition to a compound disclosed herein. A compound disclosed herein may be administered either simultaneously with, or before or after, one or more other active agent(s). A compound disclosed herein may be administered separately, by the same or different route of administration, or together in the same pharmaceutical composition as the other agent(s).

Products provided as combinations may include a composition comprising a compound of Formula (I), compound of Formula (II), compound of Formula (III), compound of Formula (IV), compound of Formula (V), compound of Formula (VI), or a pharmaceutically acceptable salt thereof, and one or more other active agent(s) together in the same pharmaceutical composition, or may include a composition comprising a compound of Formula (I), compound of Formula (II), compound of Formula (III), compound of Formula (IV), compound of Formula (V), compound of Formula (VI), or a pharmaceutically acceptable salt thereof, and a composition comprising one or more other active agent(s) in separate form, e.g. in the form of a kit or in any form designed to enable separate administration either concurrently or on separate dosing schedules.

The weight ratio of a compound of Formula (I), compound of Formula (II), compound of Formula (III), compound of Formula (IV), compound of Formula (V), compound of Formula (VI), or a pharmaceutically acceptable salt thereof, to a second active agent may be varied and will depend upon the therapeutically effective dose of each agent. Generally, a therapeutically effective dose of each will be used. Combinations of a compound disclosed herein and other active agents will generally also be within the aforementioned range, but in each case, a therapeutically effective dose of each active agent should be used. In such combinations, the compound disclosed herein and other active agents may be administered separately or in conjunction. In addition, the administration of one element may be prior to, concurrent to, or subsequent to the administration of other agent(s).

In some embodiments, this disclosure provides a composition comprising a compound of Formula (I), compound of Formula (II), compound of Formula (III), compound of Formula (IV), compound of Formula (V), compound of Formula (VI), or a pharmaceutically acceptable salt thereof, and at least one other active agent as a combined preparation for simultaneous, separate, or sequential use in therapy. In some embodiments, the therapy is the treatment of a cell proliferation disorder, such as cancer.

In some embodiments, the disclosure provides a kit comprising two or more separate pharmaceutical compositions, at least one of which contains a compound of Formula (I), compound of Formula (II), compound of Formula (III), compound of Formula (IV), compound of Formula (V), compound of Formula (VI), or a pharmaceutically acceptable salt thereof. In some embodiments, the kit comprises means for separately retaining said compositions, such as a container, divided bottle, or divided foil packet. An example of such a kit is a blister pack, as typically used for the packaging of tablets, capsules, and the like.

A kit of this disclosure may be used for administration of different dosage forms, for example, oral and parenteral, for administration of the separate compositions at different dosage intervals, or for titration of the separate compositions against one another. To assist with compliance, a kit of the disclosure typically comprises directions for administration.

Disclosed herein is a use of a compound of Formula (I), compound of Formula (II), compound of Formula (III), compound of Formula (IV), compound of Formula (V), compound of Formula (VI), or a pharmaceutically acceptable salt thereof, for treating a cell proliferation disorder, wherein the medicament is prepared for administration with another active agent. The disclosure also provides the use of another active agent for treating a cell proliferation disorder, wherein the medicament is administered with a compound of Formula (I), compound of Formula (II), compound of Formula (III), compound of Formula (IV), compound of Formula (V), compound of Formula (VI), or a pharmaceutically acceptable salt thereof.

The disclosure also provides the use of a compound of Formula (I), compound of Formula (II), compound of Formula (III), compound of Formula (IV), compound of Formula (V), compound of Formula (VI), or a pharmaceutically acceptable salt thereof, for treating a cell proliferation disorder, wherein the patient has previously (e.g., within 24 h) been treated with another active agent. The disclosure also provides the use of another active agent for treating a cell proliferation disorder, wherein the patient has previously (e.g., within 24 h) been treated with a compound of Formula (I), compound of Formula (II), compound of Formula (III), compound of Formula (IV), compound of Formula (V), compound of Formula (VI), or a pharmaceutically acceptable salt thereof. The second agent may be administered a week, several weeks, a month, or several months after the administration of a compound disclosed herein.

STING agonist compounds that may be used in combination with the compounds of Formula (I), compounds of Formula (II), compounds of Formula (III), compounds of Formula (IV), compounds of Formula (V), compounds of Formula (VI), or pharmaceutically acceptable salts thereof, disclosed herein include but are not limited to cyclic di-nucleotide compounds, such as those disclosed, for example, in International Patent Application Publication Nos. WO2014093936, WO2014189805, WO2014189806, WO2015185565, WO2016120305, WO2016096174, WO2016096577, WO2017027645, WO2017027646, WO2017075477, WO2017093933, and WO2018009466.

Anti-viral compounds that may be used in combination with the compounds of Formula (I), compounds of Formula (II), compounds of Formula (III), compounds of Formula (IV), compounds of Formula (V), compounds of Formula (VI), or pharmaceutically acceptable salts thereof, disclosed herein include hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors. Such anti-viral compounds may be provided as a pharmaceutically acceptable salt, wherein appropriate.

Antigens and adjuvants that may be used in combination with the compounds of Formula (I), compounds of Formula (II), compounds of Formula (III), compounds of Formula (IV), compounds of Formula (V), compounds of Formula (VI), or the pharmaceutically acceptable salts thereof, include B7 costimulatory molecule, interleukin-2, interferon-y, GM-CSF, CTLA-4 antagonists, OX-40/OX-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. Adjuvants, such as aluminum hydroxide or aluminum phosphate, can be added to increase the ability of the vaccine to trigger, enhance, or prolong an immune response. Additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, LPS, monophosphoryl lipid A, lipoteichoic acid, imiquimod, resiquimod, and in addition retinoic acid-inducible gene I (RIG-I) agonists such as poly I.C, used separately or in combination with the described compositions are also potential adjuvants. Such antigens and adjuvants may be provided as a pharmaceutically acceptable salt, wherein appropriate.

CLTA-4 and PD-1 pathways are important negative regulators of immune response. Activated T-cells up-regulate CTLA-4, which binds on antigen-presenting cells and inhibits T-cell stimulation, IL-2 gene expression, and T-cell proliferation; these anti-tumor effects have been observed in mouse models of colon carcinoma, metastatic prostate cancer, and metastatic melanoma. PD-1 binds to active T-cells and suppresses T-cell activation; PD-1 antagonists have demonstrated anti-tumor effects as well. CTLA-4 and PD-1 pathway antagonists that may be used in combination with the compounds of Formula (I), compounds of Formula (II), compounds of Formula (III), compounds of Formula (IV), compounds of Formula (V), compounds of Formula (VI), or the pharmaceutically acceptable salts thereof, disclosed herein, include ipilimumab, tremelimumab, nivolumab, pembrolizumab, CT-011, AMP-224, and MDX-1106.

“PD-1 antagonist” or “PD-1 pathway antagonist” means any chemical compound or biological molecule that blocks binding of PD-L1 expressed on a cancer cell to PD-1 expressed on an immune cell (T-cell, B-cell, or NKT-cell) and preferably also blocks binding of PD-L2 expressed on a cancer cell to the immune-cell expressed PD-1. Alternative names or synonyms for PD-1 and its ligands include: PDCD1, PD1, CD279, and SLEB2 for PD-1; PDCD1L1, PDL1, B7H1, B7-4, CD274, and B7-H for PD-L1; and PDCD1L2, PDL2, B7-DC, Btdc, and CD273 for PD-L2. In any of the treatment method, medicaments and uses of the present disclosure in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in any of the treatment method, medicaments and uses of the present disclosure include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody, or a chimeric antibody and may include a human constant region. In some embodiments, the human constant region is selected from the group consisting of IgG1, IgG2, IgG3, and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv, and Fv fragments.

Examples of mAbs that bind to human PD-1, and useful in the treatment method, medicaments and uses of the present disclosure, are described in, for example, for example, U.S. Pat. Nos. 7,488,802, 7,521,051, 8,008,449, 8,354,509, and 8,168,757, PCT International Patent Application Publication Nos. WO2004/004771, WO2004/072286, and WO2004/056875, and U.S. Patent Application Publication No. US2011/0271358.

Examples of mAbs that bind to human PD-L1, and useful in the treatment method, medicaments and uses of the present disclosure, are described in, for example, for example, PCT International Patent Application Nos. WO2013/019906 and WO2010/077634 A1 and in U.S. Pat. No. 8,383,796. Specific anti-human PD-L1 mAbs useful as the PD-1 antagonist in the treatment method, medicaments and uses of the present disclosure include MPDL3280A, BMS-936559, MEDI4736, MSB0010718C, and an antibody that comprises the heavy chain and light chain variable regions of SEQ ID NO:24 and SEQ ID NO:21, respectively, of WO2013/019906.

Other PD-1 antagonists useful in any of the treatment method, medicaments, and uses of the present disclosure include an immune-adhesion that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immune-adhesion molecules that specifically bind to PD-1 are described in, for example, for example, PCT International Patent Application Publication Nos. WO2010/027827 and WO2011/066342. Specific fusion proteins useful as the PD-1 antagonist in the treatment method, medicaments, and uses of the present disclosure include AMP-224 (also known as B7-DCIg), which is a PD-L2-FC fusion protein and binds to human PD-1.

The disclosure further provides a method of treating cancer in a human patient comprising administration of a compound disclosed herein (i.e., a compound of Formula (I), a compound of Formula (II), a compound of Formula (III), a compound of Formula (IV), a compound of Formula (V), a compound of Formula (VI), or a pharmaceutically acceptable salt thereof) and a PD-1 antagonist to the patient. The compound of the disclosure and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is pembrolizumab (KEYTRUDA™, Merck & Co., Inc., Kenilworth, N.J., USA), nivolumab (OPDIVO™, Bristol-Myers Squibb Company, Princeton, N.J., USA), cemiplimab (LIBTAYO™ Regeneron Pharmaceuticals, Inc., Tarrytown, N.Y., USA), atezolizumab (TECENTRIQ™ Genentech, San Francisco, Calif., USA), durvalumab (IMFINZI™, AstraZeneca Pharmaceuticals LP, Wilmington, Del.), or avelumab (BAVENCIO™, Merck KGaA, Darmstadt, Germany).

In some embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In some embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks.

In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

Examples of cytotoxic agents that may be used in combination with the compounds of Formula (I), compounds of Formula (II), compounds of Formula (III), compounds of Formula (IV), compounds of Formula (V), compounds of Formula (VI), or pharmaceutically acceptable salts thereof, include, but are not limited to, arsenic trioxide (sold under the tradename TRISENOX®), asparaginase (also known as L-asparaginase, and Erwinia L-asparaginase, sold under the tradenames ELSPAR® and KIDROLASE®).

Chemotherapeutic agents that may be used in combination with the compounds of Formula (I), compounds of Formula (II), compounds of Formula (III), compounds of Formula (IV), compounds of Formula (V), compounds of Formula (VI), or pharmaceutically acceptable salts thereof, disclosed herein include abiraterone acetate, altretamine, anhydrovinblastine, auristatin, bexarotene, bicalutamide, BMS 184476, 2,3,4,5,6-pentafluoro-N-(3-fluoro-4-methoxyphenyl) benzene sulfonamide, bleomycin, N,N-dimethyl-L-valyl-L-valyl-N-methyl-L-valyl-L-prolyl-1-Lproline-t-butylamide, cachectin, cemadotin, chlorambucil, cyclophosphamide, 3′,4′-didehydro-4′deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea andtaxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, nivolumab, onapristone, paclitaxel, pembrolizumab, prednimustine, procarbazine, RPR109881, stramustine phosphate, tamoxifen, tasonermin, taxol, tretinoin, vinblastine, vincristine, vindesine sulfate, and vinflunine. Such chemotherapeutic agents may be provided as a pharmaceutically acceptable salt, wherein appropriate.

Examples of vascular endothelial growth factor (VEGF) receptor inhibitors include, but are not limited to, bevacizumab (sold under the trademark AVASTIN), axitinib (described in, for example, for example, PCT International Patent Publication No. WO01/002369), Brivanib Alaninate ((S)—((R)-1-(4-(4-Fluoro-2-methyl-1H-indol-5-yloxy)-5-methylpyrrolo[2,1-f][1,2,4]triazin-6-yloxy) propan-2-yl)₂-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide. and described in, for example, for example, PCT International Patent Application Publication No. WO02/068470), pasireotide (also known as SO 230, and described in, for example, PCT International Patent Publication No. WO02/010192), and sorafenib (sold under the tradename NEXAVAR). Such inhibitors may be provided as a pharmaceutically acceptable salt, wherein appropriate.

Examples of topoisomerase II inhibitors, include but are not limited to, etoposide (also known as VP-16 and Etoposide phosphate, sold under the tradenames TOPOSAR, VEPESID, and ETOPOPHOS), and teniposide (also known as VM-26, sold under the tradename VUMON). Such inhibitors may be provided as a pharmaceutically acceptable salt, wherein appropriate.

Examples of alkylating agents, include but are not limited to, 5-azacytidine (sold under the trade name VIDAZA), decitabine (sold under the trade name of DECOGEN), temozolomide (sold under the trade names TEMCAD, TEMODAR, and TEMODAL), dactinomycin (also known as actinomycin-D and sold under the tradename COSMEGEN), melphalan (also known as L-PAM, L-sarcolysin, and phenylalanine mustard, sold under the tradename ALKERAN), altretamine (also known as hexamethylmelamine (HMM), sold under the tradename HEXALEN), carmustine (sold under the tradename BCNU), bendamustine (sold under the tradename TREANDA), busulfan (sold under the tradenames BUSULFEX® and MYLERAN®), carboplatin (sold under the tradename PARAPLATIN®), lomustine (also known as CCNU, sold under the tradename CEENU®), cisplatin (also known as CDDP, sold under the tradenames PLATINOL® and PLATINOL®-AQ), chlorambucil (sold under the tradename LEUKERAN®), cyclophosphamide (sold under the tradenames CYTOXAN® and NEOSAR®), dacarbazine (also known as DTIC, DIC and imidazole carboxamide, sold under the tradename DTIC-DOME®), altretamine (also known as hexamethylmelamine (HMM) sold under the tradename HEXALEN®), ifosfamide (sold under the tradename IFEX®), procarbazine (sold under the tradename MATULANE®), mechlorethamine (also known as nitrogen mustard, mustine and mechloroethamine hydrochloride, sold under the tradename MUSTARGEN®), streptozocin (sold under the tradename ZANOSAR®), thiotepa (also known as thiophosphoamide, TESPA and TSPA, and sold under the tradename THIOPLEX®. Such alkylating agents may be provided as a pharmaceutically acceptable salt, wherein appropriate.

Examples of anti-tumor antibiotics include, but are not limited to, doxorubicin (sold under the tradenames ADRIAMYCIN® and RUBEX®), bleomycin (sold under the tradename LENOXANE®), daunorubicin (also known as dauorubicin hydrochloride, daunomycin, and rubidomycin hydrochloride, sold under the tradename CERUBIDINE®), daunorubicin liposomal (daunorubicin citrate liposome, sold under the tradename DAUNOXOME®), mitoxantrone (also known as DHAD, sold under the tradename NOVANTRONE®), epirubicin (sold under the tradename ELLENCE™), idarubicin (sold under the tradenames IDAMYCIN®, IDAMYCIN PFS®), and mitomycin C (sold under the tradename MUTAMYCIN®). Such anti-tumor antibiotics may be provided as a pharmaceutically acceptable salt, wherein appropriate.

Examples of anti-metabolites include, but are not limited to, claribine (2-chlorodeoxyadenosine, sold under the tradename LEUSTATIN®), 5-fluorouracil (sold under the tradename ADRUCTL®), 6-thioguanine (sold under the tradename PURINETHOL®), pemetrexed (sold under the tradename ALIMTA®), cytarabine (also known as arabinosylcytosine (Ara-C), sold under the tradename CYTOSAR-U®), cytarabine liposomal (also known as Liposomal Ara-C, sold under the tradename DEPOCYT™), decitabine (sold under the tradename DACOGEN®), hydroxyurea and (sold under the tradenames HYDREA®, DROXIA™ and MYLOCEL™) fludarabine (sold under the tradename FLUDARA®), floxuridine (sold under the tradename FUDR®), cladribine (also known as 2-chlorodeoxyadenosine (2-CdA) sold under the tradename LEUSTATIN™), methotrexate (also known as amethopterin, methotrexate sodium (MTX), sold under the tradenames RHEUMATREX® and TREXALL™), and pentostatin (sold under the tradename NIPENT®). Such anti-metabolites may be provided as a pharmaceutically acceptable salt, wherein appropriate.

Examples of retinoids include, but are not limited to, alitretinoin (sold under the tradename PANRETIN®), tretinoin (all-trans retinoic acid, also known as ATRA, sold under the tradename VESANOID®), Isotretinoin (13-c/s-retinoic acid, sold under the tradenames ACCUTANE®, AMNESTEEM®, CLARAVIS®, CLARUS®, DECUTAN®, ISOTANE®, IZOTECH®, ORATANE®, ISOTRET®, and SOTRET®), and bexarotene (sold under the tradename TARGRETIN®). Such compounds may be provided as a pharmaceutically acceptable salt, wherein appropriate.

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays are may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

STING Biochemical [3H]cGAMP Competition Assays: In some embodiments, the ability of the compound of the present disclosure to bind STING can be quantified by the ability to compete with tritiated cGAMP ligand for human STING receptor membrane using a radioactive filter-binding assay. The binding assay can employ STING receptor obtained from Hi-Five cell membranes overexpressing full-length HAQ STING and tritiated cGAMP ligand. It is understood that, in some embodiments, a compound of the present disclosure is considered as a STING agonist by (i) binding to the STING protein as evidenced by a reduction in binding of tritiated cGAMP ligand to the STING protein by at least 20% at 20 μM (concentration of compound being tested) in a STING Biochemical [3H]cGAMP Competition Assay and/or (ii) demonstrating interferon production with a 6% or greater induction of IFN-3 secretion at 30 μM in the THP1 cell assay (wherein induction caused by cGAMP at 30 μM was set at 100%).

### Definitions

Unless otherwise stated, the following terms used in the specification and claims have the following meanings set out below.

In the embodiments of the compound provided above, it is to be understood that each embodiment may be combined with one or more other embodiments, to the extent that such a combination provides a stable compound and is consistent with the description of the embodiments. It is further to be understood that the embodiments of compositions and methods provided as (a) through (m) above are understood to include all embodiments of the compounds, including such embodiments as result from combinations of embodiments.

As used herein, the term “alkyl” refers to a monovalent straight or branched chain, saturated aliphatic hydrocarbon radical having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and tert-butyl, n- and iso-propyl, ethyl, and methyl. As another example, “C₁₄ alkyl” refers to n-, iso-, sec- and tert-butyl, n- and isopropyl, ethyl, and methyl.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

As used herein, the term “halogen” (or “halo”) refers to fluorine, chlorine, bromine, and iodine (alternatively fluoro, chloro, bromo, and iodo or F, Cl, Br, and I).

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, and adamantyl.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like.

As used herein, the term “aryl” includes groups with aromaticity, including “conjugated,” or multicyclic systems with one or more aromatic rings and do not contain any heteroatom in the ring structure. In the case of multicyclic aryl, only one of the rings in the aryl needs to be aromatic. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, isothiazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl). In some embodiments, the heteroaryl is thiophenyl or benzothiophenyl. In some embodiments, the heteroaryl is thiophenyl. In some embodiments, the heteroaryl benzothiophenyl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

In the compounds of Formula (I), compounds of Formula (II), compounds of Formula (III), compounds of Formula (IV), compounds of Formula (V), compounds of Formula (VI), and pharmaceutically acceptable salts thereof, the atoms may exhibit their natural isotopic abundances, or one or more of the atoms may be artificially enriched in a particular isotope having the same atomic number, but an atomic mass or mass number different from the atomic mass or mass number predominantly found in nature. In some embodiments, the present disclosure is meant to include all suitable isotopic variations of the compounds of Formula (I), compounds of Formula (II), compounds of Formula (III), compounds of Formula (IV), compounds of Formula (V), compounds of Formula (VI), and pharmaceutically acceptable salts thereof. For example, different isotopic forms of hydrogen (H) include protium (¹H), deuterium (²H), and tritium (³H). Protium is the predominant hydrogen isotope found in nature. Enriching for deuterium may afford some therapeutic advantages, such as increasing in vivo half-life or reducing dosage requirements, or may provide a compound useful as a standard for characterization of biological samples.

Isotopically-enriched the compounds of Formula (I), compounds of Formula (II), compounds of Formula (III), compounds of Formula (IV), compounds of Formula (V), compounds of Formula (VI), and pharmaceutically acceptable salts thereof, can be prepared without undue experimentation by conventional techniques well known to those skilled in the art or by processes analogous to those described in, for example, the Schemes and Examples herein using appropriate isotopically-enriched reagents and/or intermediates.

In specific embodiments of the compounds of Formula (I), compounds of Formula (II), compounds of Formula (III), compounds of Formula (IV), compounds of Formula (V), compounds of Formula (VI), and pharmaceutically acceptable salts thereof, the compounds are isotopically enriched with deuterium. In some embodiments, one or more of R¹, R², R³, R⁶, R⁷, and R⁹ may include deuterium.

As shown in the Formulas and the structures of specific compounds as provided herein, a straight line at a chiral center includes both (R) and (S) stereoisomers and mixtures thereof. Also, unless otherwise specified (e.g., 100% purified compound), reference to a particular stereochemistry at a position provides a compound having the indicated stereochemistry but does not exclude the presence of stereoisomers having different stereochemistry at the indicated position.

Recitation or depiction of a specific compound in the claims (i.e., a species) without a specific stereo configuration designation, or with such a designation for less than all chiral centers, is intended to encompass, for such undesignated chiral centers, the racemate, racemic mixtures, each individual enantiomer, a diastereoisomeric mixture and each individual diastereomer of the compound wherein such forms are possible due to the presence of one or more asymmetric centers. The separation of a mixture of stereoisomers can be carried out at an intermediate step during the synthesis of a compound of Formula (I), compound of Formula (II), compound of Formula (III), compound of Formula (IV), compound of Formula (V), compound of Formula (VI), or a pharmaceutically acceptable salt thereof, or it can be done on a final racemic product. Absolute stereochemistry may be determined by X-ray crystallography of crystalline products or crystalline intermediates, which are derivatized, if necessary, with a reagent containing a stereogenic center of known configuration. Alternatively, absolute stereochemistry may be determined by Vibrational Circular Dichroism (VCD) spectroscopy analysis. The present invention includes all such isomers, as well as salts, solvates (including hydrates), and solvated salts of such racemates, enantiomers, diastereomers, tautomers, and mixtures thereof.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism, the invention includes both the cis form and the trans form, as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula (I), compound of Formula (II), compound of Formula (III), compound of Formula (IV), compound of Formula (V), compound of Formula (VI), or a pharmaceutically acceptable salt thereof, or it can be done on a final racemic product. Absolute stereochemistry may be determined by X-ray crystallography of crystalline products or crystalline intermediates that are derivatized, if necessary, with a reagent containing a stereogenic center of known configuration. Unless a particular isomer, salt, solvate (including hydrates)d salt of such racemate, enantiomer, or diastereomer is indicated, the present invention includes all such isomers, as well as salts, solvates (including hydrates), and solvated salts of such racemates, enantiomers, diastereomers, and mixtures thereof.

The term “compound” refers to the compound and, in some embodiments, to the extent they are stable, any hydrate thereof. A hydrate is the compound complexed with water, and a solvate is the compound complexed with a solvent, which may be an organic solvent or an inorganic solvent.

A “stable” compound is a compound that can be prepared and isolated and whose structure and properties remain or can be caused to remain essentially unchanged for a period of time sufficient to allow use of the compound for the purposes described herein (e.g., therapeutic administration to a patient). The compounds of the present invention are limited to stable compounds embraced by Formula (I), Formula (II), Formula (III), Formula (IV), Formula (V), Formula (VI), or pharmaceutically acceptable salts thereof. As indicated herein, the compounds of the present invention can be administered in the form of pharmaceutically acceptable salts. Those skilled in the art will recognize those instances in which the compounds of the invention may form salts. Examples of such compounds are described herein by reference to possible salts. Such reference is for illustration only.

Pharmaceutically acceptable salts can be used with compounds for treating patients. Non-pharmaceutical salts may, however, be useful in the preparation of intermediate compounds.

The term “pharmaceutically acceptable salt” refers to a salt (including an inner salt such as a zwitterion) that possesses effectiveness similar to the parent compound and that is not biologically or otherwise undesirable (e.g., is neither toxic nor otherwise deleterious to the recipient thereof). Thus, an embodiment of the invention provides pharmaceutically acceptable salts of the compounds of the invention. The term “salt(s)”, as used herein, denotes any of the following: acidic salts formed with inorganic and/or organic acids, as well as basic salts formed with inorganic and/or organic bases. Salts of compounds of the invention may be formed by methods known to those of ordinary skill in the art, for example, by reacting a compound of the invention with an amount of acid or base, such as an equivalent amount, in a medium such as one in which the salt precipitates or in aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (“tosylates”) and the like. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.), Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts within the scope of the invention and all acid and base salts are considered equivalent to the free forms of the corresponding compounds for purposes of the invention.

In addition, when a compound of the invention contains both a basic moiety, such as, but not limited to an aliphatic primary, secondary, tertiary or cyclic amine, an aromatic or heteroaryl amine, pyridine or imidazole, and an acidic moiety, such as, but not limited to tetrazole or carboxylic acid, zwitterions (“inner salts”) may be formed and are included within the terms “salt(s)” as used herein. It is understood that some compounds of the invention may exist in zwitterionic form, having both anionic and cationic centers within the same compound and a net neutral charge. Such zwitterions are included within the invention.

As used herein, the term “treat,” “treating,” or “treatment” refers to all processes in which there may be a slowing, interrupting, arresting, controlling, or stopping of the progression of a disease or disorder described herein. The terms do not necessarily indicate a total elimination of all disease or disorder symptoms. The term also include treatment of a cell in vitro or an animal model.

As used herein, the term “prevent,” “preventing,” or “prevention” refers to reducing or eliminating the onset of the symptoms or complications of a disease, condition or disorder.

The terms “administration of” and or “administering” a compound should be understood to include providing a compound described herein, or a pharmaceutically acceptable salt thereof, and compositions thereof to a patient.

The term “subject” (alternatively “patient”) as used herein refers to a subject that has been the object of treatment, observation, or experiment. In some embodiments, the subject is a mammal. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), Lagomorpha (rabbits), rodents (e.g., rats or mice), Procyon lotor (e.g., raccoons). In particular embodiments, the subject is human.

As used herein, the term “immune response” provides any one or more of the following: specific immune response, non-specific immune response, both specific and non-specific response, innate response, primary immune response, adaptive immunity, secondary immune response, memory immune response, immune cell activation, immune cell proliferation, immune cell differentiation, and cytokine expression. In some embodiments, a compound of the present disclosure is administered in conjunction with one or more additional therapeutic agents including anti-viral compounds, vaccines intended to stimulate an immune response to one or more predetermined antigens, adjuvants, CTLA-4 and PD-1 pathway antagonists and other immunomodulatory agents, lipids, liposomes, peptides, anti-cancer agents, and chemotherapeutic agents, etc. In some embodiments, a compound of the present disclosure is administered in conjunction with one or more additional compositions including anti-viral compounds, vaccines intended to stimulate an immune response to one or more predetermined antigens, adjuvants, CTLA-4 and PD-1 pathway antagonists and other immunomodulatory agents, lipids, liposomes, peptides, anti-cancer agents, and chemotherapeutic agents, etc.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of 1%, +2%, +3%, +4%, +5%, +10%, +15%, and +20% and their numerical equivalents.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

The following experimental procedures detail the preparation of specific examples of the instant disclosure. The compounds of the examples are drawn in their neutral forms in the procedures and tables below. In some cases, the compounds were isolated as salts depending on the method used for their final purification and/or intrinsic molecular properties. The examples are for illustrative purposes only and are not intended to limit the scope of the instant disclosure in any way.

### EXAMPLES

The following examples illustrate the disclosure. These examples are not intended to limit the scope of the present disclosure, but rather to provide guidance to the skilled artisan to prepare and use the Compounds, compositions, and methods of the present disclosure. While particular embodiments of the present disclosure are described, the skilled artisan will appreciate that various changes and modifications can be made without departing from the spirit and scope of the disclosure.

It will be understood that certain Compounds of the disclosure may be potent immunomodulators and accordingly, care should be exercised in their handling.

The reactions described herein are applicable for producing Compounds of the disclosure having a variety of different substituent groups (e.g., R¹, R², etc.), as defined herein. The skilled artisan will appreciate that if a particular substituent is not compatible with the synthetic methods described herein, the substituent may be protected with a suitable protecting group that is stable to the reaction conditions. Suitable protecting groups and the methods for protecting and de-protecting different substituents using such suitable protecting groups are well known to those skilled in the art; examples of which may be found in T. W. Greene ‘Protective Groups in Organic Synthesis’ (4th edition, J. Wiley and Sons, 2006). Unless otherwise noted, all starting materials were obtained from commercial suppliers and used without further purification.

### Abbreviations

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples.
ACN Acetonitrile
BINAP Diphenylphosphino
DCM Dichloromethane
DPPD N,N′-Diphenyl-p-phenylenediamine
DMF Dimethylformamide
DMSO Dimethylsulfoxide
ESI Electrospray ionization
EtOAc Ethyl acetate
FBS Fetal bovine serum
HATU 2-(1H-7-Azabenzotriazol-1-yl)-1,1,3,3-tetramethyluronoium hexafluorphosphate
HPLC High pressure liquid chromatography
LDA Lithium diisopropylamide
LiOH Lithium hydroxide
MeOH Methanol
MS Mass spectrophotometry
PyBOP (benzotriazol-1-yl-oxytripyrrolidinophosphonium hexafluorophosphate)
¹H NMR Proton nuclear magnetic resonance spectroscopy
TBAF Tetra-n-butylammonium fluoride
TEMPO (2,2,6,6-Tetramethylpiperidin-1-yl)oxyl or (2,2,6,6-tetramethylpiperidin-1-yl)oxidanyl,
TFA Trifluoroacetic acid
THF Tetrahydrofuran
Xphos 2-Dicyclohexylphosphino-2′,4′,6′-triisopropylbiphenyl

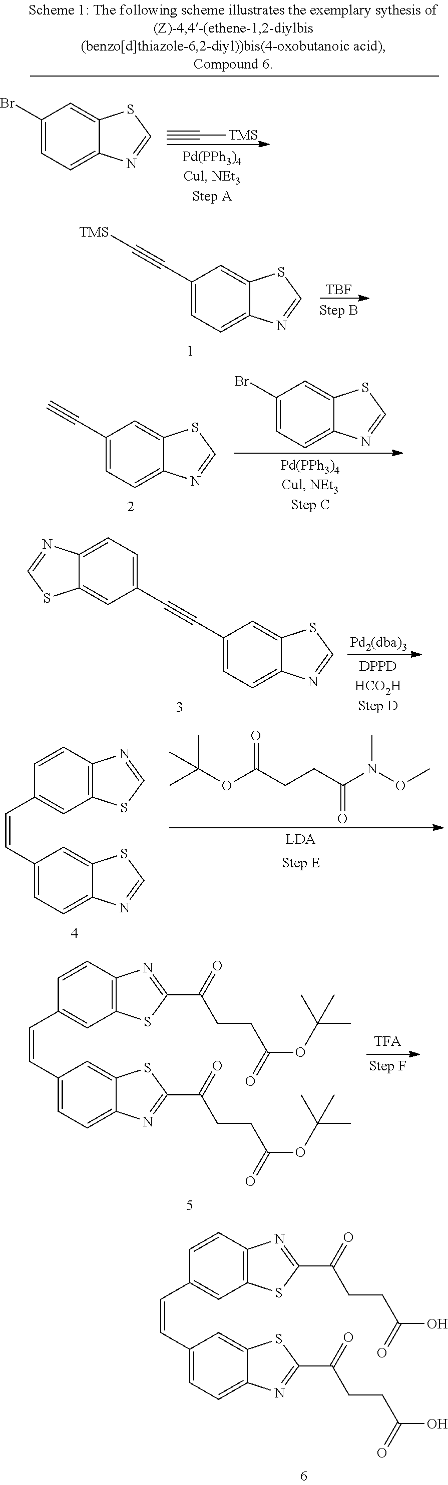

### Example 1: (Z)-4,4′-(ethene-1,2-diylbis(benzo[d]thiazole-6,2-diyl))bis(4-oxobutanoic acid), Compound 6

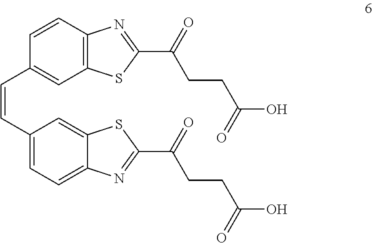 [SMILES: O=C(O)CCC(=O)c1nc2ccc(/C=C\c3ccc4nc(C(=O)CCC(=O)O)sc4c3)cc2s1]

Step A: A mixture of 6-bromobenzo[d]thiazole (500 mg, 2.34 mmol), ethynyltrimethylsilane (0.49 mL, 3.50 mmol), Pd(PPh₃)₄ (135 mg, 0.12 mmol), CuI (22 mg, 0.12 mmol), and Et₃N (1 mL, 0.71 mmol) in THE (12 mL) under N₂ was heated to 70° C. and stirred for 12 hours. The mixture was then cooled to room temperature, diluted in water, and extracted with EtOAc. The organic layer was dried over MgSO₄ and concentrated in vacuo. The residue was purified over silica gel (hexane:EtOAc 80:20 v/v) to afford Compound 1 (200 mg, 37% yield) as a yellow solid. ¹H NMR (400 MHz, DMSO-d₆) δ 9.46 (s, 1H), 8.34 (d, J=1.6 Hz, 1H), 8.04 (d, J=8.4 Hz, 1H), 7.57 (dd, J=8.4, 1.7 Hz, 1H), 0.24 (s, 9H).

Step B: To a stirred solution of Compound 1 (200 mg, 0.86 mmol) in THE (9 mL) was added TBAF (1M in THF, 1.73 mL, 1.73 mmol). The mixture was stirred at room temperature for 15 minutes, then diluted in water and extracted with EtOAc. The organic layer was dried over MgSO₄ and concentrated in vacuo. The residue was purified over silica gel (hexane:EtOAc 75:25 v/v) to afford Compound 2 (89 mg, 54% yield) as a yellow oil. ¹H NMR (400 MHz, DMSO-d₆) δ 9.47 (s, 1H), 8.36 (d, J=1.6 Hz, 1H), 8.06 (d, J=8.4 Hz, 1H), 7.60 (dd, J=8.4, 1.7 Hz, 1H), 4.29 (s, 1H).

Step C: A mixture of Compound 2 (80 mg, 0.50 mmol), 6-bromobenzo[d]thiazole (129 mg, 0.60 mmol), Pd(PPh₃)₄ (29 mg, 0.025 mol), CuI (5 mg, 0.025 mmol), and Et₃N (0.14 mL, 1.00 mmol) in THE (5 mL) under N₂ was heated to 60° C. and stirred for 12 hours. The mixture was then cooled to room temperature, diluted in water, and extracted with DCM. The organic layer was dried over MgSO₄ and concentrated in vacuo. The residue was purified over silica gel (hexane:EtOAc 1:1 v/v) to afford Compound 3 (53 mg, 27% yield) as a light brown solid. ¹H NMR (400 MHz, DMSO-d₆) δ 9.49 (s, 1H), 8.46 (d, J=1.7 Hz, 1H), 8.13 (d, J=8.5 Hz, 1H), 7.73 (dd, J=8.4, 1.7 Hz, 1H). ESI-MS: C₁₆H₉N₂S₂ (M+H): calc. 293.01, found: 293.01.

Step D: A mixture of Compound 3 (50 mg, 0.17 mmol), Pd₂(dba)₃ (7.7 mg, 0.017 mmol), and DPPD (29 mg, 0.068 mmol) in dioxane (5 mL) under N₂ was stirred at room temperature for 15 minutes. Then, formic acid (32 μL, 0.86 mmol) was added, and the mixture was heated to 80° C. and stirred for 12 hours. The mixture was cooled to room temperature, quenched with water, and extracted with DCM. The organic layer was dried over MgSO₄ and concentrated in vacuo. The residue was purified over silica gel (hexane:EtOAc 1:1 v/v) to afford Compound 4 (30 mg, 60% yield) as a yellow solid. ¹H NMR (500 MHz, Chloroform-d) δ 8.95 (s, 2H), 7.97 (d, J=8.5 Hz, 2H), 7.84 (d, J=1.7 Hz, 2H), 7.40 (dd, J=8.5, 1.7 Hz, 2H), 6.81 (s, 2H). ESI-MS: C₁₆H₁₁N₂S₂ (M+H): calc. 295.03, found: 295.04.

Step E: To a stirred solution of Compound 4 (30 mg, 0.10 mmol) in THE (5 mL) under N₂ at −78° C. was added LDA (1M in THF/hexanes, 0.26 mL, 0.26 mmol), and the mixture was stirred at −78° C. for 30 minutes. Then, tert-butyl 4-(methoxy(methyl)amino)-4-oxobutanoate (66 mg, 0.31 mmol) dissolved in THE (2 mL) was added, and the mixture was allowed to slowly warm to room temperature. After stirring for 1 hour, the mixture was quenched with water and extracted with DCM. The organic layer was dried over MgSO₄ and concentrated in vacuo. The residue was purified over silica gel (hexane:EtOAc 70:30 v/v) to afford Compound 5 (32 mg, 52% yield) as a yellow solid. ¹H NMR (500 MHz, Chloroform-d) δ 8.00 (d, J=8.6 Hz, 2H), 7.84 (d, J=1.7 Hz, 2H), 7.42 (dd, J=8.6, 1.7 Hz, 2H), 6.85 (s, 2H), 3.52 (t, J=6.6 Hz, 5H), 2.74 (t, J=6.6 Hz, 5H), 1.44 (s, 19H). ESI-MS: C₃₂H₃₅N₂O₆S₂ (M+H): calc. 607.19, found 607.17.

Step F: To a stirred solution of Compound 5 (30 mg, 0.05 mmol) in DCM (5 mL) was added TFA (1 mL, 13.1 mmol), and the mixture was stirred at room temperature for 12 hours, then it was concentrated in vacuo. The residue was purified over silica gel (hexane:3:1 EtOAc/EtOH, 50:50 v/v) to afford Compound 6 (24 mg, 100% yield) as a yellow solid. ¹H NMR (400 MHz, DMSO-d₆) δ 12.29 (s, 2H), 8.14 (d, J=1.7 Hz, 2H), 8.09 (d, J=8.7 Hz, 2H), 7.42 (dd, J=8.6, 1.7 Hz, 2H), 6.96 (s, 2H), 3.43 (t, J=6.5 Hz, 4H), 2.66 (t, J=6.4 Hz, 4H). ESI-MS: C₂₄H₁₉N₂O₆S₂ (M+H): calc. 495.06, found: 495.06.

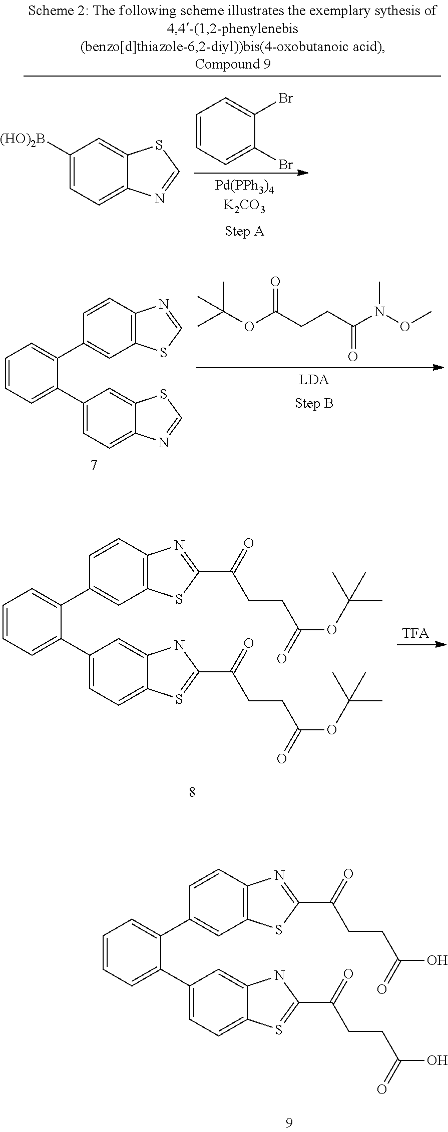

### Example 2: 4,4′-(1,2-phenylenebis(benzo[d]thiazole-6,2-diyl))bis(4-oxobutanoic acid), Compound 9

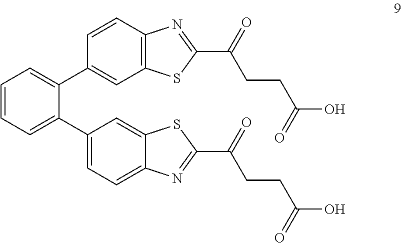 [SMILES: O=C(O)CCC(=O)c1nc2ccc(-c3ccccc3-c3ccc4nc(C(=O)CCC(=O)O)sc4c3)cc2s1]

Step A: A mixture of 1,2-dibromobenzene (100 mg, 0.42 mmol), benzo[d]thiazol-6-ylboronic acid (190 mg, 1.06 mmol), Pd(PPh₃)₄ (49 mg, 0.04 mmol) and K₂CO₃ (293 mg, 2.12 mmol) in degassed toluene:EtOH:water (3:2:1 v/v/v, 6 mL) under N₂ was heat to 100° C. and stirred for 12 hours. The mixture was cooled to room temperature, diluted in DCM, and washed with water. The organic layer was dried over MgSO₄ and concentrated in vacuo. The residue was purified over silica gel (hexane:EtOAc 1:1 v/v) to afford Compound 7 (78 mg, 53% yield) as a yellow solid. ¹H NMR (500 MHz, DMSO-d₆) δ 9.33 (s, 2H), 8.06 (d, J=1.7 Hz, 2H), 7.84 (d, J=8.4 Hz, 2H), 7.53 (s, 4H), 7.15 (dd, J=8.5, 1.8 Hz, 2H). ESI-MS: C₂₀H₁₃N₂S₂ (M+H): calc. 345.04, found: 345.04.

Step B: To a stirred solution of Compound 7 (80 mg, 0.23 mmol) in THE (5 mL) at −78° C. was added LDA (1M in THF/hexanes, 0.58 mL, 0.58 mmol), and the mixture was stirred at −78° C. for 30 minutes. Then, tert-butyl 4-(methoxy(methyl)amino)-4-oxobutanoate (151 mg, 0.70 mmol) was added, and the mixture was slowly warmed to room temperature and stirred for 1 hour. The mixture was quenched with 1N citric acid and extracted with DCM. The combined organic layers were dried over MgSO₄ and concentrated in vacuo. The residue was purified over silica gel (hexane:EtOAc 70:30 v/v) to afford Compound 8 (65 mg, 43% yield) as a yellow solid. ¹H NMR (500 MHz, Chloroform-d) δ 7.94 (d, J=8.6 Hz, 2H), 7.82 (d, J=1.8 Hz, 2H), 7.53 (p, J=2.5 Hz, 4H), 7.27-7.25 (m, 2H), 3.51 (t, J=6.6 Hz, 4H), 2.72 (t, J=6.6 Hz, 4H), 1.43 (s, 18H). ESI-MS: C₃₆H₃₆N₂O₆NaS₂ (M+Na): calc. 679.19, found: 679.19.

Step C: To a stirred solution of Compound 8 (50 mg, 0.08 mmol) in DCM (5 mL) was added TFA (1 mL, 13.1 mmol), and the mixture was stirred at room temperature for 4 hours. The mixture was concentrated in vacuo, and the residue was purified over silica gel (hexane:EtOAc/EtOH 1:1 v/v) to afford Compound 9 (30 mg, 72% yield) as a white solid. ¹H NMR (400 MHz, DMSO-d₆) δ 12.27 (s, 2H), 8.17 (d, J=1.7 Hz, 2H), 8.01 (d, J=8.5 Hz, 2H), 7.58 (d, J=1.8 Hz, 4H), 7.24 (dd, J=8.6, 1.8 Hz, 2H), 3.42 (t, J=6.5 Hz, 4H), 2.65 (t, J=6.4 Hz, 4H). ESI-MS: C₂₈H₂₁N₂O₆S₂ (M+H): calc. 545.08, found: 545.07.

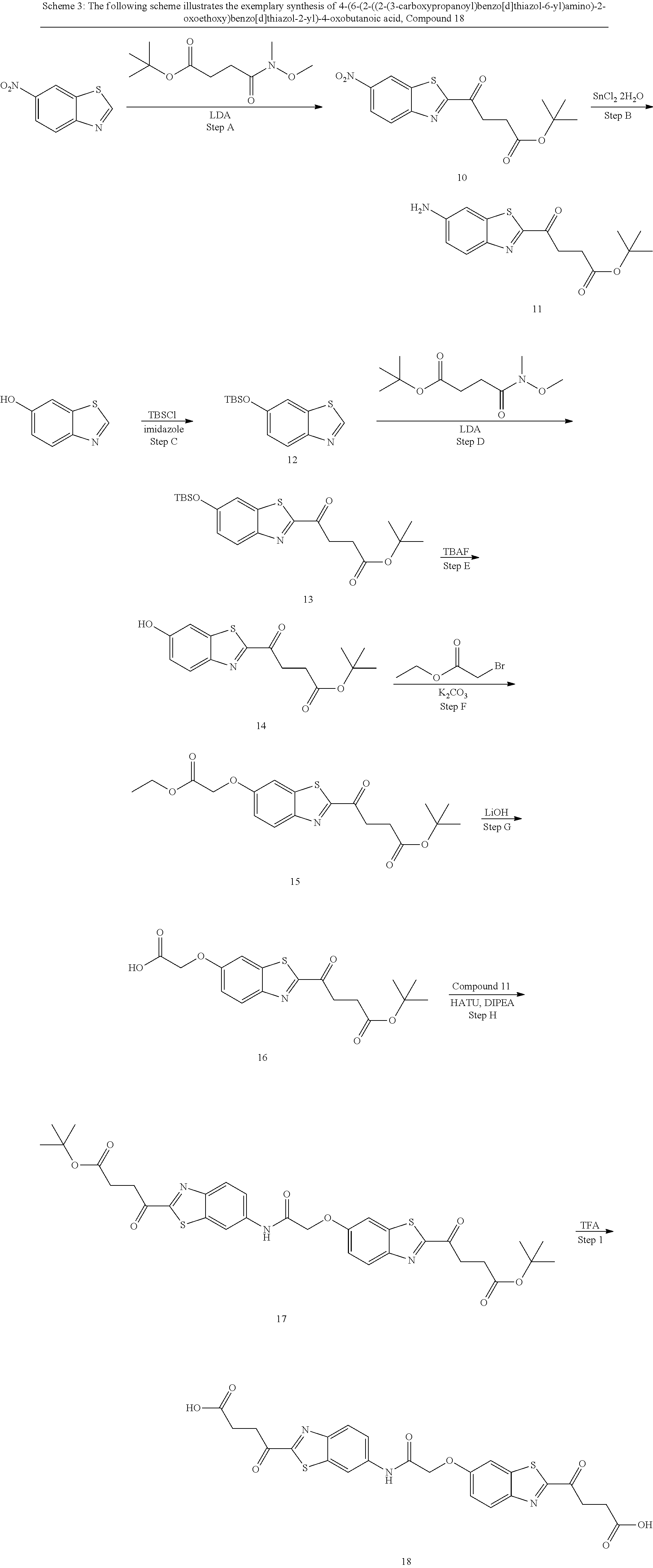

### Example 3: 4-(6-(2-((2-(3-carboxypropanoyl)benzo[d]thiazol-6-yl)amino-2-oxoethoxy)-benzo[d]thiazol-2-yl)-4-oxobutanoic acid, Compound 18

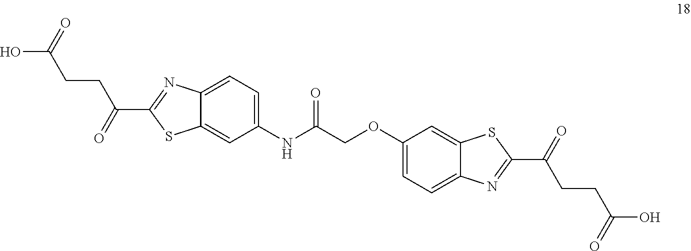 [SMILES: O=C(O)CCC(=O)c1nc2ccc(NC(=O)COc3ccc4nc(C(=O)CCC(=O)O)sc4c3)cc2s1]

Step A: To a stirred solution of 6-nitrobenzo[d]thiazole (500 mg, 2.77 mmol) in THF (14 mL) at −78° C. was added LDA (1M in THIF/hexanes, 4.2 mL, 4.16 mmol), and the mixture was stirred at −78° C. for 30 minutes. Then, tert-butyl 4-(methoxy(methyl)amino)-4-oxobutanoate (904 mg, 4.16 mmol) in THF (5 mL) was added. The mixture was allowed to warm to room temperature and stirred for 2 hours, then quenched with 1N citric acid and extracted with DCM. The combined organic layers were dried over MgSO₄ and concentrated in vacuo. The residue was purified over silica gel (hexane:EtOAc 70:30 v/v) to afford Compound 10 (446 mg, 48% yield) as a yellow solid. ¹H NMR (400 MHz, Chloroform-d) δ 8.93 (dd, J=2.3, 0.5 Hz, 1H), 8.43 (dd, J=9.0, 2.3 Hz, 1H), 8.31 (dd, J=9.1, 0.5 Hz, 1H), 3.55 (dd, J=6.9, 6.1 Hz, 2H), 2.78 (dd, J=6.9, 6.1 Hz, 2H). ESI-MS: C₁₁H₈N₂O₅S (M-tButyl): calc. 280.02, found: 280.04

Step B: To a stirred solution of Compound 10 (100 mg, 0.30 mmol) in ethanol (5 mL) was added Pd/C (10 mg, 10% w/w). Hydrogen gas was bubbled through the solution for 10 minutes, then the mixture was stirred under a hydrogen atmosphere for 2 hours. The mixture was then filtered over Celite and concentrated in vacuo. The residue was purified over silica gel (hexane:EtOAc 1:1 v/v) to afford Compound 11 (45 mg, 49% yield) as a yellow solid. ¹H NMR (400 MHz, Chloroform-d) δ 7.93 (dd, J=8.8, 0.5 Hz, 1H), 7.16-7.08 (m, 1H), 6.90 (dd, J=8.8, 2.3 Hz, 1H), 4.05 (s, 2H), 3.49 (t, J=6.7 Hz, 2H), 2.72 (t, J=6.7 Hz, 2H), 1.43 (s, 9H). ESI-MS: C₁₅H₁₉N₂O₃S (M+H): calc. 307.10, found: 307.09.

Step C: A mixture of benzo[d]thiazol-6-ol (500 mg, 3.31 mmol), imidazole (450 mg, 6.61 mmol) and TBSCl (748 mg, 4.96 mmol) in DCM (17 mL) was stirred at room temperature for 2 hours. The mixture was diluted washed with water, and the organic layer was dried over MgSO₄ and concentrated in vacuo. The residue was purified over silica gel (hexane:EtOAc 70:30 v/v) to afford Compound 12 (805 mg, 92% yield) as a colorless oil. ¹H NMR (400 MHz, Chloroform-d) δ 8.83 (s, 1H), 7.97 (dd, J=8.8, 0.5 Hz, 1H), 7.37 (dd, J=2.4, 0.5 Hz, 1H), 7.03 (dd, J=8.8, 2.4 Hz, 1H), 1.01 (s, 9H), 0.23 (s, 6H). ESI-MS: C₁₃H₂₀NOSSi (M+H): calc. 266.10, found: 266.08.

Step D: To a stirred solution of Compound 12 (800 mg, 3.01 mmol) in THF (15 mL) at −78° C. was added LDA (1M in THF/hexanes, 4.52 mL, 4.52 mmol), and the mixture was stirred at −78° C. for 30 minutes. Then, tert-butyl 4-(methoxy(methyl)amino)-4-oxobutanoate (982 mg, 4.52 mmol) in THE (5 mL) was added. The mixture was allowed to warm to room temperature, stirred for 2 hours, then quenched with 1N citric acid and extracted with DCM. The combined organic layers were dried over MgSO₄ and concentrated in vacuo. The residue was purified over silica gel (hexane:EtOAc 70:30 v/v) to afford Compound 13 (895 mg, 70% yield) as a colorless oil. ¹H NMR (400 MHz, Chloroform-d) δ 8.02 (dd, J=8.9, 0.5 Hz, 1H), 7.35 (dd, J=2.4, 0.5 Hz, 1H), 7.08 (dd, J=8.9, 2.4 Hz, 1H), 3.51 (dd, J=7.0, 6.3 Hz, 2H), 2.73 (t, J=6.6 Hz, 2H), 1.01 (s, 9H), 0.25 (s, 6H). ESI-MS: C₁₇H₂₄NO₄SSi (M-tButyl): calc. 366.11, found: 366.09.

Step E: To a stirred solution of Compound 13 (875 mg, 2.08 mmol) in THE (10 mL) under N₂ was added TBAF (1M in THF, 4.15 mL, 4.15 mmol), and the mixture was stirred at room temperature for 30 minutes. The mixture was diluted with DCM and washed with aqueous NH₄Cl. The combined organic layers were dried over MgSO₄ and concentrate in vacuo. The residue was purified over silica gel (hexane:EtOAc 1:1 v/v) to afford Compound 14 (557 mg, 87% yield) as a white solid. ¹H NMR (400 MHz, DMSO-d₆) δ 7.87 (d, J=9.0 Hz, 1H), 7.25 (d, J=2.4 Hz, 1H), 7.02 (dd, J=9.0, 2.4 Hz, 1H), 3.36 (t, J=6.6 Hz, 2H), 2.60 (t, J=6.6 Hz, 2H), 1.33 (s, 9H). ESI-MS: C₁₅H₁₇NO₄Sna (M+Na): calc. 330.08, found: 330.05.

Step F: A mixture of Compound 14 (540 mg, 1.76 mmol), ethyl 2-bromoacetate (0.58 mL, 5.27 mmol) and potassium carbonate (728 mg, 5.27 mmol) in ACN (9 mL) was stirred at room temperature for 12 hours. The mixture was then diluted in DCM and washed with water, and the combined organic layers were dried over MgSO₄ and concentrated in vacuo. The residue was purified over silica gel (hexane:EtOAc 60:40 v/v) to afford Compound 15 (275 mg, 40% yield) as a white solid. ESI-MS: C₁₅H₁₆NO₆S (M-tBu): calc. 338.06, found: 338.05.

Step G: To a stirred suspension of Compound 15 (600 mg, 1.53 mmol) in THF:EtOH:Water (12 mL, 3:2:1 v/v/v) was added LiGH (183 mg, 7.62 mmol), and the mixture was stirred at room temperature for 30 minutes. The mixture was quenched with acetic acid and concentrated in vacuo. The residue was purified over silica gel (hexane:EtOAc 30:70 v/v) to afford Compound 16 (157 mg, 28% yield) as a white solid. ¹H NMR (400 MHz, Chloroform-d) δ 8.14-8.05 (m, 1H), 7.37 (d, J=2.5 Hz, 1H), 7.23 (dd, J=9.1, 2.6 Hz, 1H), 4.78 (s, 2H), 3.51 (dd, J=6.9, 6.3 Hz, 2H), 2.74 (dd, J=6.9, 6.3 Hz, 2H), 1.43 (s, 9H). ESI-MS: C₁₃H₁₂NO₆S (M-tBu): calc. 310.03, found: 310.01.

Step H: To a stirred solution of Compound 16 (30 mg, 0.08 mmol), Compound 11 (28 mg, 0.09 mmol), and PyBOP (47 mg, 0.09 mmol) in DMF (2 mL) was added DIPEA (0.04 mL, 0.25 mmol), and the mixture was stirred at room temperature for 12 hours. The mixture was concentrated in vacuo, and the residue was purified over silica gel (hexane:EtOAc 1:1 v/v) to afford Compound 17 (25 mg, 47% yield) as a yellow solid. ¹H NMR (400 MHz, Chloroform-d) δ 8.61 (d, J=2.1 Hz, 1H), 8.48 (s, 1H), 8.22-8.12 (m, 2H), 7.60-7.47 (m, 2H), 7.31 (dd, J=9.1, 2.6 Hz, 1H), 4.77 (s, 2H), 3.57-3.48 (m, 4H), 2.74 (t, J=6.6 Hz, 4H), 1.44 (d, J=1.3 Hz, 18H). ESI-MS: C₃₂H₃₆N₃O₈S₂ (M+H): calc. 654.19, found: 654.17.

Step I: To a stirred solution of Compound 17 (10 mg, 0.015 mmol) in DCM (5 mL) was added trifluoroacetic acid (20% v/v), and the mixture was stirred at room temperature for 1 hour. The mixture was concentrated in vacuo, and the residue was purified by HPLC (ACN:water) to afford Compound 18 (2.5. mg, 30% yield) as a yellow solid. ¹H NMR (400 MHz, DMSO-d₆) δ 10.68 (s, 1H), 8.65 (d, J=2.0 Hz, 1H), 8.19 (t, J=8.5 Hz, 2H), 7.84 (d, J=2.6 Hz, 1H), 7.78 (dd, J=9.0, 2.1 Hz, 1H), 7.39 (dd, J=9.0, 2.6 Hz, 1H), 4.94 (s, 2H), 3.43-3.40 (m, 4H), 2.63 (td, J=6.5, 2.1 Hz, 4H). ESI-MS: C₂₄H₂₀N₃O₈S₂ (M+H): calc. 542.06, found: 542.11

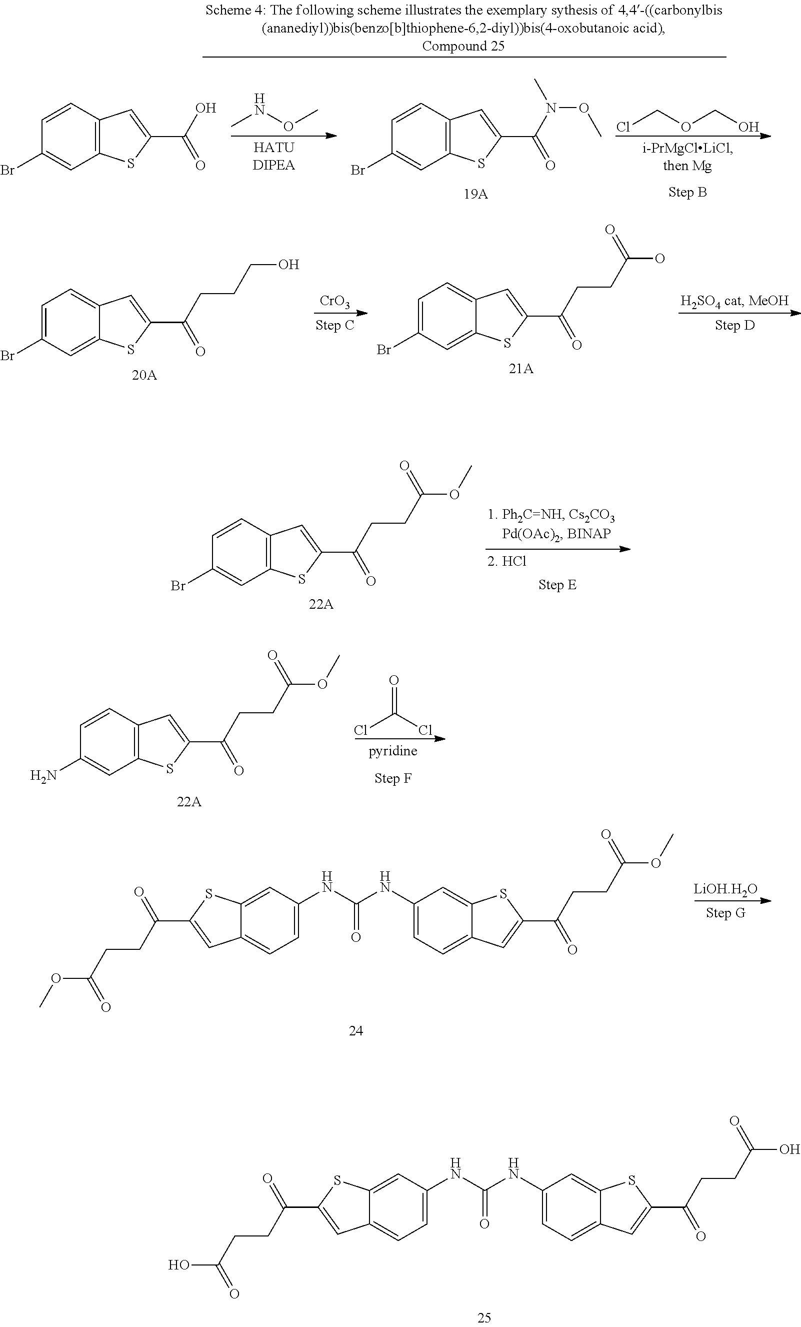

### Example 4: 4,4′-((carbonylbis(azanediyl))bis(benzo[b]thiophene-6,2-diyl))bis(4-oxobutanoic acid), Compound 25

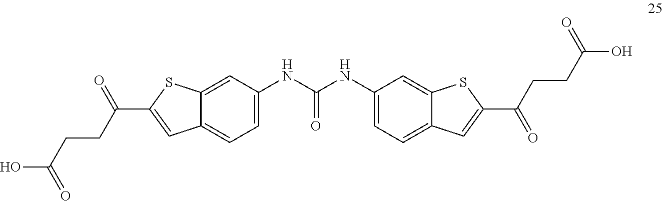 [SMILES: O=C(O)CCC(=O)c1cc2ccc(NC(=O)Nc3ccc4cc(C(=O)CCC(=O)O)sc4c3)cc2s1]

Step A: To a solution of 6-bromobenzo[b]thiophene-2-carboxylic acid (15 g, 58.6 mmol) in DMF (100 mL), N,O-dimethylhydroxylamine (HCl salt, 6.3 g, 64.5 mmol), HATU (44.6 g, 117 mmol) and DIPEA (30.6 mL, 176 mmol) were added sequentially at room temperature. The resulting mixture was stirred at room temperature for 16 hours. Once the reaction was judged to be complete by TLC, the reaction was quenched with ice water and the resulting mixture was extracted with EtOAc. The combined extracts were dried over Na₂SO₄, filtered and concentrated in vacuo. The residue was purified over silica gel (25% EtOAc in hexanes) to afford Compound 19A (14.4 g, 82% yield) as an off white solid. ¹H NMR (400 MHz, DMSO-d₆) δ 8.31 (d, J=1.8 Hz, 1H), 8.17 (s, 1H), 7.93 (d, J=8.5 Hz, 1H), 7.56 (dd, J=8.6, 1.9 Hz, 1H), 3.78 (s, 4H).

Step B: To a solution of 3-chloropropan-1-ol (2.8 mL, 33.4 mmol) in THE (80 mL) at −20° C. under argon was added a solution of i-PrMgCl.LiCl (1.3 M in THF, 26 mL, 33.4 mmol), and the resulting mixture was stirred at −20° C. for 10 minutes Mg (1.0 g, 41.5 mmol) followed by 1,2-dirbromoethane (1.0 mL) were then added and the mixture was allowed to warm to room temperature over 20 minutes. The reaction mixture was then heated at 70° C. for 2 hours, cooled to room temperature and added by cannula over 10 minutes to a solution of Compound 19A (2.0 g, 6.7 mmol) in THF at 0° C. The reaction mixture was warmed to room temperature and stirred for 1 hour. Once the reaction was judged to be complete by TLC, the reaction mixture was quenched with an ice cold saturated NH₄Cl solution and extracted with ethyl acetate. The combined organic extracts were washed with brine, dried over anhydrous sodium sulfate, filtered and concentrated in vacuo. The residue was purified over silica gel (45% EtOAc in hexanes) to afford Compound 20A (1.2 g, 60% yield) as a white solid. ¹H NMR (400 MHz, DMSO-d₆) δ 8.41-8.31 (m, 2H), 7.97 (d, J=8.6 Hz, 1H), 7.63 (dd, J=8.6, 1.9 Hz, 1H), 3.47 (q, J=6.0 Hz, 2H), 3.11 (t, J=7.3 Hz, 2H), 1.81 (p, J=6.8 Hz, 2H).

Step C: CrO₃ (1.43 g, 14.3 mmol) in water (7.1 mL) was cooled to 0° C. then H₂SO₄ (1.43 mL) was added dropwise. The resulting mixture was added dropwise to a solution of Compound 20A in acetone (10 mL) at 0° C. The resulting mixture was stirred at 0° C. for 1 hour, then quenched with water and the separated solids were filtered and dried under reduced pressure to give Compound 21A (0.8 g) as an off white solid, which was used as is. A small amount of Compound 21A was purified over silica gel (50% EtOAc in hexanes) to afford an analytical sample of Compound 21A as a white solid. ¹H NMR (400 MHz, DMSO-d₆) δ 12.19 (s, 1H), 8.37 (d, J=8.4 Hz, 2H), 7.94 (d, J=8.6 Hz, 1H), 7.65-7.58 (m, 1H), 3.27 (d, J=6.3 Hz, 2H), 2.58 (t, J=6.3 Hz, 2H).

Step D: H₂SO₄ (0.6 mL) was added dropwise to a solution of Compound 21A (0.6 g, 1.9 mmol) in MeOH (20 mL). The resulting mixture was heated at 60° C. for 3 hours. Once the reaction was complete, the mixture was cooled to room temperature and concentrated in vacuo. The residue was basified with a saturated NaHCO₃ solution and extracted with EtOAc. The combined organic layers were dried over Na₂SO₄, filtered and concentrated in vacuo to give Compound 22A (0.55 g, 87% yield) as an off-white solid. ¹H NMR (400 MHz, DMSO-d₆) δ 8.39 (s, 1H), 8.36 (d, J=1.8 Hz, 1H), 7.95 (d, J=8.6 Hz, 1H), 7.61 (dd, J=8.6, 1.8 Hz, 1H), 3.57 (s, 3H), 3.34 (t, J=6.4 Hz, 2H), 2.66 (t, J=6.4 Hz, 2H).

Step E: Diphenylmethanimine (2.7 mL, 16.1 mmol), Cs₂CO₃ (7.0 g, 21.5 mmol) and BINAP (racemic, 0.67 g, 1.1 mmol) were added to a solution of Compound 22A in toluene (30 mL) in a sealed tube. The reaction vessel was flushed with nitrogen for 10 minutes before Pd(OAc)₂ (0.12 g, 0.54 mmol) was added and then the vessel was sealed. The resulting mixture was heated at 120° C. for 16 hours, then cooled to room temperature, diluted with EtOAc and filtered through a Celite bed. The Celite was washed with EtOAc and the combined filtrate was concentrated in vacuo. The residue was purified over silica gel (30% EtOAc in hexanes) to give 1.7 g of a yellow sticky solid, which was dissolved in THE (30 mL). HCl (4N in dioxane, 25 mL) was added at room temperature. The mixture was stirred for 20 minutes before concentrated in vacuo. The resulting solid was washed with pentane and dried under reduced pressure to give Compound 23A (0.8 g, 28% yield over 2 steps) as a light yellow solid. ¹H NMR (400 MHz, DMSO-d₆) δ 8.26 (s, 1H), 7.85 (d, J=8.8 Hz, 1H), 7.42 (s, 1H), 7.05 (d, J=8.5 Hz, 1H), 3.60 (s, 3H), 3.31 (t, J=6.4 Hz, 2H), 2.67 (t, J=6.4 Hz, 2H).

Step F: Pyridine (1.1 mL, 13.4 mmol) and phosgene (20% in toluene, 0.66 mL, 1.3 mmol) were added to a solution of Compound 23A (0.5 g, 1.7 mmol) in THE (10 mL) at 0° C. The resulting mixture was stirred at 0° C. for 1 hour and then at room temperature for 3 hours. The pH of the reaction was maintained between 7-8 and progress of the reaction was monitored by TLC. Once complete, the reaction was concentrated in vacuo. Water was added and the resulting solid was collected, dried under reduced pressure and triturated with 20% EtOAc in ether to give Compound 24 (0.25 g, 27% yield) as a yellow solid. ¹H NMR (400 MHz, DMSO-d₆) δ 9.17 (s, 2H), 8.28 (d, J=10.2 Hz, 4H), 7.92 (d, J=8.7 Hz, 2H), 7.48-7.39 (m, 2H), 3.57 (s, 6H), 2.65 (t, J=6.6 Hz, 4H).

Step G: LiOH.H₂O (0.15 g, 3.6 mmol) was added to a solution of Compound 24 in DMSO (6 mL) and water (3 mL) at room temperature. The resulting mixture was stirred at room temperature for 16 h. The reaction mixture was acidified with 2 N HCl solution. The resulting solids were filtered and dried under reduced pressure to give Compound 25 (0.16 g, 80% yield) as a yellow solid. ¹H NMR (400 MHz, DMSO-d₆) δ 12.17 (s, 2H), 9.16 (s, 2H), 8.31-8.23 (m, 4H), 7.91 (d, J=8.8 Hz, 2H), 7.41 (dd, J=8.8, 2.0 Hz, 2H), 3.25 (t, J=6.4 Hz, 4H), 2.57 (t, J=6.3 Hz, 4H).

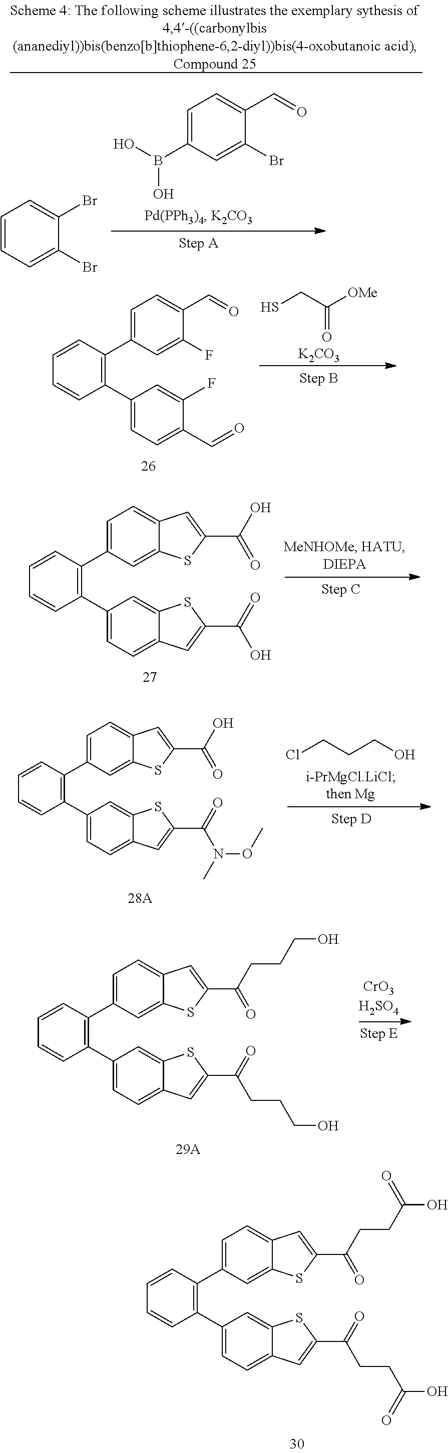

### Example 5: 4,4′-(1,2-phenylenebis(benzo[b]thiophene-6,2-diyl))bis(4-oxobutanoic acid), Compound 30

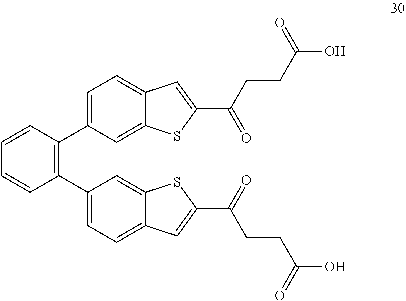 [SMILES: O=C(O)CCC(=O)c1cc2ccc(-c3ccccc3-c3ccc4cc(C(=O)CCC(=O)O)sc4c3)cc2s1]

Step A: To a solution of 1,2-dibromo-benzene (1.0 g, 4.2 mmol) and (3-fluoro-4-formylphenyl)boronic acid (1.6 g, 9.3 mmol) in THF (50 mL) and water (20 mL) in a sealed tube was added K₂CO₃ (2.9 g, 21.2 mmol). The vessel was flushed with nitrogen gas for 15 min before Pd(PPh₃)₄ (0.34 g, 0.30 mmol) was added. The vessel was sealed then heated to reflux at 80° C. for 16 hours. Once the reaction was complete, the mixture was cooled, quenched with water and extracted with DCM. The combined organic layers were dried over Na₂SO₂, filtered and concentrated in vacuo. The residue was purified over silica gel (DCM) to afford Compound 26 (1.0 g, 73% yield) as an off-white solid. ¹H NMR (400 MHz, Chloroform-d) δ 10.30 (d, J=0.8 Hz, 2H), 7.73 (dd, J=8.0, 7.2 Hz, 2H), 7.57-7.49 (m, 2H), 7.48-7.39 (m, 2H), 7.06-6.92 (m, 4H).

Step B: K₂CO₃ (2.1 g, 14.9 mmol) was added to a solution of Compound 26 and methyl 2-mercaptoacetate (1.3 mL, 14.9 mmol) in DMF (10 mL). The mixture was heated at 90° C. for 16 hours. Once the reaction was complete, the mixture was quenched with water and extracted with EtOAc. The aqueous layer was acidified with 1 N HCl and the resulting solids were collected and dried under reduced pressure to give Compound 27 (0.7 g, 65% yield) as an off-white solid. ¹H NMR (400 MHz, DMSO-d₆) δ 7.96-7.88 (m, 4H), 7.70 (d, J=8.4 Hz, 2H), 7.50 (d, J=1.4 Hz, 4H), 7.01 (dd, J=8.3, 1.6 Hz, 2H).

Step C: To a solution of Compound 27 (0.2 g, 0.47 mmol) in DMF (5 mL), DIPEA (0.49 mL, 2.8 mmol) followed by HATU (0.53 g, 1.4 mmol) were added. The mixture was stirred for 10 minutes before N,O-dimethylhydroxylamine (HCl salt, 0.136 g, 1.4 mmol) was added. The reaction was stirred at room temperature for 16 hours, then quenched with water and extracted with EtOAc. The combined organic extracts were washed with brine solution, dried over Na₂SO₄, filtered and concentrated in vacuo. The residue was purified over silica gel (40% EtOAc in hexanes) to afford Compound 28A (0.2 g, 83% yield) as an off-white solid. ¹H NMR (400 MHz, DMSO-d₆) δ 8.11 (s, 2H), 7.95 (s, 2H), 7.77 (d, J=8.3 Hz, 2H), 7.54 (s, 4H), 7.11-7.02 (m, 2H), 3.80 (s, 6H).

Step D: i-PrMgCl.LiCl (9.2 mL, 9.7 mmol) was added to a solution of 3-chloropropanol (0.9 g, 9.7 mmol) in THE (15 mL) at −20° C. and stirred for 20 minutes. Magnesium turnings (0.35 g, 1.9 mmol) and 1,2-dibromoethane (0.3 mL) were added and the reaction was stirred at room temperature for 10 min, then refluxed for 2 h and then cooled to room temperature. This solution was then slowly added to a solution of Compound 28A (0.2 g, 0.4 mmol) in THE (5 mL) at 0° C. The reaction was stirred at this temperature until complete and then quenched with a saturated ammonium chloride solution. The mixture was extracted with EtOAc. The combined organic extracts were washed with a brine solution, dried over Na₂SO₄, filtered and concentrated in vacuo. The residue was purified over silica gel (100% EtOAc) to afford Compound 29A (0.1 g, 50% yield) as an off-white solid. ¹H NMR (400 MHz, DMSO-d₆) δ 8.25 (s, 2H), 7.95 (s, 2H), 7.78 (d, J=8.3 Hz, 2H), 7.55 (s, 4H), 7.07 (d, J=8.5 Hz, 2H), 3.48-3.42 (m, 4H), 3.08 (t, J=7.3 Hz, 4H), 1.79 (p, J=7.2 Hz, 4H).

Step E: CrO₃ (0.097 g, 0.98 mmol) in water (0.5 mL) was cooled to 0° C. then H₂SO₄ (0.1 mL) was added dropwise. The resulting mixture was added dropwise to a solution of Compound 29A in acetone (8 mL) at 0° C., then stirred at 0° C. for 2 hours. The reaction was quenched with water and extracted with EtOAc. The combined organic extracts were dried over Na₂SO₄, filtered and concentrated in vacuo. The residue was purified over silica gel (10-20% MeOH in DCM) to afford Compound 30 (0.1 g, 50% yield) as an off-white solid. ¹H NMR (400 MHz, DMSO-d₆) δ 12.19 (s, 2H), 8.31 (s, 1H), 7.95 (s, 1H), 7.79 (d, J=8.3 Hz, 1H), 7.56 (s, 2H), 7.07 (d, J=8.3 Hz, 1H), 2.61 (d, J=6.3 Hz, 2H), 1.24 (s, 2H).

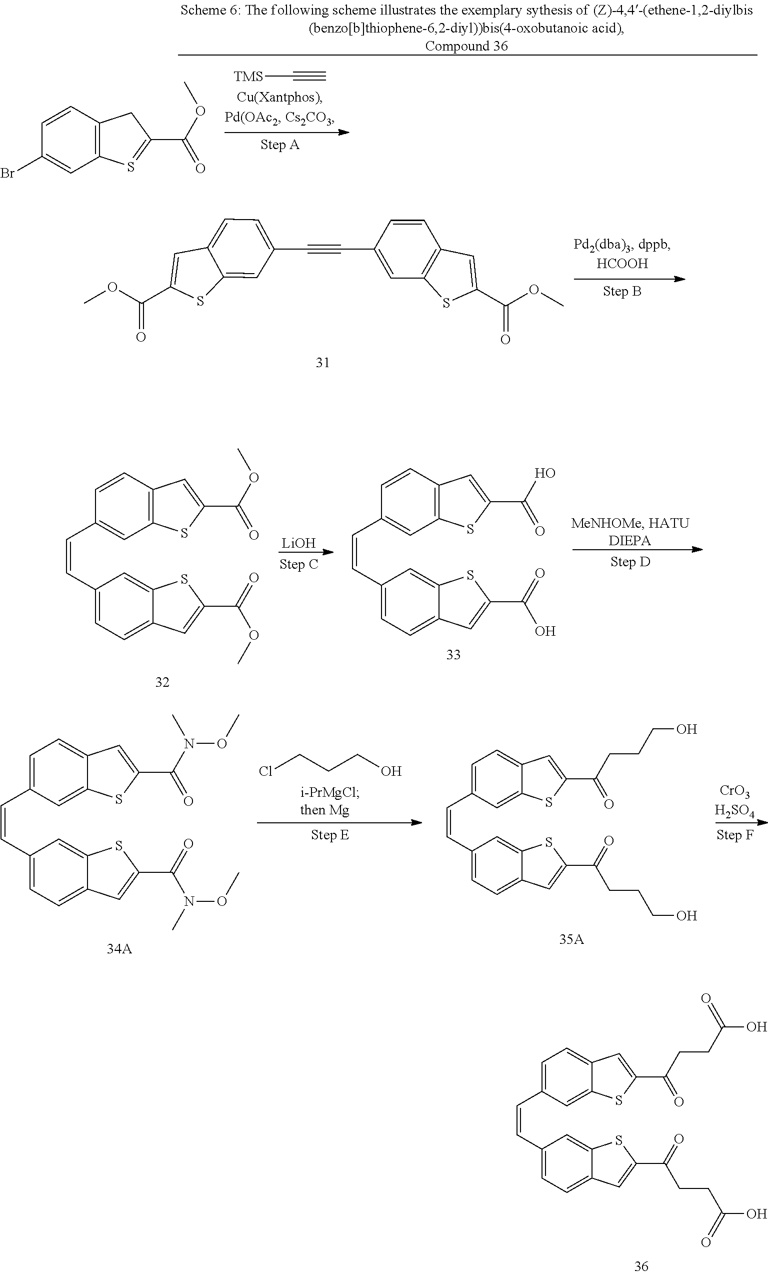

### Example 6: (Z)-4,4′-(ethene-1,2-diylbis(benzo[b]thiophene-6,2-diyl))bis(4-oxobutanoic acid), Compound 36

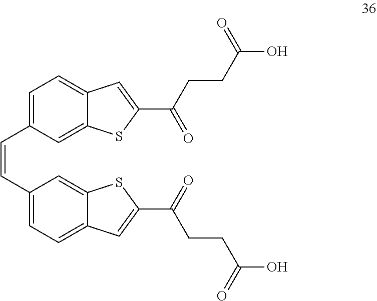 [SMILES: O=C(O)CCC(=O)c1cc2ccc(/C=C\c3ccc4cc(C(=O)CCC(=O)O)sc4c3)cc2s1]

Step A: To a solution of methyl 6-bromobenzo[b]thiophene-2-carboxylate (1.0 g, 3.7 mmol) in DMF (3.3 mL) in a sealed tube were added Cs₂CO₃ (2.4 g, 7.4 mmol), Cu (Xanthphos) (0.028 g, 0.037 mmol), Pd(OAc)₂ (0.008 mg, 0.037 mmol) and trimethylsilylacetylene (0.32 mL, 2.2 mmol) sequentially while purging the vessel with nitrogen gas. The vessel was sealed and the reaction mixture was heated at 60° C. for 24 hours, then cooled, quenched with water and extracted with EtOAc. The combined organic extracts were dried over Na₂SO₄, filtered and concentrated in vacuo and evaporated to dryness to afford a pale brown solid crude. The residue was purified over silica gel (20-30% EtOAc in hexanes) to afford crude Compound 31 (0.6 g), which was used as is in the next step. ¹H NMR (400 MHz, DMSO-d₆) δ 8.35 (s, 2H), 8.23 (s, 2H), 8.06 (d, J=8.3 Hz, 2H), 7.66-7.61 (m, 2H), 3.87 (s, 6H).

Step B: Compound 31 (1.5 g, 3.7 mmol), Pd₂(dba)₃ (0.033 g, 0.037 mmol) and 1,4-bis(diphenylphosphino)butane (0.063 g, 0.015 mmol) were added to a sealed tube and the vessel purged with nitrogen. 1,4-Dioxane (25 mL) was added and the reaction was stirred at room temperature for 15 minutes Formic acid (0.28 mL, 7.4 mmol) was added and the vessel was sealed. The mixture was stirred at 80° C. for 16 hours then cooled. The resulting solid was filtered and washed with DCM. The filtrate was concentrated in vacuo and the resulting residue was purified over silica gel (10-30% EtOAc in hexanes) to afford Compound 32 (0.4 g, 26% yield) as a pale yellow solid. ¹H NMR (400 MHz, Chloroform-d) δ 7.99 (d, J=0.9 Hz, 2H), 7.75 (dt, J=1.5, 0.8 Hz, 2H), 7.68 (d, J=8.4 Hz, 2H), 7.29-7.25 (m, 2H), 6.78 (s, 2H), 3.92 (s, 6H).

Step C: LiOH.H₂O (0.144 g, 3.4 mmol) was added to a solution of Compound 32 (0.28 g, 0.69 mmol) in MeOH (5 mL), THE (5 mL) and water (2.5 mL). The reaction was stirred for 4 hours at room temperature then the solvent was removed in vacuo. The crude residue was taken up in 5 mL of water and the solution was acidified with 1 N aqueous HCl solution. The resulting solids were collected, dried under reduced pressure to afford Compound 33 (0.24 g, 92%) as an off-white solid. ¹H NMR (400 MHz, DMSO-d₆) δ 8.05 (s, 2H), 7.94 (s, 2H), 7.84 (d, J=8.4 Hz, 2H), 7.27 (d, J=8.4 Hz, 2H), 6.87 (s, 2H).

Step D: To a solution of Compound 33 (0.24 g, 0.63 mmol) in DMF (5 mL), DIPEA (0.65 mL, 3.8 mmol) followed by HATU (0.96 g, 2.5 mmol) were added. The mixture was stirred for 10 min before N,O-dimethylhydroxylamine (HCl salt, 0.25 g, 2.5 mmol) was added. The reaction was stirred at room temperature for 16 hours then quenched with water and extracted with EtOAc. The combined organic extracts were washed with brine solution, dried over Na₂SO₄, filtered and concentrated in vacuo. The residue was purified over silica gel (60-80% EtOAc in hexanes) to afford Compound 34A (0.25 g, 85% yield) as an off-white solid. ¹H NMR (400 MHz, DMSO-d₆) δ 8.11 (s, 2H), 7.88 (d, J=1.4 Hz, 2H), 7.81 (d, J=8.4 Hz, 2H), 7.23 (dd, J=8.4, 1.5 Hz, 2H), 6.83 (s, 2H), 2.65 (s, 12H).

Step E: i-PrMgCl.LiCl (1.3M in THF, 9.2 mL, 12 mmol) was added to a solution of 3-chloropropanol (1 mL, 12 mmol) in THE (15 mL) at −20° C. and stirred for 20 minutes. Magnesium turnings (0.35 g, 14.4 mmol) and 1,2-dibromoethane (0.3 mL) were added and the reaction was stirred at room temperature for 10 min then refluxed for 2 h and then cooled to room temperature. This solution was then slowly added to a solution of Compound 34A (0.25 g, 0.54 mmol) in THE (5 mL) at 0° C., stirred until complete and then quenched with a saturated ammonium chloride solution. The mixture was extracted with EtOAc. The combined organic extracts were washed with a brine solution, dried over Na₂SO₄, filtered and concentrated in vacuo. The residue was purified over silica gel (5% MeOH in DCM) to afford Compound 35A (0.1 g, 40% yield) as an off-white solid. ¹H NMR (400 MHz, DMSO-d₆) δ 8.29 (s, 2H), 7.93 (s, 2H), 7.86 (d, J=8.4 Hz, 2H), 7.27 (dd, J=8.4, 1.5 Hz, 2H), 6.89 (s, 2H), 3.48 (p, J=6.5, 6.1 Hz, 4H), 3.09 (t, J=7.3 Hz, 4H), 1.80 (p, J=6.9 Hz, 4H).

Step F: CrO₃ (0.215 g, 2.15 mmol) in water (1 mL) was cooled to 0° C. then H₂SO₄ (0.21 mL) was added dropwise. The resulting mixture was added dropwise to a solution of Compound 35 in acetone (10 mL) and THF (10 mL) at 0° C., then stirred for 2 hour. The reaction was concentrated in vacuo to a quarter of the total volume and then quenched with 15 mL of water and stirred for 10 minutes. The solids were filtered, washed with additional water and then dried under reduced pressure. The solids were then taken up in 2 mL of EtOH, heated to reflux for 30 minutes and then filtered at elevated temperature, and the filtrate was extracted with EtOAc. The combined organic extracts were dried over Na₂SO₄, filtered and concentrated in vacuo. The residue was purified over silica gel (10-20% MeOH in DCM) to afford Compound 36 (0.040 g, 38% yield) as a yellow solid. ¹H NMR (400 MHz, DMSO-d₆) δ 12.18 (s, 2H), 8.31 (s, 2H), 7.91 (s, 2H), 7.83 (d, J=8.4 Hz, 2H), 7.23 (d, J=8.4 Hz, 2H), 6.86 (s, 2H), 2.58 (d, J=6.4 Hz, 4H).

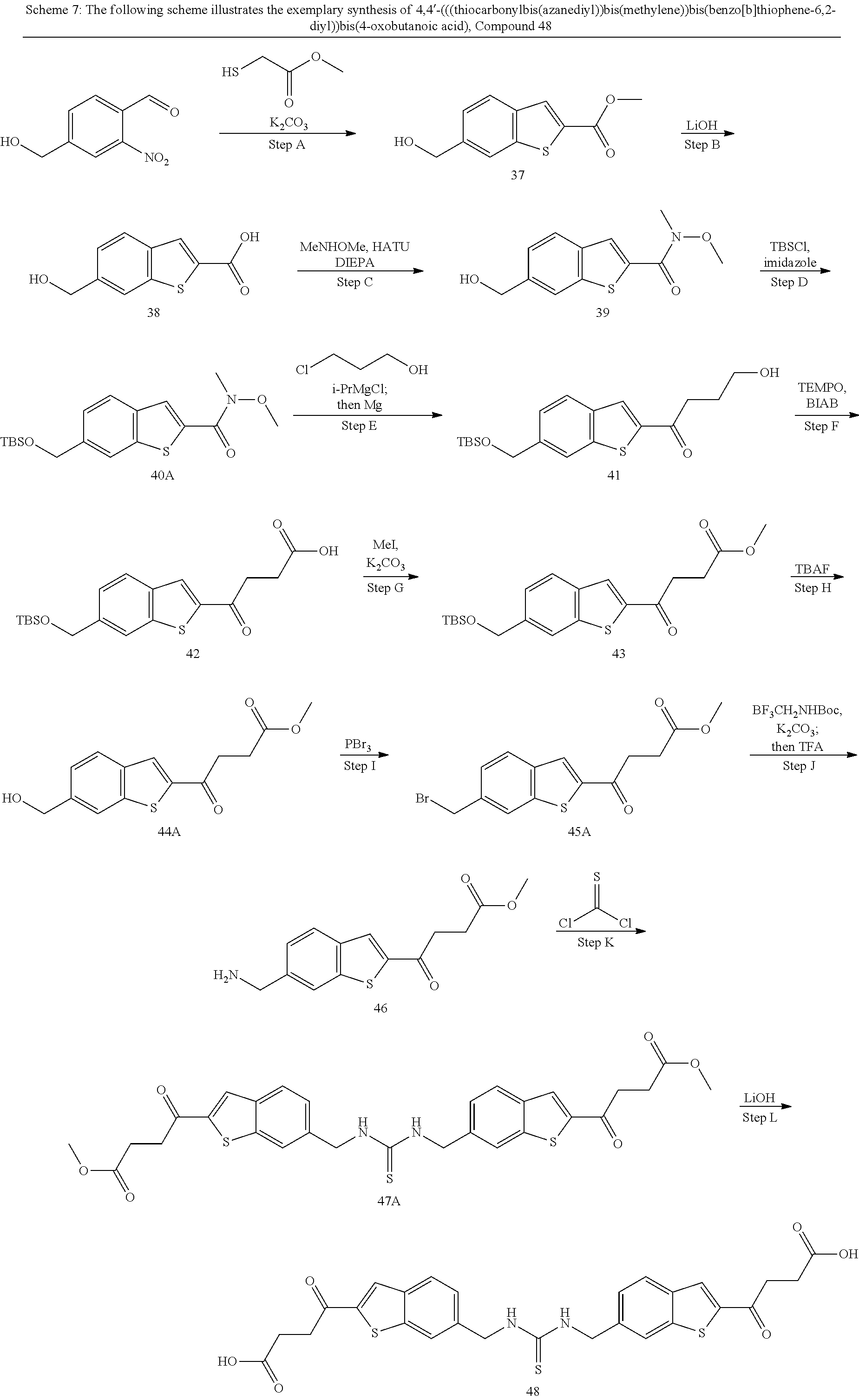

### Example 7: 4,4′-(((thiocarbonylbis(azanediyl))bis(methylene))bis(benzo[b]thiophene-6,2-diyl))bis(4-oxobutanoic acid), Compound 48

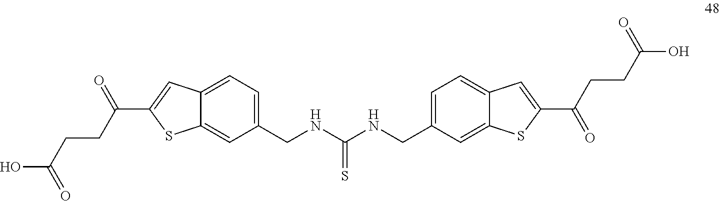 [SMILES: O=C(O)CCC(=O)c1cc2ccc(CNC(=S)NCc3ccc4cc(C(=O)CCC(=O)O)sc4c3)cc2s1]

Step A: To a stirred solution of 4-(hydroxymethyl)-2-nitrobenzaldehyde (5.5 g, 30.4 mmol) in DMF (30 mL) was added K₂CO₃ (5.0 g, 36.5 mmol) and methyl thioglycolate (2.7 mL, 30.4 mmol) and the resulting mixture was stirred at 80° C. for 5 hours. After the reaction was completed, the mixture was cooled, diluted with water and extracted with EtOAc. The combined organic extracts were washed with a brine solution, dried over Na₂SO₄, filtered and concentrated in vacuo. The residue was purified over silica gel (30-50% EtOAc in hexanes) to afford Compound 37 (4.5 g, 66% yield). ¹H NMR (400 MHz, DMSO-d₆) δ 8.15 (s, 1H), 7.93 (d, J=8.1 Hz, 2H), 7.38 (dd, J=8.1, 1.5 Hz, 1H), 4.60 (d, J=5.9 Hz, 2H), 3.84 (s, 3H).

Step B: LiOH.H₂O (3.2 g, 76 mmol) was added to a solution of Compound 37 (4.2 g, 18.9 mmol) in MeOH (30 mL), THE (30 mL) and water (30 mL). The reaction was stirred for 3 hours at room temperature, then diluted with additional water and extracted with diethyl ether. The aqueous layer was acidified with 1 N aqueous HCl solution. The resulting solids were collected and dried under reduced pressure to give Compound 38 (3.6 g, 91% yield). ¹H NMR (400 MHz, DMSO-d₆) δ 8.09 (s, 1H), 8.00-7.89 (m, 2H), 7.41 (d, J=8.6 Hz, 1H), 4.64 (s, 2H).

Step C: To a solution of Compound 38 (3.6 g, 17.3 mmol) in DMF (20 mL), DIPEA (9.0 mL, 52 mmol) followed by HATU (10.5 g, 28 mmol) and N,O-dimethylhydroxylamine (HCl salt, 2.5 g, 26 mmol) were added at 0° C. The reaction was stirred at room temperature for 16 hours, then quenched with water and extracted with EtOAc. The combined organic extracts were washed with brine solution, dried over Na₂SO₄, filtered and concentrated in vacuo. The residue was purified over silica gel (35-40% EtOAc in hexanes) to afford Compound 39 (3.2 g, 73% yield). ¹H NMR (400 MHz, DMSO-d₆) δ 8.17 (s, 1H), 7.98-7.89 (m, 2H), 7.42-7.34 (m, 1H), 4.64 (d, J=5.7 Hz, 2H), 3.82 (s, 3H), 3.34 (s, 3H).

Step D: Imidazole (1.6 g, 23 mmol) and TBSCl (2.9 g, 19 mmol) were added to a solution of Compound 39 (3.2 g, 12.7 mmol) in DCM (40 mL). The resulting mixture was stirred at room temperature for 16 hours, diluted with water and extracted with DCM. The combined organic extracts were washed with a brine solution, dried over Na₂SO₄, filtered and concentrated in vacuo. The residue was purified over silica gel (35-40% EtOAc in hexanes) to afford Compound 40A (3.6 g, 77% yield). ¹H NMR (400 MHz, DMSO-d₆) δ 8.17 (s, 1H), 7.97 (d, J=8.3 Hz, 1H), 7.92 (s, 1H), 7.43-7.36 (m, 1H), 4.85 (s, 2H), 3.82 (s, 3H), 0.92 (s, 9H), 0.10 (s, 6H).

Step E: i-PrMgCl.LiCl (1.3M in THF, 114 mL, 148 mmol) was added to a solution of 3-chloropropanol (12.3 mL, 148 mmol) in THE (80 mL) at −20° C. and stirred for 20 minutes, Magnesium turnings (3.8 g, 158 mmol) and 1,2-dibromoethane (1.3 mL) were added and the reaction was stirred at room temperature for 20 minutes, then refluxed for 2 hours and then cooled to room temperature. This solution was then slowly added to a solution of Compound 40A (3.6 g, 9.9 mmol) in THE (40 mL) at 0° C., stirred at this temperature until complete and then quenched with a saturated ammonium chloride solution. The mixture was extracted with EtOAc. The combined organic extracts were washed with a brine solution, dried over Na₂SO₄, filtered and concentrated in vacuo. The residue was purified over silica gel (25-30% EtOAc in hexanes) to afford Compound 41 (3.0 g, 83% yield). ¹H NMR (400 MHz, DMSO-d₆) δ 8.29 (s, 1H), 7.95 (d, J=8.2 Hz, 1H), 7.90 (s, 1H), 7.37 (dd, J=8.3, 1.5 Hz, 1H), 4.81 (s, 2H), 3.46-3.41 (m, 2H), 3.07 (t, J=7.3 Hz, 2H), 1.81-1.75 (m, 2H), 0.88 (s, 9H), 0.06 (s, 6H).

Step F: To a stirred solution of Compound 41 (0.1 g, 0.27 mmol) in THF:Water (3:1, 10 mL) was added TEMPO (0.013 g, 0.08 mmol) followed by (Diacetoxyiodo)benzene (0.3 g, 0.96 mmol) at 0° C. The reaction mixture was stirred at room temperature for 3 hours, quenched with a saturated NH₄Cl solution and extracted with EtOAc. The combined organic extracts were washed with a brine solution, dried over Na₂SO₄, filtered and concentrated in vacuo. The residue was purified over silica gel (25-30% EtOAc in hexanes) to afford Compound 42 (0.06 g, 58% yield). ¹H NMR (400 MHz, DMSO-d₆) δ 12.18 (s, 1H), 8.35 (s, 1H), 7.96 (d, J=8.3 Hz, 1H), 7.91 (s, 1H), 7.38 (dd, J=8.3, 1.5 Hz, 1H), 4.81 (s, 2H), 3.27 (d, J=6.3 Hz, 2H), 2.58 (t, J=6.4 Hz, 2H), 0.88 (s, 9H), 0.06 (s, 6H).

Step G: Mel (0.25 mL, 4.0 mmol) and K₂CO₃ (0.73 g, 5.3 mmol) were added to a stirred solution of Compound 42 (1.0 g, 2.6 mmol) in DMF (5 mL) at 0° C. The reaction mixture was stirred at room temperature for 3 hours, quenched with water and extracted with EtOAc. The combined organic extracts were washed with a brine solution, dried over Na₂SO₄, filtered and concentrated in vacuo. The residue was purified over silica gel (10-15% EtOAc in hexanes) to afford Compound 43 (0.9 g, 86% yield). ¹H NMR (400 MHz, DMSO-d₆) δ 8.39 (s, 1H), 8.00 (d, J=8.4 Hz, 1H), 7.95 (s, 1H), 7.42 (dd, J=8.4, 1.5 Hz, 1H), 4.85 (s, 2H), 3.61 (s, 3H), 3.38 (t, J=6.4 Hz, 2H), 2.70 (t, J=6.4 Hz, 2H), 0.92 (s, 9H), 0.10 (s, 6H).

Step H: TBAF (1M in THF, 6.1 mL, 6.1 mmol) was added to a stirred solution of Compound 43 (0.8 g, 2.0 mmol) in THE (20 mL) at 0° C. The reaction mixture was stirred at room temperature for 1 hour, quenched with water and extracted with EtOAc. The combined organic extracts were washed with a brine solution, dried over Na₂SO₄, filtered and concentrated in vacuo. The residue was purified over silica gel (35-40% EtOAc in hexanes) to afford Compound 44A (0.44 g, 77% yield). ¹H NMR (400 MHz, DMSO-d₆) δ 8.39 (s, 1H), 8.01-7.94 (m, 2H), 7.42 (dd, J=8.3, 1.5 Hz, 1H), 4.64 (d, J=5.4 Hz, 2H), 3.61 (s, 3H), 3.38 (t, J=6.4 Hz, 2H), 2.70 (t, J=6.4 Hz, 2H).

Step I: PBr₃ (0.068 mL, 0.72 mmol) was added to a stirred solution of Compound 44A (0.2 g, 0.72 mmol) in DCM (10 mL) at 0° C. The reaction mixture was stirred at room temperature for 2 hours, quenched with a saturated NaHCO₃ solution and extracted with DCM. The combined organic extracts were washed with a brine solution, dried over Na₂SO₄, filtered and concentrated in vacuo to give Compound 45A (0.14 g, 57% yield), which was used as is in the next step. ¹H NMR (400 MHz, DMSO-d₆) δ 8.42 (s, 1H), 8.15 (s, 1H), 8.03 (d, J=8.3 Hz, 1H), 7.55 (dd, J=8.3, 1.6 Hz, 1H), 4.86 (s, 2H), 3.39 (t, J=6.3 Hz, 2H), 2.70 (t, J=6.3 Hz, 2H).

Step J: Compound 45A (1.0 g, 3.1 mmol), potassium (((tert-butoxycarbonyl)amino)methyl) trifluoroborate (1.6 g, 6.7 mmol), Cs₂CO₃ (3.0 g, 9.2 mmol) and Xphos (0.15 g, 0.31 mmol) were combined in a tube and 1,4-dioxane (10 ml) and water (1 mL) were added. The vessel was purged with nitrogen before Pd(OAc)₂ (0.034 g, 0.15 mmol) was added. The tube was sealed and the mixture stirred at 100° C. for 24 hours. The reaction was cooled to room temperature, quenched with ice cold water and extracted with EtOAc. The combined organic extracts were washed with a brine solution, dried over Na₂SO₄, filtered and concentrated in vacuo. The residue was purified over silica gel (25-30% EtOAc in hexanes) to afford 0.11 g of the Boc protected benzyl amine, which was taken up in dioxane (2 mL) and cooled to 0° C. HCl (4M in dioxane, 2 mL) was added and the reaction was stirred for 3 hours at room temperature. The mixture was concentrated in vacuo to give Compound 46 (0.1 g), which was used as is in the next step. ¹H NMR (400 MHz, DMSO-d₆) δ 8.42 (s, 1H), 8.11 (s, 1H), 8.05 (d, J=8.3 Hz, 1H), 7.61-7.53 (m, 1H), 4.14 (q, J=5.8 Hz, 2H), 3.57 (s, 3H), 3.37 (d, J=6.3 Hz, 2H), 2.67 (t, J=6.3 Hz, 2H).

Step K: Triethylamine (0.09 mL, 0.64 mmol) was added to Compound 46 (0.05 g, 0.16 mmol) in DCM (5 mL) at 0° C. Thiophosgene (0.013 mL, 0.17 mmol) was added and the reaction mixture was stirred at room temperature for 16 hours. The mixture was concentrated in vacuo then purified over silica gel (1-2% MeOH in DCM) to afford Compound 47A (0.05 g, 52% yield). ¹H NMR (400 MHz, DMSO-d₆) δ 8.36 (s, 2H), 8.19 (d, J=36.1 Hz, 2H), 7.94 (d, J=8.3 Hz, 2H), 7.38 (d, J=8.0 Hz, 2H), 4.81 (s, 4H), 3.57 (s, 6H), 3.35 (dd, J=6.6, 3.8 Hz, 4H), 2.66 (t, J=6.4 Hz, 4H).

Step L: LiOH.H₂O (0.035 g, 0.84 mmol) was added to a solution of Compound 47A (0.05 g, 0.08 mmol) in MeOH (5 mL), THE (5 mL) and water (5 mL). The reaction was stirred for 3 hours at room temperature then the solvent was removed in vacuo. The crude residue was taken up in 5 mL of water and the solution was acidified with 1N aqueous HCl solution. The resulting solids were collected and dried under reduced pressure. Recrystallization with EtOAc gave Compound 48 (0.04 g, 88% yield). ¹H NMR (400 MHz, DMSO-d₆) δ 8.38 (s, 2H), 8.21 (s, 2H), 7.97 (d, J=7.8 Hz, 2H), 7.41 (d, J=8.4 Hz, 2H), 4.84 (s, 4H), 3.32 (d, J=12.8 Hz, 4H), 2.62 (t, J=6.4 Hz, 4H).

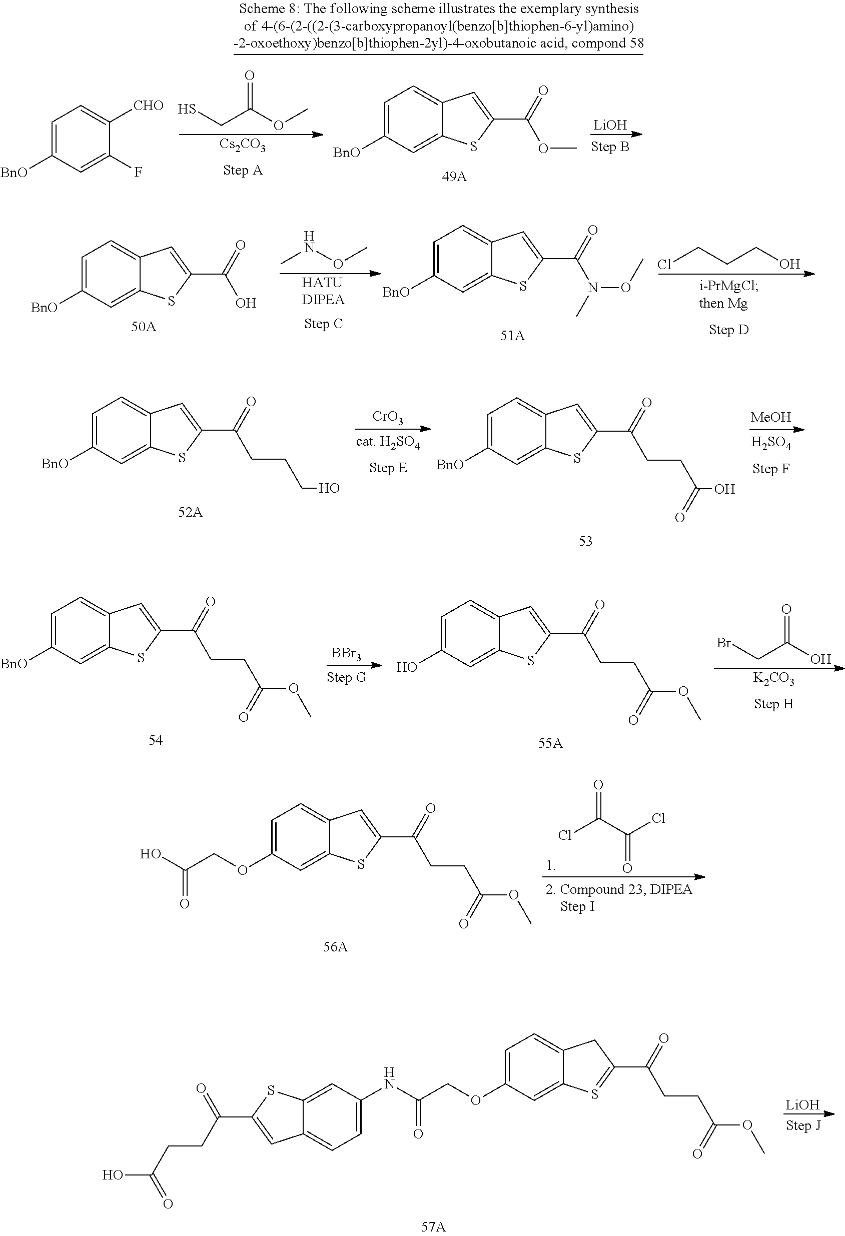

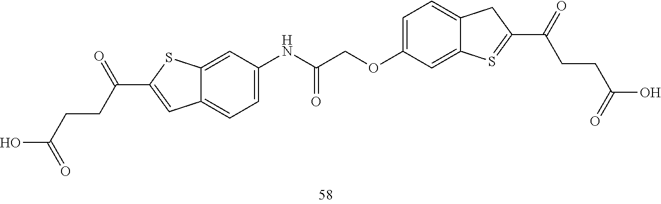

### Example 8: 4-(6-(2-((2-(3-carboxypropanoyl)benzo[b]thiophen-6-yl)amino)-2-oxoethoxy)-benzo[b]thiophen-2-yl)-4-oxobutanoic acid, Compound 58

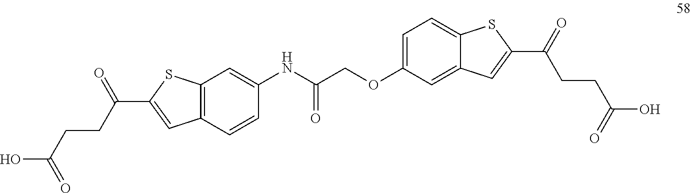 [SMILES: O=C(O)CCC(=O)c1cc2cc(OCC(=O)Nc3ccc4cc(C(=O)CCC(=O)O)sc4c3)ccc2s1]

Step A: To a stirred solution of 4-(benzyloxy)-2-fluorobenzaldehyde (30 g, 130.32 mmol) in DMF (250 mL) was added methyl thioglycolate (15.4 mL, 169.41 mL) and Cs₂CO₃ (63.8 g, 195.5 mmol), and the mixture was stirred at room temperature for 16 hours, then quenched with water and cooled to 0° C. The resulting solid was filtered and purified over silica gel (1:1 EtOAc:hexane v/v) to afford Compound 49A (28.1 g, 73% yield) as a white solid. ¹H NMR (400 MHz, DMSO-d₆) δ 8.09 (s, 1H), 7.89 (d, J=8.8 Hz, 1H), 7.70 (d, J=2.3 Hz, 1H), 7.48-7.43 (m, 2H), 7.42-7.36 (m, 2H), 7.34-7.29 (m, 1H), 7.13 (dd, J=8.8, 2.4 Hz, 1H), 5.17 (s, 2H), 3.83 (s, 3H).

Step B: To a stirred solution of Compound 49A (28 g, 93.84 mmol) in THF:water (200 mL, 7:3 v/v) was added LiOH.H₂O (19.7 g, 469.2 mmol) and the mixture was stirred at room temperature for 16 hours. The mixture was cooled to 0° C. and the resulting solid was filtered to afford Compound 50A (20.1 g, 76% yield) as a white solid.

Step C: To a stirred solution of Compound 50A (6.6 g, 23.24 mmol) in DMF (40 mL) was added N,O-dimethylhydroxylamine HCl (2.48 g, 25.56 mmol), HATU (17.67 g, 46.47 mmol), and DIPEA (12.1 mL, 69.71 mmol), then the mixture was stirred at room temperature for 16 hours. The mixture was quenched with water and extracted with EtOAc, and the combined organic layers were dried over Na₂SO₄ and concentrated in vacuo. The residue was purified over silica gel (hexane:EtOAc 70:30 v/v) afforded Compound 51A (6.2 g, 81% yield) as a yellow solid. ¹H NMR (400 MHz, DMSO-d₆) δ 8.11 (s, 1H), 7.91 (d, J=8.8 Hz, 1H), 7.67 (d, J=2.3 Hz, 1H), 7.49 (d, J=6.9 Hz, 2H), 7.41 (t, J=7.4 Hz, 2H), 7.35 (d, J=7.3 Hz, 1H), 7.14 (dd, J=8.8, 2.4 Hz, 1H), 5.20 (s, 2H), 3.81 (s, 3H).

Step D: To a solution of 3-chloropropan-1-ol (8.6 mL, 104.25 mmol) in THE (150 mL) under N₂ at −20° C. was added i-PrMgCl.LiCl (1.3 M in THF, 80 mL, 104.25 mmol), and the mixture was stirred at −20° C. for 10 minutes. Then Mg (2.86 g, 117.52 mmol) and 1,2-dibromoethane (3.0 mL, catalytic) were added, and the mixture was heated to 60° C. for 2 hours, cooled to room temperature, then the mixture was transferred via cannula to a solution of Compound 51A (6.2 g, 18.96 mmol) in THE (50 mL) under N₂ at 0° C. The mixture was warmed to room temperature and stirred for 1 hour, then quenched with aqueous NH₄Cl and extracted with EtOAc. The combined organic layers were dried over Na₂SO₄ and concentrated in vacuo. The residue was purified over silica gel (hexane:EtOAc 1:1 v/v) to afford Compound 52A (3.8 g, 61% yield) as a white solid. ¹H NMR (400 MHz, DMSO-d₆) δ 8.22 (s, 1H), 7.88 (d, J=8.9 Hz, 1H), 7.67 (d, J=2.3 Hz, 1H), 7.50-7.44 (m, 2H), 7.42-7.28 (m, 3H), 7.12 (dd, J=8.8, 2.3 Hz, 1H), 5.16 (s, 2H), 3.43 (td, J=6.4, 5.1 Hz, 2H), 3.02 (t, J=7.3 Hz, 2H), 1.76 (p, J=6.8 Hz, 2H).

Step E: To a stirred solution of CrO₃ (5.83 g, 58.26 mmol) in water (29.1 mL) was added H₂SO₄ (5.82 mL) at 0° C. The mixture was added dropwise to a stirred solution of Compound 52A (3.8 g, 11.65 mmol) in acetone (30 mL) at 0° C., then warmed to room temperature and stirred for 1 hour. The mixture was quenched with water and the resulting solid was filtered and dried to afford Compound 53 (2.7 g, 68% yield) as a white solid. ¹H NMR (400 MHz, DMSO-d₆) δ 12.16 (s, 1H), 8.28 (s, 1H), 7.89 (d, J=8.9 Hz, 1H), 7.68 (d, J=2.4 Hz, 1H), 7.50-7.44 (m, 2H), 7.43-7.30 (m, 3H), 7.13 (dd, J=8.8, 2.3 Hz, 1H), 5.17 (s, 2H), 3.28-3.20 (m, 2H), 2.61-2.54 (m, 2H).

Step F: To a stirred solution of Compound 53 (2.7 g, 7.94 mmol) in MeOH (30 mL) was added dropwise H₂SO₄ (2 mL) at 0° C. The mixture was heated to 65° C. then stirred for 3 hours, cooled to room temperature and basified with NaHCO₃ then extracted with EtOAc. The combined organic layers were dried over Na₂SO₄ and concentrated in vacuo to afford Compound 54 (2.7 g, crude) as a white solid. ¹H NMR (400 MHz, DMSO-d₆) δ 8.29 (s, 1H), 7.90 (d, J=8.9 Hz, 1H), 7.68 (d, J=2.3 Hz, 1H), 7.50-7.44 (m, 2H), 7.42-7.35 (m, 2H), 7.35-7.27 (m, 1H), 7.13 (dd, J=8.8, 2.3 Hz, 1H), 5.17 (s, 2H), 3.57 (s, 3H), 2.65 (t, J=6.4 Hz, 2H).

Step G: To a stirred solution of Compound 54 (2.7 g, 7.63 mmol) in DCM (50 mL) was added BBr₃ (1M in DCM, 22.8 mL, 22.8 mmol) at −78° C., and the mixture was stirred for 1 hour. The mixture was quenched with MeOH and diluted with water, then extracted with DCM, washed with NaHCO₃, dried over Na₂SO₄, and concentrated in vacuo. The residue was purified over silica gel (hexane:EtOAc 3:1 v/v) to afford Compound 55A (1.4 g, 68% yield) as a white solid. ¹H NMR (400 MHz, DMSO-d₆) δ 10.12 (s, 1H), 8.22 (d, J=0.8 Hz, 1H), 7.80 (d, J=8.7 Hz, 1H), 7.26 (d, J=2.2 Hz, 1H), 6.92 (dd, J=8.7, 2.2 Hz, 1H), 3.56 (s, 3H), 3.29-3.25 (m, 2H), 2.63 (dd, J=7.0, 5.9 Hz, 2H).

Step H: To a stirred solution of Compound 55A (550 mg, 2.08 mmol) in DMF (5 mL) was added K₂CO₃ (719 mg, 5.21 mmol) and 2-bromoacetic acid (434 mg, 3.12 mmol), then the mixture was heated to 90° C. and stirred for 16 hours. The mixture was cooled to room temperature, poured over ice, and the aqueous layer was acidified with 2N HCl to pH<1. The resulting mixture was extracted with EtOAc, and the combined organic layers were dried over Na₂SO₄ and concentrated in vacuo. The residue was purified over silica gel (hexane:EtOAc 1:1 v/v) to afford Compound 56A (150 mg, 22% yield) as a white solid. ¹H NMR (400 MHz, DMSO-d₆) δ 13.09 (s, 1H), 8.32 (s, 1H), 7.93 (d, J=8.8 Hz, 1H), 7.58 (d, J=2.4 Hz, 1H), 7.12 (dd, J=8.9, 2.3 Hz, 1H), 4.80 (s, 2H), 3.60 (s, 3H), 2.68 (t, J=6.3 Hz, 2H).

Step I: To a stirred solution of Compound 56A (150 mg, 0.46 mmol) in DCM (35 mL) under N₂ at 0° C. was added oxalyl chloride (0.06 mL, 0.70 mmol) and DMF (5 μL). The mixture was warmed to room temperature then stirred for 1 hour, then concentrated in vacuo. The residue was dissolved in THE (10 mL) then added to a stirred solution of Compound 23 (prepared as described in Example 4, 98 mg, 0.33 mmol) and DIPEA (0.2 mL, 1.16 mmol) in THE (25 mL) at 0° C. The mixture was warmed to room temperature, stirred for 1 hour. then concentrated in vacuo, and the residue was triturated in water and filtered to afford crude Compound 57A (110 mg) as a brown solid. ¹H NMR (400 MHz, DMSO-d₆) δ 10.48 (s, 1H), 8.47 (s, 1H), 8.35 (d, J=4.2 Hz, 2H), 7.99 (t, J=8.7 Hz, 2H), 7.69-7.61 (m, 2H), 7.24 (dd, J=8.8, 2.3 Hz, 1H), 4.89 (s, 2H), 3.60 (s, 6H), 2.69 (td, J=6.5, 2.9 Hz, 4H).

Step J. To a stirred solution of Compound 57A (110 mg, 0.19 mmol) in DMSO (3 mL) was added LiOH.H₂O (81 mg, 1.94 mmol) in water (1.5 mL), and the mixture was stirred at room temperature for 6 hours, then diluted in water and acidified to pH<1 with 2N HCl. The resulting solid was filtered and dried to afford Compound 58 (25 mg, 24% yield) as a brown solid. ¹H NMR (400 MHz, DMSO-d₆) δ 10.14 (s, 1H), 8.37 (d, J=1.8 Hz, 1H), 8.22 (d, J=3.3 Hz, 2H), 7.94 (dd, J=8.8, 4.2 Hz, 2H), 7.62 (t, J=2.1 Hz, 1H), 7.21 (dd, J=8.8, 2.4 Hz, 1H), 4.78 (d, J=40.2 Hz, 2H), 3.29-3.22 (m, 4H), 2.60 (dt, J=6.5, 3.2 Hz, 4H).

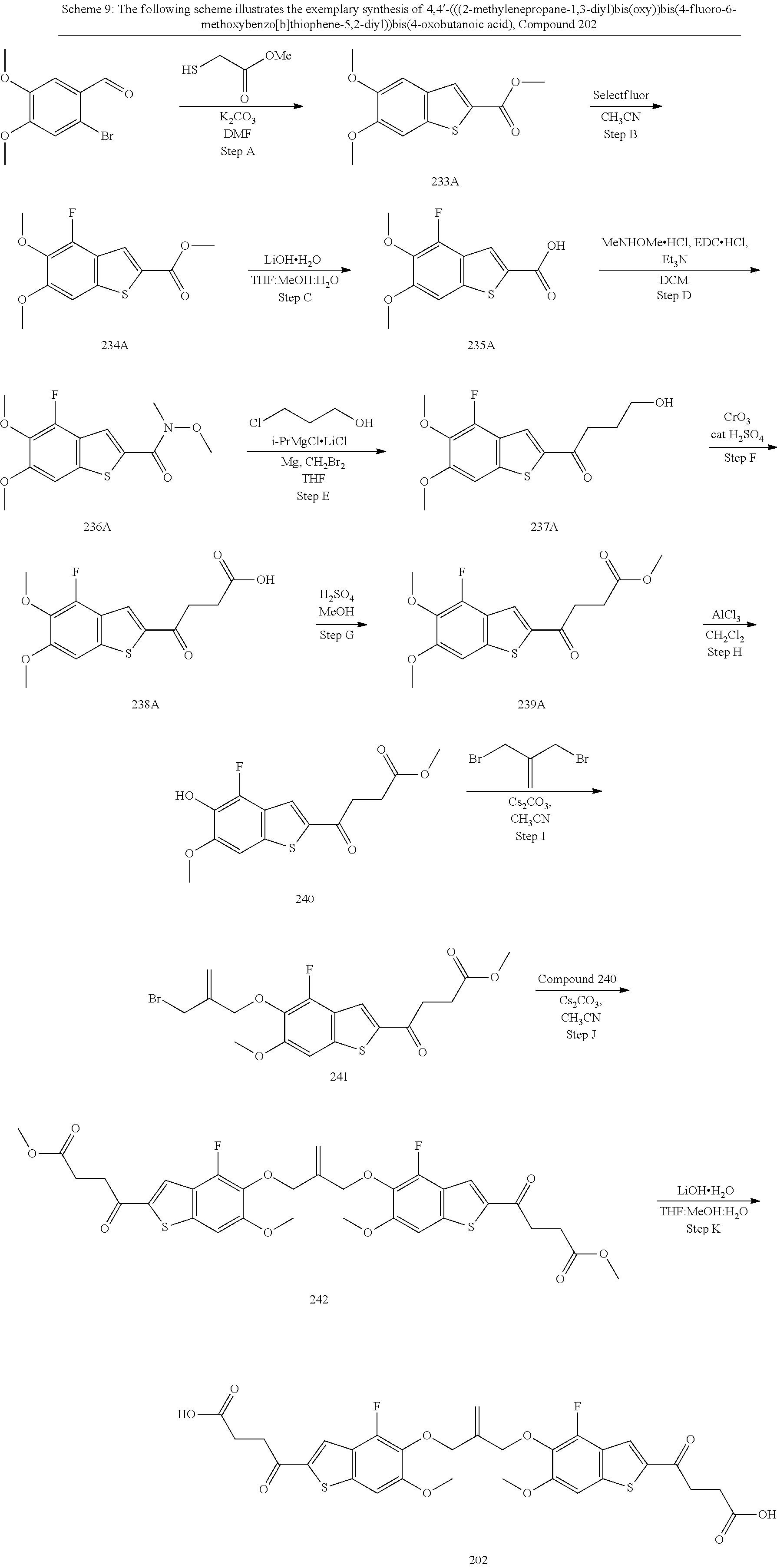

### Example 9: 4,4′-(((2-methylenepropane-1,3-diyl)bis(oxy))bis(4-fluoro-6-methoxybenzo[b]-thiophene-5,2-diyl))bis(4-oxobutanoic acid), Compound 202

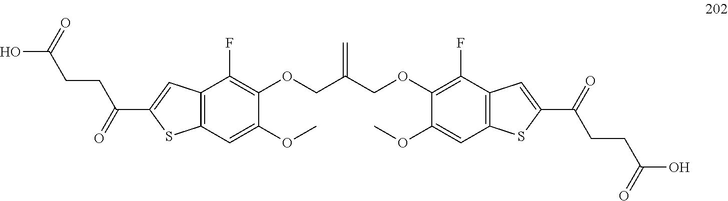 [SMILES: C=C(COc1c(OC)cc2sc(C(=O)CCC(=O)O)cc2c1F)COc1c(OC)cc2sc(C(=O)CCC(=O)O)cc2c1F]

Step A: To a stirred solution of 6-bromoveratraldehyde (12.5 g, 67.9 mmol) in anhydrous DMF (400 mL) was added anhydrous potassium carbonate (28.1 g, 203.75 mmol), methyl 2-mercaptoacetate (9.20 mL, 101.9 mmol) under nitrogen atmosphere at 0° C. The temperature was slowly raised to room temperature, then to 80° C. and maintained at that temperature for 16 hours. The reaction was cooled to room temperature, water (150 mL) was added and then the mixture was filtered to afford Compound 233A (11.0 g, 64% yield) as an off-white solid. ¹H NMR (400 MHz, DMSO-d₆) δ 8.03 (s, 1H), 7.61 (s, 1H), 7.50 (s, 1H), 3.86 (s, 6H), 3.83 (s, 3H). ESI-MS: C12H1304S (M+H): calc. 253.05, found: 253.00.

Step B: To a stirred solution of Compound 233A (4 g, 15.87 mmol) in CH₃CN (100 mL) was added Selectfluor (6.74 g, 19.1 mmol) at room temperature. The reaction mixture was stirred at 45° C. for 18 hours, then cooled to room temperature, diluted with sat. NaHCO₃ (150 mL) and extracted with ethyl acetate (2×100 mL). The combined organic layers were washed with brine (2×100 mL), dried over anhydrous sodium sulfate, filtered and concentrated under reduced pressure to give crude residue which was purified over silica gel (7:93 EtOAc:hexane v/v) to afford Compound 234A (350 mg, 8% yield) as a white solid. ¹H NMR (400 MHz, DMSO-d₆) δ 8.02 (s, 1H), 7.61 (d, J=1.3 Hz, 1H), 3.92 (s, 3H), 3.88 (s, 3H), 3.86 9s, 3H). ESI-MS: C₁₂H₁₂FO₄S (M+H): calc. 271.04, found: 271.00.

Step C: To a stirred solution of Compound 234A (3.9 g, 14.4 mmol) in THF:MeOH:H₂O (400 mL, 3:1:1) was added lithium hydroxide monohydrate (6.1 g, 144.4 mmol) at 0° C. and the reaction mixture was stirred at room temperature for 16 hours. The reaction mixture was adjusted to pH=4 using ice cold 4N HCl then extracted with ethyl acetate (2×30 mL). The organic layer was washed with brine (2×15 mL), dried over anhydrous sodium sulfate, concentrated to give Compound 235A (3.40 g, 91% yield) as an off-white solid. ¹H NMR (400 MHz, DMSO-d₆) δ 13.45 (s, 1H), 7.88 (s, 1H), 7.54 (d, J=1.3 Hz, 1H), 3.87 (s, 3H), 3.81 (s, 3H). ESI-MS: C₁₁H₈FO₄S (M−H): calc. 255.01, found: 255.00.

Step D: To a stirred solution of Compound 235A (3.20 g, 12.5 mmol) in DCM (300 mL), 1-ethyl-3-(3-dimethylaminopropyl)carbodiimide hydrochloride (7.18 g, 37.5 mmol), N,O-dimethylhydroxylamine hydrochloride (2.44 g, 25.0 mmol) and triethylamine (10.5 mL, 74.9 mmol) were added sequentially at 0° C. Then, the reaction mixture was stirred at room temperature for 16 hours, then quenched with ice cold water (25 mL), diluted with EtOAc (50 mL) and separated. The organic layer was washed with brine (2×20 mL), dried over anhydrous sodium sulfate, filtered and concentrated under reduced pressure to give the crude compound which was purified over silica gel (30:70 EtOAc:hexane v/v) to give Compound 236A (2.30 g, 62% yield) as an off-white solid. ¹H NMR (400 MHz, DMSO-d₆) δ 7.98 (s, 1H), 7.57 (d, J=1.4 Hz, 1H), 3.88 (s, 3H), 3.84 (s, 3H), 3.81 (s, 3H), 3.29 (s, 3H). ESI-MS: C₁₃H₁₅FNO₄S (M+H): calc. 300.07, found: 300.00.

Step E: To a solution of 3-chloro-1-propanol (5.42 mL, 65.2 mmol) in THE (40 mL) at −20° C. under argon was added a solution of isopropylmagnesium chloride lithium chloride complex (1.3 M in THF; 50.0 mL, 65.2 mmol) and the resulting mixture was stirred at −20° C. for 10 min. Magnesium turnings (1.58 g, 65.2 mmol) were added, followed by the dropwise addition of 1,2-dibromoethane (1 mL). The mixture was allowed to warm up to room temperature over 20 min, then heated at 70° C. for 2 hours, before being cooled to room temperature and then added over 10 min through a cannula to a solution of Compound 236A (1.3 g, 4.34 mmol) in THE (40 mL) at 0° C. The reaction mixture was warmed to room temperature and stirred for 2 hours, then quenched with ice cold sat. NH₄Cl solution (50 mL) and extracted with ethyl acetate (2×100 mL). The organic layer was washed with brine (2×50 mL), dried over anhydrous sodium sulfate and concentrated to give a residue which was purified over silica gel (20:80 EtOAc:hexane v/v) to afford Compound 237A (750 mg, 58% yield) as an off-white solid. ¹H NMR (400 MHz, DMSO-d₆) δ 8.24 (s, 1H), 7.57 (s, 1H), 3.92 (s, 3H), 3.86 (s, 3H), 3.47 (q, J=6.1 Hz, 2H), 3.10 (t, J=7.3 Hz, 2H), 1.79 (p, J=6.9 Hz, 2H). ESI-MS: C₁₄H₁₆FO₄S (M+H): calc. 299.07, found: 299.10.

Step F: To a stirred solution of CrO₃ (3.86 g, 38.58 mmol) in water (19.3 mL) was added H₂SO₄ (3.86 mL) at 0° C. The resulting mixture was added dropwise to a stirred solution of Compound 237A (2.30 g, 7.72 mmol) in acetone (25 mL) at 0° C., then warmed to room temperature and stirred for 1 hour. The mixture was quenched with water and the resulting solid was filtered and dried to afford Compound 238A (1.90 g, 80% yield) as a white solid. ¹H NMR (400 MHz, DMSO-d₆) δ 12.17 (s, 1H), 8.31 (s, 1H), 7.58 (s, 1H), 3.92 (s, 3H), 3.86 (s, 3H), 3.29 (t, J=6.3 Hz, 2H), 2.60 (t, J=6.3 Hz, 2H). ESI-MS: C₁₄H₁₄FO₅S (M+H): calc. 313.05, found: 312.85.

Step G: To a stirred solution of Compound 238A (1.9 g, 6.09 mmol) in MeOH (15 mL) was added dropwise H₂SO₄ (1.5 mL) at 0° C. The mixture was heated to 70° C. then stirred for 3 hours, cooled to room temperature and basified with NaHCO₃ then extracted with EtOAc. The combined organic layers were dried over Na₂SO₄ and concentrated in vacuo to afford Compound 239A (1.60 g, 80% yield) as a white solid. ¹H NMR (400 MHz, DMSO-d₆) δ 8.33 (s, 1H), 7.58 (s, 1H), 3.93 (s, 3H), 3.86 (s, 3H), 3.60 (s, 3H), 3.38 (t, J=6.3 Hz, 2H), 2.68 (t, J=6.3 Hz, 2H). ESI-MS: ESI-MS: C₁₅H₁₆FO₅S (M+H): calc. 327.07, found: 326.95.

Step H: To a stirred solution of Compound 239A (1.60 g, 4.91 mmol) in DCM (50 mL) was added AlCl₃ (1.96 g, 14.7 mmol) at 27° C., and the mixture was stirred for 16 hour. The mixture was quenched with MeOH and concentrated to give a residue which was purified over silica gel (40:60 EtOAc:hexane v/v) to afford Compound 240 (1.30 g, 84% yield) as an off-white solid. ¹H NMR (400 MHz, DMSO-d₆) δ 9.52 (s, 1H), 8.25 (s, 1H), 7.47 (s, 1H), 3.91 (s, 3H), 3.60 (s, 3H), 3.37 (t, J=6.3 Hz, 2H), 2.67 (t, J=6.3 Hz, 2H). ESI-MS: C₁₄H₁₄FO₅S (M+H): calc. 313.05, found: 312.90.

Step I: To the solution of Compound 240 (50 mg, 0.16 mmol) in CH₃CN (5 mL) was added methyl 3-bromo-2-(bromomethyl)prop-1-ene (135 mg, 0.64 mmol) and Cs₂CO₃ (208 mg, 0.64 mmol) and the resulting mixture was stirred at 60° C. for 1 hour. The reaction mixture was cooled to room temperature, then filtered through a pad of Celite and washed with THF. The filtrate was concentrated to give a residue which was purified over silica gel (25:75 EtOAc:hexane v/v) to afford Compound 241 (45 mg, 63% yield). ¹H NMR (400 MHz, DMSO-d₆) δ 8.31 (s, 1H), 7.58 (d, J=4.1 Hz, 1H), 5.47 (s, 1H), 5.34 (s, 1H), 4.66 (s, 2H), 4.31 (s, 2H), 3.91 (d, J=4.3 Hz, 3H), 3.58 (s, 3H), 3.36 (t, J=6.4 Hz, 2H), 2.65 (t, J=6.3 Hz, 2H). ESI-MS: C₁₈H₁₉BrFO₅S (M+H): calc. 445.01, found: 445.00.

Step J: To the solution of Compound 241 (70 mg, 0.22 mmol) in CH₃CN (8 mL) was added Compound 240 (90 mg, 0.20 mmol) and Cs₂CO₃ (365 mg, 1.120 mmol) and the resulting mixture was stirred at 70° C. for 3 hours. The reaction mixture was cooled to room temperature, then filtered through a pad a celite and washed with THF. The filtrate was concentrated to give a residue which was purified over silica gel (50:50 EtOAc:hexane v/v) to afford Compound 242 (60 mg, 58% yield). ESI-MS: C₃₂H₃₁F₂O₁₀S₂ (M+H): calc. 677.13, found: 677.00.

Step K: To the solution of Compound 242 (60 mg, 0.09 mmol) in THF:MeOH:H₂O (4.0 mL, 2:1:1) was added lithium hydroxide monohydrate (18 mg, 0.44 mmol) at 0° C. and the reaction mixture was stirred for 2 hours at 27° C., then concentrated, acidified with 1N HCl and then filtered. The collected solid was washed with water and pentane to afford Compound 202 (11 mg, 20% yield). ¹H NMR (400 MHz, DMSO-d₆) δ 12.16 (s, 2H), 8.21 (s, 2H), 7.51 (s, 2H), 5.28 (s, 2H), 4.71 (s, 4H), 3.84 (s, 6H), 3.25 (d, J=6.3 Hz, 4H), 2.55 (t, J=6.3 Hz, 4H). ESI-MS: C₃₀H₂₇F₂O₁₀S₂ (M+H): calc. 649.10, found: 649.05.

### Example 10: (E)-4,4′-((but-2-ene-1,4-diylbis(oxy))bis(4-fluoro-6-methoxybenzo[b]thiophene-5,2-diyl))bis(4-oxobutanoic acid), Compound 203

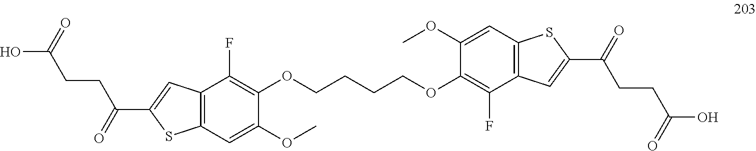 [SMILES: COc1cc2sc(C(=O)CCC(=O)O)cc2c(F)c1OCCCCOc1c(OC)cc2sc(C(=O)CCC(=O)O)cc2c1F]

Synthesis of the title compound was carried out as described in Example 9, except in Step I, trans-1,4-dibromo-2-butene was used instead of methyl 3-bromo-2-(bromomethyl)prop-1-ene. Compound 203 was isolated as a beige solid. ¹H NMR (400 MHz, DMSO-d₆) δ 12.16 (s, 2H), 8.26 (s, 2H), 7.51 (s, 2H), 6.01-5.95 (m, 2H), 4.58-4.52 (m, 4H), 3.86 (s, 6H), 3.27 (d, J=6.5 Hz, 4H), 2.56 (t, J=6.3 Hz, 4H). ESI-MS: C₃₀H₂₇F₂O₁₀S₂ (M+H): calc. 649.10, found: 649.05.

### Example 11: (Z)-4,4′-((but-2-ene-1,4-diylbis(oxy))bis(4-fluoro-6-methoxybenzo[b]-thiophene-5,2-diyl))bis(4-oxobutanoic acid), Compound 204

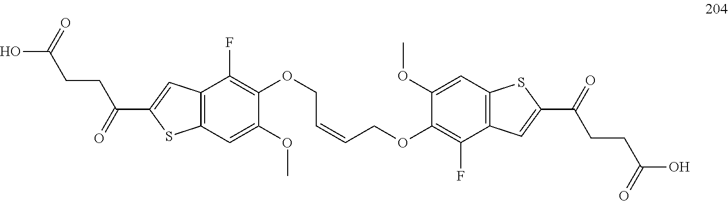 [SMILES: COc1cc2sc(C(=O)CCC(=O)O)cc2c(F)c1OC/C=C\COc1c(OC)cc2sc(C(=O)CCC(=O)O)cc2c1F]

Synthesis of the title compound was carried out as described in Example 9, except in Step I, cis-1,4-dibromo-2-butene was used instead of methyl 3-bromo-2-(bromomethyl)prop-1-ene. Compound 204 was isolated as a beige solid. ¹H NMR (400 MHz, DMSO-d₆) δ 12.17 (s, 1H), 8.24 (s, 1H), 7.50 (s, 1H), 5.86 (t, J=4.4 Hz, 1H), 4.58 (d, J=4.8 Hz, 2H), 3.85 (s, 3H), 3.26 (t, J=6.3 Hz, 2H), 2.55 (t, J=6.2 Hz, 2H). ESI-MS: C₃₀H₂₇F₂O₁₀S₂ (M−H): calc. 649.10, found: 649.05.

### Example 12: 4,4′-(((2-(2-hydroxyethylidene)propane-1,3-diyl)bis(oxy))bis(4-fluoro-6-methoxybenzo[b]thiophene-5,2-diyl))bis(4-oxobutanoic acid), Compound 210

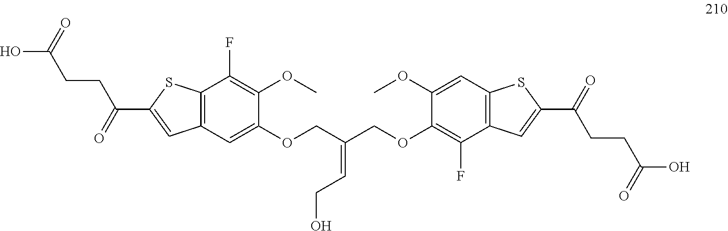 [SMILES: COc1cc2sc(C(=O)CCC(=O)O)cc2c(F)c1OC/C(=C/CO)COc1cc2cc(C(=O)CCC(=O)O)sc2c(F)c1OC]

Synthesis of the title compound was carried out as described in Example 9, except in Step I, ((4-bromo-3-(bromomethyl)but-2-en-1-yl)oxy)(tert-butyl)dimethylsilane was used instead of methyl 3-bromo-2-(bromomethyl)prop-1-ene. Compound 210 was isolated as a white solid. ESI-MS: C31H26F2O11S2 (M+H): calc. 679.11, found: 678.97.

### Example 13: 4,4′-(((1,2-phenylenebis(methylene))bis(oxy))bis(4-fluoro-6-methoxybenzo[b]thiophene-5,2-diyl))bis(4-oxobutanoic acid), Compound 211

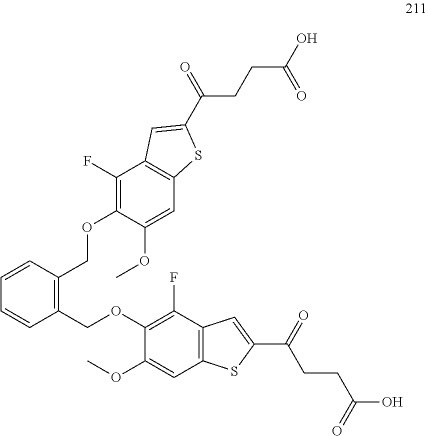 [SMILES: COc1cc2sc(C(=O)CCC(=O)O)cc2c(F)c1OCc1ccccc1COc1c(OC)cc2sc(C(=O)CCC(=O)O)cc2c1F]

Synthesis of the title compound was carried out as described in Example 9, except in Step I, 1,2-bis(bromomethyl)benzene was used instead of methyl 3-bromo-2-(bromomethyl)prop-1-ene. Compound 211 was isolated as a white solid. ESI-MS: C34H29F2O10S2 (M+H): calc. 699.12, found: 698.97.

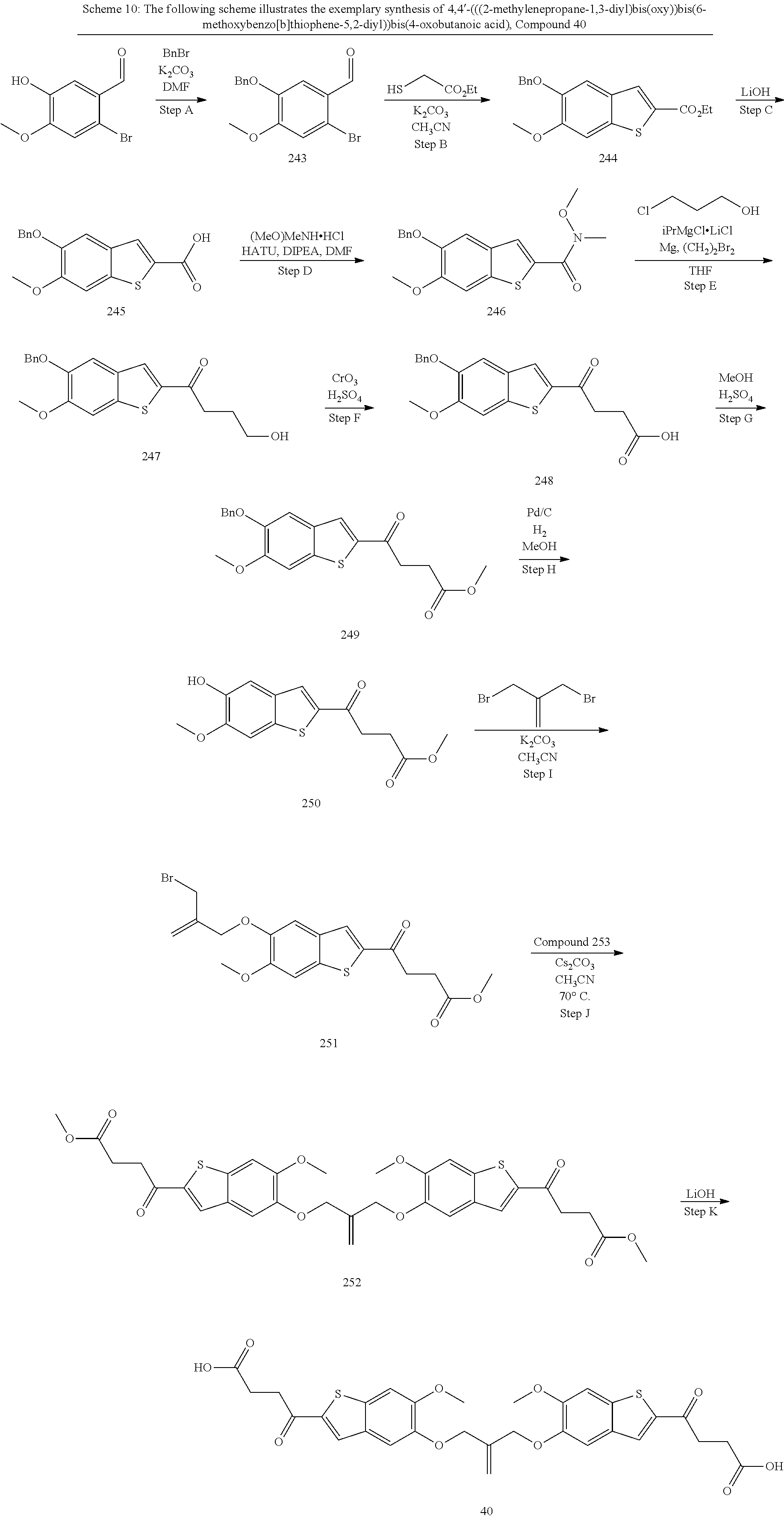

### Example 14: 4,4′-(((2-methylenepropane-1,3-diyl)bis(oxy))bis(6-methoxybenzo[b]-thiophene-5,2-diyl))bis(4-oxobutanoic acid), Compound 40

Step A: To a solution of 2-bromo-5-hydroxy-4- methoxybenzaldehyde (5.0 g, 21.7 mmol) and potassium carbonate (4.5 g, 32.6 mmol) in DMF (50 mL) was added benzyl bromide (3.1 mL, 26.1 mmol) and stirred at 27° C. for 3 hours. The reaction mixture was quenched with diluted with sat. NaHCO₃ (200 mL) and extracted with ethyl acetate (3×150 mL). The combined organic layers were washed with brine (2×100 mL), dried over anhydrous sodium sulfate, filtered and concentrated under reduced pressure to Compound 243 (5.5 g, 79% yield) as an off-white solid. ¹H NMR (400 MHz, CDCl3) δ 10.15 (s, 1H), 7.49-7.39 (m, 3H), 7.41-7.33 (m, 2H), 7.36-7.27 (m, 1H), 7.06 (s, 1H), 5.15 (s, 2H), 3.94 (s, 3H). ESI-MS: C₁₅H₁₄BrO₃ (M+H): calc. 321.01, found: 320.85.

Step B: To the solution of Compound 243 (5.5 g, 17.2 mmol) and ethyl thioglycolate (4.13 g, 34.4 mmol) in DMF (50 mL) was added potassium carbonate (4.75 g, 34.4 mmol) and copper iodide (0.5 g) then the mixture was stirred at 80° C. for 16 hours. The reaction was cooled to room temperature and then filtered. The filtrate was concentrated and dissolved in ethyl acetate (300 mL). The organic layer was washed with brine (50 mL), dried over anhydrous sodium sulfate, concentrated and then purified over silica gel (20:80 EtOAc:hexane v/v) to afford Compound 244 (3.5 g, 59% yield). ¹H NMR (400 MHz, CHLOROFORM-D) δ 7.86 (s, 1H), 7.49-7.42 (m, 2H), 7.42-7.22 (m, 5H), 5.20 (s, 2H), 4.36 (q, J=7.2 Hz, 2H), 3.96 (s, 3H), 1.38 (t, J=7.1 Hz, 3H). ESI-MS: C₁₉H₁₉O₄S (M+H): calc. 343.10, found: 342.95.

Step C: To the solution of Compound 244 (3.5 g, 10.2 mmol) in THF (20 mL), and MeOH (20 mL) was added lithium hydroxide monohydrate (4.3 g, 102.0 mmol) in H₂O (20 mL) and stirred at 50° C. for 6 hours. The reaction mixture was concentrated, acidified with 1N HCl and then filtered. The solid was washed with water and pentane to afford Compound 245 (3.0 g, 93% yield). ¹H NMR (400 MHz, DMSO-D6) δ 13.15 (s, 1H), 7.87 (s, 1H), 7.56 (d, J=6.7 Hz, 2H), 7.48-7.26 (m, 5H), 5.09 (s, 2H), 3.82 (s, 3H). ESI-MS: C₁₇H₁₅O₄S (M+H): calc. 315.07, found: 314.90.

Step D: To the solution of Compound 245 (3.0 g, 9.55 mmol) in DMF (0.5 mL) was added N-methoxy-N-methyl amine hydrochloride (1.4 g, 14.3 mmol) and HATU (5.5 g, 14.3 mmol) and then stirred at 27° C. for 16 hours. The reaction mixture was quenched with H₂O (100 mL) and extracted with ethyl acetate (3×100 mL). The combined organic layers were washed with brine (2×100 mL), dried over anhydrous sodium sulfate, filtered and concentrated under reduced pressure. The resulting crude material was purified over silica gel (30:70 EtOAc:hexane v/v) to afford Compound 246 (2.2 g, 64% yield) as a pale yellow solid. ¹H NMR (400 MHz, CDCl₃) δ 8.01 (d, J=0.7 Hz, 1H), 7.49-7.43 (m, 2H), 7.41-7.30 (m, 2H), 7.34-7.26 (m, 1H), 7.26 (d, J=7.0 Hz, 2H), 5.20 (s, 2H), 3.96 (s, 3H), 3.79 (s, 3H), 3.38 (s, 3H), 2.79 (s, 3H).

Step E: To a solution of 3-chloro-1-propanol (8.6 mL, 92.4 mmol) in THE (60 mL) at −20° C. and magnesium turnings (2.4 g, 101.7 mmol) under argon was added a solution of isopropylmagnesium chloride lithium chloride complex (1.3 M in THF; 50.0 mL) and 1,2-dibromoethane (2.0 mL). The mixture was allowed to warm up to room temperature over 20 min and then heated at 80° C. for 2 hours, before being cooled to room temperature and then added over 10 min through cannula to a solution of Compound 246 (2.2 g, 6.16 mmol) in THF (40 mL) at 0° C. and stirred for 2 hours. The reaction mixture was quenched with ice cold sat. NH₄Cl solution (50 mL) and extracted with ethyl acetate (3×50 mL). The organic layer was washed with brine (2×50 mL), dried over anhydrous sodium sulfate and concentrated to give a residue which was purified over silica gel (20:80 EtOAc:hexane v/v) to afford Compound 247 (1.8 g, 82% yield). ¹H NMR (400 MHz, DMSO) δ 8.12 (s, 1H), 7.60 (s, 1H), 7.54 (s, 1H), 7.47 (d, J=7.4 Hz, 2H), 7.40 (t, J=7.4 Hz, 2H), 7.33 (t, J=7.3 Hz, 1H), 5.14 (s, 2H), 4.50 (t, J=5.2 Hz, 1H), 3.86 (s, 3H), 3.45 (q, J=6.0 Hz, 2H), 3.02 (t, J=7.3 Hz, 2H), 1.77 (p, J=6.9 Hz, 2H). ESI-MS: C₂₀H₂₁O₄S (M+H): calc. 357.12, found: 356.95.

Step F: To a stirred solution of CrO₃ (2.5 g, 25.3 mmol) in water (12.5 mL) was added H₂SO₄ (2.5 mL) at 0° C. The mixture was added dropwise to a stirred solution of Compound 247 (1.8 g, 5.05 mmol) in acetone (50 mL) at 0° C. for 1 hour, then warmed to room temperature and stirred for 2 hours. The mixture was quenched with water (150 mL) and the resulting solid was filtered and dried to afford Compound 248 (1.3 g, 70% yield). ¹H NMR (400 MHz, DMSO) δ 12.05 (s, 1H), 8.18 (s, 1H), 7.61 (s, 1H), 7.56 (s, 1H), 7.47 (d, J=7.4 Hz, 2H), 7.40 (t, J=7.4 Hz, 2H), 7.34 (d, J=7.2 Hz, 1H), 5.15 (s, 2H), 3.86 (s, 3H), 2.66 (t, J=6.3 Hz, 2H). ESI-MS: C₂₀H₁₉O₅S (M+H): calc. 371.09, found: 370.95.

Step G: To a stirred solution of Compound 248 (1.3 g, 3.5 mmol) in MeOH (100 mL) was added dropwise H₂SO₄ (0.5 mL). The mixture was heated to 80° C., stirred for 3 hours, then cooled to room temperature. The reaction mixture was concentrated, then quenched with H₂O (100 mL) and the resulting solid was filtered and dried to afford Compound 249 (1.2 g, 89% yield). ¹H NMR (400 MHz, DMSO) δ 8.18 (s, 1H), 7.61 (s, 1H), 7.56 (s, 1H), 7.47 (d, J=7.4 Hz, 2H), 7.40 (t, J=7.4 Hz, 2H), 7.34 (d, J=7.2 Hz, 1H), 5.15 (s, 2H), 3.86 (s, 3H), 3.59 (s, 3H), 2.66 (t, J=6.3 Hz, 2H). ESI-MS: C₂₁H₂₁O₅S (M+H): calc. 385.11, found: 385.00.

Step H: To a stirred solution of Compound 249 (1.2 g, 3.1 mmol) in MeOH (25 mL) was added Pd/C (200 mg, 10% w/w) in THE (25 mL). Hydrogen gas was bubbled through the solution for 10 minutes, then the mixture was stirred under a hydrogen atmosphere for 2 hours. The mixture was then filtered over Celite and concentrated to give a residue which was purified over silica gel (30:70 EtOAc:hexane v/v) to afford Compound 250 (480 mg, 52% yield). ¹H NMR (400 MHz, DMSO) δ 9.38 (s, 1H), 8.17 (s, 1H), 7.54 (s, 1H), 7.32 (s, 1H), 3.87 (s, 3H), 3.60 (s, 3H), 3.29 (d, J=6.2 Hz, 1H), 2.67 (t, J=6.4 Hz, 2H). ESI-MS: C₁₄H₁₅O₅S (M+H): calc. 295.06, found: 294.90.

Step I: To the solution of Compound 250 (50 mg, 0.10 mmol) and methyl 3-bromo-2-(bromomethyl)prop-1-ene (21 mg, 0.10 mmol) in CH₃CN (5 mL) was added and Cs₂CO₃ (166 mg, 0.51 mmol) and the resulting mixture was stirred at 70° C. for 1 hour, then cooled to room temperature, diluted with EtOAc (10 mL) and filtered with THF. The filtrate was concentrated to give a residue that was purified over silica gel (20:80 EtOAc:hexane v/v) to afford Compound 251 (8 mg, 18% yield). ESI-MS: C₁₈H₂₁BrO₅S (M+2H): calc. 428.03, found: 428.85.

Step J: To a solution of Compound 251 (30 mg, 0.07 mmol) in CH₃CN (5 mL) was added Compound 251 (20 mg, 0.70 mmol) and Cs₂CO₃ (114 mg, 0.35 mmol) and the resulting mixture was stirred at 70° C. for 3 hours. The reaction was cooled to room temperature, diluted with EtOAc (10 mL) and then filtered with THF. The filtrate was concentrated to give a residue which was purified over silica gel (20:80 EtOAc:hexane v/v) to afford Compound 252 (30 mg, 67% yield). ESI-MS: C₃₂H₃₃O₁₀S₂ (M+H): calc. 641.15, found: 641.10.

Step K: To the solution of Compound 252 (30 mg, 0.047 mmol) in THE (2.0 mL) and MeOH (2.0 mL) was added lithium hydroxide monohydrate (20 mg, 0.47 mmol) at 0° C. and the reaction mixture was stirred for 2 hours at 27° C. The reaction mixture was concentrated to remove volatiles, dissolved in H₂O (5 mL), acidified with 1N HCl and then filtered. The solid was washed with water and pentane to afford Compound 40 (22 mg, 78% yield). ESI-MS: C₃₀H₂₉O₁₀S₂ (M+H): calc. 613.12, found: 613.10.

### Example 15: 4-(5-((2-(((2-(3-carboxypropanoyl)-4-fluoro-6-methoxybenzo[b]thiophen-5-yl)oxy)methyl)allyl)oxy)-6-methoxybenzo[b]thiophen-2-yl)-4-oxobutanoic acid, Compound 207

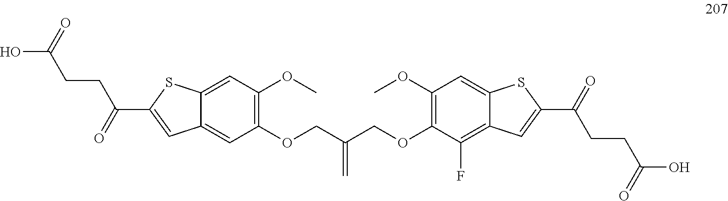 [SMILES: C=C(COc1cc2cc(C(=O)CCC(=O)O)sc2cc1OC)COc1c(OC)cc2sc(C(=O)CCC(=O)O)cc2c1F]

Synthesis of the title compound was carried out as described in Example 9, except in Step J, Compound 253 was used instead of methyl 4-(4-fluoro-5-hydroxy-6-methoxybenzo[b]thiophen-2-yl)-4-oxobutanoate ESI-MS: C₃₀H₂₈FO₁₀S₂ (M+H): calc. 631.11, found: 631.05.

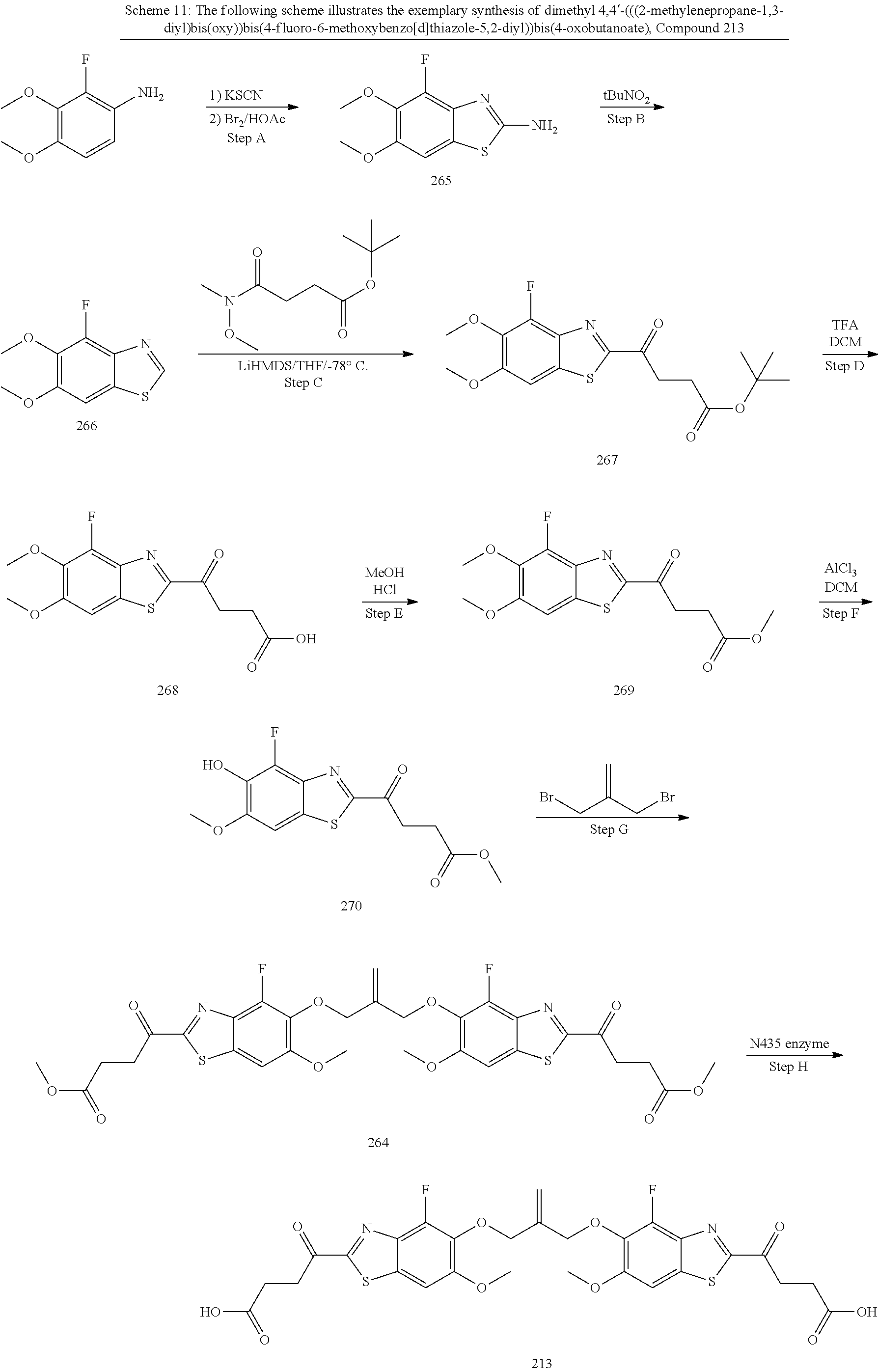

### Example 16: Dimethyl 4,4′-(((2-methylenepropane-1,3-diyl)bis(oxy))bis(4-fluoro-6-methoxybenzo[d]thiazole-5,2-diyl))bis(4-oxobutanoate), Compound 213

Step A: To a solution of 2-fluoro-3,4-dimethoxyaniline (980 mg, 5.73 mmol) in HOAc (20 mL) was added potassium thiocyanate (2.22 g, 22.9 mmol), then, dropwise, a solution of bromine (590 μL, 11.4 mmol) in acetic acid (2.5 mL). The reaction mixture was stirred for 2.5 hours, then concentrated and quenched with saturated NaHCO₃ solution. The mixture was concentrated and filtered through Celite. The filtrate was concentrated to give a residue that was purified over silica gel (0-20% MeOH-DCM), to afford Compound 265 as a dark, orange oil (1.75 g, 100% yield). ¹H NMR (400 MHz, DMSO-d₆) δ 7.46 (s, 2H), 7.23 (d, J=1.7 Hz, 1H), 3.77 (d, J=6.3 Hz, 6H).

Step B: To a mixture of Compound 265 (1.75 g, 5.73 mmol) in DMF (20 mL) was added tBuONO (2.04 mL, 17.1 mmol) and the resulting mixture was heated at 60° C. for 1 hour. The reaction mixture was diluted with DCM and the organic layer was washed with water (3×10 mL), dried over anhydrous sodium sulfate and concentrated to give a residue which was purified over silica gel (10-20% EtOAc-hexanes), to afford Compound 266 as a light green solid (495 mg, 41% yield). ¹H NMR (400 MHz, DMSO-d₆) δ 9.22 (d, J=0.5 Hz, 1H), 7.66 (d, J=1.7 Hz, 1H), 4.03-3.67 (m, 6H).

Step C: To a solution of Compound 266 (495 mg, 2.32 mmol) in THE (20 mL) at −78° C. was added 1M LiHMDS in THE (18.6 mL, 18.6 mmol) and the resulting mixture was stirred cold for 25 minutes. Then, tert-butyl 4-(methoxy(methyl)amino)-4-oxobutanoate (1.40 g, 6.50 mmol) in THF (2 mL) was added dropwise over 10 minutes, then the mixture was warmed to 0° C. and then to room temperature. After 2.5 hours, the reaction mixture was quenched with saturated citric acid solution and washed with DCM (3×25 mL). The combined organic layers were washed with brine (2×100 mL), dried over anhydrous sodium sulfate, filtered and concentrated under reduced pressure. The resulting crude material was purified over silica gel (5-20% EtOAc-hexanes). Compound 267 was isolated as a green oil, (126 mg, 15% yield). ¹H NMR (400 MHz, DMSO-d₆) δ 8.26 (s, 1H), 4.28 (s, 3H), 3.86 (s, 3H), 3.44 (dd, J=7.0, 5.8 Hz, 2H), 2.66 (dd, J=6.9, 5.9 Hz, 2H), 1.36 (s, 9H).

Step D: A solution of Compound 267 (130 mg, 352 μmol) in 10% TFA-DCM (3 mL) was stirred for 2 hours. The reaction mixture was concentrated in vacuo. Compound 268 was isolated as a brown solid (130 mg, 100% yield). ¹H NMR (500 MHz, DMSO-d₆) δ 12.25 (s, 1H), 7.73 (d, J=1.5 Hz, 1H), 3.92 (d, J=20.7 Hz, 6H), 3.48-3.37 (m, 2H), 2.66 (dd, J=7.3, 5.7 Hz, 2H). ESI-MS: C₁₃H₁₃FNO₅S (M+H): calc. 314.04, found: 314.04.

Step E: A solution of Compound 268 (113 mg, 352 μmol) in MeOH (4 mL) and conc HCl_{aq} (1 mL) was stirred for 3.5 hours. The reaction mixture was concentrated in vacuo. Compound 269 was isolated as a brown solid (104 mg, 90% yield). ¹H NMR (500 MHz, DMSO-d₆) δ 7.73 (d, J=1.5 Hz, 1H), 3.92 (d, J=20.1 Hz, 6H), 3.60 (s, 3H), 3.48 (dd, J=6.9, 6.1 Hz, 2H), 2.74 (t, J=6.5 Hz, 2H). ESI-MS: C₁₄H₁₅FNO₅S (M+H): calc. 328.06, found: 328.06.

Step F: To a mixture of Compound 269 (85 mg, 260 μmol) in DCM (10 mL), was added AlCl₃ (104 mg, 780 μmol) and the resulting mixture was stirred for 17 hours. Then, AlCl₃ (104 mg, 780 μmol) was added. After 2 hours, the reaction mixture was quenched carefully with water, then 1N HCl_{aq} and the mixture was washed with DCM (3×25 mL). The combined organic layers were washed with brine (2×100 mL), dried over anhydrous sodium sulfate, filtered and concentrated under reduced pressure. The resulting crude material was purified over silica gel (0-40% MeOH-DCM), to afford Compound 270 as a yellow powder (38 mg, 47% yield). ¹H NMR (400 MHz, DMSO-d₆) δ 9.87 (d, J=1.0 Hz, 1H), 7.62 (d, J=1.6 Hz, 1H), 3.93 (s, 3H), 3.60 (s, 3H), 3.55-3.40 (m, 2H), 2.73 (dd, J=7.3, 5.6 Hz, 2H). ESI-MS: C₁₃H₁₃FNO₅S (M+H): calc. 314.04, found: 314.03.

Step G: A solution of Compound 270 (26 mg, 83 μmol), dibromide (11.5 μL, 100 μmol), Cs₂CO₃ (54 mg, 166 μmol) and acetonitrile (3 mL) was heated at 60° C. for 1 hour. The reaction mixture was concentrated, then diluted with DCM (25 mL) and the organic layer was washed with water (3×10 mL), dried over anhydrous sodium sulfate and concentrated to give a residue that was purified by prep HPLC, to afford Compound 263 as a white solid (9 mg, 11% yield). ESI-MS: C₃₀H₂₉F₂N₂O₁₀S₂ (M+H): calc. 679.12, found: 679.07.

Step H; To a suspension of Compound 263 (3 mg, 4.4 μmol), DMF (400 μL), water (400 μL) was added Novozyme435 (6 mg), then 1N NaHCO₃ (1 eq, 4.4 μL). After 1 hour, additional Novozyme 435 (12 mg) and 1N NaHCO₃ (9 μL) were added. The reaction mixture was filtered through a pad a celite and the filtrate was concentrated to give a residue which was purified by prep HPLC column. Compound 213 was isolated, after lyophilization, as a white solid, (0.44 mg, 15%). ESI-MS: C28H25F2N2010S (M+H): calc. 651.09, found: 651.03.

### Example 17: methyl 4-(5-((3-(((4-fluoro-6-methoxy-2-(4-methoxy-4-oxobutanoyl)benzo[b]thiophen-5-yl)oxy)methyl)oxetan-3-yl)methoxy)-6-methoxybenzo[b]thiophen-2-yl)-4-oxobutanoate, Compound 262

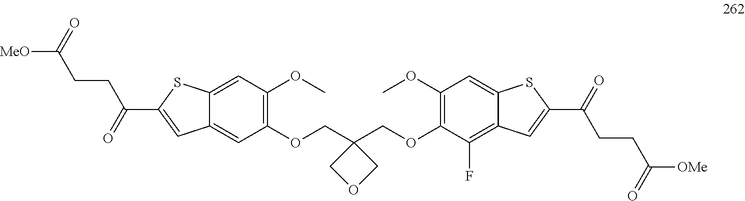 [SMILES: COC(=O)CCC(=O)c1cc2cc(OCC3(COc4c(OC)cc5sc(C(=O)CCC(=O)OC)cc5c4F)COC3)c(OC)cc2s1]

Synthesis of the title compound was carried out as described in Example 9, except in Step J, 3,3-bis(bromomethyl)oxetane was used instead methyl 3-bromo-2-(bromomethyl)prop-1-ene. Compound 262 was isolated as a white solid. ¹H NMR (400 MHz, DMSO-d₆) δ 8.23 (d, J=0.7 Hz, 2H), 7.53 (s, 2H), 4.55-4.39 (m, 4H), 3.98-3.88 (m, 4H), 3.87 (s, 6H), 3.60 (s, 6H), 3.35 (dd, J=7.0, 5.8 Hz, 4H), 2.66 (dd, J=7.3, 5.5 Hz, 4H). ESI-MS: C₃₃H₃₄F₂O₁₁S₂ (M+H): calc. 707.14, found: 707.05.

### Example 18 Activation of Human STING Signaling in THP1 Cells

THP1-Dual™ Human Monocytes cells with the homozygous HAQ allele, known to be a natural variant allele of STING occurring in ˜20% of the human population (Invivogen) have been engineered with two independent gene reporter systems: a secreted embryonic alkaline phosphatase (SEAP) reporter gene for NFkB activity, and secreted luciferase reporter for interferon response gene 3 (IRF3) activity were used. 4×10⁵ THP1-Dual™ cells were incubated with test Compounds in a 5-fold titration steps from 1 to 0.0128 μM in RPMI media with 10% FBS, for 24 hours at 37° C. with 5% CO₂. Cell culture supernatants (20 μl) from each incubated sample was added to resuspended QUANTI-Blue™ Solution (Invivogen, 180 μl) in each well of a flat-bottom 96-well plate, then incubated for 2 h. NFkB levels were evaluated using a spectrophotometer at 620-655 nm.

To evaluate IRF3 reporter levels, cell culture supernatant (20 μl) from each incubated sample was added to QUANTI-Luc™ assay solution (Invivogen, 50 μl), and the luminescence was measured with a SpectraMax M3 spectrophotometer (Molecular Devices).

NFkB and IRF3 reporter levels were also determined for THP1-Dual™ KI-hSTING-H232 Cells and THP1-Dual™ KI-hSTING-R232 Cells. THP1-Dual™ KI-hSTING-H232 Cells (R232H Haplotype) were generated from THP1-Dual™ KO-STING cells by knockin of the intronless coding sequence (from the ATG to TGA) of the R232H human STING variant (Invivogen). R232H has been identified as a natural variant allele of STING occurring in ˜14% of the human population. THP1-Dual™ KI-hSTING-R232 Cells (R232 Haplotype) were generated from THP1-Dual™ KO-STING cells by knockin of the intronless coding sequence (from the ATG to the TGA) of the R232 hSTING variant. Genomic studies indicate that this variant, which contains an arginine at position 232 (R232), is the most prevalent variant with an occurrence (homozygous allele) of ˜45-58% in the human population.

The EC50 value was determined from the dose response curve based on reference Compounds. Table A provides the results for IRF3 reporter induction THP1-Dual™ Human Monocytes cells and Table B provides the results for IRF3 reporter induction in THP1-Dual™ KI-hSTING-R232 Cells (“A” means <1 μM; “B” means ≥1 μM and <10 μM; “C” means ≥10 μM).

TABLE A

| Example No. | Compound No. | EC₅₀ |
|---|---|---|
| 1 | 6 | C |
| 2 | 9 | C |
| 3 | 18 | A |
| 4 | 25 | C |
| 5 | 30 | B |
| 6 | 36 | C |
| 7 | 48 | C |
| 9 | 202 | A |
| 10 | 203 | A |
| 11 | 204 | A |
| 14 | 40 | A |
| 15 | 207 | A |
| 16 | 213 | C |
| | 264 | A |
| 17 | 262 | C |
| | 205 | C |
| | 206 | C |
| | 208 | C |
| | 209 | C |
| | 210 | A |
| | 211 | A |
| | 218 | C |
| | 219 | C |
| | 220 | C |
| | 221 | C |
| | 222 | C |
| | 223 | C |
| | 224 | C |
| | 226 | C |
| | 227 | C |
| | 229 | C |

TABLE B

| Example No. | Compound No. | EC₅₀ |
|---|---|---|
| 9 | 202 | A |
| 10 | 203 | B |
| 11 | 204 | A |
| 12 | 40 | C |
| 13 | 207 | A |
| 16 | 213 | A |
| | 264 | C |
| | 205 | C |
| | 206 | A |
| | 218 | C |
| | 219 | C |
| | 220 | C |
| | 221 | C |
| | 222 | C |
| | 223 | C |
| | 224 | C |
| | 229 | C |
| | 226 | C |
| | 227 | C |
| | 208 | C |
| | 209 | C |
| | 210 | A |
| | 211 | A |

### Example 19: Activation of Human STING Signaling in Permeabilized THP1 Cells

4×10⁵ THP1-Dual™ cells (NF-κB-SEAP and IRF-Lucia luciferase Reporter Monocytes, Invivogen) were incubated with test Compounds in a 5-fold titration steps from 1 to 12.8 nM in permeabilized buffer (50 mM HEPES pH 7.0, 100 mM KCl, 3 mM MgCl₂, 85 mM sucrose, 0.2% BSA, 1 mM ATP, 0.1 mM GTP, 0.1 mM TTP, 1 g/ml digitonin) for 30 minutes on ice. Cells were then washed and incubated in a fresh RPMI media with 10% FBS at 37° C. with 5% CO₂ for 24 h. Cell culture supernatants from each sample were collected and NFkB and IRF3 were evaluated as above. Table B provides the results for IRF3 induction (“A” means <1 μM; “B” means ≥1 μM and <10 μM; “C” means ≥10 μM).

TABLE C

| Example No. | Compound No. | IRF3 EC₅₀ |
|---|---|---|
| 1 | 6 | C |
| 2 | 9 | C |
| 4 | 25 | C |
| 5 | 30 | B |
| 6 | 36 | C |
| 7 | 48 | C |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I), (II), (III), (IV), (V) or (VI): 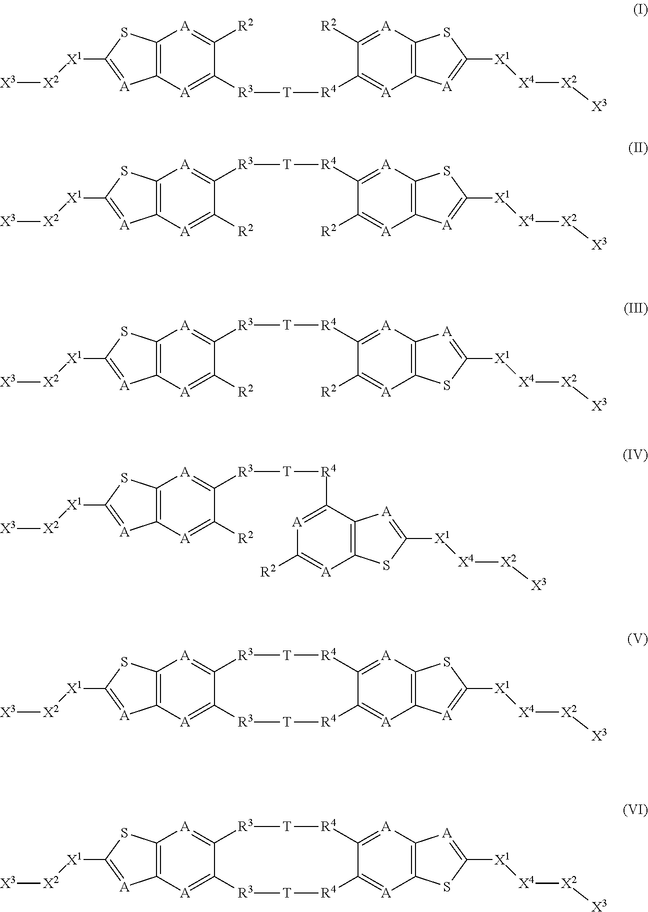
or a pharmaceutically acceptable salt thereof, wherein:
each A independently is —C(R¹)— or —N—;
each R¹ independently is hydrogen, halogen, OR⁶, N(R⁶)₂, CN, or C₁-C₆ alkyl, wherein the C₁-C₆ alkyl is optionally substituted with one or more halogen, OR⁶, N(R⁶)₂, C(═O)OR⁶, or C(═O)N(R⁶)₂;
each R² independently is hydrogen, halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C₁-C₆ alkyl, C₂-C₆ alkenyl, or C₂-C₆ alkynyl is optionally substituted with one more halogen or OR⁶;
R³ and R⁴ each independently are absent, C₁-C₆ alkyl, —N(R_{S})—, or —O—;
each T independently is absent, -Tₐ-C₂-C₆ alkyl-T_{b}-, —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S};
Tₐ and T_{b} each independently are absent, —N(R_{S})—, —O—, C₁-C₆ alkyl, —N(R_{S})—(C₁-C₆ alkyl)-, —(C₁-C₆ alkyl)-N(R_{S})—, —N(R_{S})—(C₁-C₆ alkyl)-N(R_{S})—, —O—(C₁-C₆ alkyl)-, —(C₁-C₆ alkyl)-O—, or —O—(C₁-C₆ alkyl)-O—; wherein the C₁-C₆ alkyl is optionally substituted with one or more halogen; and
each R_{S} independently is H or C₁-C₆ alkyl optionally substituted with one or more halogen;
each R⁶ independently is H or C₁-C₆ alkyl optionally substituted with one or more halogen;
each X¹ independently is —C(═O)—, —CH₂—, —CHF—, or —CF₂—;
each X² independently is —(C(R⁸)₂)₁₋₃—, wherein each R⁸ independently: (a) is hydrogen, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂; or (2) together with another R⁸ and the one or more atoms to which they are attached, form C₃-C₆ cycloalkyl or 3- to 6-membered heterocycloalkyl;
each X³ independently is C(═O)OR⁶, C(═O)SR⁶, C(═S)OR⁶, 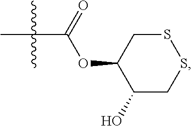 [SMILES: *C(=O)O[C@H]1CSSC[C@@H]1O]
SO₂R⁶, C(═O)N(R⁹)₂, or CN;
each R⁹ independently is hydrogen, C(═O)OR⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₁-C₆ alkyl)-OR⁶, O(C₆-C₁₀ aryl), S(C₁-C₆ alkyl), S(C₆-C₁₀ aryl), S(═O)₂R⁶, S(═O)₂OR⁶, P(═O)(R⁶)₂, C₁-C₆ alkyl, C₂-C₆ alkenyl, C₂-C₆ alkynyl, C₃-C₅ cycloalkyl, C₆-C₁₀ aryl, 3-8 membered heterocycloalkyl, or 3-10 membered heteroaryl; and
X⁴ is absent or —NR¹¹, wherein each R¹¹ independently is H or C₁-C₆ alkyl optionally substituted with one or more halogen;
provided that:
(1) when one of R₃ and R₄ is C₁ alkyl, and T is —N(R_{S})— or —O—, then the other one of R₃ and R₄ is absent;
(2) when one of R₃ and R₄ is C₁-C₂ alkyl, and T is absent, then the other one of R₃ and R₄ is absent;
(3) when one of R₃ and R₄ is C₂-C₆ alkyl, then T is not —N(R_{S})— or —O—;
(4) when at least one of R₃ and R₄ is C₃-C₆ alkyl, then T is not absent;
(5) when X⁴ is —NR¹¹, at least one A is —N—; and/or
(6) when T is -Tₐ-C₂-C₆ alkyl-T_{b}, and X⁴ is absent, then R₁ is CN.

2. The compound of any one of the preceding claims, wherein: (1) when one of R₃ and R₄ is C₁ alkyl, and T is —N(R_{S})— or —O—, then the other one of R₃ and R₄ is absent; (2) when one of R₃ and R₄ is C₁-C₂ alkyl, and T is absent, then the other one of R₃ and R₄ is absent; (3) when one of R₃ and R₄ is C₂-C₆ alkyl, then T is not —N(R_{S})— or —O—; (4) when at least one of R₃ and R₄ is C₃-C₆ alkyl, then T is not absent; 5) when X⁴ is —NR¹¹, at least one A is —N—; and (6) when T is -Tₐ-C₂-C₆ alkyl-T_{b}, and X⁴ is absent, then R₁ is CN.

3. The compound of any one of the preceding claims, wherein when T is -Tₐ-(C₃-C₁₂ cycloalkyl)-T_{b}- or -Tₐ-(3- to 12-membered heterocycloalkyl)-T_{b}-, then the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl.

4. The compound of any one of the preceding claims, being of Formula (I) or the pharmaceutically acceptable salt thereof.

5. The compound of any one of the preceding claims, wherein each 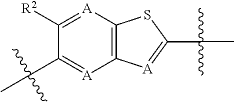
independently is

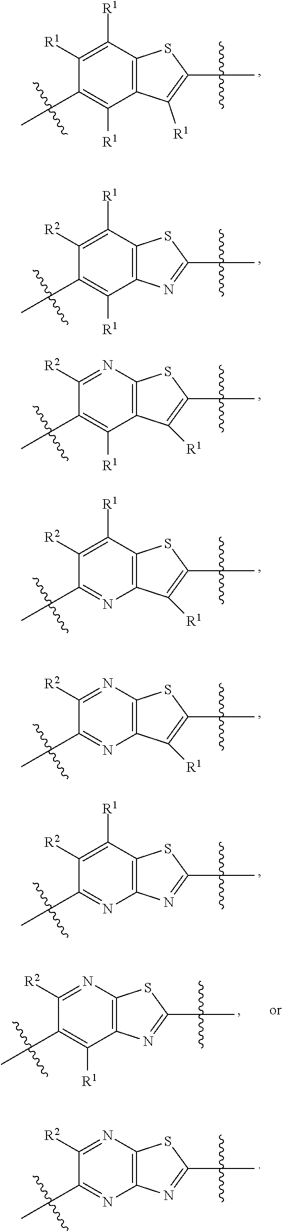

6. The compound of any one of the preceding claims, wherein each 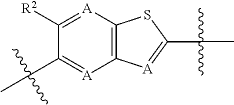
independently is

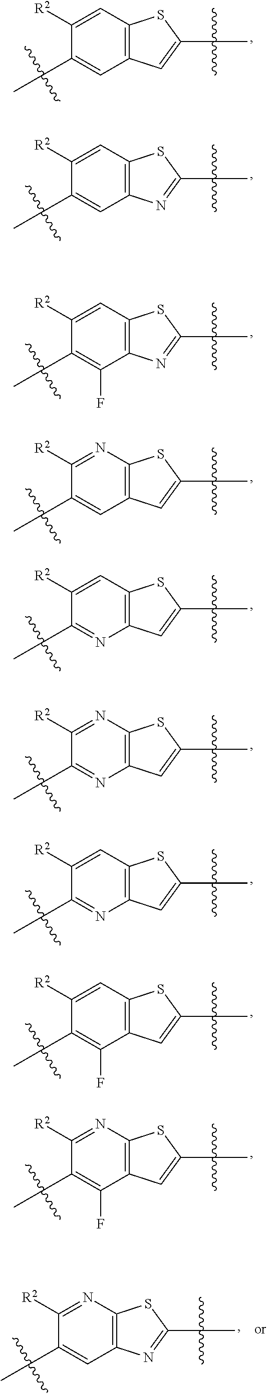

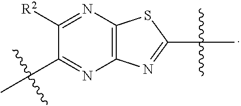

7. The compound of any one of the preceding claims, being of Formula (II) or the pharmaceutically acceptable salt thereof.

8. The compound of any one of the preceding claims, wherein each 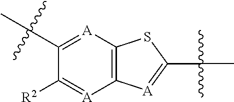
independently is

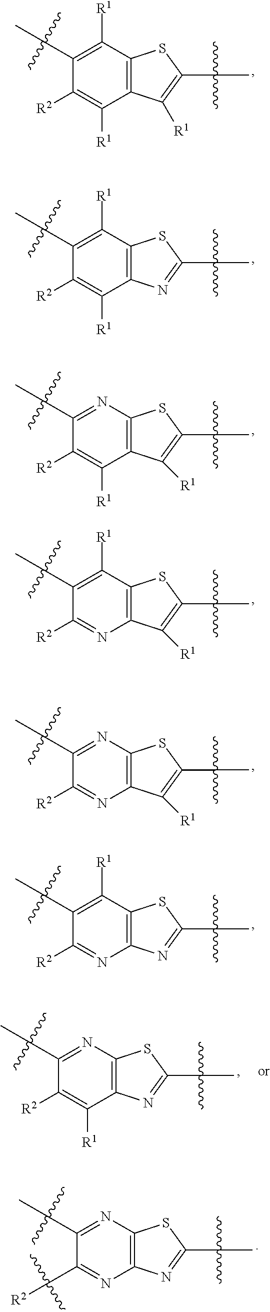

9. The compound of any one of the preceding claims, wherein each 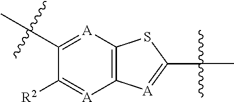
independently is

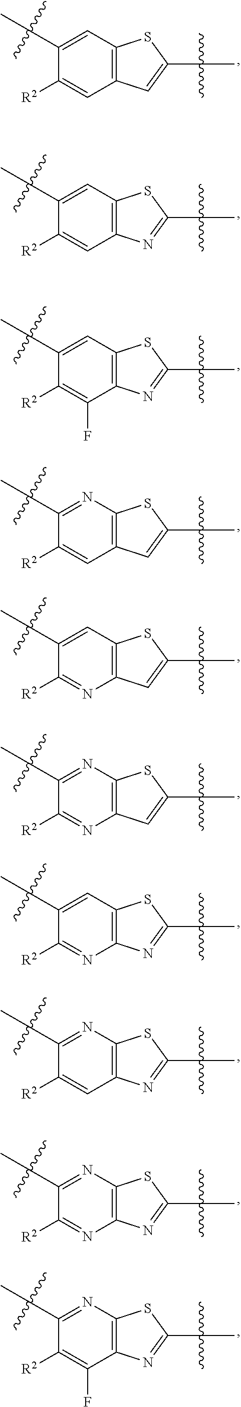

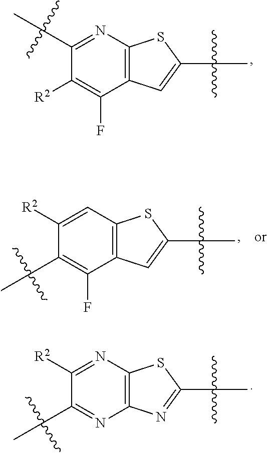

10. The compound of any one of the preceding claims, being of Formula (III) or the pharmaceutically acceptable salt thereof.

11. The compound of any one of the preceding claims, wherein each 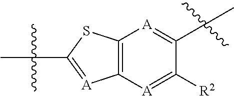
independently is

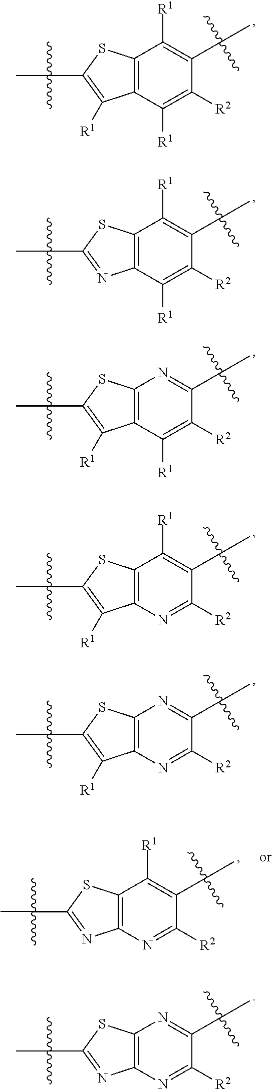

12. The compound of any one of the preceding claims, wherein each 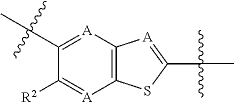
independently is

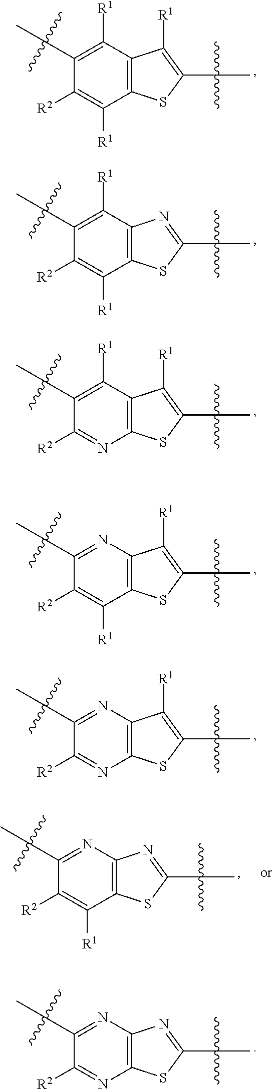

13. The compound of any one of the preceding claims, wherein each 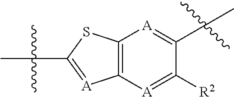
independently is,

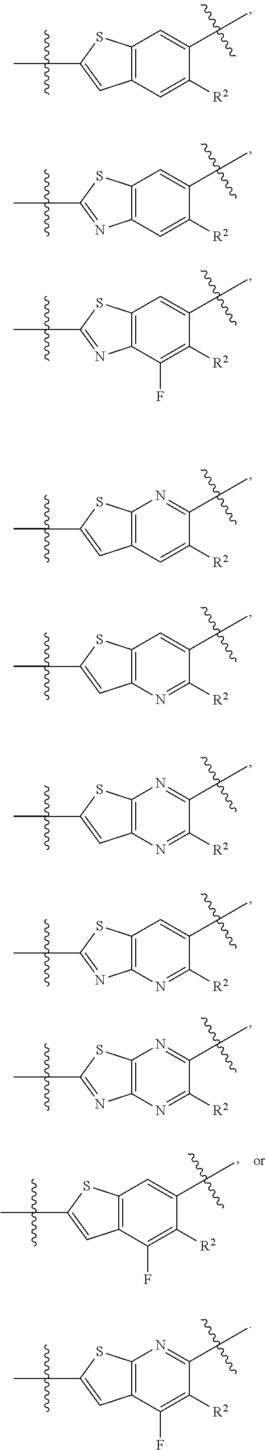

14. The compound of any one of the preceding claims, wherein each 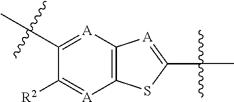
independently is

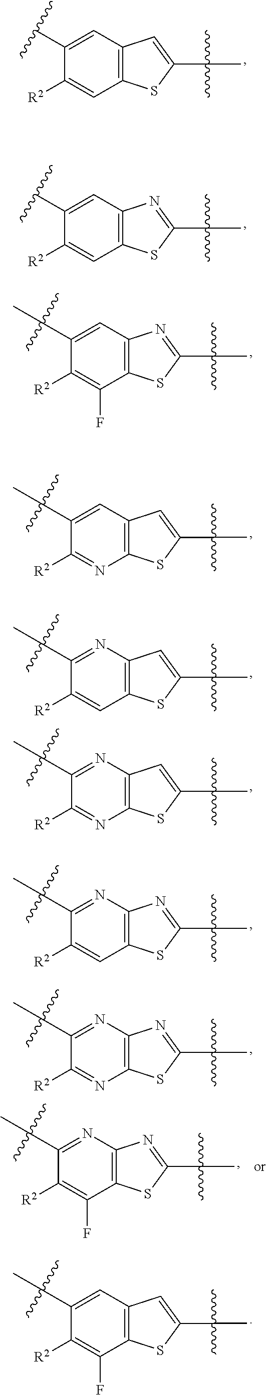

15. The compound of any one of the preceding claims, being of Formula (IV) or the pharmaceutically acceptable salt thereof.

16. The compound of any one of the preceding claims, wherein each 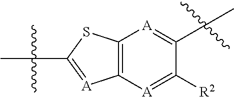
independently is

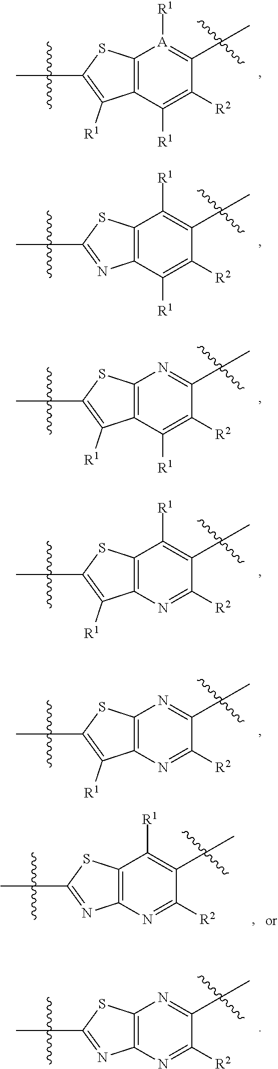

17. The compound of any one of the preceding claims, wherein each 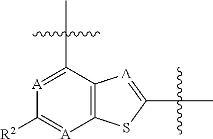
independently is

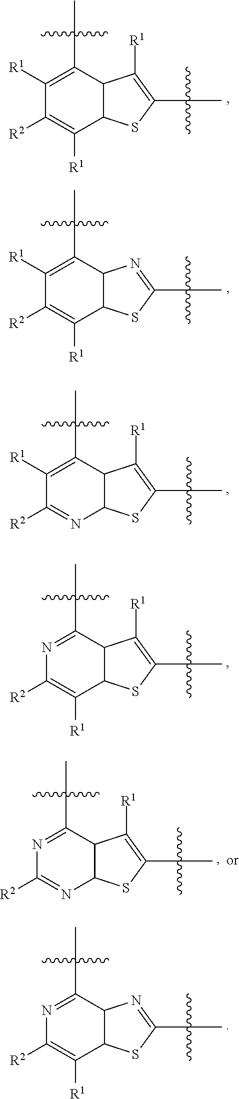

18. The compound of any one of the preceding claims, wherein each 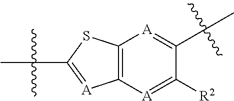
independently is

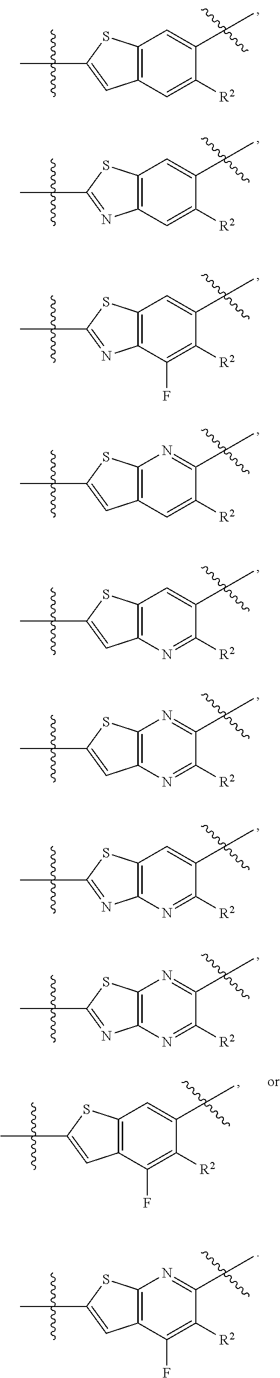

19. The compound of any one of the preceding claims, wherein each 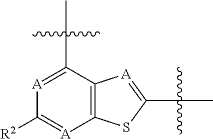
independently is

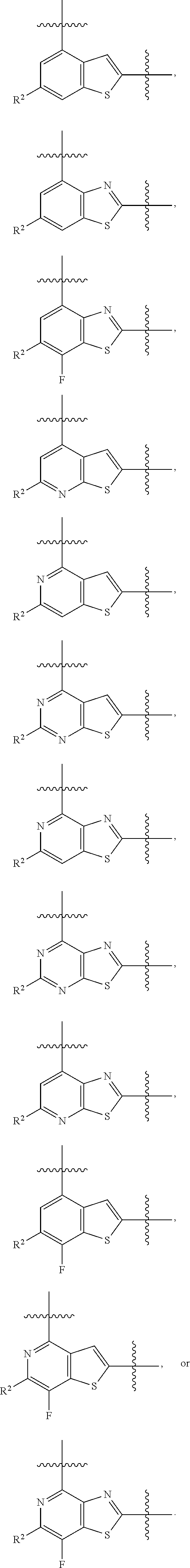

20. The compound of any one of the preceding claims, being of Formula (V) or the pharmaceutically acceptable salt thereof.

21. The compound of any one of the preceding claims, wherein each 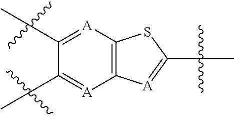
independently is

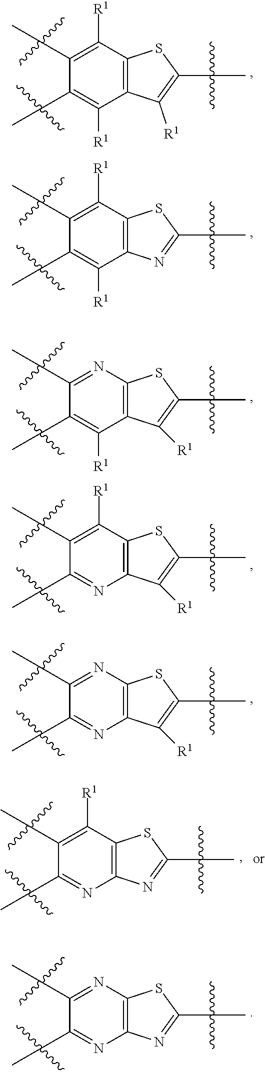

22. The compound of any one of the preceding claims, wherein each 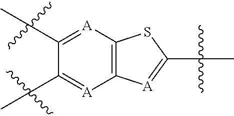
independently is

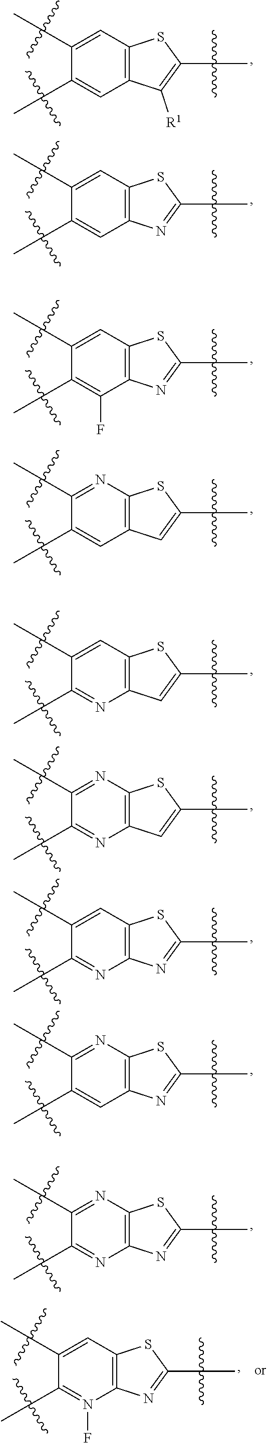

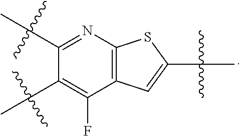 [SMILES: *c1cc2c(F)c(*)c(*)nc2s1]

23. The compound of any one of the preceding claims, wherein the compound is of Formula (VI) or the pharmaceutically acceptable salt thereof.

24. The compound of any one of the preceding claims, wherein each 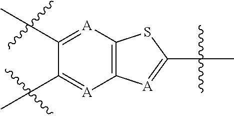
independently is

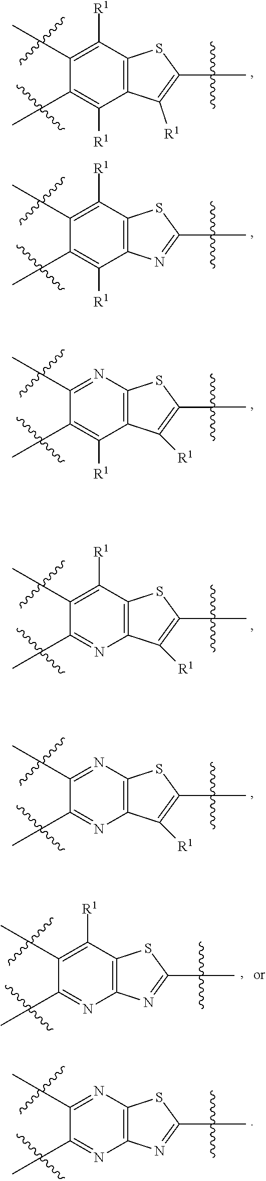

25. The compound of any one of the preceding claims, wherein each 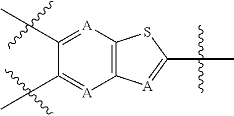
independently is

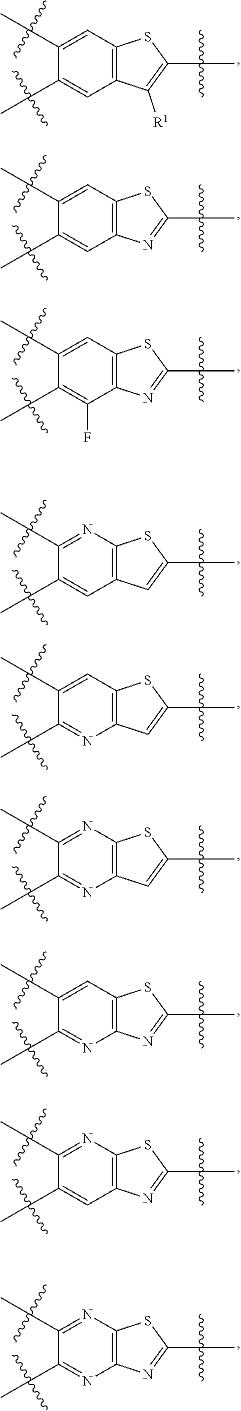

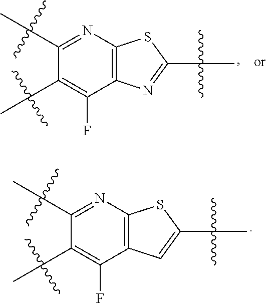 [SMILES: *c1nc2c(F)c(*)c(*)nc2s1 ; *c1cc2c(F)c(*)c(*)nc2s1]

26. The compound of any one of the preceding claims, wherein at least one A is —C(R¹)—.

27. The compound of any one of the preceding claims, wherein each A is —C(R¹)—.

28. The compound of any one of the preceding claims, wherein at least two A is —N—.

29. The compound of any one of the preceding claims, wherein two A are —N—, and the other A each independently are —C(R¹)—.

30. The compound of any one of the preceding claims, wherein two A are —N—, and the other A each independently are —CH— or —CF—.

31. The compound of any one of the preceding claims, wherein at least one R¹ is hydrogen.

32. The compound of any one of the preceding claims, wherein at least one R¹ is halogen, OR⁶, N(R⁶)₂, or C₁-C₆ alkyl, wherein the C₁-C₆ alkyl is optionally substituted with one or more halogen, OR⁶, N(R⁶)₂, C(═O)OR⁶, or C(═O)N(R⁶)₂.

33. The compound of any one of the preceding claims, wherein each R¹ independently is hydrogen or halogen.

34. The compound of any one of the preceding claims, wherein at least one R² is halogen, CN, OR⁶, N(R⁶)₂, C(═O)OR⁶, C(═O)N(R⁶)₂, S(═O)₂R⁶, C₁-C₆ alkyl, C₂-C₆ alkenyl, C₂-C₆ alkynyl, C₃-C₆ aryl, C₃-C₆ cycloalkyl, 3- to 6-membered heteroaryl, or 3- to 6-membered heterocycloalkyl, wherein the C₁-C₆ alkyl, C₂-C₆ alkenyl, or C₂-C₆ alkynyl is optionally substituted with one more halogen or OR⁶.

35. The compound of any one of the preceding claims, wherein at least one R² is OR⁶.

36. The compound of any one of the preceding claims, wherein each R² is OR⁶.

37. The compound of any one of the preceding claims, wherein each R² is OCH₃.

38. The compound of any one of the preceding claims, wherein at least one of R³ and R⁴ is absent.

39. The compound of any one of the preceding claims, wherein one of R³ and R⁴ is absent, and the other one of R³ and R⁴ is C₁-C₆ alkyl, —N(R_{S})—, or —O—.

40. The compound of any one of the preceding claims, wherein one of R³ and R⁴ is absent, and the other one of R³ and R⁴ is methyl.

41. The compound of any one of the preceding claims, wherein one of R³ and R⁴ is absent, the other one of R³ and R⁴ is methyl, and T is —N(R_{S})— or —O—.

42. The compound of any one of the preceding claims, wherein one of R³ and R⁴ is absent, and the other one of R³ and R⁴ is methyl or ethyl.

43. The compound of any one of the preceding claims, wherein one of R³ and R⁴ is absent, the other one of R³ and R⁴ is methyl or ethyl, and T is absent.

44. The compound of any one of the preceding claims, wherein R³ and R⁴ each are absent.

45. The compound of any one of the preceding claims, wherein R³ and R⁴ each independently are C₁-C₆ alkyl, —N(R_{S})—, or —O—.

46. The compound of any one of the preceding claims, wherein R³ and R⁴ each are absent, and each T independently is absent, —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}.

47. The compound of any one of the preceding claims, wherein R³ and R⁴ each are absent, and each T independently is —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ—C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}.

48. The compound of any one of the preceding claims, wherein R³ and R⁴ each are absent, and each T independently is —N(R_{S})—, —O—, -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}.

49. The compound of any one of the preceding claims, wherein R³ and R⁴ each are absent, and each T independently is -Tₐ-N(R_{S})—N(R_{S})-T_{b}-, -Tₐ-C₂-C₆ alkenyl-T_{b}-, -Tₐ-C₂-C₆ alkynyl-T_{b}-, -Tₐ-C(═O)-T_{b}, -Tₐ-C(═CH₂)-T_{b}-, -Tₐ-C(═O)—C(═O)-T_{b}, -Tₐ-C(═O)—(C₁-C₆ alkyl)-C(═O)-T_{b}, -Tₐ-C(═O)—(C₃-C₁₂ cycloalkyl)-C(═O)-T_{b}, -Tₐ-C(═O)—(C₁-C₆ alkyl)-(C₃-C₁₂ cycloalkyl)-(C₁-C₆ alkyl)-C(═O)-T_{b}, -Tₐ-C(═O)—(3- to 12-membered heterocycloalkyl)-C(═O)-T_{b}, -Tₐ-C(═O)—(C₁-C₆ alkyl)-(3- to 12-membered heterocycloalkyl)-(C₁-C₆ alkyl)-C(═O)-T_{b}, -Tₐ-C(═S)-T_{b}, -Tₐ-S(═O)₂-T_{b}-, -Tₐ-S(═O)-T_{b}, -Tₐ-P(═O)(—OR_{S})-T_{b}-, -Tₐ-(C₃-C₁₂ cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}.

50. The compound of any one of the preceding claims, wherein R₃ and R₄ each independently are absent, C₁-C₆ alkyl, —N(R_{S})—, or —O—; and each T independently is —NH—, —N(C₁₋₆ alkyl)-, —O—, C₂-C₆ alkenyl, C₂-C₆ alkynyl, —C₀₋₆ alkyl-C(═O)—C₀₋₆ alkyl-, —C₀₋₆ alkyl-C(═CH₂)—C₀₋₆ alkyl-, —C₀₋₆ alkyl-C(═O)—C(═O)—C₀₋₆ alkyl-, —C₀₋₆ alkyl-C(═S)—C₀₋₆ alkyl-, —C₀₋₆ alkyl-S(═O)₂—C₀₋₆ alkyl-, —C₀₋₆ alkyl-S(═O)—C₀₋₆ alkyl-, —C₀₋₆ alkyl-P(═O)(—OH)—C₀₋₆ alkyl-, —C₀₋₆ alkyl-C₃-C₁₂ cycloalkyl-C₀₋₆ alkyl-, —C₀₋₆ alkyl-C₆-C₁₂ aryl-C₀₋₆ alkyl-, —C₀₋₆ alkyl-(3- to 12-membered heterocyclyl)-C₀₋₆ alkyl-, —C₀₋₆ alkyl-(5- to 12-membered heteroaryl)-C₀₋₆ alkyl-, —C₀₋₆ alkyl-O-(5- to 12-membered heteroaryl)-O—C₀₋₆ alkyl-, —C₀₋₆ alkyl-O—C(═O)—NH—C₀₋₆ alkyl-, —C₀₋₆ alkyl-O—C(═O)—C₀₋₆ alkyl-, —C₀₋₆ alkyl-NH—C(═O)—C₀₋₆ alkyl-, —O—C(═O)—O—, —NH—C(═O)—NH—, or —NH—C(═S)—NH—; wherein the C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more halo, —OR_{S}, —N(R_{S})₂, or —C(═O)OR_{S}: (1) when one of R₃ and R₄ is C₁ alkyl, and T is —NH—, —N(C₁₋₆ alkyl)-, or —O—, then the other one of R₃ and R₄ is absent; and/or (2) when one of one of R₃ and R₄ is C₂-C₆ alkyl, then T is not —NH—, —N(C₁₋₆ alkyl)-, or —O—.

51. The compound of any one of the preceding claims, wherein each 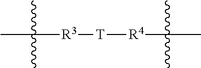
independently is: 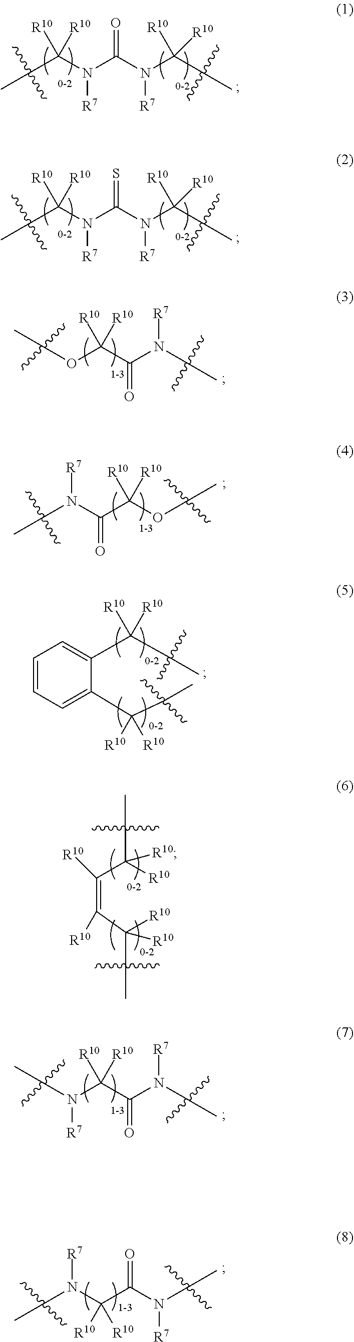 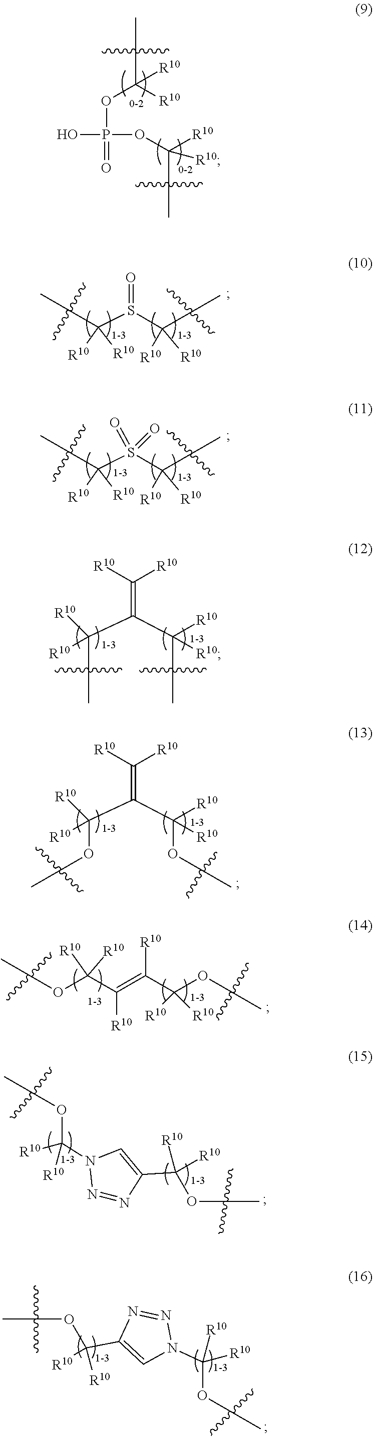 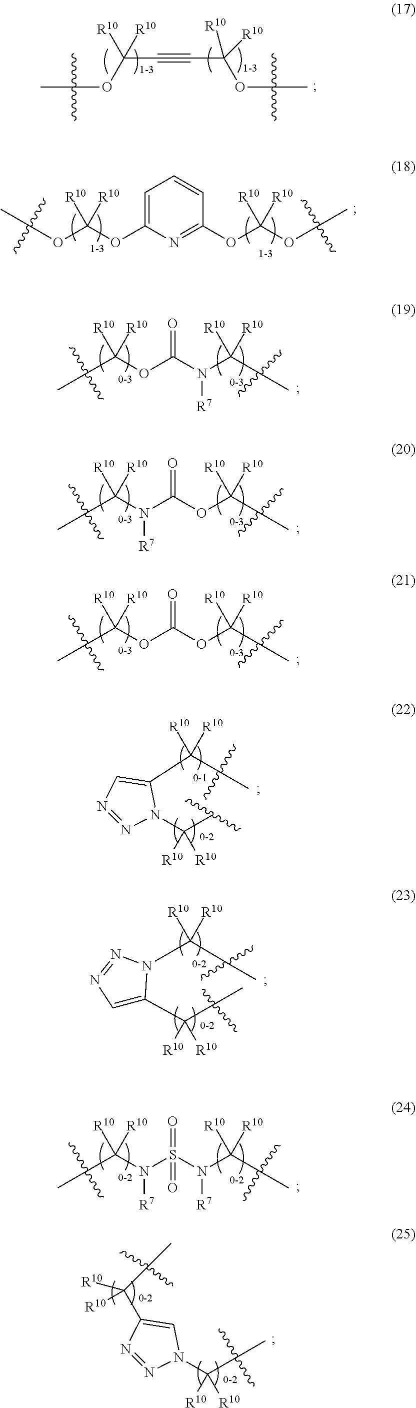 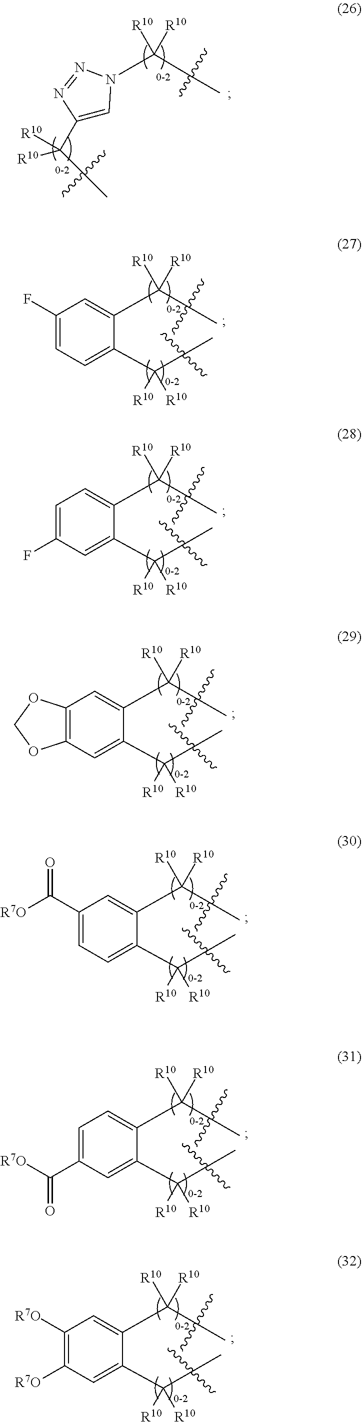 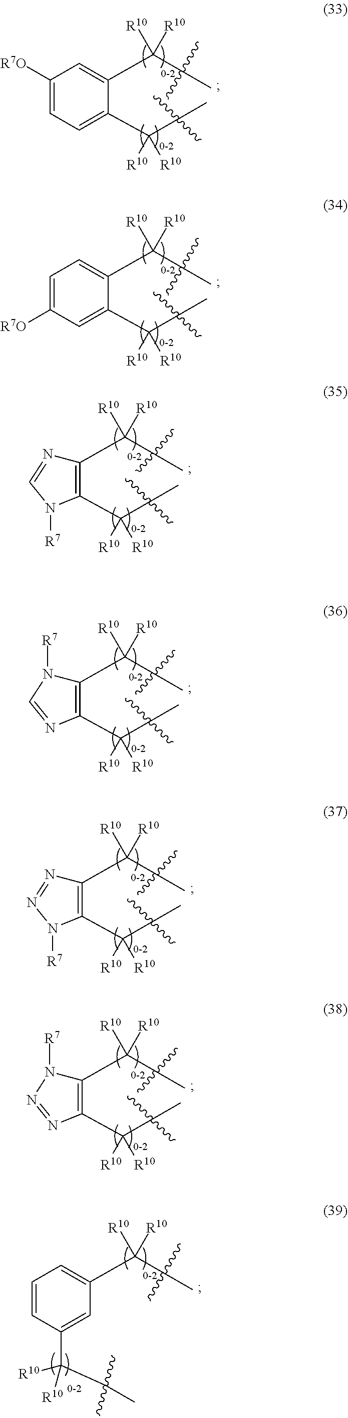 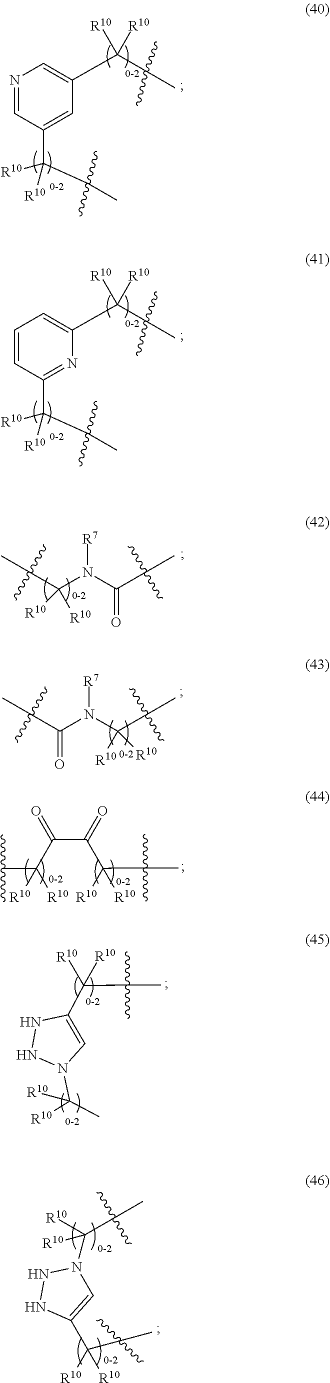 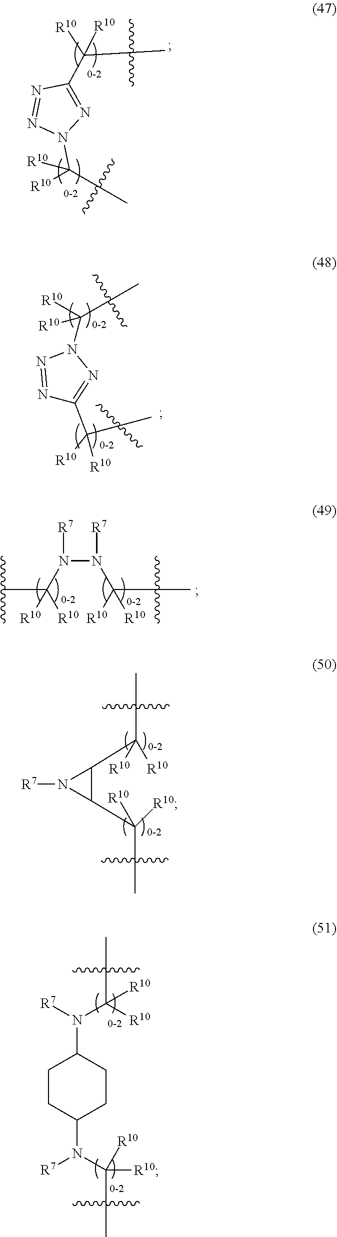 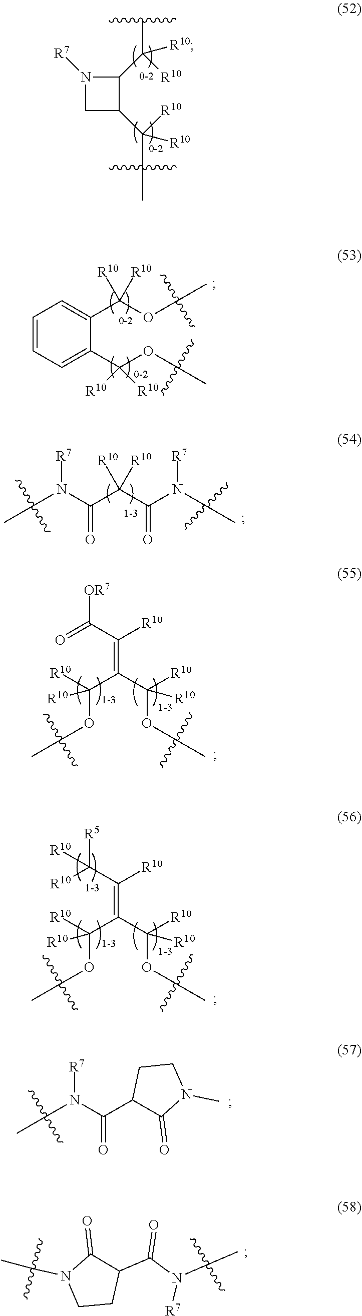 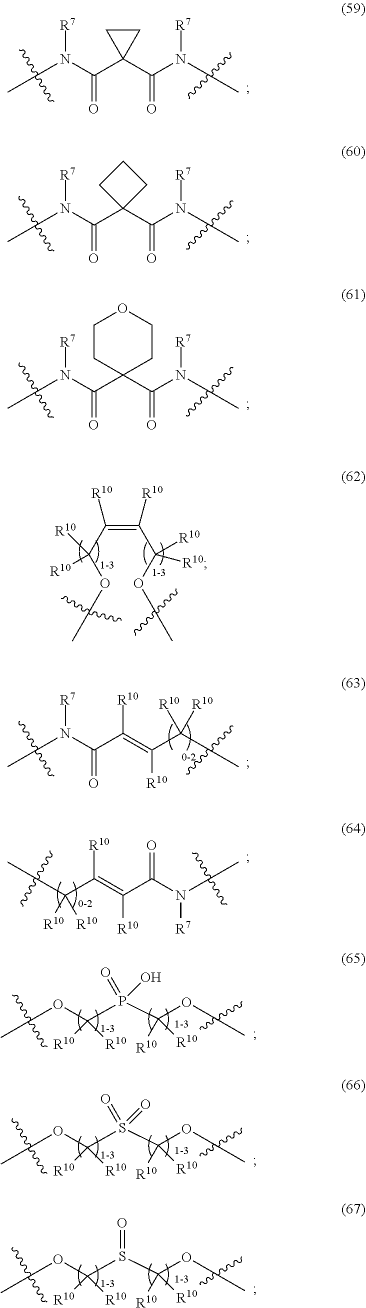 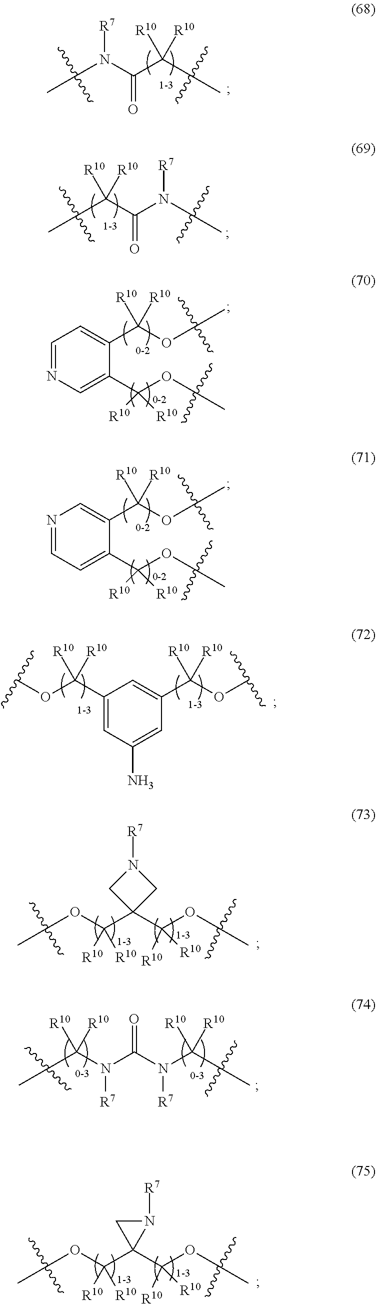 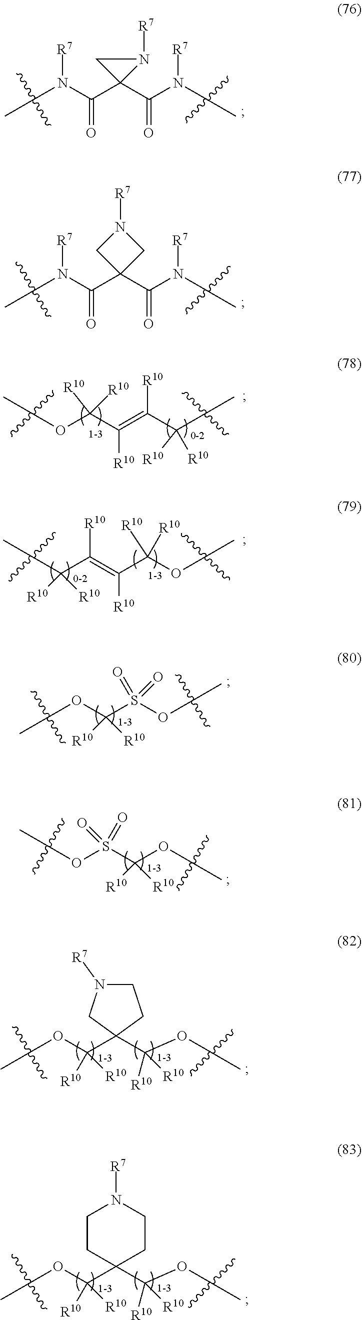 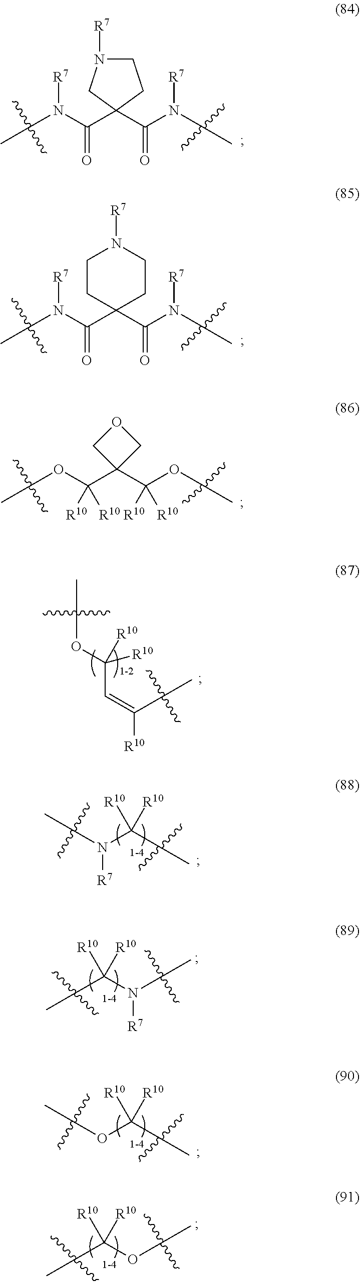 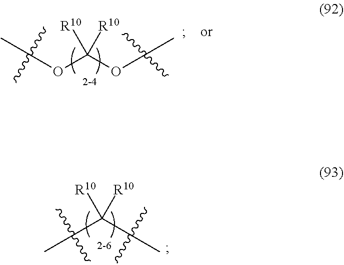
wherein:
each R⁵ is independently —OR⁷, NR⁷ or —C(O)OR⁷
each R⁷ is independently hydrogen or C₁₋₂ alkyl; and
each R¹⁰ is independently hydrogen, C₁₋₂ alkyl or halogen.

52. The compound of any one of the preceding claims, wherein each R⁷ is independently hydrogen or methyl

53. The compound of any one of the preceding claims, wherein each R¹⁰ is independently hydrogen, methyl or fluorine.

54. The compound of any one of the preceding claims, wherein one R¹⁰ is hydrogen, and the other R¹⁰ is methyl or fluorine.

55. The compound of any one of the preceding claims, wherein each 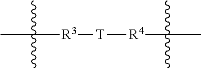
independently is:

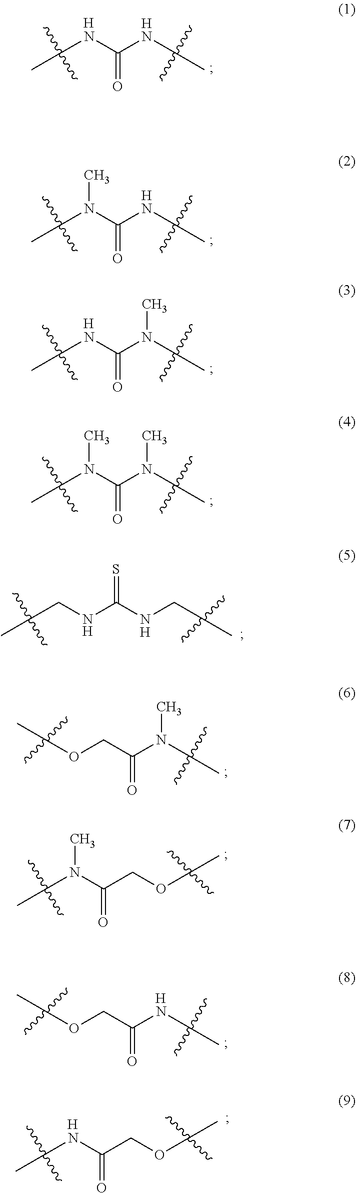

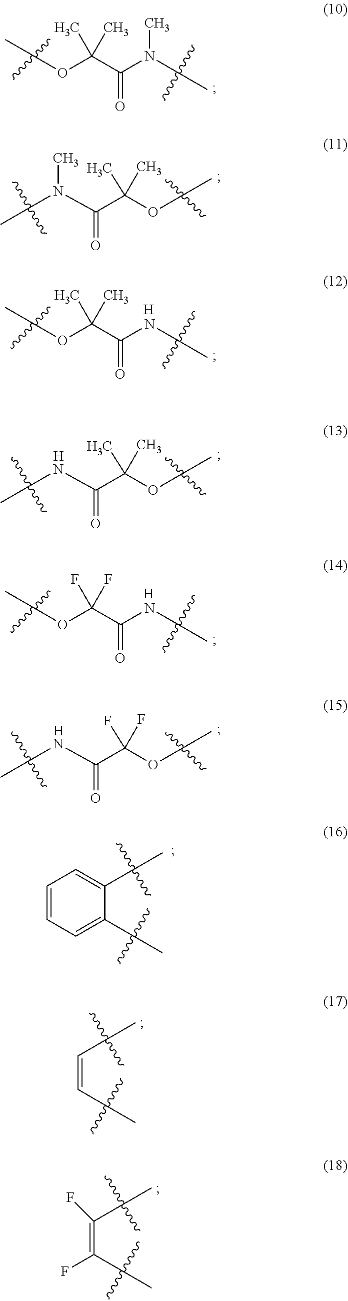

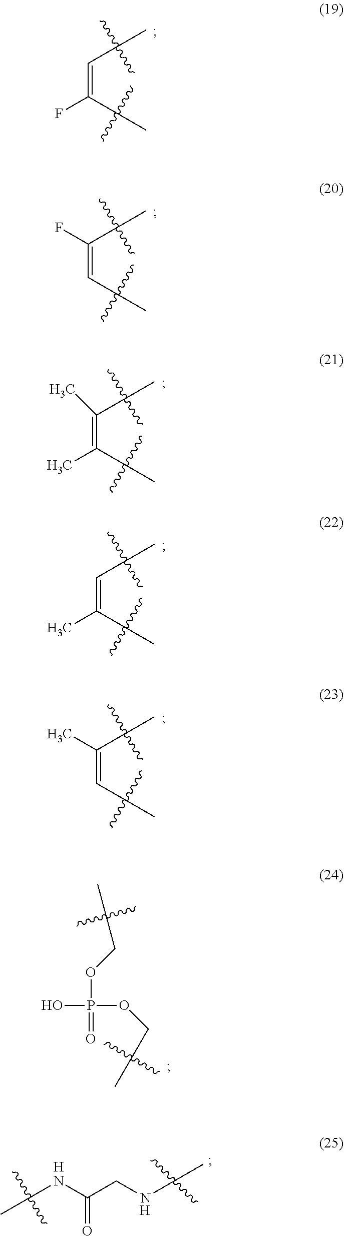

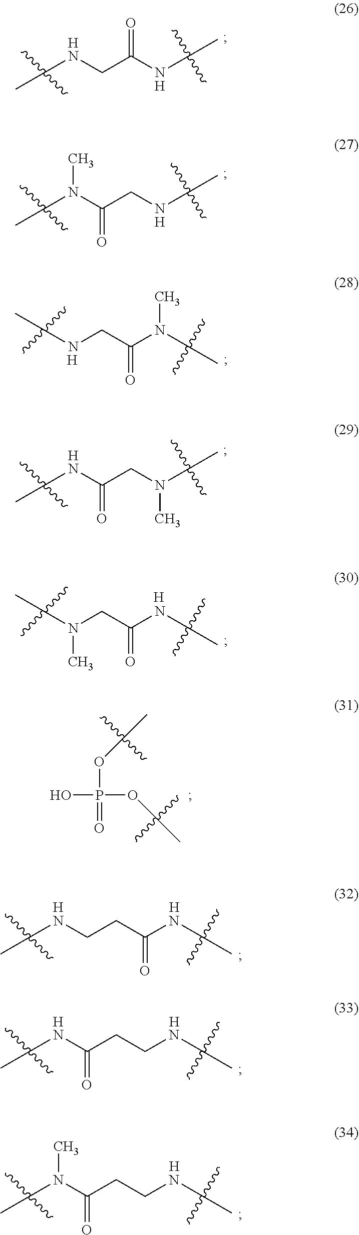

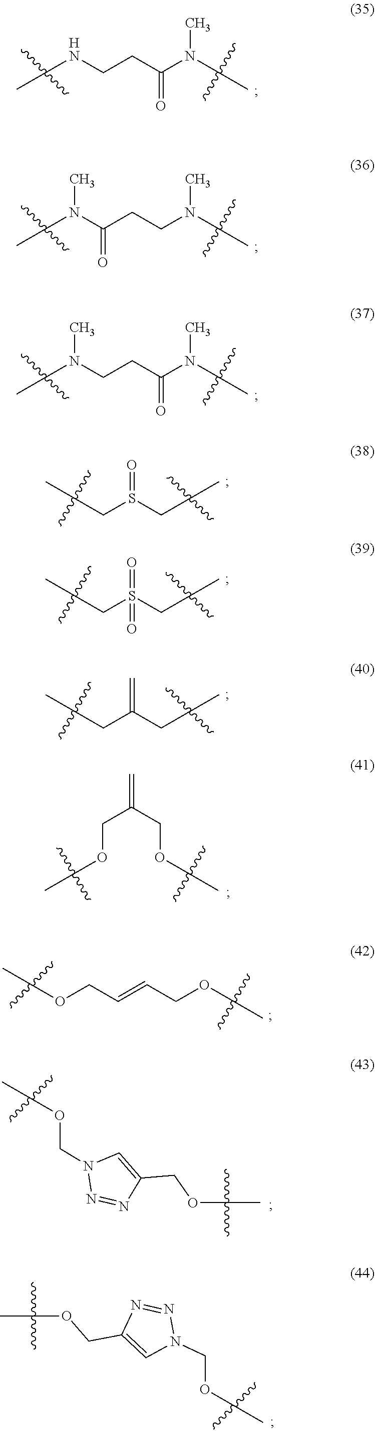

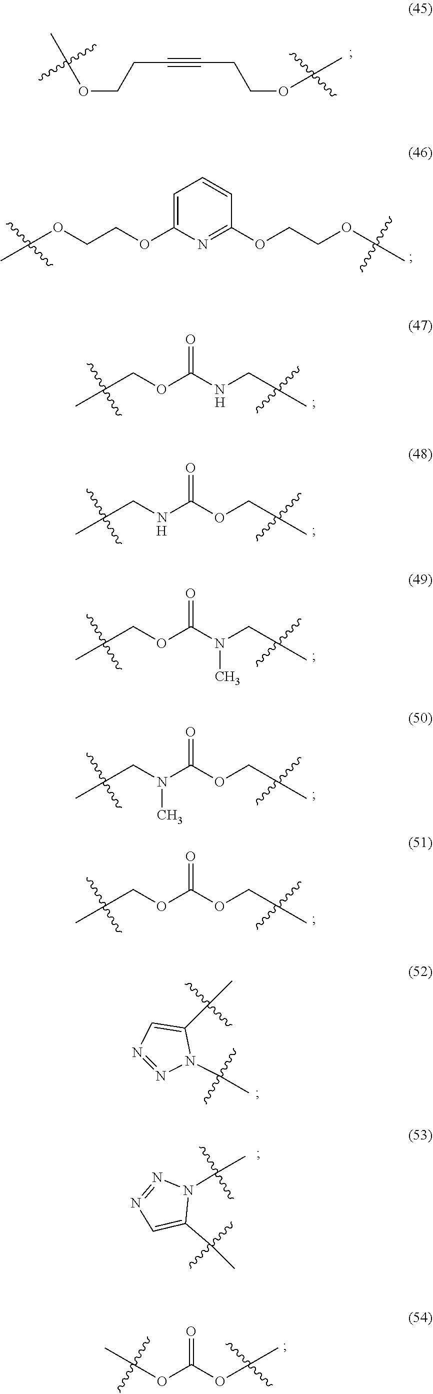

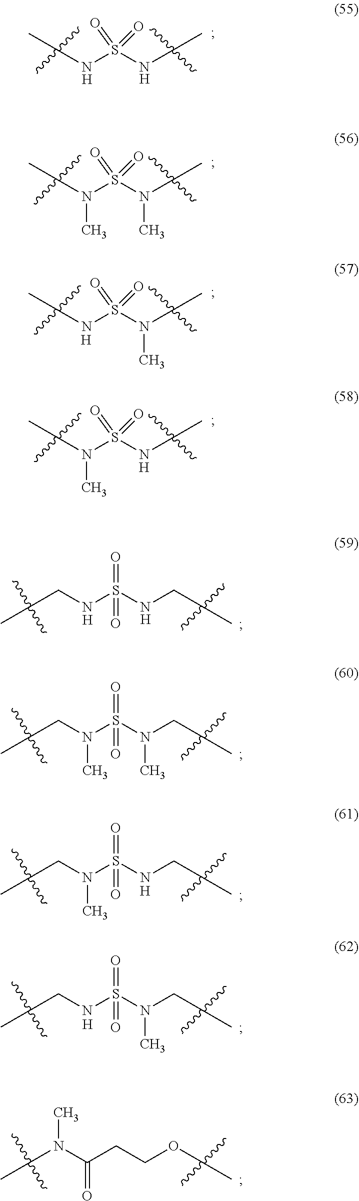

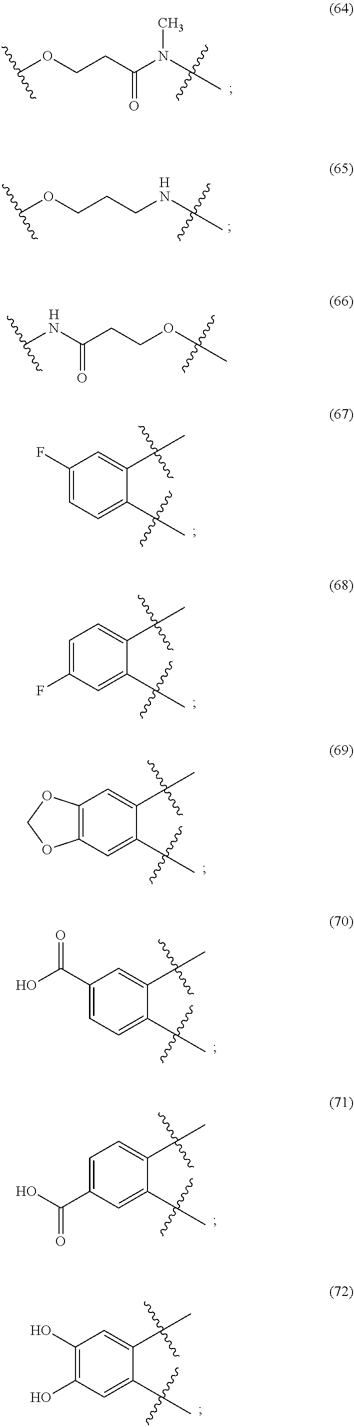

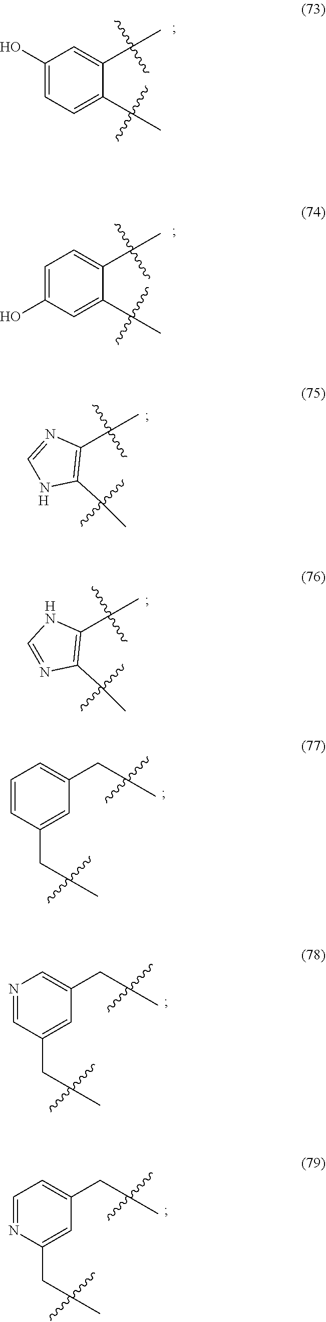

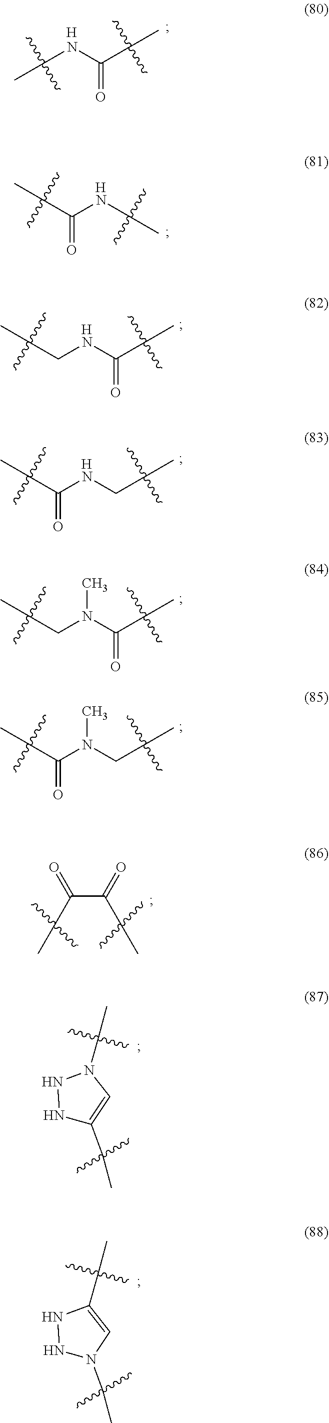

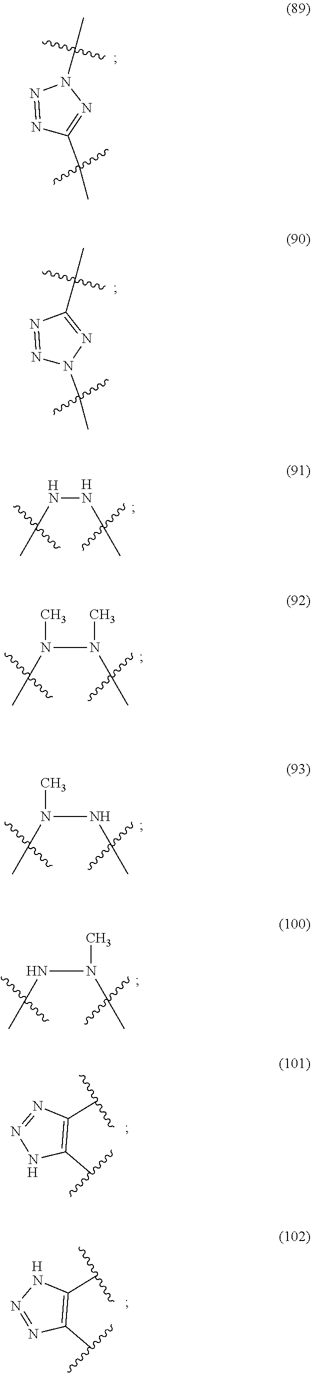

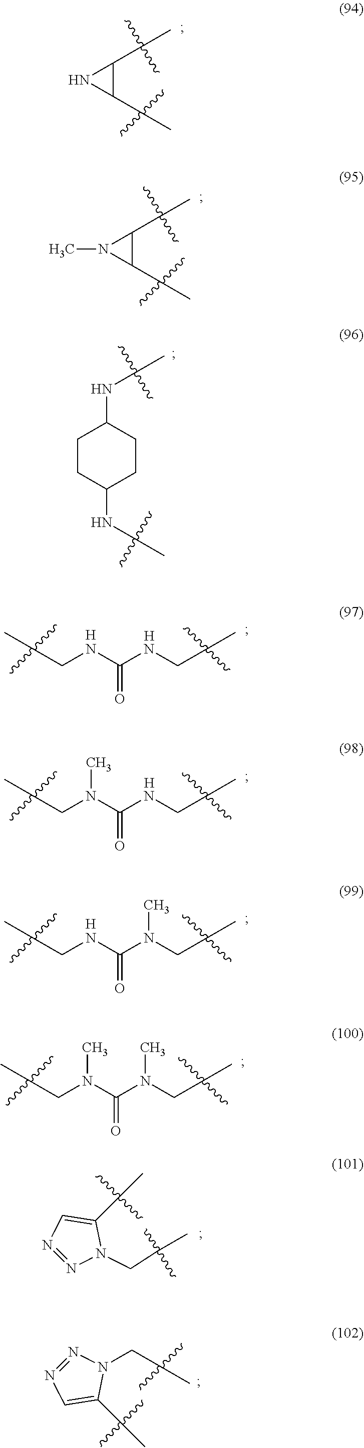

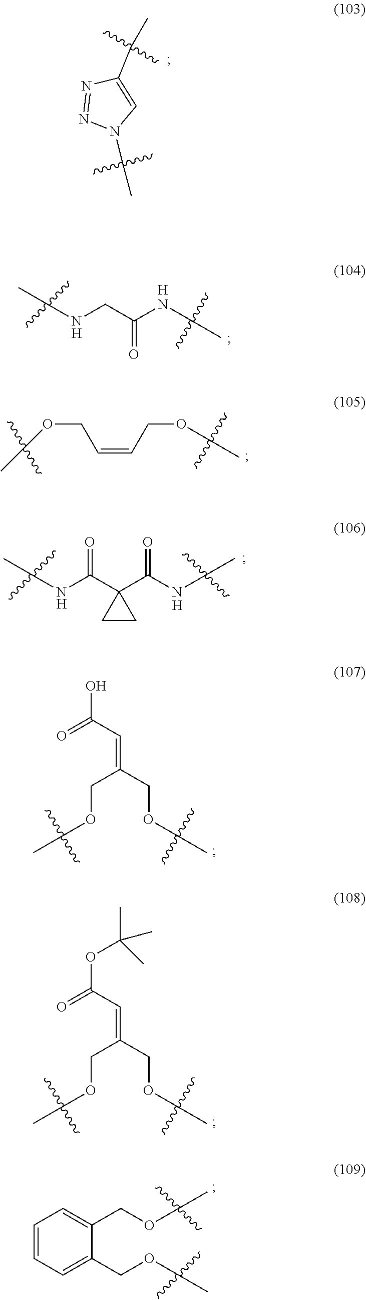

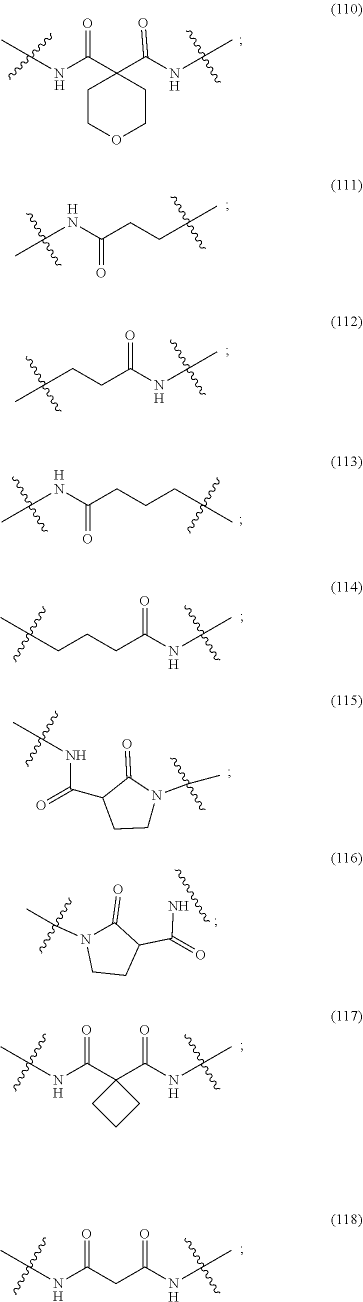

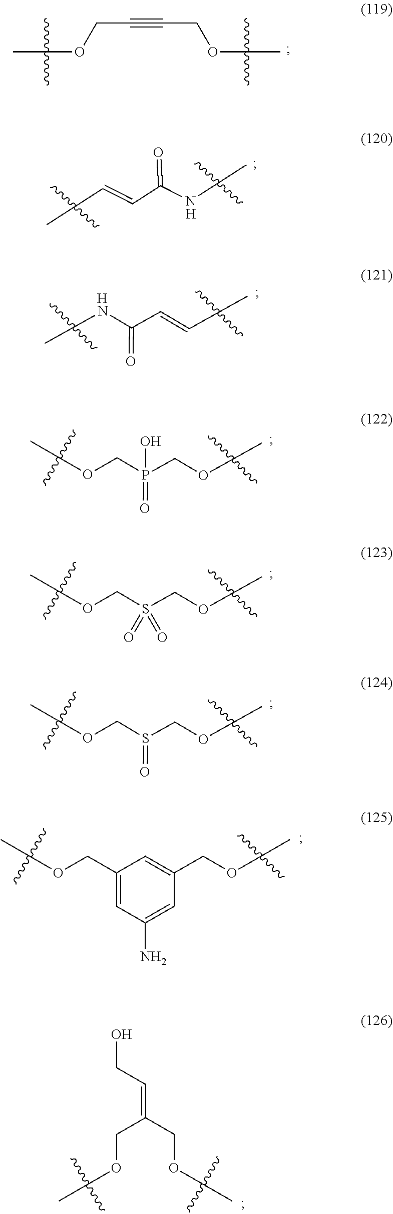

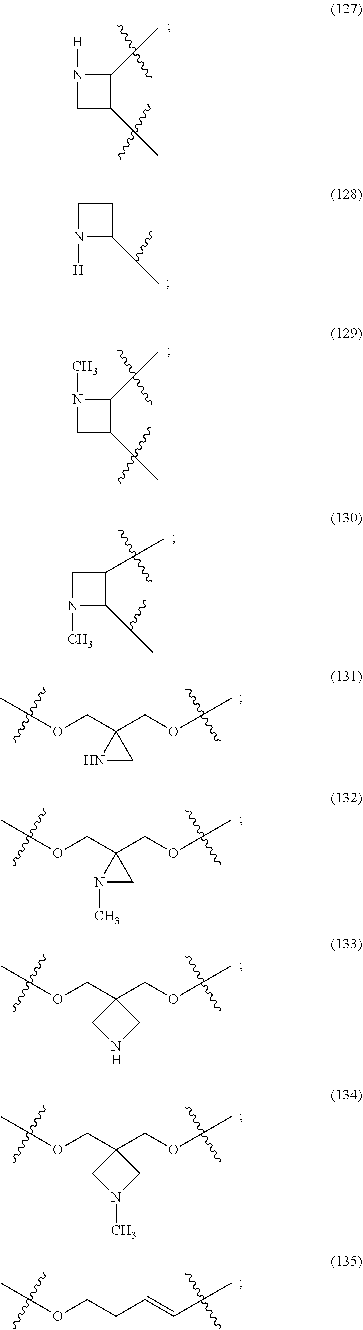

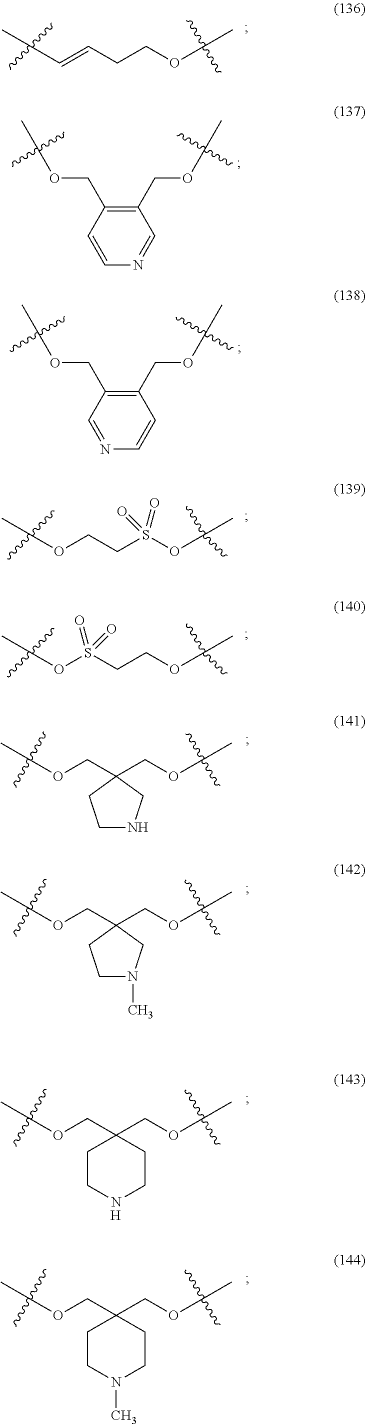

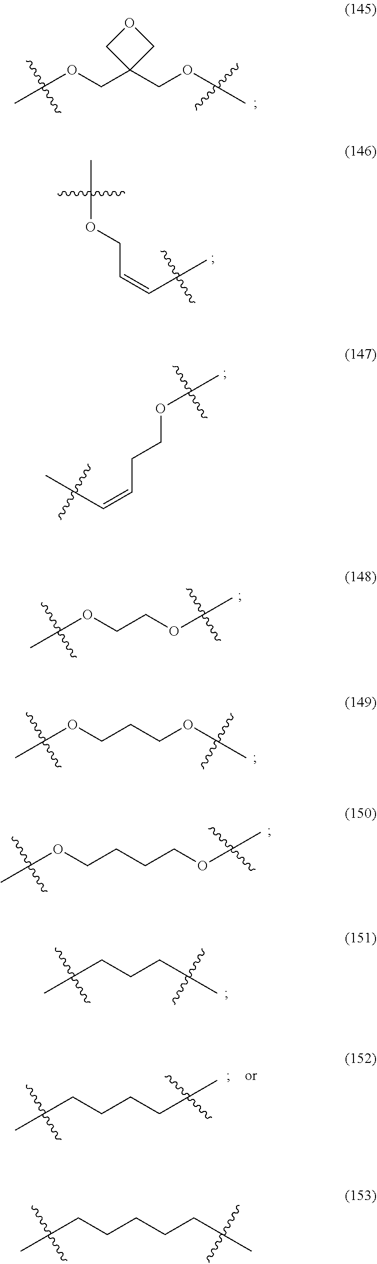

56. The compound of any one of the preceding claims, wherein each 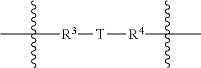
independently is

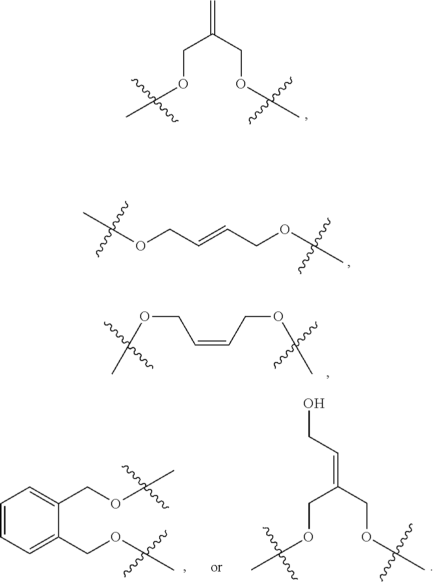

57. The compound of any one of the preceding claims, wherein at least one R⁶ is hydrogen.

58. The compound of any one of the preceding claims, wherein at least one R⁶ is C₁-C₆ alkyl optionally substituted with one or more halogen.

59. The compound of any one of the preceding claims, wherein each R⁶ independently is CH₃.

60. The compound of any one of the preceding claims, wherein at least one X¹ is —C(═O)—.

61. The compound of any one of the preceding claims, wherein each X¹ is —C(═O)—.

62. The compound of any one of the preceding claims, wherein each X² independently is —(C(R⁸)₂)₁₋₃—.

63. The compound of any one of the preceding claims, wherein each X² independently is —C(R⁸)₂C(R⁸)₂—.

64. The compound of any one of the preceding claims, wherein each X² independently is —CH₂CH₂—.

65. The compound of any one of the preceding claims, wherein at least one X³ is C(═O)OR⁶.

66. The compound of any one of the preceding claims, wherein at least one X³ is C(═O)OH.

67. The compound of any one of the preceding claims, wherein each X³ is C(═O)OH.

68. The compound of any one of the preceding claims, wherein each X¹ is —C(═O)—, each X² independently is —(C(R⁸)₂)₁₋₃—, and each X³ is C(═O)OR⁶.

69. The compound of any one of the preceding claims, wherein each —X¹—X²—X³ independently is —C(═O)—CH₂CH₂—C(═O)OH.

70. The compound of any one of the preceding claims, being of Formula (I-a), (II-a), (III-a), (IV-a), (V-a) or (VI-a): 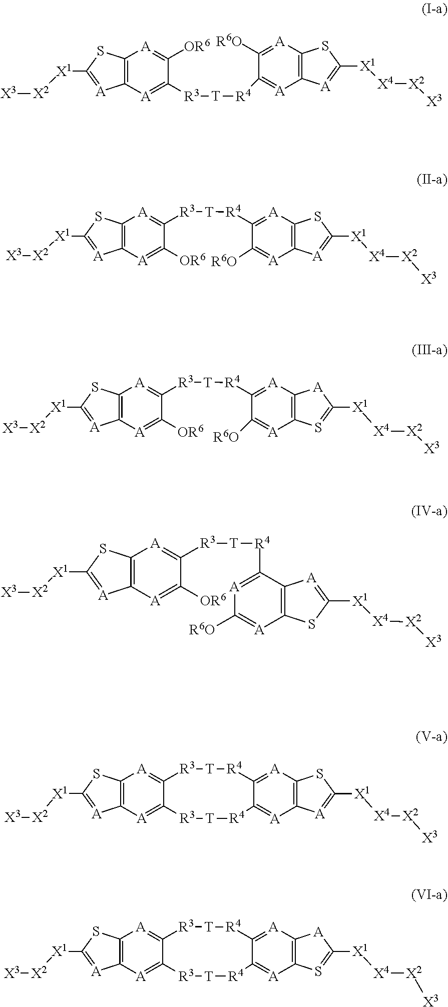
or a pharmaceutically acceptable salt thereof.

71. The compound of any one of the preceding claims, being of Formula (I-b), (II-b), (III-b), (IV-b), (V-b) or (VI-b): 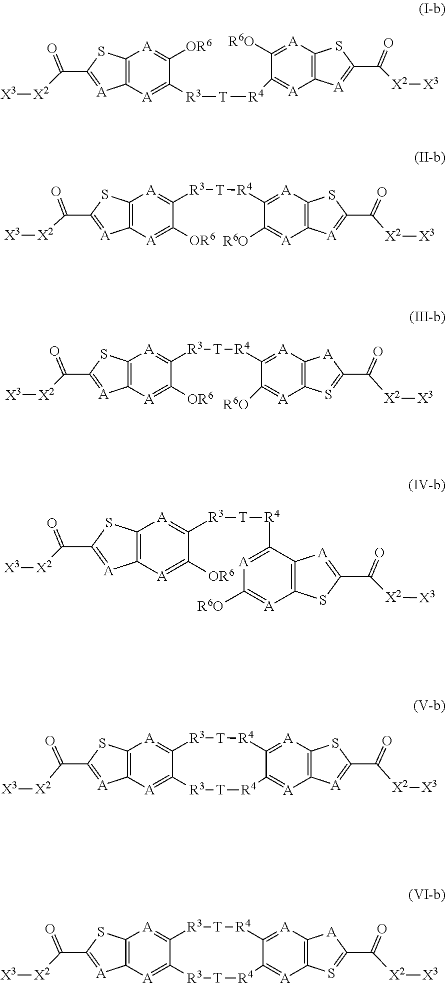
or a pharmaceutically acceptable salt thereof.

72. The compound of any one of the preceding claims, being of Formula (I-c), (II-c), (III-c), (IV-c), (V-c) or (VI-c): 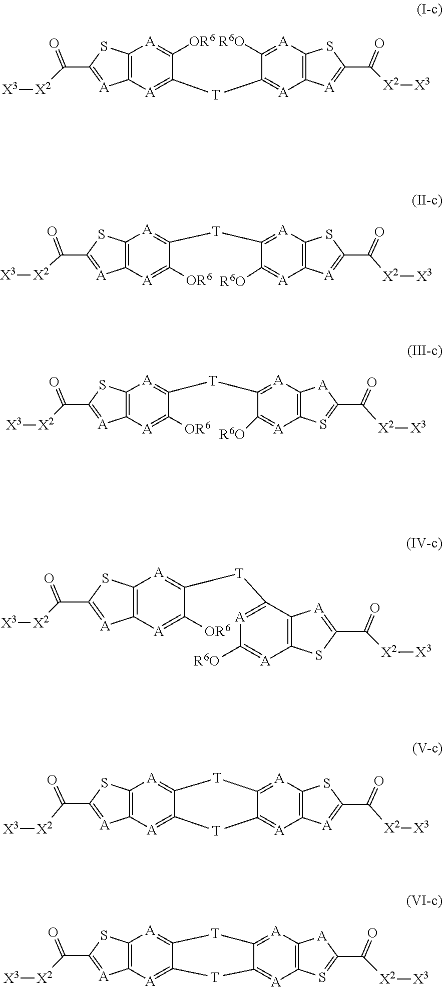
or a pharmaceutically acceptable salt thereof.

73. The compound of any one of the preceding claims, being of Formula (I-d), (II-d), (III-d), (IV-d), (V-d) or (VI-d): 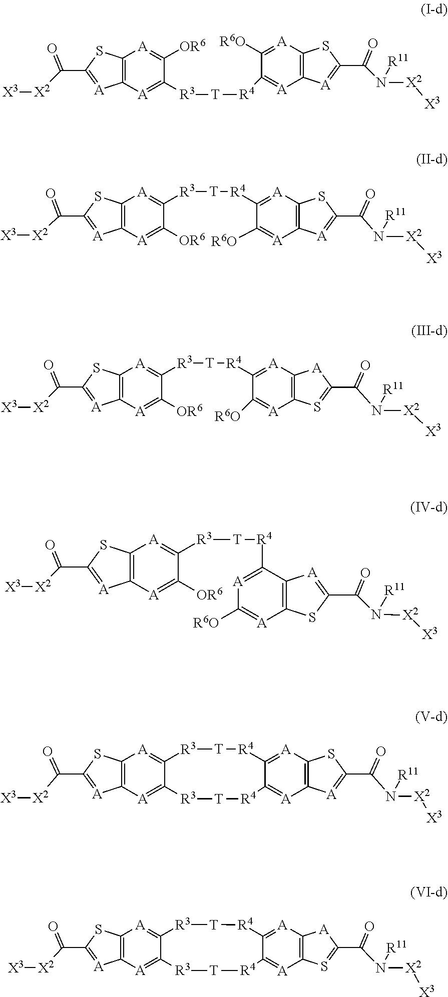
or a pharmaceutically acceptable salt thereof.

74. The compound of any one of the preceding claims, being of Formula (I-e, (II-e), (III-e), (IV-e), (V-e) or (VI-e): 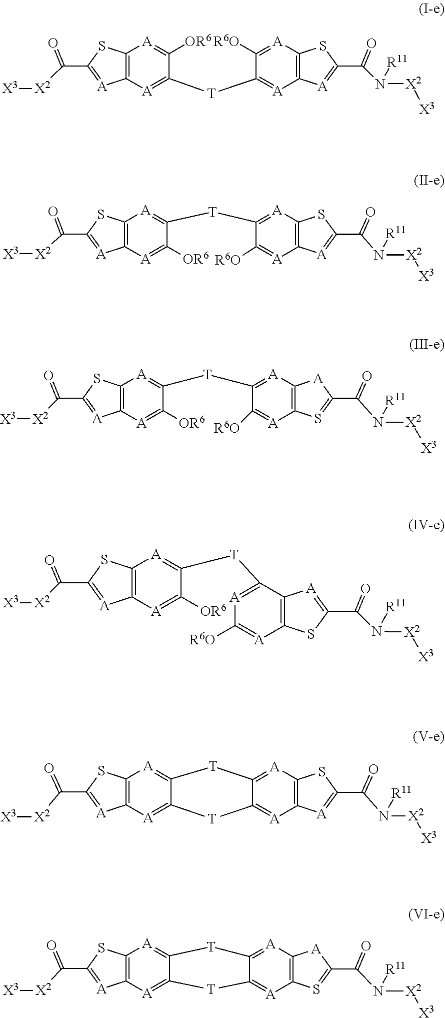
or a pharmaceutically acceptable salt thereof.

75. The compound of any one of the preceding claims, being selected from the compounds described in Table 1 and pharmaceutically acceptable salts thereof.

76. The compound of any one of the preceding claims, being selected from the compounds described in Table 1.

77. The compound of any one of the preceding claims, being selected from the compounds: 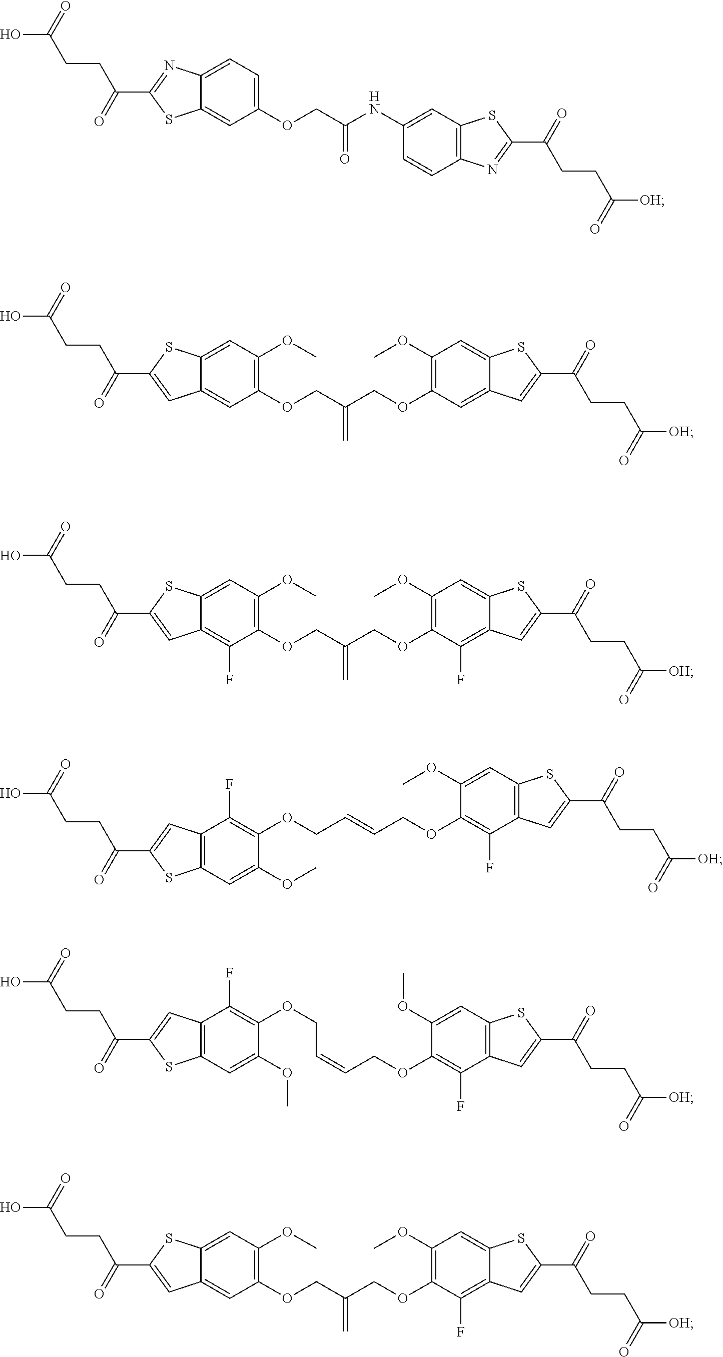 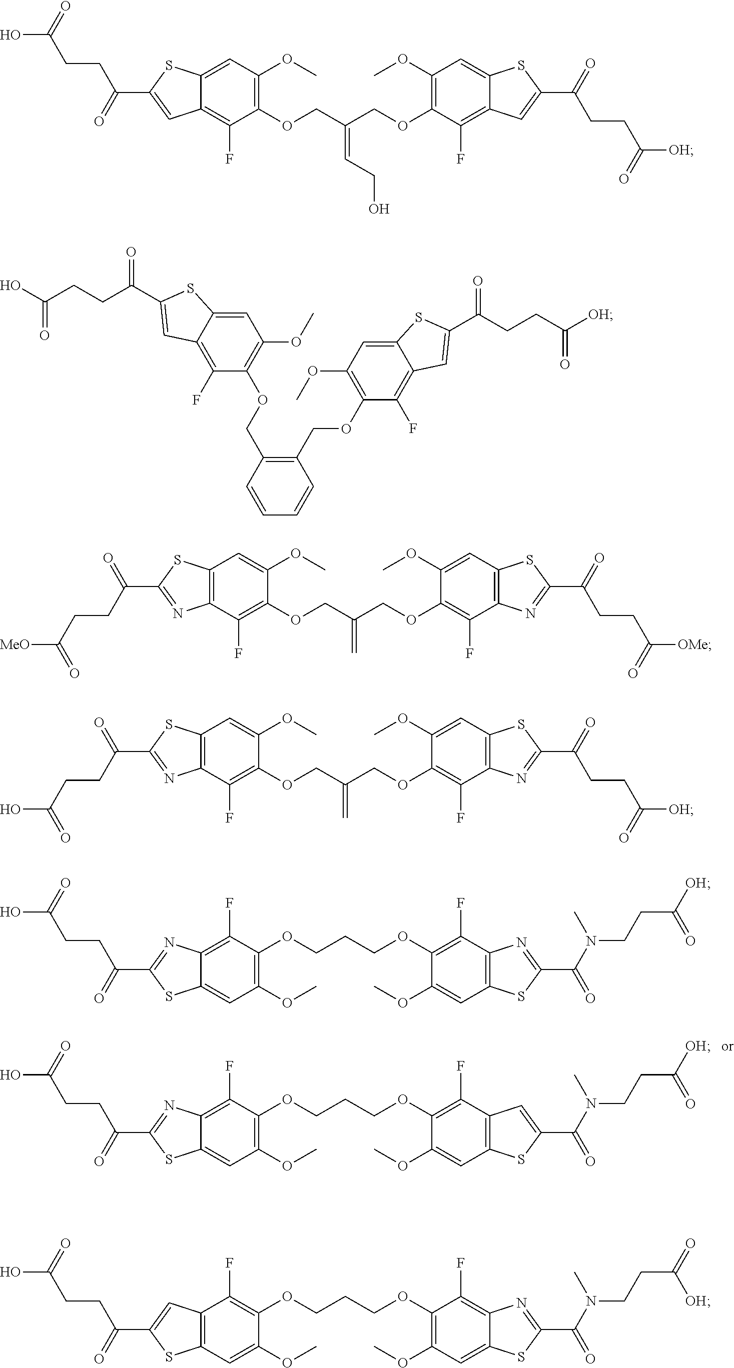
or a pharmaceutically acceptable salt thereof.

78. A compound being an isotopic derivative of the compound of any one of the preceding claims.

79. A compound being an isotopic derivative of any one of the compounds described in Table 1 and pharmaceutically acceptable salts thereof.

80. A compound being an isotopic derivative of any one of the compounds described in Table 1.

81. A method of preparing the compound of any one of the preceding claims.

82. A compound being an intermediate suitable for use in a method for preparing the compound of any one of the preceding claims.

83. A pharmaceutical composition comprising an effective amount of the compound of any one of the preceding claims and a pharmaceutically acceptable carrier.

84. The pharmaceutical composition of any one of the preceding claims, comprising at least one compound selected from the compounds described in Table 1.

85. A method of treating or preventing a STING mediated disease or disorder in a subject in need thereof, comprising administering to the subject a pharmaceutically effective amount of the compound of any one of the preceding claims, or an antibody-STING agonist conjugate thereof.

86. A method of inducing an immune response in a subject, comprising administering to the subject a therapeutically effective amount of the compound of any one of the preceding claims.

87. A method of inducing STING-dependent type I interferon production in a subject, comprising administering to the subject a therapeutically effective amount of the compound of any one of the preceding claims.

88. A method of inducing STING-dependent cytokine production in a subject, comprising administering to the subject a therapeutically effective amount of the compound of any one of the preceding claims.

89. A method of treating or preventing a cell proliferation disorder in a subject, comprising administering to the subject a therapeutically effective amount of the compound of any one of the preceding claims.

90. The compound of any one of the preceding claims for inducing an immune response in a subject.

91. The compound of any one of the preceding claims for inducing STING-dependent type I interferon production in a subject.

92. The compound of any one of the preceding claims for inducing STING-dependent cytokine production in a subject.

93. The compound of any one of the preceding claims, or an antibody-STING agonist conjugate thereof, for use in treating or preventing a STING mediated disease or disorder in a subject in need thereof.

94. Use of the compound of any one of the preceding claims, or an antibody-STING agonist conjugate thereof, in the manufacture of a medicament for treating or preventing a STING mediated disease or disorder in a subject in need thereof.

95. The method, compound, conjugate, or use of any one of the preceding claims, wherein the STING mediated disease or disorder is cancer.
